(19) United States
(12) Patent Application Publication
(10) Pub. No.: US 2024/0384299 A1
(43) Pub. Date: Nov. 21, 2024

(54) TARGETING MOIETIES PROMOTING TRANSDUCTION OF CENTRAL NERVOUS SYSTEM CELLS AND TISSUES AND METHODS OF USE

(71) Applicant: The Broad Institute, Inc., Cambridge, MA (US)

(72) Inventors: Benjamin Deverman, Cambridge, MA (US); Ken Chan, Cambridge, MA (US)

(21) Appl. No.: 18/791,629

(22) Filed: Aug. 1, 2024

Related U.S. Application Data
(63) Continuation of application No. PCT/US2023/061872, filed on Feb. 2, 2023.
(60) Provisional application No. 63/306,009, filed on Feb. 2, 2022.

(51) Int. Cl. C12N 15/86; C07K 14/005

(52) U.S. Cl. CPC C12N 15/86; C07K 14/005; C12N 2750/14122; C12N 2750/14143; C12N 2750/14145

(57) ABSTRACT

An engineered AAV capsid is provided, in which at least one protein on the capsid is modified to include a n-mer motif, which promotes transduction of the capsid into the central nervous system (CNS). Further embodiments provide a vector system comprising one or more vectors encoding AAV capsids and a method of delivering cargo to the CNS. The method comprises administering, in vivo or in vitro, a AAV capsid according to embodiments described herein and the AVV capsid comprises one or more cargo molecules.

### CROSS-REFERENCE TO RELATED APPLICATIONS

This application is a continuation application of PCT/US2023/061872, filed Feb. 2, 2023, which claims the benefit of priority to U.S. Provisional Patent Application No. 63/306,009, filed on Feb. 2, 2022, the contents of which are incorporated by reference herein in its entirety.

### STATEMENT REGARDING FEDERALLY SPONSORED RESEARCH

This invention was made with government support under Grant No. NS111689 and Grant No. MH120096 awarded by the National Institutes of Health. The government has certain rights in the invention.

### REFERENCE TO AN ELECTRONIC SEQUENCE LISTING

This application contains a sequence listing filed in electronic form as an xml file entitled BROD-5520US_ST26.xml, created on Jul. 31, 2024, and having a size of 706,005 bytes. The content of the sequence listing is incorporated herein in its entirety.

### TECHNICAL FIELD

The subject matter disclosed herein relates generally to enhancing transduction of an engineered AA capsid into the central nervous system (CNS). In particular examples described herein, at least one protein on the capsid is modified to include an n-mer motif. Particular examples relate to a vector system having one or more vectors encoding AAV capsids and a method of delivering cargo to the CNS, in which an AAV capsid according to the examples described herein is administered in vivo or in vitro, and the AAV capsid comprises one or more cargo molecules.

### BACKGROUND

The development of gene therapies for neurodevelopmental and neurological disorders has been constrained by the inability to efficiently deliver genes throughout the central nervous system (CNS). Several studies have reported engineered AAV9 capsids, such as AAV-PHP.B and related sequences, that are capable of highly effective gene transfer throughout the CNS after intravenous (IV) administration in adult mice. However, these capsids have not shown the same degree of enhanced transduction when tested in other species, including nonhuman primates. As such, there exists a need for capsids that improved transduction in the CNS.

Citation or identification of any document in this application is not an admission that such a document is available as prior art to the present invention.

### SUMMARY

In certain example embodiments provided herein, a composition comprises a targeting moiety effective to increase transduction of central nervous system tissues (CNS), optionally further comprising a cargo couple to or otherwise associated with the targeting moiety. In an example embodiment, the targeting moiety comprises a n-mer motif, the n-mer motif is Y₁-Y₂-Y₃-P-Y₄-E-Y₅ wherein Y₁-Y₅ are independently selected amino acids. In an example embodiment, Y₁ or Y₂ is independently selected from an amino acid with a single positive charge, and Y₅ is an amino acid with a single aromatic ring. In an example embodiment, Y₁ is selected from I, T, Q, H, K, R, V, G, E, or L; Y₂ is selected from R, N, A, K, or G; Y₃ is selected from N, I, Q, M, F, L, D, T, R, V, or S; Y₄ is selected from Q, N, V, S, or T; and Y₅ is selected from F, Y, or H.

In an example embodiment, the n-mer motif is selected from the group consisting of QRIPQEY (SEQ ID NO: 13), KNSPSEF (SEQ ID NO: 10), TRTPVEF (SEQ ID NO: 16), IRVPNEF (SEQ ID NO: 8), IRMPTEF (SEQ ID NO: 6), IRQPQEF (SEQ ID NO: 7), GKLPREF (SEQ ID NO: 4), LALPQEF (SEQ ID NO: 11), ELNPREF (SEQ ID NO: 3), HNFPNEF (SEQ ID NO: 5), TARPNEY (SEQ ID NO: 15), QNIPSEY (SEQ ID NO: 12), and RSQPQEH (SEQ ID NO: 14). In an example embodiment, the n-mer motif is selected from the group consisting of KGPGYEH (SEQ ID NO: 9), QSRDLYR (SEQ ID NO: 20), MNSTISK (SEQ ID NO: 19), VNTVREF (SEQ ID NO: 21), ENHTRDK (SEQ ID NO: 17), and GNDTRST (SEQ ID NO: 18). In an example embodiment, the n-mer motif is selected from the group consisting of IRVPNEF (SEQ ID NO: 8), IRQPQEF (SEQ ID NO: 7), KNSPSEF (SEQ ID NO: 10), QRIPQEY (SEQ ID NO: 13), TRTPVEF (SEQ ID NO: 16), IRMPTEF (SEQ ID NO: 6), RSQPQEH (SEQ ID NO: 14), KGPGYEH (SEQ ID NO: 9) and HNFPNEF (SEQ ID NO: 5).

In an example embodiment, the n-mer is selected from Table 1 to Table 3. In an example embodiment, the n-mer motif is selected from SEQ ID: 3-21. In an example embodiment, the targeting moiety is part of a viral capsid protein. In an example embodiment, the AAV capsid is selected from SEQ ID: 1, 22-40. In an example embodiment, the n-mer is IRVPNEF (SEQ ID NO: 8).

In an example embodiment, the n-mer is inserted between two amino acids of the one or more capsid proteins such that the n-mer is external to the AAV capsid. In an example embodiment, the viral capsid protein is an AAV viral capsid protein. In an example embodiment, the n-mer is inserted between amino acids 588 and 589 of a capsid protein of AAV9, or in an analogous position of a capsid protein from AAV1, AAV2, AAV3, AAV4, AAV5, AAV6, AAV7, AAV8, AAVrh8, AAV rh.74, or AAV rh. 10. In an example embodiment, the capsid protein is VP1, VP2, VP3, or a combination thereof.

In an example embodiment, the cargo is a polynucleotide, one or more polypeptides, a ribonucleoprotein complex. In an example embodiment, the polynucleotide encodes one or more polypeptides and/or a RNAi oligonucleotide. In an example embodiment, the polynucleotide encodes one or more polypeptides. In an example embodiment, the one or more polypeptides comprise enzymes or antibodies. In an example embodiment, the polynucleotide encodes a CRISPR-Cas system.

In one aspect, provided herein is a composition comprising one or more nucleic acid vectors (including a plasmid or other construct) encoding a targeting moiety effective to increase transduction of central nervous system tissues. (CNS). Also provided are viral vectors, such as recombinant AAV (rAAV) particles having capsids comprising the targeting moiety described herein and also comprising a nucleic acid encoding a payload. Also included are embodiments where the targeting moiety is coupled directly to a payload independent of that payload's incorporation into a rAAV. In an example embodiment, provided herein are vectors wherein the targeting moiety comprises a n-mer motif, the n-mer motif is Y₁-Y₂-Y₃-P-Y₄-E-Y₅ wherein Y₁-Y₅ are independently selected amino acids. In an example embodiment, provided herein are vectors wherein Y₁ or Y₂ is independently selected from an amino acid with a single positive charge, and Y₅ is an amino acid with a single aromatic ring. In an example embodiment, provided herein are vectors wherein Y₁ is selected from I, T, Q, H, K, R, V, G, E, or L; Y₂ is selected from R, N, A, K, or G; Y₃ is selected from N, I, Q, M, F, L, D, T, R, V, or S; Y₄ is selected from Q, N, V, S, or T; and Y₅ is selected from F, Y, or H.

In an example embodiment, provided herein are vectors wherein the n-mer motif is selected from the group consisting of QRIPQEY (SEQ ID NO: 13), KNSPSEF (SEQ ID NO: 10), TRTPVEF (SEQ ID NO: 16), IRVPNEF (SEQ ID NO: 8), IRMPTEF (SEQ ID NO: 6), IRQPQEF (SEQ ID NO: 7), GKLPREF (SEQ ID NO: 4), LALPQEF (SEQ ID NO: 11), ELNPREF (SEQ ID NO: 3), HNFPNEF (SEQ ID NO: 5), TARPNEY (SEQ ID NO: 15), QNIPSEY (SEQ ID NO: 12), and RSQPQEH (SEQ ID NO: 14). In an example embodiment, provided herein are vectors wherein the n-mer motif is selected from the group consisting of KGPGYEH (SEQ ID NO: 9), QSRDLYR (SEQ ID NO: 20), MNSTISK (SEQ ID NO: 19), VNTVREF (SEQ ID NO: 21), ENHTRDK (SEQ ID NO: 17), and GNDTRST (SEQ ID NO: 18). In an example embodiment, provided herein are vectors wherein the n-mer motif is selected from the group consisting of IRVPNEF (SEQ ID NO: 8), IRQPQEF (SEQ ID NO: 7), KNSPSEF (SEQ ID NO: 10), QRIPQEY (SEQ ID NO: 13), TRTPVEF (SEQ ID NO: 16), IRMPTEF (SEQ ID NO: 6), RSQPQEH (SEQ ID NO: 14), KGPGYEH (SEQ ID NO: 9) and HNFPNEF (SEQ ID NO: 5).

In an example embodiment, provided herein are vectors wherein the n-mer is selected from Table 1 to Table 3. In an example embodiment, provided herein are vectors wherein the n-mer motif is selected from SEQ ID: 3-21. In an example embodiment, provided herein are vectors wherein the targeting moiety is part of a viral capsid protein. In an example embodiment, provided herein are vectors wherein the AAV capsid comprises capsid proteins having an amino acid sequence selected from SEQ ID: 1, 22-40. In an example embodiment, provided herein are vectors wherein the n-mer is IRVPNEF (SEQ ID NO: 8).

In an example embodiment, provided herein are vectors wherein the n-mer is inserted between two amino acids of the one or more capsid proteins such that when the modified capsid proteins are assembled into an AAV capsid, the n-mer is external to the AAV capsid. In an example embodiment, provided herein are vectors wherein the viral capsid protein is an AAV viral capsid protein. In an example embodiment, provided herein are vectors wherein the n-mer is inserted between amino acids 588 and 589 of a capsid protein of AAV9, or in an analogous position of a capsid protein from AAV1, AAV2, AAV3, AAV4, AAV5, AAV6, AAV7, AAV8, AAVrh8, AAV rh.74, or AAV rh. 10. In an example embodiment, provided herein are vectors wherein the capsid protein is VP1, VP2, VP3, or a combination thereof.

In an example embodiment, provided herein are viral vectors wherein the cargo is a polynucleotide, one or more polypeptides, a ribonucleoprotein complex. In an example embodiment, provided herein are vectors wherein the polynucleotide encodes one or more polypeptides and/or a RNAi oligonucleotide. In an example embodiment, provided herein are vectors wherein the polynucleotide encodes one or more polypeptides. In an example embodiment, provided herein are vectors wherein the one or more polypeptides comprise enzymes or antibodies. In an example embodiment, the polynucleotide encodes a CRISPR-Cas system.

In one aspect, provided herein is a composition comprising a polypeptide encoded or produced by the vector systems described herein. In one aspect, provided herein is a composition comprising a particle produced by the vector systems described herein. In one aspect, provided herein is a composition comprising a cell comprising the composition, vector, polypeptide, or particle described herein.

In one aspect, provided herein is a method of delivering one or more cargos to the CNS, comprising: administering, in vivo or in vitro, the engineered AAV capsid as described herein, or the vector as described herein. In an example embodiment, provided herein a method of delivery wherein the cargo is a polynucleotide encoding a RNAi oligonucleotide, a polynucleotide encoding a polypeptide, or a polypeptide. In an example embodiment, provided herein a method of delivery wherein the cargo polynucleotide encodes a polypeptide, including an enzyme or antibody. In an example embodiment, provided herein a method of delivery wherein the cargo is a nucleic acid encoding a Cas polypeptide, a guide molecule, or both. In an example embodiment, provided herein a method of delivery wherein the cargo is a nucleic acid encoding a nuclease or a nucleic acid component of an RNA-guided nuclease. In an example embodiment, provided herein a method of delivery wherein the cargo is one or more polynucleotides encoding the nuclease and nucleic acid component of an RNA-guided nuclease.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1—Enrichment of AAV capsid sequences containing the [RK] 1/2-XPXE [FYH] motif in the marmoset brain and C57BL/6J and BALB/cJ brain and liver. The heatmap shows the enrichment of assessed by NGS sequencing of the capsid mRNAs in the indicated tissue samples from the third round of selection. Enrichment is defined as the Log2 (capsid variant RPM in the indicated tissue sample/RPM in the virus library). 10 replicates with unique nucleotide sequences encoding the 7-mer are shown.

FIG. 2—Enrichment of additional AAV capsid sequences in the marmoset brain, and C57BL/6J and BALB/cJ brain and liver. The heatmap shows the enrichment assessed by NGS sequencing of the capsid mRNAs in the indicated tissue samples from the third round of selection. Enrichment is defined as the Log2 (capsid variant RPM in the indicated tissue sample/RPM in the virus library). 10 replicates with unique nucleotide sequences encoding the 7-mer are shown.

FIG. 3A-3B—Titers and protein purity assessments of individual AAV batches. A) Nuclease resistant vg/L in cell pellets prepared from HEK293 suspension cell cultures. B) SyproRuby stained SDS-PAGE gel. 1E10 vg/lane was loaded.

FIG. 4A-4B—AAV BI103 is more effective than AAV9 at gene delivery throughout the marmoset brain after intravenous administration. AAV9: CAG-NLS-GFP-3×-miR122-WPRE-pA and AAV BI103: CAG-NLS-mScarlet-3x-miR122-WPRE-pA were co-administered in an adult marmoset at 4E13 per virus (8E13 total AAV dose). Brain expression was assessed 3 weeks later. A) Image shows mScarlet expression (magenta) and GFP expression (green) in a sagittal section of the marmoset brain. B) The images separately show expression of mScarlet expression in sagittal sections taken from the marmoset brain. The sections show progress from medial to lateral sections (left to right). GFP expression from the same sections is shown in the bottom set of panels.

FIG. 5A-5F—AAV-BI103 more efficiently transduces cells in the marmoset brain. AAV9: CAG-NLS-GFP-3×-miR 122-WPRE-pA and BI103: CAG-NLS-mScarlet-3×-miR122-WPRE-pA were co-administered to an adult marmoset at 4×1013 vg/kg per AAV and express was assessed 3 weeks later. A) The percentage of total cells (DAPI+) that express GFP delivered by AAV9 or mScarlet delivered by BI103. B) The percentage of NeuN+ neurons that express GFP delivered by AAV9 or mScarlet delivered by BI103. C-F) Individual data points represent data from different images. N=1 marmoset. Images show GFP and mScarlet transduction together with anti-NeuN immunostaining and DNA labeling using DAPI in the cerebral cortex (C), Thalamus (D), Striatum (E), and Cerebellum (F). Cells were counted using Cellprofiler. Individual data points represent data from different images. N=1 animal.

FIG. 6—AAV BI-103 transduces cells in the marmoset spinal cord after intravenous administration. AAV9: CAG-NLS-GFP-3×-miR122-WPRE-pA and AAV BI-103: CAG-NLS-mScarlet-3×-miR122-WPRE-pA were co-administered in an adult marmoset at 4E13 per virus (8E13 total AAV dose). Spinal cord expression was assessed 3 weeks later.

FIG. 7—Intravenous delivery of BI103 resulted in detectable liver hepatocyte transduction from a vector designed to reduce hepatocyte expression. AAV9: CAG-NLS-GFP-3×-miR122-WPRE-pA and AAV BI-103: CAG-NLS-mScarlet-3×-miR122-WPRE-pA were co-administered in an adult marmoset at 4E13 per virus (8E13 total AAV dose). (The miR122 target sequences were included within the 3′UTR of both transgene cassettes to reduce expression in liver, which expresses high levels of miR122. (B) mScarlet fluorescence is weakly detectable in the marmoset liver after co-administration of AAV9 and AAV-BI103.

FIG. 8—BI103 outperformed AAV9 when administered intravenously to an adult marmoset. AAV9: CAG-NLS-GFP-3×-miR122-BS and BI103: CAG-NLS-mScarlet-3×-miR122-BS were intravenously administered to an adult marmoset at 4×1013 vg/kg and reporter expression was assessed after 21 days.

FIG. 9—BI103 outperformed AAV9 when administered intravenously to an adult marmoset. High magnification images of transgene expression from AAV BI-103 and AAV9

FIG. 10—Logo analysis for P×EF motif found in capsids recovered after round 2 and round 3 in vivo selection in the marmoset brain.

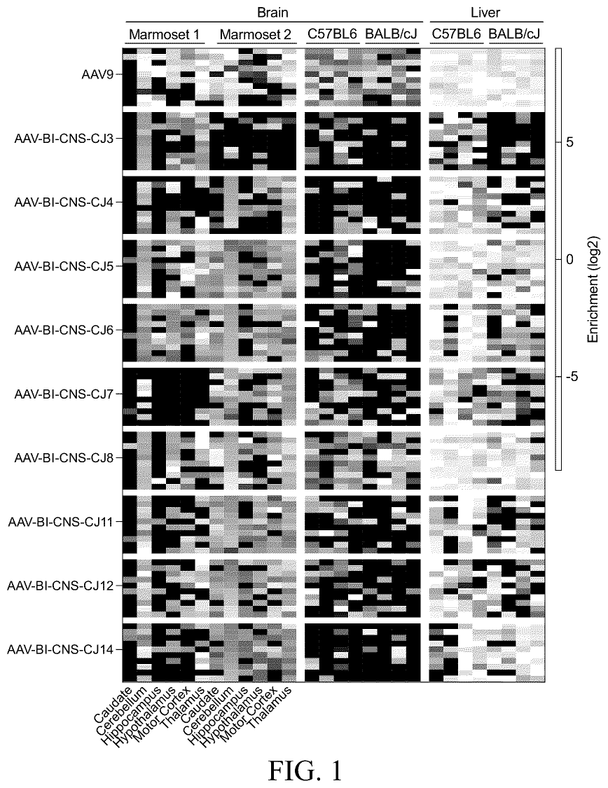

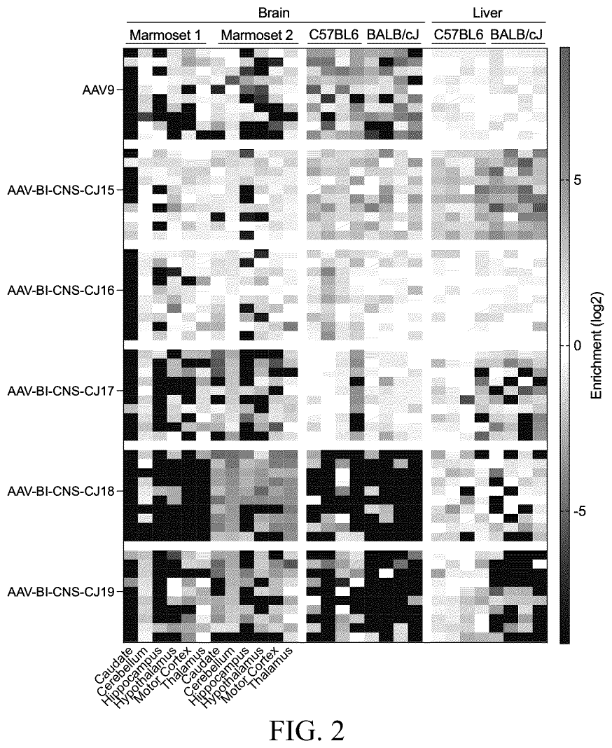

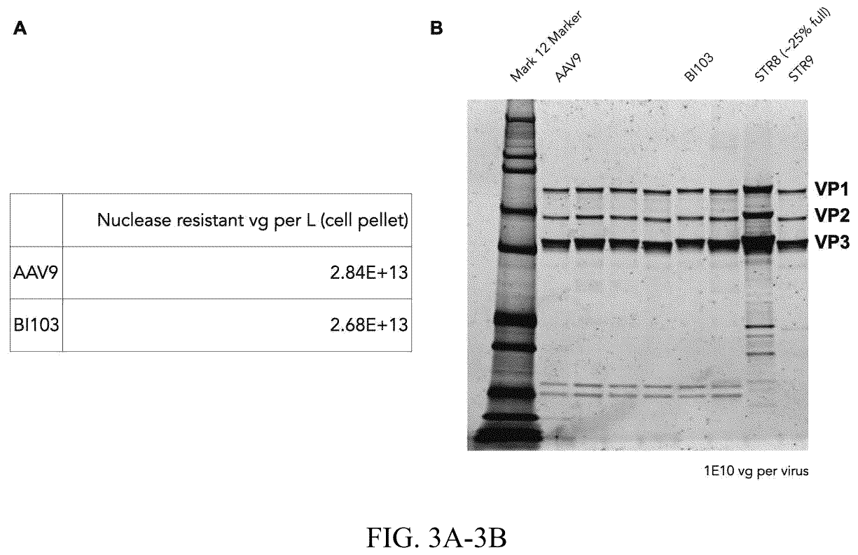

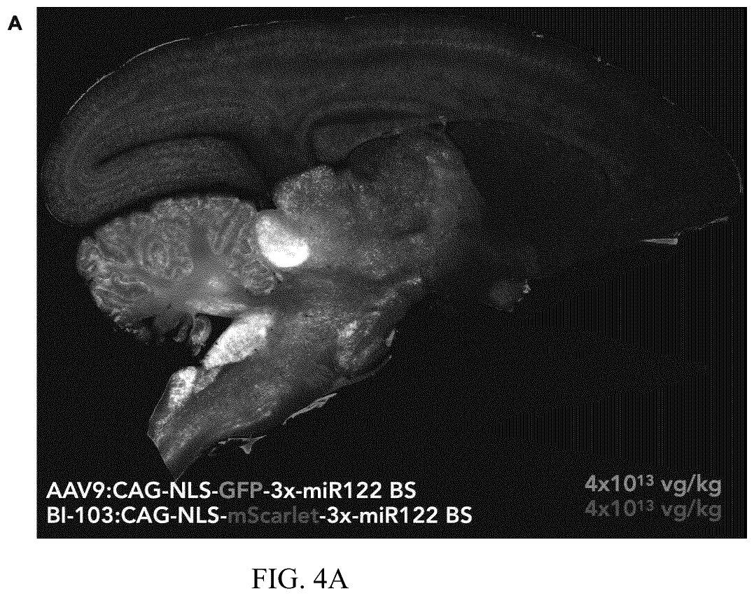

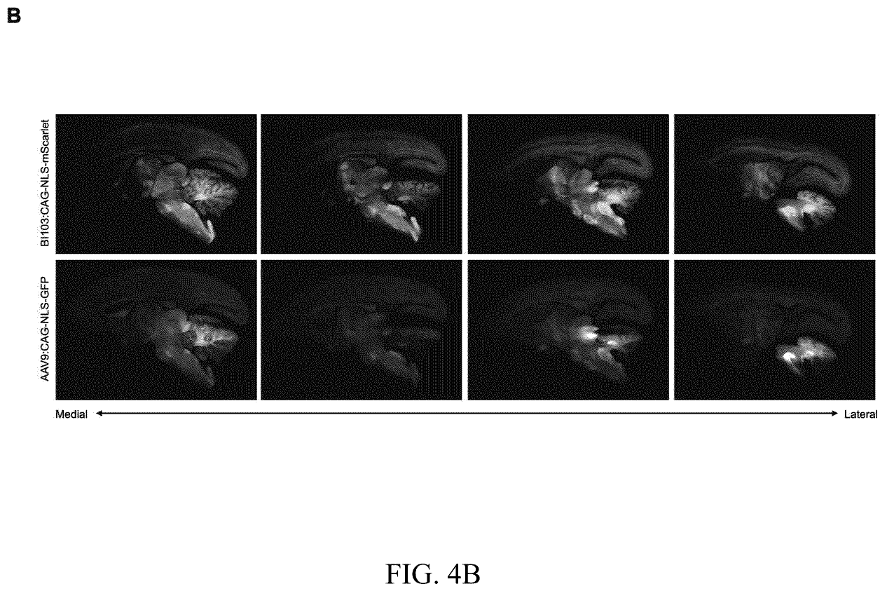

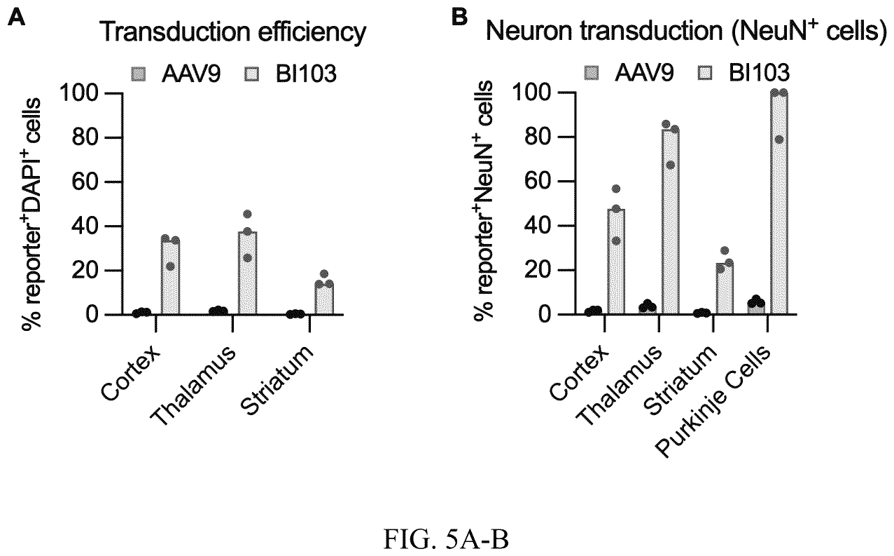

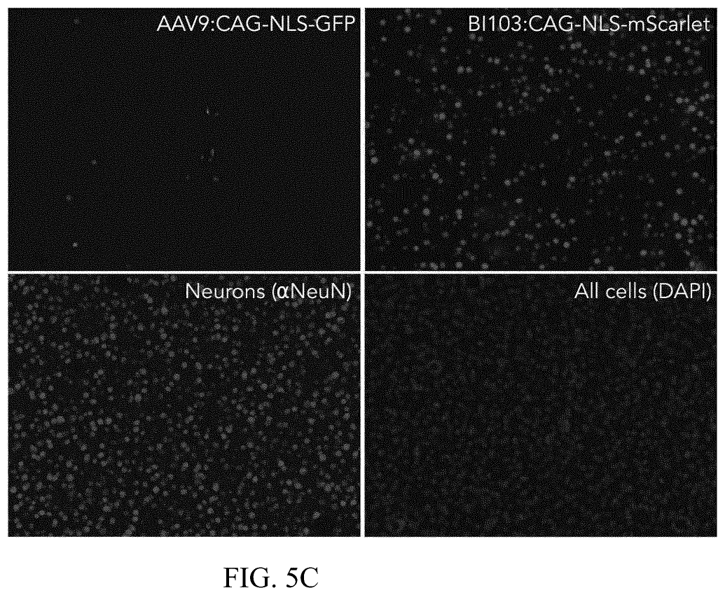

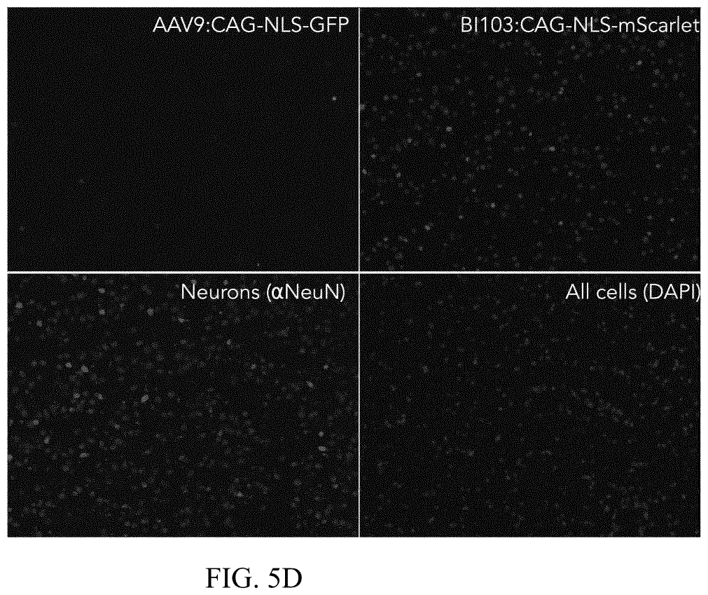

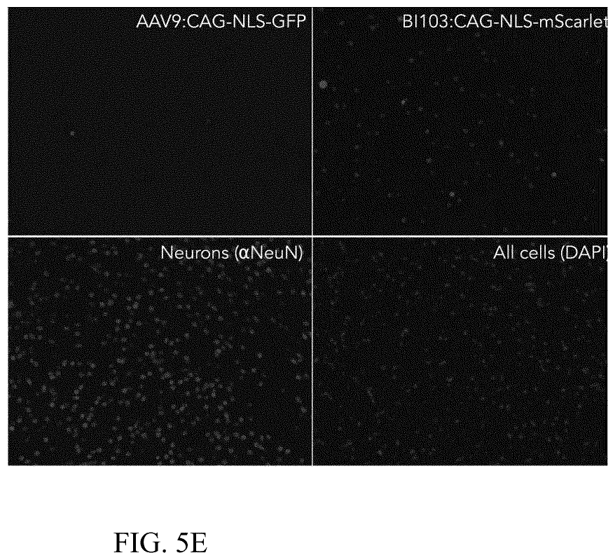

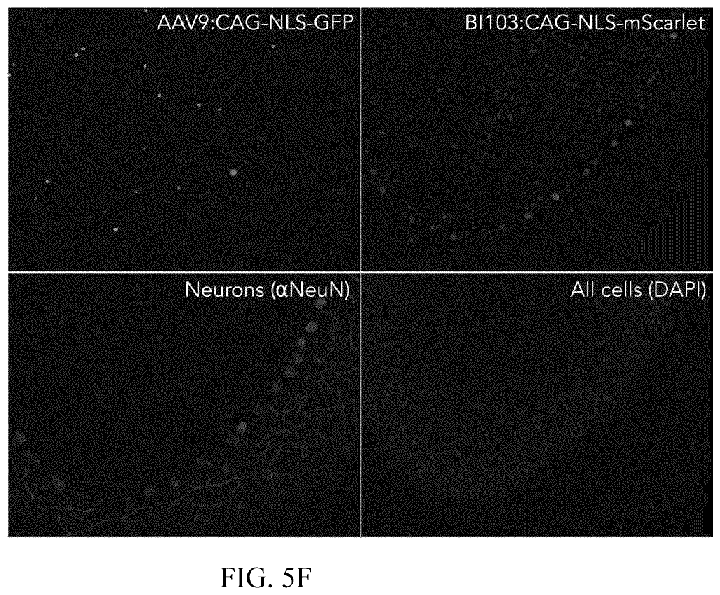

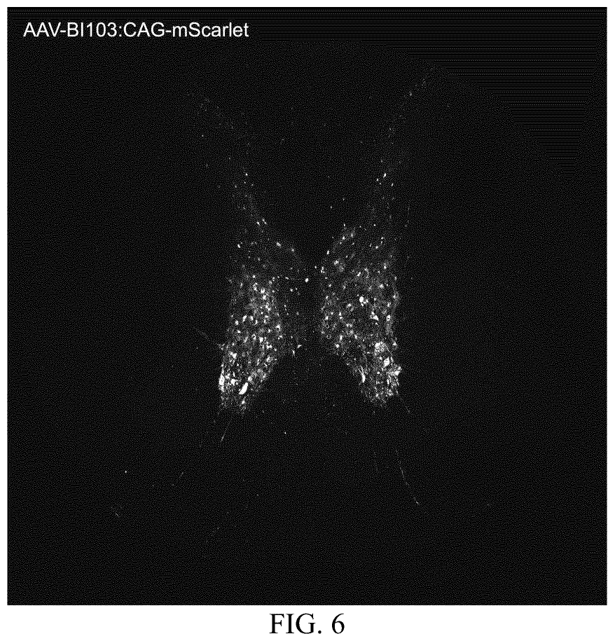

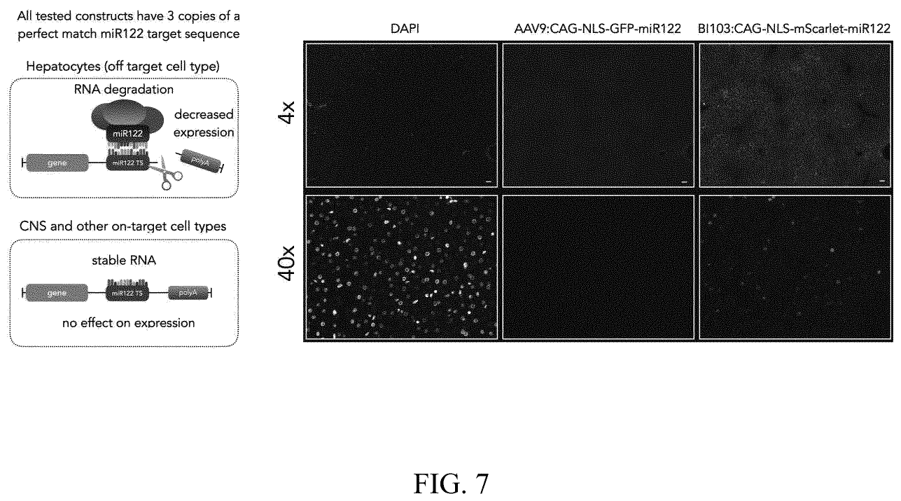

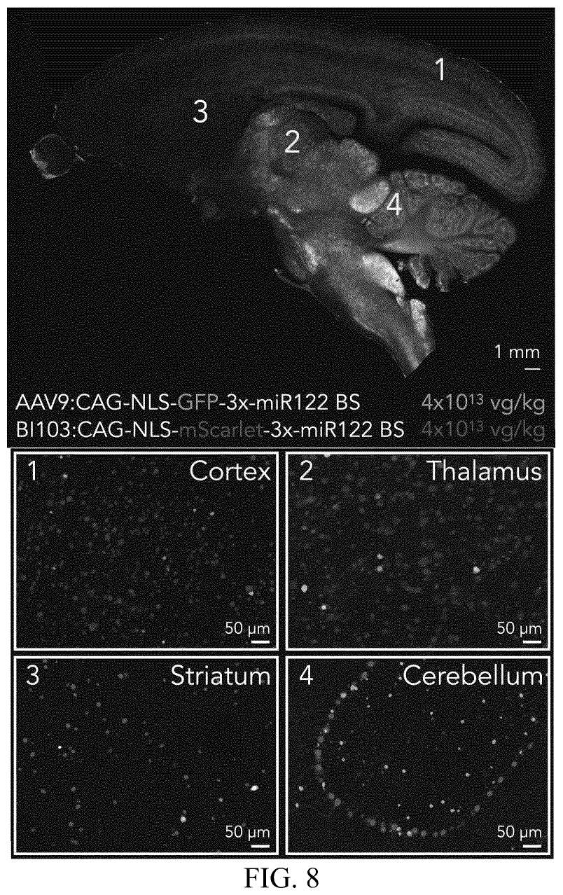

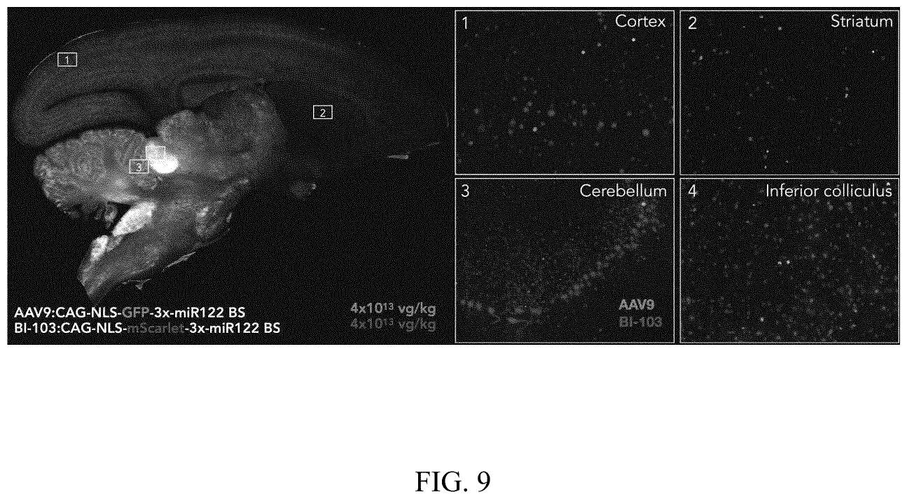

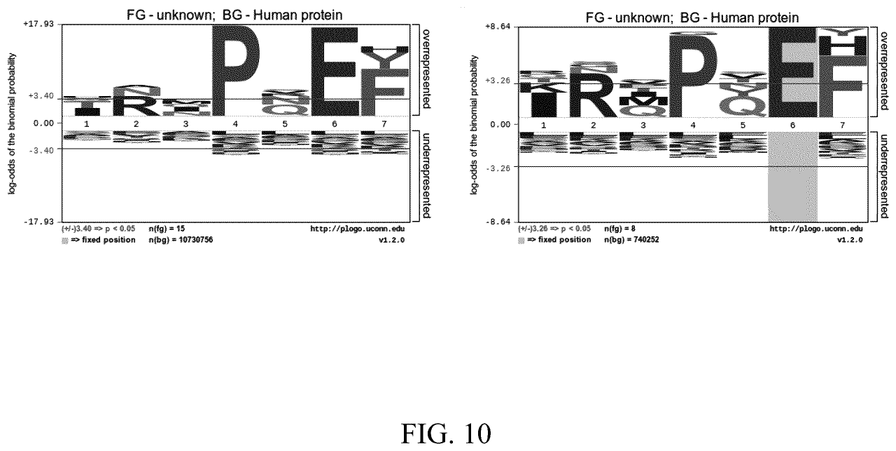

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2nd edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2nd edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide targeting moieties which promote transduction into the CNS. These targeting moieties may be incorporated into particles, such as viral capsid delivery particles, to confer tropism on the delivery particles and promote transduction of CNS. Example CNS tissue include brain and spinal cord tissue. Example CNS cell types include neurons and glial cells. Further embodiments disclosed herein provide a vector system comprising one or more vectors encoding AAV capsids for use in generating rAAV particles according to embodiments described herein as well as vectors encoding cargo molecules such as polypeptides and polynucleotides for delivery via the rAVV particles. Accordingly, embodiments disclosed herein provide compositions capable of delivering cargos with enhanced selectivity and efficiency to the CNS vasculature. Embodiments disclosed herein also provide vector systems for the generation and loading of such delivery particles with a cargo. Likewise, embodiments disclosed herein provide methods for use of such compositions to target CNS endothelial cells, in vitro and in vivo, with implications for both therapeutic and research purposes.

Additional feature and advantages of the aforementioned embodiments are further described below.

### Targeting Moieties and Compositions Thereof

In example embodiments, compositions are provided herein comprising a targeting moiety with an enhanced tropism for endothelial cells of the CNS. This targeting moiety may be coupled directly to a cargo to be delivered such as an oligonucleotide or polypeptide. Alternatively, the targeting molecule may be incorporated into a delivery particle to confer tropism for endothelial cells of the CNS on the delivery particle. A non-limiting example of delivery particle is a viral capsid particle. In such embodiments, the targeting moiety may be incorporated into a viral capsid polypeptide such that the targeting moiety is incorporated into the assembled viral capsid. However, other particle delivery systems where the targeting moiety may be incorporated or attached, for example on exosomes or liposomes, are also envisioned and encompassed as alternative embodiments herein.

The targeting moiety may comprise a n-mer motif. The n-mer motif may comprise Y₁ -Y₂-Y₃-P-Y₄-E-Y₅ wherein Y₁-Y₅ are independently selected amino acids. Y₁-Y₅ may comprise any combination of alanine (ala; A), arginine (arg; R), asparagine (asn; N), aspartic acid (asp; D), cysteine (cys; C), glutamine (gln; Q), glutamic acid (glu; E), glycine (gly; G), histidine (his; H), isoleucine (ile; I); leucine (leu; L); lysine (lys; K), methionine (met; M), phenylalanine (phe; F), proline (pro; P), serine (ser; S); threonine (thr; T), tryptophan (trp; W), tyrosine (tyr; Y); valine (val; V) or artificial amino acids. It should be understood that any reference to any amino acid is intended to encompass any natural amino acid as well as any amino acid mimetic having similar physical and chemical characteristics to naturally occurring amino acids. In an example embodiment, Y₁ or Y₂ is independently selected from an amino acid with a single positive charge, and Y₅ is an amino acid with a single aromatic ring. Positively charged amino acids may comprise R, H, and K. Amino acids with a single aromatic ring may comprise F, W, and Y.

In example embodiments, Y₁ is selected from I, T, Q, H, K, R, V, G, E, or L; Y₂ is selected from R, N, A, K, or G; Y₃ is selected from N, I, Q, M, F, L, D, T, R, V, or S; Y₄ is selected from Q, N, V, S, or T; and Y₅ is selected from F, Y, or H. In a preferred embodiment, the n-mer motif is selected from the group consisting of QRIPQEY (SEQ ID NO: 13), KNSPSEF (SEQ ID NO: 10), TRTPVEF (SEQ ID NO: 16), IRVPNEF (SEQ ID NO: 8), IRMPTEF (SEQ ID NO: 6), IRQPQEF (SEQ ID NO: 7), GKLPREF (SEQ ID NO: 4), LALPQEF (SEQ ID NO: 11), ELNPREF (SEQ ID NO: 3), HNFPNEF (SEQ ID NO: 5), TARPNEY (SEQ ID NO: 15), QNIPSEY (SEQ ID NO: 12), RSQPQEH (SEQ ID NO: 14), KGPGYEH (SEQ ID NO: 9), QSRDLYR (SEQ ID NO: 20), MNSTISK (SEQ ID NO: 19), VNTVREF (SEQ ID NO: 21), ENHTRDK (SEQ ID NO: 17), and GNDTRST (SEQ ID NO: 18). In a preferred embodiment, the n-mer motif is selected from the group consisting of IRVPNEF (SEQ ID NO: 8), IRQPQEF (SEQ ID NO: 7), KNSPSEF (SEQ ID NO: 10), QRIPQEY (SEQ ID NO: 13), TRTPVEF (SEQ ID NO: 16), IRMPTEF (SEQ ID NO: 6), RSQPQEH (SEQ ID NO: 14), KGPGYEH (SEQ ID NO: 9) and HNFPNEF (SEQ ID NO: 5). In a preferred embodiment, the n-mer is IRVPNEF (SEQ ID NO: 8).

In an example embodiment, the n-mer is selected from Table 1 to Table 3. In an example embodiment, the n-mer is selected from SEQ ID: 3-21.

In example embodiments, the n-mer can be used to increase transduction in target cells i.e. CNS cells and tissues. The increase in transduction efficiency of the n-mer to a cell may be compared to a composition that does not contain the targeting moiety for example inclusion of one or more targeting moieties in a composition can result in an increase in transduction and or transduction efficiency by 10%, 20%, 30%, 40%, 50%, 60%, 70%, 80%, 90%, 100% or more. In an exemplary embodiment the increase in transduction and or transduction efficiency is one and a half fold, two-fold, three-fold, four-fold, five-fold, six-fold, seven-fold, eight-fold, nine-fold, ten-fold, 20-fold, 30-fold, 40-fold, 50-fold, 60-fold, 70-fold, 80-fold or more relative to a composition lacking the n-mer. In one embodiment, the transduction and/or transduction efficiency is increased or enhanced in endothelial cells, in one embodiment increase in endothelial cells of the vasculature, for example, the central nervous system vasculature. In embodiments, the transduction and/or transduction efficiency is increased or enhanced in cells of the central nervous system. In embodiments, the transduction and/or transduction efficiency is increased or enhanced in neurons and glial cells. In an embodiment, the composition comprising a n-mer is selective to a target cell as compared to other cell types and/or other virus particles. As used herein, ‘selective’ and ‘cell-selective’ refers to preferential targeting for cells as compared to other cell types. Preferably, the targeting moiety is selective for a desired target (e.g. cell, organ, system e.g. large diameter arteries and veins, brain, retina and spinal cord microvasculature, species) or set of targets by at least 2:1, 3:1, 4:1, 5:1, 6:1 7:1, 8:1, 9:1, 10:1 or more; or 10%, 20%, 30%, 40%, 50%, 60%, 70%, 75% 80%, 85%, 90% or more, relative to other targets or cells (e.g., microvasculature of peripheral organs such as the kidney). In an example embodiment, the composition comprising a targeting moiety described herein can have an increased uptake, delivery rate, transduction rate, efficiency, amount, or a combination thereof in a target cell (e.g., endothelial cells across the arterio-venous axis in brain, retina, and spinal cord vasculature) as compared to other cells types (e.g., muscle cells) and/or other virus particles (e.g., AAVs not containing the targeting moiety) and other compositions that do not contain the cell-selective n-mer motif of the present invention.

### Engineered Viral Capsids

Described herein are various embodiments of engineered viral capsids, such as adeno-associated virus (AAV) capsids, that can be engineered to confer cell-selective tropism, such as CNS tissue- and cell-specific tropism, to an engineered viral particle. Engineered viral capsids can be lentiviral, retroviral, adenoviral, or AAV capsids. The engineered capsids can be included in an engineered virus particle (e.g., an engineered lentiviral, retroviral, adenoviral, or AAV virus particle), and can confer cell-selective tropism to the engineered viral particle. The engineered viral capsids described herein can include one or more engineered viral capsid proteins described herein. The engineered viral capsids described herein can include one or more engineered viral capsid proteins described herein that can contain one or more target moiety as described elsewhere herein.

The engineered viral capsids can be variants of wild-type viral capsid. For example, in some embodiments, the engineered AAV capsids can be variants of wild-type AAV capsids. In some embodiments, the wild-type AAV capsids can be composed of VP1, VP2, VP3 capsid proteins or a combination thereof. In other words, the engineered AAV capsids can include one or more variants of a wild-type VP1, wild-type VP2, and/or wild-type VP3 capsid proteins. In some embodiments, the serotype of the reference wild-type AAV capsid can be AAV-1, AAV-2, AAV-3, AAV-4, AAV-5, AAV-6, AAV-8, AAV-9 or any combination thereof. In some embodiments, the serotype of the wild-type AAV capsid can be AAV-9. The engineered AAV capsids can have a different tropism than that of the reference wild-type AAV capsid.

In some embodiments, the target moiety is incorporated into a viral protein, such as a capsid protein, including but not limited to lentiviral, adenoviral, AAV, bacteriophage, retroviral proteins. In some embodiments, the target moiety is located between two amino acids of the viral protein such that the target moiety is external (i.e., is presented on the surface of) to a viral capsid. In an example embodiment, the target moiety disclosed herein can be inserted between two amino acids in the wild-type viral protein (VP) (or capsid protein). In some embodiments, the target moiety can be inserted between two amino acids in a variable amino acid region in a viral capsid protein.

In some embodiments, the target moiety can be inserted between two amino acids in a variable amino acid region in an AAV capsid protein. The core of each wild-type AAV viral protein contains an eight-stranded beta-barrel motif (betaB to betaI) and an alpha-helix (alphaA) that are conserved in autonomous parvovirus capsids (see e.g., DiMattia et al. 2012. J. Virol. 86 (12): 6947-6958). Structural variable regions (VRs) occur in the surface loops that connect the beta-strands, which cluster to produce local variations in the capsid surface. AAVs have 12 variable regions (also referred to as hypervariable regions) (see e.g., Weitzman and Linden. 2011. “Adeno-Associated Virus Biology.” In Snyder, R. O., Moullier, P. (eds.) Totowa, NJ: Humana Press). In one example embodiment, one or more target moiety can be inserted between two amino acids in one or more of the 12 variable regions in the wild-type AVV capsid proteins. In one example embodiment, the one or more target moieties can be each be inserted between two amino acids in VR-I, VR-II, VR-III, VR-IV, VR-V, VR-VI, VR-VII, VR-III, VR-IX, VR-X, VR-XI, VR-XII, or a combination thereof. In one example embodiment, the engineered capsid is a modified AAV1 capsid and can have a target moiety motif inserted after or a neighbor of amino acid 590, e.g., between amino acid 590 and 591 (SEQ ID NO: 1). In one example embodiment, the engineered capsid is a modified AAV3 capsid and can have a target moiety motif inserted after or a neighbor of amino acid 586, e.g., between amino acid 586 and 587. In one example embodiment, the engineered capsid is a modified AAV4 capsid and can have a target moiety motif inserted after or a neighbor of amino acid 586. In one example embodiment, the engineered capsid is a modified AAV5 capsid and can have a target moiety motif inserted after or a neighbor of amino acid 575. In one example embodiment, the engineered capsid is a modified AAV6 capsid and can have a target moiety inserted at or a neighbor of amino acid 585 and optionally immediately after one of positions Y705-731, T492V, K531E. In one example embodiment, the engineered capsid is a modified AAV8 capsid and can have a target moiety inserted after or a neighbor of amino acid 585 and 590. In one example embodiment, the engineered capsid is a modified AAV9 capsid and can have a target moiety inserted after or a neighbor of amino acid 588 and 589 or between amino acid 588 and 589. (Büning, H.; Srivastava, A. Capsid Modifications for Targeting and Improving the Efficacy of AAV Vectors. Molecular Therapy—Methods & Clinical Development 2019, 12, 248-265). In one example embodiment, the engineered capsid can have a 7-mer peptide inserted between amino acids 588 and 589 of an AAV9 viral protein. SEQ ID NO: 1 is a reference AAV9 capsid sequence for at least referencing the insertion sites discussed above. It will be appreciated that target moieties can be inserted in analogous positions in AAV viral proteins of other serotypes, such as but not limited to, AAV1, AAV2, AAV3, AAV4, AAV5, AAV6, AAV7, AAV8, AAV9, AAV rh.74, AAV rh.10 capsid polypeptide. In some embodiments as previously discussed, the target moiety(s) can be inserted between any two contiguous amino acids within the AAV viral protein and in some embodiments the insertion is made in a variable region.

In one example embodiment, the first 1, 2, 3, or 4 amino acids of a target moiety can replace 1, 2, 3, or 4 amino acids of a polypeptide into which it is inserted and preceding the insertion site. Using an AAV as another non-limiting example, one or more of the target moieties can be inserted into e.g., an AAV9 capsid polypeptide between amino acids 588 and 589 and the insert can replace amino acids 586, 587, and 588 such that the amino acid immediately preceding the target moiety after insertion is residue 585. It will be appreciated that this principle can apply in any other insertion context and is not necessarily limited to insertion between residues 588 and 589 of an AAV9 capsid or equivalent position in another AAV capsid. It will further be appreciated that in some embodiments, no amino acids in the polypeptide into which the target moiety is inserted are replaced by the target moiety. In an example embodiment, the AAV capsid is selected from SEQ ID: 1, 22-40.

In some embodiments, in addition to the n-mer motif(s) the targeting moiety can include a polypeptide, a polynucleotide, a lipid, a polymer, a sugar, or a combination thereof.

The engineered viral capsid and/or capsid proteins can be encoded by one or more engineered viral capsid polynucleotides. In some embodiments, the engineered viral capsid polynucleotide is an engineered AAV capsid polynucleotide, engineered lentiviral capsid polynucleotide, engineered retroviral capsid polynucleotide, or engineered adenovirus capsid polynucleotide. In some embodiments, an engineered viral capsid polynucleotide (e.g, an engineered AAV capsid polynucleotide, engineered lentiviral capsid polynucleotide, engineered retroviral capsid polynucleotide, or engineered adenovirus capsid polynucleotide) can include a 3′ polyadenylation signal. The polyadenylation signal can be an SV40 polyadenylation signal.

In some embodiments, the engineered polynucleotide can be included in a polynucleotide that is configured to be a viral genome donor in a viral vector system that can be used to generate engineered viral particles described elsewhere herein.

In some embodiments, the engineered AAV capsid encoding polynucleotide can be included in a polynucleotide that is configured to be an AAV genome donor in an AAV vector system that can be used to generate engineered AAV particles described elsewhere herein. In some embodiments, the engineered AAV capsid encoding polynucleotide can be operably coupled to a poly adenylation tail. In some embodiments, the poly adenylation tail can be an SV40 poly adenylation tail. In some embodiments, the AAV capsid encoding polynucleotide can be operably coupled to a promoter. The promoter may be selected based on the cell-based system used for production of the AAV capsid and optimized for expression in that particular host cell system. Delivery of vectors encoding the AAV capsid in vivo for in vivo production of rAAVs is also contemplated In some embodiments, the promoter can be a tissue specific promoter depending on the cell-type targeted for in vivo AAV production. In some embodiments, the tissue specific promoter is specific for muscle (e.g., cardiac, skeletal, and/or smooth muscle), neurons and supporting cells (e.g., astrocytes, glial cells, Schwann cells, etc.), fat, spleen, liver, kidney, immune cells, spinal fluid cells, synovial fluid cells, skin cells, cartilage, tendons, connective tissue, bone, pancreas, adrenal gland, blood cell, bone marrow cells, placenta, endothelial cells, and combinations thereof. In some embodiments, the promoter can be a constitutive promoter. Suitable tissue specific promoters and constitutive promoters are discussed elsewhere herein and are generally known in the art and can be commercially available. Suitable neuronal tissue/cell specific promoters include, but are not limited to, GFAP promoter (astrocytes), SYN1 promoter (neurons), and NSE/RU5′ (mature neurons).

### Further Capsid Modifications

In one example embodiment, the viral capsid protein may comprise one or more mutations relative to wild type. In an example embodiment, the one or more mutations comprise a K449R substitution in a capsid polypeptide of AAV 9, or a substitution in an analogous position of a capsid polypeptide from AAV1, AAV2, AAV3, AAV4, AAV5, AAV6, AAV7, AAV8, AAVrh8, AAV rh.74, or AAV rh. 10.

### Engineered Vectors and Vector Systems

Also provided herein are vectors and vector systems that can contain one or more of the engineered polynucleotides described herein that can encode one or more of the target moiety of the present invention, including but not limited to engineered viral polynucleotides (e.g., engineered AAV polynucleotides). As used in this context, engineered viral capsid polynucleotides refers to any one or more of the polynucleotides described herein capable of encoding an engineered viral capsid as described elsewhere herein and/or polynucleotide(s) capable of encoding one or more engineered viral capsid proteins described elsewhere herein. Further, where the vector includes an engineered viral capsid polynucleotide described herein, the vector can also be referred to and considered an engineered vector or system thereof although not specifically noted as such. In embodiments, the vector can contain one or more polynucleotides encoding one or more elements of an engineered viral capsid described herein. The vectors and systems thereof can be useful in producing bacterial, fungal, yeast, plant cells, animal cells, and transgenic animals that can express one or more components of the engineered viral capsid, particle, or other compositions described herein. Within the scope of this disclosure are vectors containing one or more of the polynucleotide sequences described herein. One or more of the polynucleotides that are part of the engineered viral capsid and system thereof described herein can be included in a vector or vector system.

In some embodiments, the vector can include an engineered viral (e.g., AAV) capsid polynucleotide having a 3′ polyadenylation signal. In some embodiments, the 3′ polyadenylation is an SV40 polyadenylation signal. In some embodiments the vector does not have splice regulatory elements. In some embodiments, the vector includes one or more minimal splice regulatory elements. In some embodiments, the vector can further include a modified splice regulatory element, wherein the modification inactivates the splice regulatory element. In some embodiments, the modified splice regulatory element is a polynucleotide sequence sufficient to induce splicing, between a rep protein polynucleotide and the engineered viral (e.g., AAV) capsid protein variant polynucleotide. In some embodiments, the polynucleotide sequence can be sufficient to induce splicing is a splice acceptor or a splice donor. In some embodiments, the viral (e.g., AAV) capsid polynucleotide is an engineered viral (e.g., AAV) capsid polynucleotide as described elsewhere herein. It some embodiments, the vector does not include one or more minimal splice regulatory elements, modified splice regulatory agent, splice acceptor, and/or splice donor.

The vectors and/or vector systems can be used, for example, to express one or more of the engineered viral (e.g., AAV) capsid and/or other polynucleotides in a cell, such as a producer cell, to produce engineered viral (e.g., AAV) particles and/or other compositions (e.g., polypeptides, particles, etc.) containing an engineered viral (e.g., AAV) capsid or other composition containing an n-mer motif of the present invention described elsewhere herein. Other uses for the vectors and vector systems described herein are also within the scope of this disclosure. In general, and throughout this specification, the term “vector” is a tool that allows or facilitates the transfer of an entity from one environment to another. In some contexts which will be appreciated by those of ordinary skill in the art, “vector” can be a term of art to refer to a nucleic acid molecule capable of transporting another nucleic acid to which it has been linked. A vector can be a replicon, such as a plasmid, phage, or cosmid, into which another DNA segment may be inserted so as to bring about the replication of the inserted segment. Generally, a vector is capable of replication when associated with the proper control elements.

Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can be composed of a nucleic acid (e.g., a polynucleotide) of the invention in a form suitable for expression of the nucleic acid in a host cell, which means that the recombinant expression vectors include one or more regulatory elements, which can be selected on the basis of the host cells to be used for expression, that is operatively-linked to the nucleic acid sequence to be expressed. Within a recombinant expression vector, “operably linked” and “operatively-linked” are used interchangeably herein and further defined elsewhere herein. In the context of a vector, the term “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). Advantageous vectors include adeno-associated viruses, and types of such vectors can also be selected for targeting particular types of cells, such as those engineered viral (e.g., AAV) vectors containing an engineered viral (e.g., AAV) capsid polynucleotide with a desired cell-specific tropism. These and other embodiments of the vectors and vector systems are described elsewhere herein.

In some embodiments, the vector can be a bicistronic vector. In some embodiments, a bicistronic vector can be used for one or more elements of the engineered viral (e.g., AAV) capsid system described herein. In some embodiments, expression of elements of the engineered viral (e.g., AAV) capsid system described herein can be driven by a suitable constitutive or tissue specific promoter. Where the element of the engineered viral (e.g., AAV) capsid system is an RNA, its expression can be driven by a Pol III promoter, such as a U6 promoter. In some embodiments, the two are combined.

### Cell-based Vector Amplification and Expression

Vectors can be designed for expression of one or more elements of the engineered viral (e.g., AAV) capsid system or other compositions containing an n-mer motif of the present invention described herein (e.g., nucleic acid transcripts, proteins, enzymes, and combinations thereof) in a suitable host cell. In some embodiments, the suitable host cell is a prokaryotic cell. Suitable host cells include, but are not limited to, bacterial cells, yeast cells, insect cells, and mammalian cells. The vectors can be viral-based or non-viral based. In some embodiments, the suitable host cell is a eukaryotic cell. In some embodiments, the suitable host cell is a suitable bacterial cell. Suitable bacterial cells include, but are not limited to, bacterial cells from the bacteria of the species Escherichia coli. Many suitable strains of E. coli are known in the art for expression of vectors. These include, but are not limited to Pir1, Stbl2, Stbl3, Stbl4, TOP10, XL1 Blue, and XL10 Gold. In some embodiments, the host cell is a suitable insect cell. Suitable insect cells include those from Spodoptera frugiperda. Suitable strains of S. frugiperda cells include, but are not limited to, Sf9 and Sf21. In some embodiments, the host cell is a suitable yeast cell. In some embodiments, the yeast cell can be from Saccharomyces cerevisiae. In some embodiments, the host cell is a suitable mammalian cell. Many types of mammalian cells have been developed to express vectors. Suitable mammalian cells include, but are not limited to, HEK293, Chinese Hamster Ovary Cells (CHOs), mouse myeloma cells, HeLa, U2OS, A549, HT1080, CAD, P19, NIH 3T3, L929, N2a, MCF-7, Y79, SO-Rb50, HepG G2, DIKX-X11, J558L, Baby hamster kidney cells (BHK), and chicken embryo fibroblasts (CEFs). Suitable host cells are discussed further in Goeddel, GENE EXPRESSION TECHNOLOGY: METHODS IN ENZYMOLOGY 185, Academic Press, San Diego, Calif. (1990).

In some embodiments, the vector can be a yeast expression vector. Examples of vectors for expression in yeast Saccharomyces cerevisiae include pYepSec1 (Baldari, et al., 1987. EMBO J. 6:229-234), pMFa (Kuijan and Herskowitz, 1982. Cell 30:933-943), pJRY88 (Schultz et al., 1987. Gene 54:113-123), pYES2 (Invitrogen Corporation, San Diego, Calif.), and picZ (In Vitrogen Corp, San Diego, Calif.). As used herein, a “yeast expression vector” refers to a nucleic acid that contains one or more sequences encoding an RNA and/or polypeptide and may further contain any desired elements that control the expression of the nucleic acid(s), as well as any elements that enable the replication and maintenance of the expression vector inside the yeast cell. Many suitable yeast expression vectors and features thereof are known in the art; for example, various vectors and techniques are illustrated in in Yeast Protocols, 2nd edition, Xiao, W., ed. (Humana Press, New York, 2007) and Buckholz, R. G. and Gleeson, M. A. (1991) Biotechnology (NY) 9 (11): 1067-72. Yeast vectors can contain, without limitation, a centromeric (CEN) sequence, an autonomous replication sequence (ARS), a promoter, such as an RNA Polymerase III promoter, operably linked to a sequence or gene of interest, a terminator such as an RNA polymerase III terminator, an origin of replication, and a marker gene (e.g., auxotrophic, antibiotic, or other selectable markers). Examples of expression vectors for use in yeast may include plasmids, yeast artificial chromosomes, 2μ plasmids, yeast integrative plasmids, yeast replicative plasmids, shuttle vectors, and episomal plasmids.

In some embodiments, the vector is a baculovirus vector or expression vector and can be suitable for expression of polynucleotides and/or proteins in insect cells. Baculovirus vectors available for expression of proteins in cultured insect cells (e.g., SF9 cells) include the pAc series (Smith, et al., 1983. Mol. Cell. Biol. 3:2156-2165) and the pVL series (Lucklow and Summers, 1989. Virology 170:31-39). rAAV (recombinant Adeno-associated viral) vectors are preferably produced in insect cells, e.g., Spodoptera frugiperda Sf9 insect cells, grown in serum-free suspension culture. Serum-free insect cells can be purchased from commercial vendors, e.g., Sigma Aldrich (EX-CELL 405).

In some embodiments, the vector is a mammalian expression vector. In some embodiments, the mammalian expression vector is capable of expressing one or more polynucleotides and/or polypeptides in a mammalian cell. Examples of mammalian expression vectors include, but are not limited to, pCDM8 (Seed, 1987. Nature 329:840) and pMT2PC (Kaufman, et al., 1987. EMBO J. 6:187-195). The mammalian expression vector can include one or more suitable regulatory elements capable of controlling expression of the one or more polynucleotides and/or proteins in the mammalian cell. For example, commonly used promoters are derived from polyoma, adenovirus 2, cytomegalovirus, simian virus 40, and others disclosed herein and known in the art. More detail on suitable regulatory elements is described elsewhere herein.

For other suitable expression vectors and vector systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1:268-277), lymphoid-specific promoters (Calame and Eaton, 1988. Adv. Immunol. 43:235-275), in particular promoters of T cell receptors (Winoto and Baltimore, 1989. EMBO J. 8:729-733) and immunoglobulins (Baneiji, et al., 1983. Cell 33:729-740; Queen and Baltimore, 1983. Cell 33:741-748), neuron-specific promoters (e.g., the neurofilament promoter; Byrne and Ruddle, 1989. Proc. Natl. Acad. Sci. USA 86:5473-5477), pancreas-specific promoters (Edlund, et al., 1985. Science 230:912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249:374-379) and the a-fetoprotein promoter (Campes and Tilghman, 1989. Genes Dev. 3:537-546). With regards to these prokaryotic and eukaryotic vectors, mention is made of U.S. Pat. No. 6,750,059, the contents of which are incorporated by reference herein in their entirety. Other embodiments can utilize viral vectors, with regards to which mention is made of U.S. patent application Ser. No. 13/092,085, the contents of which are incorporated by reference herein in their entirety. Tissue-specific regulatory elements are known in the art and in this regard, mention is made of U.S. Pat. No. 7,776,321, the contents of which are incorporated by reference herein in their entirety. In some embodiments, a regulatory element can be operably linked to one or more elements of an engineered AAV capsid system so as to drive expression of the one or more elements of the engineered AAV capsid system described herein.

Vectors may be introduced and propagated in a prokaryote or prokaryotic cell. In some embodiments, a prokaryote is used to amplify copies of a vector to be introduced into a eukaryotic cell or as an intermediate vector in the production of a vector to be introduced into a eukaryotic cell (e.g., amplifying a plasmid as part of a viral vector packaging system). In some embodiments, a prokaryote is used to amplify copies of a vector and express one or more nucleic acids, such as to provide a source of one or more proteins for delivery to a host cell or host organism.

In some embodiments, the vector can be a fusion vector or fusion expression vector. In some embodiments, fusion vectors add a number of amino acids to a protein encoded therein, such as to the amino terminus, carboxy terminus, or both of a recombinant protein. Such fusion vectors can serve one or more purposes, such as: (i) to increase expression of recombinant protein; (ii) to increase the solubility of the recombinant protein; and (iii) to aid in the purification of the recombinant protein by acting as a ligand in affinity purification. In some embodiments, expression of polynucleotides (such as non-coding polynucleotides) and proteins in prokaryotes can be carried out in Escherichia coli with vectors containing constitutive or inducible promoters directing the expression of either fusion or non-fusion polynucleotides and/or proteins. In some embodiments, the fusion expression vector can include a proteolytic cleavage site, which can be introduced at the junction of the fusion vector backbone or other fusion moiety and the recombinant polynucleotide or protein to enable separation of the recombinant polynucleotide or protein from the fusion vector backbone or other fusion moiety subsequent to purification of the fusion polynucleotide or protein. Such enzymes, and their cognate recognition sequences, include Factor Xa, thrombin and enterokinase. Example fusion expression vectors include pGEX (Pharmacia Biotech Inc; Smith and Johnson, 1988. Gene 67:31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein. Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

In some embodiments, one or more vectors driving expression of one or more elements of an engineered viral (e.g., AAV) capsid system or other composition containing an n-mer motif described herein are introduced into a host cell such that expression of the elements of the engineered delivery system described herein direct formation of an engineered viral (e.g., AAV) capsid system or other composition containing an n-mer motif described herein (including but not limited to an engineered gene transfer agent particle, which is described in greater detail elsewhere herein). For example, different elements of the engineered viral (e.g., AAV) capsid system or other composition containing an n-mer motif described herein can each be operably linked to separate regulatory elements on separate vectors. RNA(s) of different elements of the engineered delivery system described herein can be delivered to an animal or mammal or cell thereof to produce an animal or mammal or cell thereof that constitutively or inducibly or conditionally expresses different elements of the engineered viral (e.g., AAV) capsid system or other composition containing an n-mer motif described herein that incorporates one or more elements of the engineered viral (e.g., AAV) capsid system or other composition containing an n-mer motif described herein or contains one or more cells that incorporates and/or expresses one or more elements of the engineered viral (e.g., AAV) capsid system or other composition containing an n-mer motif described herein.

In some embodiments, two or more of the elements expressed from the same or different regulatory element(s) can be combined in a single vector, with one or more additional vectors providing any components of the system not included in the first vector. Engineered polynucleotides of the present invention that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a transcript encoding one or more engineered viral (e.g., AAV) capsid proteins or other composition containing an n-mer motif described herein, embedded within one or more intron sequences (e.g., each in a different intron, two or more in at least one intron, or all in a single intron). In some embodiments, the engineered polynucleotides of the present invention (including but not limited to engineered viral polynucleotides) can be operably linked to and expressed from the same promoter.

### Vector Features

The vectors can include additional features that can confer one or more functionalities to the vector, the polynucleotide to be delivered, a virus particle produced there from, or polypeptide expressed thereof. Such features include, but are not limited to, regulatory elements, selectable markers, molecular identifiers (e.g., molecular barcodes), stabilizing elements, and the like. It will be appreciated by those skilled in the art that the design of the expression vector and additional features included can depend on such factors as the choice of the host cell to be transformed, the level of expression desired, etc.

### Regulatory Elements

In embodiments, the polynucleotides and/or vectors thereof described herein (including, but not limited to, the engineered AAV capsid polynucleotides of the present invention) can include one or more regulatory elements that can be operatively linked to the polynucleotide. The term “regulatory element” is intended to include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter can direct expression primarily in a desired tissue of interest, such as muscle, neuron, bone, skin, blood, specific organs (e.g., liver, brain), or particular cell types (e.g., lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985)), the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8 (1), p. 466-472, 1988); SV40 enhancer; and the intron sequence between exons 2 and 3 of rabbit B-globin (Proc. Natl. Acad. Sci. USA., Vol. 78 (3), p. 1527-31, 1981).

In some embodiments, the regulatory sequence can be a regulatory sequence described in U.S. Pat. No. 7,776,321, U.S. Pat. Pub. No. 2011/0027239, and PCT publication WO 2011/028929, the contents of which are incorporated by reference herein in their entirety. In some embodiments, the vector can contain a minimal promoter. In some embodiments, the minimal promoter is the Mecp2 promoter, tRNA promoter, or U6. In a further embodiment, the minimal promoter is tissue specific. In some embodiments, the length of the vector polynucleotide the minimal promoters and polynucleotide sequences is less than 4.4Kb.

To express a polynucleotide, the vector can include one or more transcriptional and/or translational initiation regulatory sequences, e.g., promoters, that direct the transcription of the gene and/or translation of the encoded protein in a cell. In some embodiments a constitutive promoter may be employed. Suitable constitutive promoters for mammalian cells are generally known in the art and include, but are not limited to SV40, CAG, CMV, EF-1α, B-actin, RSV, and PGK. Suitable constitutive promoters for bacterial cells, yeast cells, and fungal cells are generally known in the art, such as a T-7 promoter for bacterial expression and an alcohol dehydrogenase promoter for expression in yeast.

In some embodiments, the regulatory element can be a regulated promoter. “Regulated promoter” refers to promoters that direct gene expression not constitutively, but in a temporally-and/or spatially-regulated manner, and includes tissue-specific, tissue-preferred and inducible promoters. In some embodiments, the regulated promoter is a tissue specific promoter as previously discussed elsewhere herein. Regulated promoters include conditional promoters and inducible promoters. In some embodiments, conditional promoters can be employed to direct expression of a polynucleotide in a specific cell type, under certain environmental conditions, and/or during a specific state of development. Suitable tissue specific promoters can include, but are not limited to, liver specific promoters (e.g., APOA2, SERPIN A1 (hAAT), CYP3A4, and MIR122), pancreatic cell promoters (e.g., INS, IRS2, Pdx1, A1x3, Ppy), cardiac specific promoters (e.g., Myh6 (alpha MHC), MYL2 (MLC-2v), TNI3 (cTn1), NPPA (ANF), Slc8a1 (Ncx1)), central nervous system cell promoters (SYN1, GFAP, INA, NES, MOBP, MBP, TH, FOXA2 (HNF3 beta)), skin cell specific promoters (e.g., FLG, K14, TGM3), immune cell specific promoters, (e.g., ITGAM, CD43 promoter, CD14 promoter, CD45 promoter, CD68 promoter), urogenital cell specific promoters (e.g., Pbsn, Upk2, Sbp, Fer114), endothelial cell specific promoters (e.g., ENG), pluripotent and embryonic germ layer cell specific promoters (e.g., Oct4, NANOG, Synthetic Oct4, T brachyury, NES, SOX17, FOXA2, MIR122), and muscle cell specific promoter (e.g., Desmin). Other tissue and/or cell specific promoters are discussed elsewhere herein and can be generally known in the art and are within the scope of this disclosure.

Inducible/conditional promoters can be positively inducible/conditional promoters (e.g., a promoter that activates transcription of the polynucleotide upon appropriate interaction with an activated activator, or an inducer (compound, environmental condition, or other stimulus) or a negative/conditional inducible promoter (e.g., a promoter that is repressed (e.g., bound by a repressor) until the repressor condition of the promotor is removed (e.g., inducer binds a repressor bound to the promoter stimulating release of the promoter by the repressor or removal of a chemical repressor from the promoter environment). The inducer can be a compound, environmental condition, or other stimulus. Thus, inducible/conditional promoters can be responsive to any suitable stimuli such as chemical, biological, or other molecular agents, temperature, light, and/or pH. Suitable inducible/conditional promoters include, but are not limited to, Tet-On, Tet-Off, Lac promoter, pBad, AlcA, LexA, Hsp70 promoter, Hsp90 promoter, pDawn, XVE/OlexA, GVG, and pOp/LhGR.

In some embodiments, the vector or system thereof can include one or more elements capable of translocating and/or expressing an engineered polynucleotide of the present invention (e.g., an engineered viral (e.g., AAV) capsid polynucleotide) to/in a specific cell component or organelle. Such organelles can include, but are not limited to, nucleus, ribosome, endoplasmic reticulum, Golgi apparatus, chloroplast, mitochondria, vacuole, lysosome, cytoskeleton, plasma membrane, cell wall, peroxisome, centrioles, etc.

### Selectable Markers and Tags

One or more of the engineered polynucleotides of the present invention (e.g., an engineered viral (e.g., AAV) capsid polynucleotide) can be operably linked, fused to, or otherwise modified to include a polynucleotide that encodes or is a selectable marker or tag, which can be a polynucleotide or polypeptide. In some embodiments, the polypeptide encoding a polypeptide selectable marker can be incorporated in the engineered polynucleotide of the present invention (e.g., an engineered viral (e.g., AAV) capsid polynucleotide) such that the selectable marker polypeptide, when translated, is inserted between two amino acids between the N- and C-terminus of an engineered polypeptide (e.g., the engineered AAV capsid polypeptide) or at the N- and/or C-terminus of the engineered polypeptide (e.g., an engineered AAV capsid polypeptide). In some embodiments, the selectable marker or tag is a polynucleotide barcode or unique molecular identifier (UMI).

It will be appreciated that the polynucleotide encoding such selectable markers or tags can be incorporated into a polynucleotide encoding one or more components of the engineered AAV capsid system described herein in an appropriate manner to allow expression of the selectable marker or tag. Such techniques and methods are described elsewhere herein and will be instantly appreciated by one of ordinary skill in the art in view of this disclosure. Many such selectable markers and tags are generally known in the art and are intended to be within the scope of this disclosure.

Suitable selectable markers and tags include, but are not limited to, affinity tags, such as chitin binding protein (CBP), maltose binding protein (MBP), glutathione-S-transferase (GST), poly (His) tag; solubilization tags such as thioredoxin (TRX) and poly (NANP), MBP, and GST; chromatography tags such as those consisting of polyanionic amino acids, such as FLAG-tag; epitope tags such as V5-tag, Myc-tag, HA-tag and NE-tag; protein tags that can allow specific enzymatic modification (such as biotinylation by biotin ligase) or chemical modification (such as reaction with FLASH-EDT2 for fluorescence imaging), DNA and/or RNA segments that contain restriction enzyme or other enzyme cleavage sites; DNA segments that encode products that provide resistance against otherwise toxic compounds including antibiotics, such as, spectinomycin, ampicillin, kanamycin, tetracycline, Basta, neomycin phosphotransferase II (NEO), hygromycin phosphotransferase (HPT)) and the like; DNA and/or RNA segments that encode products that are otherwise lacking in the recipient cell (e.g., tRNA genes, auxotrophic markers); DNA and/or RNA segments that encode products which can be readily identified (e.g., phenotypic markers such as B-galactosidase, GUS; fluorescent proteins such as green fluorescent protein (GFP), cyan (CFP), yellow (YFP), red (RFP), luciferase, and cell surface proteins); polynucleotides that can generate one or more new primer sites for PCR (e.g., the juxtaposition of two DNA sequences not previously juxtaposed), DNA sequences not acted upon or acted upon by a restriction endonuclease or other DNA modifying enzyme, chemical, etc.; epitope tags (e.g., GFP, FLAG- and His-tags), and, DNA sequences that make a molecular barcode or unique molecular identifier (UMI), DNA sequences required for a specific modification (e.g., methylation) that allows its identification. Other suitable markers will be appreciated by those of skill in the art.

Selectable markers and tags can be operably linked to one or more components of the engineered AAV capsid system or other compositions and/or systems described herein via suitable linker, such as a glycine or glycine serine linkers as short as GS or GG up to (GGGGG)₃ (SEQ ID NO: 251) or (GGGGS)₃ (SEQ ID NO: 252). Other suitable linkers are described elsewhere herein.

The vector or vector system can include one or more polynucleotides encoding one or more n-mers. In some embodiments, the n-mer encoding polynucleotides can be included in the vector or vector system, such as a viral vector system, such that they are expressed within and/or on the virus particle(s) produced such that the virus particles can be targeted to specific cells, tissues, organs, etc. In some embodiments, the n-mer encoding polynucleotides can be included in the vector or vector system such that the engineered polynucleotide(s) of the present invention (e.g., an engineered viral (e.g., AAV) capsid polynucleotide(s)) and/or products expressed therefrom include the n-mer and can be targeted to specific cells, tissues, organs, etc. In some embodiments, such as non-viral carriers, the n-mer can be attached to the carrier (e.g., polymer, lipid, inorganic molecule etc.) and can be capable of targeting the carrier and any attached or associated engineered polynucleotide(s) of the present invention, the engineered polypeptides, or other compositions of the present invention described herein, to specific cells, tissues, organs, etc. In some embodiments, the specific cells are CNS cells.

### Cell-Free Vector and Polynucleotide Expression

In some embodiments, the polynucleotide(s) encoding an n-mer motif of the present invention can be expressed from a vector or suitable polynucleotide in a cell-free in vitro system. In some embodiments, the polynucleotide encoding one or more features of the engineered AAV capsid system can be expressed from a vector or suitable polynucleotide in a cell-free in vitro system. In other words, the polynucleotide can be transcribed and optionally translated in vitro. In vitro transcription/translation systems and appropriate vectors are generally known in the art and commercially available. Generally, in vitro transcription and in vitro translation systems replicate the processes of RNA and protein synthesis, respectively, outside of the cellular environment. Vectors and suitable polynucleotides for in vitro transcription can include T7, SP6, T3, promoter regulatory sequences that can be recognized and acted upon by an appropriate polymerase to transcribe the polynucleotide or vector.

In vitro translation can be stand-alone (e.g., translation of a purified polyribonucleotide) or linked/coupled to transcription. In some embodiments, the cell-free (or in vitro) translation system can include extracts from rabbit reticulocytes, wheat germ, and/or E. coli. The extracts can include various macromolecular components that are needed for translation of exogenous RNA (e.g., 70S or 80S ribosomes, tRNAs, aminoacyl-tRNA, synthetases, initiation, elongation factors, termination factors, etc.). Other components can be included or added during the translation reaction, including but not limited to, amino acids, energy sources (ATP, GTP), energy regenerating systems (creatine phosphate and creatine phosphokinase (eukaryotic systems)) (phosphoenol pyruvate and pyruvate kinase for bacterial systems), and other co-factors (Mg2+, K+, etc.). As previously mentioned, in vitro translation can be based on RNA or DNA starting material. Some translation systems can utilize an RNA template as starting material (e.g., reticulocyte lysates and wheat germ extracts). Some translation systems can utilize a DNA template as a starting material (e.g., E coli-based systems). In these systems transcription and translation are coupled and DNA is first transcribed into RNA, which is subsequently translated. Suitable standard and coupled cell-free translation systems are generally known in the art and are commercially available.

### Codon Optimization of Vector Polynucleotides

As described elsewhere herein, the polynucleotide encoding an n-mer motif of the present invention and/or other polynucleotides described herein can be codon optimized. In some embodiments, polynucleotides of the engineered AAV capsid system described herein can be codon optimized. In some embodiments, one or more polynucleotides contained in a vector (“vector polynucleotides”) described herein that are in addition to an optionally codon optimized polynucleotide encoding an n-mer motif, including but not limited to, embodiments of the engineered AAV capsid system described herein, can be codon optimized. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In some embodiments, one or more codons (e.g., 1, 2, 3, 4, 5, 10, 15, 20, 25, 50, or more, or all codons) in a sequence encoding a DNA/RNA-targeting Cas protein corresponds to the most frequently used codon for a particular amino acid. As to codon usage in yeast, reference is made to the online Yeast Genome database available at http://www.yeastgenome.org/community/codon_usage.shtml, or Codon selection in yeast, Bennetzen and Hall, J Biol Chem. 1982 Mar. 25;257 (6): 3026-31. As to codon usage in plants including algae, reference is made to Codon usage in higher plants, green algae, and cyanobacteria, Campbell and Gowri, Plant Physiol. 1990 January; 92 (1): 1-11.; as well as Codon usage in plant genes, Murray et al, Nucleic Acids Res. 1989 Jan. 25; 17 (2): 477-98; or Selection on the codon bias of chloroplast and cyanelle genes in different plant and algal lineages, Morton BR, J Mol Evol. 1998 April; 46 (4): 449-59.

The vector polynucleotide can be codon optimized for expression in a specific cell-type, tissue type, organ type, and/or subject type. In some embodiments, a codon optimized sequence is a sequence optimized for expression in a eukaryote, e.g., humans (i.e., being optimized for expression in a human or human cell), or for another eukaryote, such as another animal (e.g., a mammal or avian) as is described elsewhere herein. Such codon optimized sequences are within the ambit of the ordinary skilled artisan in view of the description herein. In some embodiments, the polynucleotide is codon optimized for a specific cell type. Such cell types can include, but are not limited to, epithelial cells (including skin cells, cells lining the gastrointestinal tract, cells lining other hollow organs), nerve cells (nerves, brain cells, spinal column cells, nerve support cells (e.g., astrocytes, glial cells, Schwann cells etc.), muscle cells (e.g., cardiac muscle, smooth muscle cells, and skeletal muscle cells), connective tissue cells (fat and other soft tissue padding cells, bone cells, tendon cells, cartilage cells), blood cells, stem cells and other progenitor cells, immune system cells, germ cells, and combinations thereof. Such codon optimized sequences are within the ambit of the ordinary skilled artisan in view of the description herein. In some embodiments, the polynucleotide is codon optimized for a specific tissue type. Such tissue types can include, but are not limited to, muscle tissue, connective tissue, nervous tissue, and epithelial tissue. Such codon optimized sequences are within the ambit of the ordinary skilled artisan in view of the description herein. In some embodiments, the polynucleotide is codon optimized for a specific organ. Such organs include, but are not limited to, muscles, skin, intestines, liver, spleen, brain, lungs, stomach, heart, kidneys, gallbladder, pancreas, bladder, thyroid, bone, blood vessels, blood, and combinations thereof. Such codon optimized sequences are within the ambit of the ordinary skilled artisan in view of the description herein.

In some embodiments, a vector polynucleotide is codon optimized for expression in particular cells, such as prokaryotic or eukaryotic cells. The eukaryotic cells may be those of or derived from a particular organism, such as a plant or a mammal, including but not limited to human, or non-human eukaryote or animal or mammal as discussed herein, e.g., mouse, rat, rabbit, dog, livestock, or non-human mammal or primate.

### Non-Viral Vectors and Carriers

In some embodiments, the vector is a non-viral vector or carrier. In some embodiments, non-viral vectors can have the advantage(s) of reduced toxicity and/or immunogenicity and/or increased bio-safety as compared to viral vectors The terms of art “Non-viral vectors and carriers” and as used herein in this context refers to molecules and/or compositions that are not based on one or more component of a virus or virus genome (excluding any nucleotide to be delivered and/or expressed by the non-viral vector) that can be capable of attaching to, incorporating, coupling, and/or otherwise interacting with an engineered capsid polynucleotide (e.g., an engineered AAV capsid polynucleotide) or other composition of the present invention described herein and can be capable of ferrying the polynucleotide to a cell and/or expressing the polynucleotide. It will be appreciated that this does not exclude the inclusion of a virus-based polynucleotide that is to be delivered. For example, if a gRNA to be delivered is directed against a virus component and it is inserted or otherwise coupled to an otherwise non-viral vector or carrier, this would not make said vector a “viral vector”. Non-viral vectors and carriers include naked polynucleotides, chemical-based carriers, polynucleotide (non-viral) based vectors, and particle-based carriers. It will be appreciated that the term “vector” as used in the context of non-viral vectors and carriers refers to polynucleotide vectors and “carriers” used in this context refers to a non-nucleic acid or polynucleotide molecule or composition that be attached to or otherwise interact with a polynucleotide to be delivered, such as an engineered AAV capsid polynucleotide of the present invention.

### Naked Polynucleotides

In some embodiments one or more engineered AAV capsid polynucleotides or other polynucleotides of the present invention described elsewhere herein can be included in a naked polynucleotide. The term of art “naked polynucleotide” as used herein refers to polynucleotides that are not associated with another molecule (e.g., proteins, lipids, and/or other molecules) that can often help protect it from environmental factors and/or degradation. As used herein, associated with includes, but is not limited to, linked to, adhered to, adsorbed to, enclosed in, enclosed in or within, mixed with, and the like. Naked polynucleotides that include one or more of the engineered AAV capsid polynucleotides or other polynucleotides of the present invention described herein can be delivered directly to a host cell and optionally expressed therein. The naked polynucleotides can have any suitable two- and three-dimensional configurations. By way of non-limiting examples, naked polynucleotides can be single-stranded molecules, double stranded molecules, circular molecules (e.g., plasmids and artificial chromosomes), molecules that contain portions that are single stranded and portions that are double stranded (e.g., ribozymes), and the like. In some embodiments, the naked polynucleotide contains only the engineered AAV capsid polynucleotide(s) or other polynucleotides of the present invention. In some embodiments, the naked polynucleotide can contain other nucleic acids and/or polynucleotides in addition to the engineered AAV capsid polynucleotide(s) or other polynucleotides of the present invention described elsewhere herein. The naked polynucleotides can include one or more elements of a transposon system. Transposons and system thereof are described in greater detail elsewhere herein.

### Non-Viral Polynucleotide Vectors

In some embodiments, one or more of the engineered AAV capsid polynucleotides or other polynucleotides of the present invention can be included in a non-viral polynucleotide vector. Suitable non-viral polynucleotide vectors include, but are not limited to, transposon vectors and vector systems, plasmids, bacterial artificial chromosomes, yeast artificial chromosomes, AR (antibiotic resistance)-free plasmids and miniplasmids, circular covalently closed vectors (e.g., minicircles, minivectors, miniknots,), linear covalently closed vectors (“dumbbell shaped”), MIDGE (minimalistic immunologically defined gene expression) vectors, MiLV (micro-linear vector) vectors, Ministrings, mini-intronic plasmids, PSK systems (post-segregationally killing systems), ORT (operator repressor titration) plasmids, and the like. See e.g., Hardee et al. 2017. Genes. 8 (2): 65.

In some embodiments, the non-viral polynucleotide vector can have a conditional origin of replication. In some embodiments, the non-viral polynucleotide vector can be an ORT plasmid. In some embodiments, the non-viral polynucleotide vector can have a minimalistic immunologically defined gene expression. In some embodiments, the non-viral polynucleotide vector can have one or more post-segregationally killing system genes. In some embodiments, the non-viral polynucleotide vector is AR-free. In some embodiments, the non-viral polynucleotide vector is a minivector. In some embodiments, the non-viral polynucleotide vector includes a nuclear localization signal. In some embodiments, the non-viral polynucleotide vector can include one or more CpG motifs. In some embodiments, the non-viral polynucleotide vectors can include one or more scaffold/matrix attachment regions (S/MARs). See e.g., Mirkovitch et al. 1984. Cell. 39:223-232, Wong et al. 2015. Adv. Genet. 89:113-152, whose techniques and vectors can be adapted for use in the present invention. S/MARs are AT-rich sequences that play a role in the spatial organization of chromosomes through DNA loop base attachment to the nuclear matrix. S/MARs are often found close to regulatory elements such as promoters, enhancers, and origins of DNA replication. Inclusion of one or S/MARs can facilitate a once-per-cell-cycle replication to maintain the non-viral polynucleotide vector as an episome in daughter cells. In embodiments, the S/MAR sequence is located downstream of an actively transcribed polynucleotide (e.g., one or more engineered AAV capsid polynucleotides or other polynucleotides or molecules of the present invention) included in the non-viral polynucleotide vector. In some embodiments, the S/MAR can be a S/MAR from the beta-interferon gene cluster. See e.g., Verghese et al. 2014. Nucleic Acid Res. 42: e53; Xu et al. 2016. Sci. China Life Sci. 59:1024-1033; Jin et al. 2016. 8:702-711; Koirala et al. 2014. Adv. Exp. Med. Biol. 801:703-709; and Nehlsen et al. 2006. Gene Ther. Mol. Biol. 10:233-244, whose techniques and vectors can be adapted for use in the present invention.

In some embodiments, the non-viral vector is a transposon vector or system thereof. As used herein, “transposon” (also referred to as transposable element) refers to a polynucleotide sequence that is capable of moving form location in a genome to another. There are several classes of transposons. Transposons include retrotransposons and DNA transposons. Retrotransposons require the transcription of the polynucleotide that is moved (or transposed) in order to transpose the polynucleotide to a new genome or polynucleotide. DNA transposons are those that do not require reverse transcription of the polynucleotide that is moved (or transposed) in order to transpose the polynucleotide to a new genome or polynucleotide. In some embodiments, the non-viral polynucleotide vector can be a retrotransposon vector. In some embodiments, the retrotransposon vector includes long terminal repeats. In some embodiments, the retrotransposon vector does not include long terminal repeats. In some embodiments, the non-viral polynucleotide vector can be a DNA transposon vector. DNA transposon vectors can include a polynucleotide sequence encoding a transposase. In some embodiments, the transposon vector is configured as a non-autonomous transposon vector, meaning that the transposition does not occur spontaneously on its own. In some of these embodiments, the transposon vector lacks one or more polynucleotide sequences encoding proteins required for transposition. In some embodiments, the non-autonomous transposon vectors lack one or more Ac elements.

In some embodiments a non-viral polynucleotide transposon vector system can include a first polynucleotide vector that contains the engineered AAV capsid polynucleotide(s) or other polynucleotides, or molecules of the present invention described herein flanked on the 5′ and 3′ ends by transposon terminal inverted repeats (TIRs) and a second polynucleotide vector that includes a polynucleotide capable of encoding a transposase coupled to a promoter to drive expression of the transposase. When both are expressed in the same cell the transposase can be expressed from the second vector and can transpose the material between the TIRs on the first vector (e.g., the engineered AAV capsid polynucleotide(s) or other polynucleotides or molecules of the present invention) and integrate it into one or more positions in the host cell's genome. In some embodiments the transposon vector or system thereof can be configured as a gene trap. In some embodiments, the TIRs can be configured to flank a strong splice acceptor site followed by a reporter and/or other gene (e.g., one or more of the engineered AAV capsid polynucleotide(s) or other polynucleotides or molecules of the present invention) and a strong poly A tail. When transposition occurs while using this vector or system thereof, the transposon can insert into an intron of a gene and the inserted reporter or other gene can provoke a mis-splicing process and as a result it in activates the trapped gene.

Any suitable transposon system can be used. Suitable transposon and systems thereof can include Sleeping Beauty transposon system (Tc1/mariner superfamily) (see e.g., Ivics et al. 1997. Cell. 91 (4): 501-510), piggyBac (piggyBac superfamily) (see e.g., Li et al. 2013 110 (25): E2279-E2287 and Yusa et al. 2011. PNAS. 108 (4): 1531-1536), Tol2 (superfamily hAT), Frog Prince (Tc1/mariner superfamily) (see e.g., Miskey et al. 2003 Nucleic Acid Res. 31 (23): 6873-6881) and variants thereof.

### Chemical Carriers

In some embodiments the engineered AAV capsid polynucleotide(s) or other polynucleotides or other molecules of the present invention described herein can be coupled to a chemical carrier. Chemical carriers that can be suitable for delivery of polynucleotides can be broadly classified into the following classes: (i) inorganic particles, (ii) lipid-based, (iii) polymer-based, and (iv) peptide based. They can be categorized as (1) those that can form condensed complexes with a polynucleotide (such as the engineered AAV capsid polynucleotide(s) of the present invention), (2) those capable of targeting specific cells, (3) those capable of increasing delivery of the polynucleotide or other molecules (such as the engineered AAV capsid polynucleotide(s)) of the present invention to the nucleus or cytosol of a host cell, (4) those capable of disintegrating from DNA/RNA in the cytosol of a host cell, and (5) those capable of sustained or controlled release. It will be appreciated that any one given chemical carrier can include features from multiple categories. The term “particle” as used herein, refers to any suitable sized particles for delivery of the compositions (including particles, polypeptides, polynucleotides, and other compositions described herein) present invention described herein. Suitable sizes include macro-, micro-, and nano-sized particles.

In some embodiments, the non-viral carrier can be an inorganic particle. In some embodiments, the inorganic particle, can be a nanoparticle. The inorganic particles can be configured and optimized by varying size, shape, and/or porosity. In some embodiments, the inorganic particles are optimized to escape from the reticulo endothelial system. In some embodiments, the inorganic particles can be optimized to protect an entrapped molecule from degradation. The suitable inorganic particles that can be used as non-viral carriers in this context can include, but are not limited to, calcium phosphate, silica, metals (e.g., gold, platinum, silver, palladium, rhodium, osmium, iridium, ruthenium, mercury, copper, rhenium, titanium, niobium, tantalum, and combinations thereof), magnetic compounds, particles, and materials, (e.g., supermagnetic iron oxide and magnetite), quantum dots, fullerenes (e.g., carbon nanoparticles, nanotubes, nanostrings, and the like), and combinations thereof. Other suitable inorganic non-viral carriers are discussed elsewhere herein.

In some embodiments, the non-viral carrier can be lipid-based. Suitable lipid-based carriers are also described in greater detail herein. In some embodiments, the lipid-based carrier includes a cationic lipid or an amphiphilic lipid that is capable of binding or otherwise interacting with a negative charge on the polynucleotide to be delivered (e.g., such as an engineered AAV capsid polynucleotide of the present invention). In some embodiments, chemical non-viral carrier systems can include a polynucleotide (such as the engineered AAV capsid polynucleotide(s)) or other composition or molecule of the present invention) and a lipid (such as a cationic lipid). These are also referred to in the art as lipoplexes. Other embodiments of lipoplexes are described elsewhere herein. In some embodiments, the non-viral lipid-based carrier can be a lipid nano emulsion. Lipid nano emulsions can be formed by the dispersion of an immiscible liquid in another stabilized emulsifying agent and can have particles of about 200 nm that are composed of the lipid, water, and surfactant that can contain the polynucleotide to be delivered (e.g., the engineered AAV capsid polynucleotide(s) of the present invention). In some embodiments, the lipid-based non-viral carrier can be a solid lipid particle or nanoparticle.

In some embodiments, the non-viral carrier can be peptide-based. In some embodiments, the peptide-based non-viral carrier can include one or more cationic amino acids. In some embodiments, 35 to 40, 45, 50, 55, 60, 65, 70, 75, 80, 85, 90, 95, 99 or 100% of the amino acids are cationic. In some embodiments, peptide carriers can be used in conjunction with other types of carriers (e.g., polymer-based carriers and lipid-based carriers to functionalize these carriers). In some embodiments, the functionalization is targeting a host cell. Suitable polymers that can be included in the polymer-based non-viral carrier can include, but are not limited to, polyethylenimine (PEI), chitosan, poly (DL-lactide) (PLA), poly (DL-Lactide-co-glycoside) (PLGA), dendrimers (see e.g., US Pat. Pub. 2017/0079916 whose techniques and compositions can be adapted for use with the engineered AAV capsid polynucleotides of the present invention), polymethacrylate, and combinations thereof.

In some embodiments, the non-viral carrier can be configured to release an engineered delivery system polynucleotide that is associated with or attached to the non-viral carrier in response to an external stimulus, such as pH, temperature, osmolarity, concentration of a specific molecule or composition (e.g., calcium, NaCl, and the like), pressure and the like. In some embodiments, the non-viral carrier can be a particle that is configured includes one or more of the engineered AAV capsid polynucleotides or other compositions of the present invention describe herein and an environmental triggering agent response element, and optionally a triggering agent. In some embodiments, the particle can include a polymer that can be selected from the group of polymethacrylates and polyacrylates. In some embodiments, the non-viral particle can include one or more embodiments of the compositions microparticles described in US Pat. Pubs. 20150232883 and 20050123596, whose techniques and compositions can be adapted for use in the present invention.

In some embodiments, the non-viral carrier can be a polymer-based carrier. In some embodiments, the polymer is cationic or is predominantly cationic such that it can interact in a charge-dependent manner with the negatively charged polynucleotide to be delivered (such as the engineered AAV capsid polynucleotide(s) of the present invention). Polymer-based systems are described in greater detail elsewhere herein.

### Viral Vectors

In some embodiments, the vector is a viral vector. The term of art “viral vector” and as used herein in this context refers to polynucleotide based vectors that contain one or more elements from or based upon one or more elements of a virus that can be capable of expressing and packaging a polynucleotide, such as an engineered AAV capsid polynucleotide, cargo, or other composition or molecule of the present invention, into a virus particle and producing said virus particle when used alone or with one or more other viral vectors (such as in a viral vector system). Viral vectors and systems thereof can be used for producing viral particles for delivery of and/or expression and/or generation of one or more compositions of the present invention described herein (including, but not limited to, any viral particle and associated cargo). The viral vector can be part of a viral vector system involving multiple vectors. In some embodiments, systems incorporating multiple viral vectors can increase the safety of these systems. Suitable viral vectors can include adenoviral-based vectors, adeno associated vectors, helper-dependent adenoviral (HdAd) vectors, hybrid adenoviral vectors, and the like. Other embodiments of viral vectors and viral particles produce therefrom are described elsewhere herein. In some embodiments, the viral vectors are configured to produce replication incompetent viral particles for improved safety of these systems.

### Adenoviral Vectors, Helper-Dependent Adenoviral Vectors, and Hybrid Adenoviral Vectors

In some embodiments, the vector can be an adenoviral vector. In some embodiments, the adenoviral vector can include elements such that the virus particle produced using the vector or system thereof can be serotype 2, 5, or 9. In some embodiments, the polynucleotide to be delivered via the adenoviral particle can be up to about 8 kb. Thus, in some embodiments, an adenoviral vector can include a DNA polynucleotide to be delivered that can range in size from about 0.001 kb to about 8 kb. Adenoviral vectors have been used successfully in several contexts (see e.g., Teramato et al. 2000. Lancet. 355:1911-1912; Lai et al. 2002. DNA Cell. Biol. 21:895-913; Flotte et al., 1996. Hum. Gene. Ther. 7:1145-1159; and Kay et al. 2000. Nat. Genet. 24:257-261. The engineered AAV capsids can be included in an adenoviral vector to produce adenoviral particles containing said engineered AAV capsids.

In some embodiments the vector can be a helper-dependent adenoviral vector or system thereof. These are also referred to in the field as “gutless” or “gutted” vectors and are a modified generation of adenoviral vectors (see e.g., Thrasher et al. 2006. Nature. 443: E5-7). In embodiments of the helper-dependent adenoviral vector system one vector (the helper) can contain all the viral genes required for replication but contains a conditional gene defect in the packaging domain. The second vector of the system can contain only the ends of the viral genome, one or more engineered AAV capsid polynucleotides, and the native packaging recognition signal, which can allow selective packaged release from the cells (see e.g., Cideciyan et al. 2009. N Engl J Med. 361:725-727). Helper-dependent Adenoviral vector systems have been successful for gene delivery in several contexts (see e.g., Simonelli et al. 2010. J Am Soc Gene Ther. 18:643-650; Cideciyan et al. 2009. N Engl J Med. 361:725-727; Crane et al. 2012. Gene Ther. 19 (4): 443-452; Alba et al. 2005. Gene Ther. 12:18-S27; Croyle et al. 2005. Gene Ther. 12:579-587; Amalfitano et al. 1998. J. Virol. 72:926-933; and Morral et al. 1999. PNAS. 96:12816-12821). The techniques and vectors described in these publications can be adapted for inclusion and delivery of the engineered AAV capsid polynucleotides described herein. In some embodiments, the polynucleotide to be delivered via the viral particle produced from a helper-dependent adenoviral vector or system thereof can be up to about 38 kb. Thus, in some embodiments, a adenoviral vector can include a DNA polynucleotide to be delivered that can range in size from about 0.001 kb to about 37 kb (see e.g., Rosewell et al. 2011. J. Genet. Syndr. Gene Ther. Suppl. 5:001).

In some embodiments, the vector is a hybrid-adenoviral vector or system thereof. Hybrid adenoviral vectors are composed of the high transduction efficiency of a gene-deleted adenoviral vector and the long-term genome-integrating potential of adeno-associated, retroviruses, lentivirus, and transposon based-gene transfer. In some embodiments, such hybrid vector systems can result in stable transduction and limited integration site. See e.g., Balague et al. 2000. Blood. 95:820-828; Morral et al. 1998. Hum. Gene Ther. 9:2709-2716; Kubo and Mitani. 2003. J. Virol. 77 (5): 2964-2971; Zhang et al. 2013. PloS One. 8 (10) e76771; and Cooney et al. 2015. Mol. Ther. 23 (4): 667-674), whose techniques and vectors described therein can be modified and adapted for use in the engineered AAV capsid system of the present invention. In some embodiments, a hybrid-adenoviral vector can include one or more features of a retrovirus and/or an adeno-associated virus. In some embodiments the hybrid-adenoviral vector can include one or more features of a spuma retrovirus or foamy virus (FV). See e.g., Ehrhardt et al. 2007. Mol. Ther. 15:146-156 and Liu et al. 2007. Mol. Ther. 15:1834-1841, whose techniques and vectors described therein can be modified and adapted for use in the engineered AAV capsid system of the present invention. Advantages of using one or more features from the FVs in the hybrid-adenoviral vector or system thereof can include the ability of the viral particles produced therefrom to infect a broad range of cells, a large packaging capacity as compared to other retroviruses, and the ability to persist in quiescent (non-dividing) cells. See also e.g., Ehrhardt et al. 2007. Mol. Ther. 156:146-156 and Shuji et al. 2011. Mol. Ther. 19:76-82, whose techniques and vectors described therein can be modified and adapted for use in the engineered AAV capsid system of the present invention.

### Adeno Associated Vectors

In an embodiment, the engineered vector or system thereof can be an adeno-associated vector (AAV). See, e.g., West et al., Virology 160:38-47 (1987); U.S. Pat. No. 4,797,368; WO 93/24641; Kotin, Human Gene Therapy 5:793-801 (1994); and Muzyczka, J. Clin. Invest. 94:1351 (1994). Although similar to adenoviral vectors in some of their features, AAVs have some deficiency in their replication and/or pathogenicity and thus can be safer that adenoviral vectors. In some embodiments the AAV can integrate into a specific site on chromosome 19 of a human cell with no observable side effects. In some embodiments, the capacity of the AAV vector, system thereof, and/or AAV particles can be up to about 4.7 kb. The AAV vector or system thereof can include one or more engineered capsid polynucleotides described herein.

The AAV vector or system thereof can include one or more regulatory molecules. In some embodiments the regulatory molecules can be promoters, enhancers, repressors and the like, which are described in greater detail elsewhere herein. In some embodiments, the AAV vector or system thereof can include one or more polynucleotides that can encode one or more regulatory proteins. In some embodiments, the one or more regulatory proteins can be selected from Rep78, Rep68, Rep52, Rep40, variants thereof, and combinations thereof. In some embodiments, the promoter can be a tissue specific promoter as previously discussed. In some embodiments, the tissue specific promoter can drive expression of an engineered capsid AAV capsid polynucleotide described herein.

The AAV vector or system thereof can include one or more polynucleotides that can encode one or more capsid proteins, such as the engineered AAV capsid proteins described elsewhere herein. The engineered capsid proteins can be capable of assembling into a protein shell (an engineered capsid) of the AAV virus particle. The engineered capsid can have a cell-, tissue-, and/or organ-specific tropism.

In some embodiments, the AAV vector or system thereof can include one or more adenovirus helper factors or polynucleotides that can encode one or more adenovirus helper factors. Such adenovirus helper factors can include, but are not limited, E1A, E1B, E2A, E4ORF6, and VA RNAs. In some embodiments, a producing host cell line expresses one or more of the adenovirus helper factors. In some embodiments the cargo is encoded on a rAAV artificial genome operably linked to regulatory sequence and flanked by AAV ITR sequences.

The AAV vector or system thereof can be configured to produce AAV particles having a modified capsid comprising a n-mer as described herein that provides the AAV with a specific serotype that differs from an unmodified AAV capsid. In some embodiments, the serotype can be AAV-1, AAV-2, AAV-3, AAV-4, AAV-5, AAV-6, AAV-8, AAV-9 or any combinations thereof. In some embodiments, the AAV can be AAV1, AAV-2, AAV-5, AAV-9 or any combination thereof. One can select the AAV of the AAV with regard to the cells to be targeted; e.g., one can select AAV serotypes 1, 2, 5, 9 or a hybrid capsid AAV-1, AAV-2, AAV-5, AAV-9 or any combination thereof for targeting brain and/or neuronal cells; and one can select AAV-4 for targeting cardiac tissue; and one can select AAV-8 for delivery to the liver. Thus, in some embodiments, an AAV vector or system thereof capable of producing AAV particles capable of targeting the brain and/or neuronal cells can be configured to generate AAV particles having serotypes 1, 2, 5 or a hybrid capsid AAV-1, AAV-2, AAV-5 or any combination thereof. In some embodiments, an AAV vector or system thereof capable of producing AAV particles capable of targeting cardiac tissue can be configured to generate an AAV particle having an AAV-4 serotype. In some embodiments, an AAV vector or system thereof capable of producing AAV particles capable of targeting the liver can be configured to generate an AAV having an AAV-8 serotype. See also Srivastava. 2017. Curr. Opin. Virol. 21:75-80.

It will be appreciated that while the different serotypes can provide some level of cell, tissue, and/or organ specificity, each serotype still is multi-tropic and thus can result in tissue-toxicity if using that serotype to target a tissue that the serotype is less efficient in transducing. Thus, in addition to achieving some tissue targeting capacity via selecting an AAV of a particular serotype, it will be appreciated that the tropism of the AAV serotype can be modified by an engineered AAV capsid described herein. As described elsewhere herein, variants of wild-type AAV of any serotype can be generated via a method described herein and determined to have a particular cell-specific tropism, which can be the same or different as that of the reference wild-type AAV serotype. In some embodiments, the cell, tissue, and/or specificity of the wild-type serotype can be enhanced (e.g., made more selective or specific for a particular cell type that the serotype is already biased towards). For example, wild-type AAV-9 is biased towards muscle and brain in humans (see e.g., Srivastava. 2017. Curr. Opin. Virol. 21:75-80.) By including an engineered AAV capsid and/or capsid protein variant of wild-type AAV-9 as described herein, the bias for e.g., brain can be reduced or eliminated and/or the septicity increased such that the brain specificity appears reduced in comparison, thus enhancing the specificity for the muscle as compared to the wild-type AAV-9. As previously mentioned, inclusion of an engineered capsid and/or capsid protein variant of a wild-type AAV serotype can have a different tropism than the wild-type reference AAV serotype. For example, an engineered AAV capsid and/or capsid protein variant of AAV-9 can have specificity for a tissue other than muscle or brain in humans.

In some embodiments, the AAV vector is a hybrid AAV vector or system thereof. Hybrid AAVs are AAVs that include genomes with elements from one serotype that are packaged into a capsid derived from at least one different serotype. For example, if it is the rAAV2/5 that is to be produced, and if the production method is based on the helper-free, transient transfection method discussed above, the 1st plasmid and the 3rd plasmid (the adeno helper plasmid) will be the same as discussed for rAAV2 production. However, the 2nd plasmid, the pRepCap will be different. In this plasmid, called pRep2/Cap5, the Rep gene is still derived from AAV2, while the Cap gene is derived from AAV5. The production scheme is the same as the above-mentioned approach for AAV2 production. The resulting rAAV is called rAAV2/5, in which the genome is based on recombinant AAV2, while the capsid is based on AAV5. It is assumed the cell or tissue-tropism displayed by this AAV2/5 hybrid virus should be the same as that of AAV5. It will be appreciated that wild-type hybrid AAV particles suffer the same specificity issues as with the non-hybrid wild-type serotypes previously discussed.

Advantages achieved by the wild-type based hybrid AAV systems can be combined with the increased and customizable cell-specificity that can be achieved with the engineered AAV capsids can be combined by generating a hybrid AAV that can include an engineered AAV capsid described elsewhere herein. It will be appreciated that hybrid AAVs can contain an engineered AAV capsid containing a genome with elements from a different serotype than the reference wild-type serotype that the engineered AAV capsid is a variant of. For example, a hybrid AAV can be produced that includes an engineered AAV capsid that is a variant of an AAV-9 serotype that is used to package a genome that contains components (e.g., rep elements) from an AAV-2 serotype. As with wild-type based hybrid AAVs previously discussed, the tropism of the resulting AAV particle will be that of the engineered AAV capsid.

A tabulation of certain wild-type AAV serotypes as to these cells can be found in Grimm, D. et al, J. Virol. 82:5887-5911 (2008) reproduced below as Table A. Further tropism details can be found in Srivastava. 2017. Curr. Opin. Virol. 21:75-80 as previously discussed.

TABLE A

| Cell Line | AAV-1 | AAV-2 | AAV-3 | AAV-4 | AAV-5 | AAV-6 | AAV-8 | AAV-9 |
|---|---|---|---|---|---|---|---|---|
| Huh-7 | 13 | 100 | 2.5 | 0.0 | 0.1 | 10 | 0.7 | 0.0 |
| HEK293 | 25 | 100 | 2.5 | 0.1 | 0.1 | 5 | 0.7 | 0.1 |
| HeLa | 3 | 100 | 2.0 | 0.1 | 6.7 | 1 | 0.2 | 0.1 |
| HepG2 | 3 | 100 | 16.7 | 0.3 | 1.7 | 5 | 0.3 | ND |
| Hep1A | 20 | 100 | 0.2 | 1.0 | 0.1 | 1 | 0.2 | 0.0 |
| 911 | 17 | 100 | 11 | 0.2 | 0.1 | 17 | 0.1 | ND |
| CHO | 100 | 100 | 14 | 1.4 | 333 | 50 | 10 | 1.0 |
| COS | 33 | 100 | 33 | 3.3 | 5.0 | 14 | 2.0 | 0.5 |
| MeWo | 10 | 100 | 20 | 0.3 | 6.7 | 10 | 1.0 | 0.2 |
| NIH3T3 | 10 | 100 | 2.9 | 2.9 | 0.3 | 10 | 0.3 | ND |
| A549 | 14 | 100 | 20 | ND | 0.5 | 10 | 0.5 | 0.1 |
| HT1180 | 20 | 100 | 10 | 0.1 | 0.3 | 33 | 0.5 | 0.1 |
| Monocytes | 1111 | 100 | ND | ND | 125 | 1429 | ND | ND |
| Immature DC | 2500 | 100 | ND | ND | 222 | 2857 | ND | ND |
| Mature DC | 2222 | 100 | ND | ND | 333 | 3333 | ND | ND |

In example embodiments, the AAV vector or system thereof is AAV rh.74 or AAV rh.10.

In example embodiments, the AAV vector or system thereof is configured as a “gutless” vector, similar to that described in connection with a retroviral vector. In some embodiments, the “gutless” AAV vector or system thereof can have the cis-acting viral DNA elements involved in genome amplification and packaging in linkage with the heterologous sequences of interest (e.g., the engineered AAV capsid polynucleotide(s)).

### Vector Construction

The vectors described herein can be constructed using any suitable process or technique. In some embodiments, one or more suitable recombination and/or cloning methods or techniques can be used to the vector(s) described herein. Suitable recombination and/or cloning techniques and/or methods can include, but not limited to, those described in U.S. Application publication No. US 2004-0171156 A1. Other suitable methods and techniques are described elsewhere herein.

Construction of recombinant AAV vectors is described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989). Any of the techniques and/or methods can be used and/or adapted for constructing an AAV or other vector described herein. AAV vectors are discussed elsewhere herein.

In some embodiments, the vector can have one or more insertion sites, such as a restriction endonuclease recognition sequence (also referred to as a “cloning site”). In some embodiments, one or more insertion sites (e.g., about or more than about 1, 2, 3, 4, 5, 6, 7, 8, 9, 10, or more insertion sites) are located upstream and/or downstream of one or more sequence elements of one or more vectors.

Delivery vehicles, vectors, particles, nanoparticles, formulations and components thereof for expression of one or more elements of an engineered AAV capsid system described herein are as used in the foregoing documents, such as International Patent Application Publication WO 2014/093622 (PCT/US2013/074667) and are discussed in greater detail herein.

Virus Particle Production from Viral Vectors

### AAV Particle Production

There are two main strategies for producing AAV particles from AAV vectors and systems thereof, such as those described herein, which depend on how the adenovirus helper factors are provided (helper v. helper free). In some embodiments, a method of producing AAV particles from AAV vectors and systems thereof can include adenovirus infection into cell lines that stably harbor AAV replication and capsid encoding polynucleotides along with AAV vector containing the polynucleotide to be packaged and delivered by the resulting AAV particle (e.g., the engineered AAV capsid polynucleotide(s)). In some embodiments, a method of producing AAV particles from AAV vectors and systems thereof can be a “helper free” method, which includes co-transfection of an appropriate producing cell line with three vectors (e.g., plasmid vectors): (1) an AAV vector that contains a polynucleotide of interest between 2 ITRs; (2) a vector that carries the AAV Rep-Cap encoding polynucleotides (i.e., including the engineered AAV capsid polynucleotide); and (3) helper polynucleotides. One of skill in the art will appreciate various methods and variations thereof that are both helper and -helper free and as well as the different advantages of each system.

The engineered AAV vectors and systems thereof described herein can be produced by any of these methods.

### Vector and Virus Particle Delivery

A vector (including non-viral carriers) described herein can be introduced into host cells to thereby produce transcripts, proteins, or peptides, including fusion proteins or peptides encoded by nucleic acids as described herein (e.g., engineered AAV capsid system transcripts, proteins, enzymes, mutant forms thereof, fusion proteins thereof, etc.), and virus particles (such as from viral vectors and systems thereof).

One or more engineered rAAVs having an engineered capsid as described herein can be delivered using adeno associated virus (AAV), adenovirus or other plasmid or viral vector types as previously described, in particular, using formulations and doses from, for example, U.S. Pat. Nos. 8,454,972 (formulations, doses for adenovirus), 8,404,658 (formulations, doses for AAV) and 5,846,946 (formulations, doses for DNA plasmids) and from clinical trials and publications regarding the clinical trials involving lentivirus, AAV and adenovirus. For examples, for AAV, the route of administration, formulation and dose can be as in U.S. Pat. No. 8,454,972 and as in clinical trials involving AAV. For Adenovirus, the route of administration, formulation and dose can be as in U.S. Pat. No. 8,404,658 and as in clinical trials involving adenovirus.

For plasmid delivery, the route of administration, formulation and dose can be as in U.S. Pat. No. 5,846,946 and as in clinical studies involving plasmids. In some embodiments, doses can be based on or extrapolated to an average 70 kg individual (e.g., a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. The viral vectors can be injected into or otherwise delivered to the tissue or cell of interest.

In terms of in vivo delivery, AAV is advantageous over other viral vectors for a couple of reasons such as low toxicity (this may be due to the purification method not requiring ultra-centrifugation of cell particles that can activate the immune response) and a low probability of causing insertional mutagenesis because it doesn't integrate into the host genome.

The vector(s) and virus particles described herein can be delivered into a host cell in vitro, in vivo, and or ex vivo. Delivery can occur by any suitable method including, but not limited to, physical methods, chemical methods, and biological methods. Physical delivery methods are those methods that employ physical force to counteract the membrane barrier of the cells to facilitate intracellular delivery of the vector. Suitable physical methods include, but are not limited to, needles (e.g., injections), ballistic polynucleotides (e.g., particle bombardment, micro projectile gene transfer, and gene gun), electroporation, sonoporation, photoporation, magnetofection, hydroporation, and mechanical massage. Chemical methods are those methods that employ a chemical to elicit a change in the cells membrane permeability or other characteristic(s) to facilitate entry of the vector into the cell. For example, the environmental pH can be altered which can elicit a change in the permeability of the cell membrane. Biological methods are those that rely and capitalize on the host cell's biological processes or biological characteristics to facilitate transport of the vector (with or without a carrier) into a cell. For example, the vector and/or its carrier can stimulate an endocytosis or similar process in the cell to facilitate uptake of the vector into the cell.

Delivery of engineered AAV capsid system components (e.g., polynucleotides encoding engineered AAV capsid and/or capsid proteins) to cells via particles. The term “particle” as used herein, refers to any suitable sized particles for delivery of the engineered AAV capsid system components described herein. Suitable sizes include macro-, micro-, and nano-sized particles. In some embodiments, any of the of the engineered AAV capsid system components (e.g., polypeptides, polynucleotides, vectors and combinations thereof described herein) can be attached to, coupled to, integrated with, otherwise associated with one or more particles or component thereof as described herein. The particles described herein can then be administered to a cell or organism by an appropriate route and/or technique. In some embodiments, particle delivery can be selected and be advantageous for delivery of the polynucleotide or vector components. It will be appreciated that in embodiments, particle delivery can also be advantageous for other engineered capsid system molecules and formulations described elsewhere herein.

### Engineered Virus Particles Including an Engineered Viral (e.g., AAV) Capsid

Also described herein are engineered virus particles (also referred to here and elsewhere herein as “engineered viral particles” that can contain an engineered viral capsid (e.g., AAV capsid, referred to as “engineered AAV particles”) as described in detail elsewhere herein. It will be appreciated that the engineered AAV particles can be adenovirus-based particles, helper adenovirus-based particles, AAV-based particles, or hybrid adenovirus-based particles that contain at least one engineered AAV capsid proteins as previously described. An engineered AAV capsid is one that that contains one or more engineered AAV capsid proteins as are described elsewhere herein. In some embodiments, the engineered AAV particles can include 1-60 engineered AAV capsid proteins described herein. In some embodiments, the engineered AAV particles can contain 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 engineered capsid proteins. In some embodiments, the engineered AAV particles can contain 0-59 wild-type AAV capsid proteins. In some embodiments, the engineered AAV particles can contain 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, or 59 wild-type AAV capsid proteins. The engineered AAV particles can thus include one or more n-mer motifs as is previously described.

The engineered AAV particle can include one or more cargo polynucleotides. Cargo polynucleotides are discussed in greater detail elsewhere herein. Methods of making the engineered AAV particles from viral and non-viral vectors are described elsewhere herein. Formulations containing the engineered virus particles are described elsewhere herein.

### Example Cargos

The n-mers can be coupled to or otherwise associated with a cargo. Cargos can include any molecule that is capable of being coupled to or associated with the n-mers described herein. Cargos can include, without limitation, nucleotides, oligonucleotides, polynucleotides, amino acids, peptides, polypeptides, riboproteins, lipids, sugars, pharmaceutically active agents (e.g., drugs, imaging and other diagnostic agents, and the like), chemical compounds, and combinations thereof. In some embodiments, the cargo is DNA, RNA, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatoires, anti-histamines, anti-infectives, radiation sensitizers, chemotherapeutics, radioactive compounds, imaging agents, and combinations thereof.

In some embodiments, the cargo is capable of treating or preventing a neurological disease or disorder, details of which are described herein.

In some embodiments, the cargo is a morpholino, a peptide-linked morpholino, an antisense oligonucleotide, a PMO, a therapeutic transgene, a polynucleotide encoding a therapeutic polypeptide or peptide, a PPMO, one or more peptides, one or more polynucleotides encoding a CRISPR-Cas protein, a guide RNA, or both, a ribonucleoprotein, wherein the ribonucleoprotein comprises a CRISPR-Cas system molecule, a therapeutic transgene RNA, or other gene modifying or therapeutic RNA and/or protein, or any combination thereof.

In some embodiments, one or more n-mers described herein is directly attached to the cargo. In some embodiments, one or more n-mers described herein is indirectly coupled to the cargo, such as via a linker molecule. In some embodiments, one or more one or more n-mers described herein is coupled to associated with a polypeptide or other particle that is coupled to, attached to, encapsulates, and/or contains a cargo.

Exemplary particles include, without limitation, viral particles (e.g., viral capsids, which is inclusive of bacteriophage capsids), polysomes, liposomes, nanoparticles, microparticles, exosomes, micelles, and the like. The term “nanoparticle” as used herein includes a nanoscale deposit of a homogenous or heterogeneous material. Nanoparticles may be regular or irregular in shape and may be formed from a plurality of co-deposited particles that form a composite nanoscale particle. Nanoparticles may be generally spherical in shape or have a composite shape formed from a plurality of co-deposited generally spherical particles. Exemplary shapes for the nanoparticles include, but are not limited to, spherical, rod, elliptical, cylindrical, disc, and the like. In some embodiments, the nanoparticles have a substantially spherical shape.

### Cargo Polynucleotides

Cargos are also described elsewhere herein. In some embodiments, the cargo is a cargo polynucleotide that can be packaged into an engineered viral particle and subsequently delivered to a cell. In some embodiments, delivery is cell selective, e.g., neurons and glial cells of the central nervous system. The engineered viral (e.g., AAV) capsid polynucleotides, other viral (e.g., AAV) polynucleotide(s), and/or vector polynucleotides can contain one or more cargo polynucleotides. In some embodiments, the one or more cargo polynucleotides can be operably linked to the engineered viral (e.g., AAV) capsid polynucleotide(s) and can be part of the engineered viral (e.g., AAV) genome of the viral (e.g., AAV) system of the present invention. The cargo polynucleotides can be packaged into an engineered viral (e.g., AAV) particle, which can be delivered to, e.g., a cell. In some embodiments, the cargo polynucleotide can be capable of modifying a polynucleotide (e.g., gene or transcript) of a cell to which it is delivered. As used herein, “gene” can refer to a hereditary unit corresponding to a sequence of DNA that occupies a specific location on a chromosome and that contains the genetic instruction for a characteristic(s) or trait(s) in an organism. The term gene can refer to translated and/or untranslated regions of a genome. “Gene” can refer to the specific sequence of DNA that is transcribed into an RNA transcript that can be translated into a polypeptide or be a catalytic RNA molecule, including but not limited to, tRNA, siRNA, piRNA, miRNA, long-non-coding RNA and shRNA. Polynucleotide, gene, transcript, etc. modification includes all genetic engineering techniques including, but not limited to, gene editing as well as conventional recombinational gene modification techniques (e.g., whole or partial gene insertion, deletion, and mutagenesis (e.g., insertional and deletional mutagenesis) techniques.

In example embodiments, the cargo molecule is a polynucleotide that is or can encode a vaccine. In example embodiments, the cargo molecule is a polynucleotide encoding an antibody.

### Gene Modification Cargo Polynucleotides

In some embodiments, the cargo molecule can be a polynucleotide or polypeptide that can alone or when delivered as part of a system, whether or not delivered with other components of the system, operate to modify the genome, epigenome, and/or transcriptome of a cell to which it is delivered. Such systems include, but are not limited to, CRISPR-Cas systems. Other gene modification systems, e.g., TALENs, Zinc Finger nucleases, Cre-Lox, morpholinos, etc. are other non-limiting examples of gene modification systems whose one or more components can be delivered by the engineered viral (e.g., AAV) particles described herein.

In some embodiments, the cargo molecule is a gene editing system or component thereof. In some embodiments, the cargo molecule is a CRISPR-Cas system molecule or a component thereof. In some embodiments, the cargo molecule is a polynucleotide that encodes one or more components of a gene modification system (such as a CRISPR-Cas system). In some embodiments the cargo molecule is a gRNA. CRISPR-Cas system as used herein is intended to encompass by Class 1 and Class 2 CRISPR-Cas systems and derivatives of CRISPR-Cas systems such as base editors, prime editors, and CRISPR-associated transposases (CAST) systems.

In some embodiments, the cargo molecule can be a polynucleotide or polypeptide that can alone or when delivered as part of a system, whether or not delivered with other components of the system, operate to modify the genome, epigenome, and/or transcriptome of a cell to which it is delivered, is such that it treats or prevents a disease, a disorder, or a symptom thereof of a neurologic disease or disorder, and/or viruses (such as single stranded RNA viruses). In some embodiments, the cargo molecule, whether or not delivered with other components of the system, operate to modify the genome, epigenome, and/or transcriptome of a cell to which it is delivered, is such that it treats or prevents a neurological disease or disorder described further herein.

In some embodiments, the cargo molecule, whether or not delivered with other components of the system, operate to modify the genome, epigenome, and/or transcriptome of a cell to which it is delivered, is such that can modify the GAA gene, such as any of those described in US Pat. App. Pub. 20190284555, the contents of which are incorporated by reference as if expressed in their entirety herein and can be adapted for use with the present invention.

In some embodiments, the cargo molecule is an antisense oligomer or RNA molecule, such as those described in U.S. Pat. App. Pub. US20160251398, US20150267202, and US20180216111, the contents of which are incorporated by reference as if expressed in their entirety herein and can be adapted for use with the present invention.

In some embodiments, the cargo molecule can be a peptide-oligomer, conjugate as described in e.g., International Patent Application Publication WO2017106304A1, the contents of which are incorporated by reference as if expressed in their entirety herein and can be adapted for use with the present invention.

An embodiment of the invention encompasses methods of modifying a genomic locus of interest to change gene expression in a cell by introducing into the cell any of the compositions described herein.

An embodiment of the invention is that the above elements are comprised in a single composition or comprised in individual compositions. These compositions may advantageously be applied to a host to elicit a functional effect on the genomic level.

Engineered Cells and Organisms Expressing said Engineered AAV capsids

Described herein are engineered cells that can include one or more of the engineered AAV capsid polynucleotides, polypeptides, vectors, and/or vector systems. In some embodiments, one or more of the engineered AAV capsid polynucleotides can be expressed in the engineered cells. In some embodiments, the engineered cells can be capable of producing engineered AAV capsid proteins and/or engineered AAV capsid particles that are described elsewhere herein. Also described herein are modified or engineered organisms that can include one or more engineered cells described herein. The engineered cells can be engineered to express a cargo molecule (e.g., a cargo polynucleotide) dependently or independently of an engineered AAV capsid polynucleotide as described elsewhere herein.

A wide variety of animals, plants, algae, fungi, yeast, etc. and animal, plant, algae, fungus, yeast cell or tissue systems may be engineered to express one or more nucleic acid constructs of the engineered AAV capsid system described herein using various transformation methods mentioned elsewhere herein. This can produce organisms that can produce engineered AAV capsid particles, such as for production purposes, engineered AAV capsid design and/or generation, and/or model organisms. In some embodiments, the polynucleotide(s) encoding one or more components of the engineered AAV capsid system described herein can be stably or transiently incorporated into one or more cells of a plant, animal, algae, fungus, and/or yeast or tissue system. In some embodiments, one or more of engineered AAV capsid system polynucleotides are genomically incorporated into one or more cells of a plant, animal, algae, fungus, and/or yeast or tissue system. Further embodiments of the modified organisms and systems are described elsewhere herein. In some embodiments, one or more components of the engineered AAV capsid system described herein are expressed in one or more cells of the plant, animal, algae, fungus, yeast, or tissue systems.

### Engineered Cells

Described herein are various embodiments of engineered cells that can include one or more of the engineered AAV capsid system polynucleotides, polypeptides, vectors, and/or vector systems described elsewhere herein. In some embodiments, the cells can express one or more of the engineered AAV capsid polynucleotides and can produce one or more engineered AAV capsid particles, which are described in greater detail herein. Such cells are also referred to herein as “producer cells”. It will be appreciated that these engineered cells are different from “modified cells” described elsewhere herein in that the modified cells are not necessarily producer cells (i.e., they do not make engineered GTA delivery particles) unless they include one or more of the engineered AAV capsid polynucleotides, engineered AAV capsid vectors or other vectors described herein that render the cells capable of producing an engineered AAV capsid particle. Modified cells can be recipient cells of an engineered AAV capsid particles and can, in some embodiments, be modified by the engineered AAV capsid particle(s) and/or a cargo polynucleotide delivered to the recipient cell. Modified cells are discussed in greater detail elsewhere herein. The term modification can be used in connection with modification of a cell that is not dependent on being a recipient cell. For example, isolated cells can be modified prior to receiving an engineered AAV capsid molecule.

In an embodiment, the invention provides a non-human eukaryotic organism; for example, a multicellular eukaryotic organism, including a eukaryotic host cell containing one or more components of an engineered delivery system described herein according to any of the described embodiments. In other embodiments, the invention provides a eukaryotic organism; preferably a multicellular eukaryotic organism, comprising a eukaryotic host cell containing one or more components of an engineered delivery system described herein according to any of the described embodiments. In some embodiments, the organism is a host of AAV.

In particular embodiments, the plants, algae, fungi, yeast, etc., cells or parts obtained are transgenic plants, comprising an exogenous DNA sequence incorporated into the genome of all or part of the cells.

The engineered cell can be a prokaryotic cell. The prokaryotic cell can be bacterial cell. The prokaryotic cell can be an archaea cell. The bacterial cell can be any suitable bacterial cell. Suitable bacterial cells can be from the genus Escherichia, Bacillus, Lactobacillus, Rhodococcus, Rodhobacter, Synechococcus, Synechoystis, Pseudomonas, Psedoaltermonas, Stenotrophamonas, and Streptomyces Suitable bacterial cells include, but are not limited to Escherichia coli cells, Caulobacter crescentus cells, Rodhobacter sphaeroides cells, Psedoaltermonas haloplanktis cells. Suitable strains of bacterial include, but are not limited to BL21 (DE3), DL21 (DE3)-pLysS, BL21 Star-pLysS, BL21-SI, BL21-AI, Tuner, Tuner pLysS, Origami, Origami B pLysS, Rosetta, Rosetta pLysS, Rosetta-gami-pLysS, BL21 CodonPlus, AD494, BL2trxB, HMS174, NovaBlue (DE3), BLR, C41 (DE3), C43 (DE3), Lemo21 (DE3), Shuffle T7, ArcticExpress and ArticExpress (DE3).

The engineered cell can be a eukaryotic cell. The eukaryotic cells may be those of or derived from a particular organism, such as a plant or a mammal, including but not limited to human, or non-human eukaryote or animal or mammal as herein discussed, e.g., mouse, rat, rabbit, dog, livestock, or non-human mammal or primate. In some embodiments the engineered cell can be a cell line. Examples of cell lines include, but are not limited to, C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, CIR, Rat6, CVI, RPTE, A10, T24, J82, A375, ARH-77, Calul, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-1 cells, BEAS-2B, bEnd.3, BHK-21, BR 293, BxPC3, C3H-10T1/2, C6/36, Cal-27, CHO, CHO-7, CHO-IR, CHO-K1, CHO-K2, CHO-T, CHO Dhfr-/-, COR-L23, COR-L23/CPR, COR-L23/5010, COR-L23/R23, COS-7, COV-434, CML TI, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepalclc7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK II, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof. Cell lines are available from a variety of sources known to those with skill in the art (see, e.g., the American Type Culture Collection (ATCC) (Manassas, Va.)).

In some embodiments, the engineered cell is a muscle cell (e.g., cardiac muscle, skeletal muscle, and/or smooth muscle), bone cell, blood cell, immune cell (including but not limited to B cells, macrophages, T-cells, CAR-T cells, and the like), kidney cells, bladder cells, lung cells, heart cells, liver cells, brain cells, neurons, skin cells, stomach cells, neuronal support cells, intestinal cells, epithelial cells, endothelial cells, stem or other progenitor cells, adrenal gland cells, cartilage cells, and combinations thereof.

In some embodiments, the engineered cell can be a fungus cell. As used herein, a “fungal cell” refers to any type of eukaryotic cell within the kingdom of fungi. Phyla within the kingdom of fungi include Ascomycota, Basidiomycota, Blastocladiomycota, Chytridiomycota, Glomeromycota, Microsporidia, and Neocallimastigomycota. Fungal cells may include yeasts, molds, and filamentous fungi. In some embodiments, the fungal cell is a yeast cell.

As used herein, the term “yeast cell” refers to any fungal cell within the phyla Ascomycota and Basidiomycota. Yeast cells may include budding yeast cells, fission yeast cells, and mold cells. Without being limited to these organisms, many types of yeast used in laboratory and industrial settings are part of the phylum Ascomycota. In some embodiments, the yeast cell is an S. cerevisiae, Kluyveromyces marxianus, or Issatchenkia orientalis cell. Other yeast cells may include without limitation Candida spp. (e.g., Candida albicans), Yarrowia spp. (e.g., Yarrowia lipolytica), Pichia spp. (e.g., Pichia pastoris), Kluyveromyces spp. (e.g., Kluyveromyces lactis and Kluyveromyces marxianus), Neurospora spp. (e.g., Neurospora crassa), Fusarium spp. (e.g., Fusarium oxysporum), and Issatchenkia spp. (e.g., Issatchenkia orientalis, a.k.a. Pichia kudriavzevii and Candida acidothermophilum). In some embodiments, the fungal cell is a filamentous fungal cell. As used herein, the term “filamentous fungal cell” refers to any type of fungal cell that grows in filaments, i.e., hyphae or mycelia. Examples of filamentous fungal cells may include without limitation Aspergillus spp. (e.g., Aspergillus niger), Trichoderma spp. (e.g., Trichoderma reesei), Rhizopus spp. (e.g., Rhizopus oryzae), and Mortierella spp. (e.g., Mortierella isabellina).

In some embodiments, the fungal cell is an industrial strain. As used herein, “industrial strain” refers to any strain of fungal cell used in or isolated from an industrial process, e.g., production of a product on a commercial or industrial scale. Industrial strain may refer to a fungal species that is typically used in an industrial process, or it may refer to an isolate of a fungal species that may be also used for non-industrial purposes (e.g., laboratory research). Examples of industrial processes may include fermentation (e.g., in production of food or beverage products), distillation, biofuel production, production of a compound, and production of a polypeptide. Examples of industrial strains can include, without limitation, JAY270 and ATCC4124.

In some embodiments, the fungal cell is a polyploid cell. As used herein, a “polyploid” cell may refer to any cell whose genome is present in more than one copy. A polyploid cell may refer to a type of cell that is naturally found in a polyploid state, or it may refer to a cell that has been induced to exist in a polyploid state (e.g., through specific regulation, alteration, inactivation, activation, or modification of meiosis, cytokinesis, or DNA replication). A polyploid cell may refer to a cell whose entire genome is polyploid, or it may refer to a cell that is polyploid in a particular genomic locus of interest.

In some embodiments, the fungal cell is a diploid cell. As used herein, a “diploid” cell may refer to any cell whose genome is present in two copies. A diploid cell may refer to a type of cell that is naturally found in a diploid state, or it may refer to a cell that has been induced to exist in a diploid state (e.g., through specific regulation, alteration, inactivation, activation, or modification of meiosis, cytokinesis, or DNA replication). For example, the S. cerevisiae strain S228C may be maintained in a haploid or diploid state. A diploid cell may refer to a cell whose entire genome is diploid, or it may refer to a cell that is diploid in a particular genomic locus of interest. In some embodiments, the fungal cell is a haploid cell. As used herein, a “haploid” cell may refer to any cell whose genome is present in one copy. A haploid cell may refer to a type of cell that is naturally found in a haploid state, or it may refer to a cell that has been induced to exist in a haploid state (e.g., through specific regulation, alteration, inactivation, activation, or modification of meiosis, cytokinesis, or DNA replication). For example, the S. cerevisiae strain S228C may be maintained in a haploid or diploid state. A haploid cell may refer to a cell whose entire genome is haploid, or it may refer to a cell that is haploid in a particular genomic locus of interest.

In some embodiments, the engineered cell is a cell obtained from a subject. In some embodiments, the subject is a healthy or non-diseased subject. In some embodiments, the subject is a subject with a desired physiological and/or biological characteristic such that when an engineered AAV capsid particle is produced it can package one or more cargo polynucleotides that can be related to the desired physiological and/or biological characteristic and/or capable of modifying the desired physiological and/or biological characteristic. Thus, the cargo polynucleotides of the produced engineered AAV capsid particle can be capable of transferring the desired characteristic to a recipient cell. In some embodiments, the cargo polynucleotides are capable of modifying a polynucleotide of the engineered cell such that the engineered cell has a desired physiological and/or biological characteristic.

In some embodiments, a cell transfected with one or more vectors described herein is used to establish a new cell line comprising one or more vector-derived sequences.

The engineered cells can be used to produce engineered viral (e.g., AAV) capsid polynucleotides, vectors, and/or particles. In some embodiments, the engineered viral (e.g., AAV) capsid polynucleotides, vectors, and/or particles are produced, harvested, and/or delivered to a subject in need thereof. In some embodiments, the engineered cells are delivered to a subject. Other uses for the engineered cells are described elsewhere herein. In some embodiments, the engineered cells can be included in formulations and/or kits described elsewhere herein.

The engineered cells can be stored short-term or long-term for use at a later time. Suitable storage methods are generally known in the art. Further, methods of restoring the stored cells for use (such as thawing, reconstitution, and otherwise stimulating metabolism in the engineered cell after storage) at a later time are also generally known in the art.

### Formulations

The compositions, polynucleotides, polypeptides, particles, cells, vector systems and combinations thereof described herein can be contained in a formulation, such as a pharmaceutical formulation. In some embodiments, the formulations can be used to generate polypeptides and other particles that include one or more CNS-specific n-mers described herein. In some embodiments, the formulations can be delivered to a subject in need thereof. In some embodiments, component(s) of the engineered AAV capsid system, engineered cells, engineered AAV capsid particles, and/or combinations thereof described herein can be included in a formulation that can be delivered to a subject or a cell. In some embodiments, the formulation is a pharmaceutical formulation. One or more of the polypeptides, polynucleotides, vectors, cells, and combinations thereof described herein can be provided to a subject in need thereof or a cell alone or as an active ingredient, such as in a pharmaceutical formulation. As such, also described herein are pharmaceutical formulations containing an amount of one or more of the polypeptides, polynucleotides, vectors, cells, or combinations thereof described herein. In some embodiments, the pharmaceutical formulation can contain an effective amount of the one or more of the polypeptides, polynucleotides, vectors, cells, and combinations thereof described herein. The pharmaceutical formulations described herein can be administered to a subject in need thereof or a cell.

In some embodiments, the amount of the one or more of the polypeptides, polynucleotides, vectors, cells, virus particles, nanoparticles, other delivery particles, and combinations thereof described herein contained in the pharmaceutical formulation can range from about 1 pg/kg to about 10 mg/kg based upon the bodyweight of the subject in need thereof or average bodyweight of the specific patient population to which the pharmaceutical formulation can be administered. The amount of the one or more of the polypeptides, polynucleotides, vectors, cells, and combinations thereof described herein in the pharmaceutical formulation can range from about 1 pg to about 10 g, from about 10 nL to about 10 ml. In embodiments where the pharmaceutical formulation contains one or more cells, the amount can range from about 1 cell to 1×10², 1×10³, 1×10⁴, 1×10⁵, 1×10⁶, 1×10⁷, 1×10⁸, 1×10⁹, 1×10¹⁰ or more cells. In embodiments where the pharmaceutical formulation contains one or more cells, the amount can range from about 1 cell to 1×10², 1×10³, 1×10⁴, 1×10⁵, 1×10⁶, 1×10⁷, 1×10⁸, 1×10⁹, 1×10¹⁰ or more cells per nL, μL, mL, or L.

In embodiments where engineered AAV capsid particles are included in the formulation, the formulation can contain 1 to 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, or 1×10²⁰ transducing units (TU)/mL of the engineered AAV capsid particles. In some embodiments, the formulation can be 0.1 to 100 mL in volume and can contain 1 to 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, or 1×10²⁰ transducing units (TU)/mL of the engineered AAV capsid particles.

### Pharmaceutically Acceptable Carriers and Auxiliary Ingredients and Agents

In embodiments, the pharmaceutical formulation containing an amount of one or more of the polypeptides, polynucleotides, vectors, cells, virus particles, nanoparticles, other delivery particles, and combinations thereof described herein can further include a pharmaceutically acceptable carrier. Suitable pharmaceutically acceptable carriers include, but are not limited to, water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with auxiliary agents, such as lubricants, preservatives, stabilizers, wetting agents, emulsifiers, salts for influencing osmotic pressure, buffers, coloring, flavoring and/or aromatic substances, and the like which do not deleteriously react with the active composition.

In addition to an amount of one or more of the polypeptides, polynucleotides, vectors, cells, engineered AAV capsid particles, nanoparticles, other delivery particles, and combinations thereof described herein, the pharmaceutical formulation can also include an effective amount of an auxiliary active agent, including but not limited to, polynucleotides, amino acids, peptides, polypeptides, antibodies, aptamers, ribozymes, hormones, immunomodulators, antipyretics, anxiolytics, antipsychotics, analgesics, antispasmodics, anti-inflammatoirenti-histamines, anti-infectives, chemotherapeutics, and combinations thereof.

Suitable hormones include, but are not limited to, amino-acid derived hormones (e.g., melatonin and thyroxine), small peptide hormones and protein hormones (e.g., thyrotropin-releasing hormone, vasopressin, insulin, growth hormone, luteinizing hormone, follicle-stimulating hormone, and thyroid-stimulating hormone), eicosanoids (e.g., arachidonic acid, lipoxins, and prostaglandins), and steroid hormones (e.g., estradiol, testosterone, tetrahydro testosterone Cortisol). Suitable immunomodulators include, but are not limited to, prednisone, azathioprine, 6-MP, cyclosporine, tacrolimus, methotrexate, interleukins (e.g., IL-2, IL-7, and IL-12), cytokines (e.g., interferons (e.g., IFN-α, IFN-β, IFN-ε, IFN-K, IFN-ω, and IFN-γ), granulocyte colony-stimulating factor, and imiquimod), chemokines (e.g., CCL3, CCL26 and CXCL7), cytosine phosphate-guanosine, oligodeoxynucleotides, glucans, antibodies, and aptamers).

Suitable antipyretics include, but are not limited to, non-steroidal anti-inflammatories (e.g., ibuprofen, naproxen, ketoprofen, and nimesulide), aspirin and related salicylates (e.g., choline salicylate, magnesium salicylate, and sodium salicylate), paracetamol/acetaminophen, metamizole, nabumetone, phenazone, and quinine.

Suitable anxiolytics include, but are not limited to, benzodiazepines (e.g., alprazolam, bromazepam, chlordiazepoxide, clonazepam, clorazepate, diazepam, flurazepam, lorazepam, oxazepam, temazepam, triazolam, and tofisopam), serotonergic antidepressants (e.g., selective serotonin reuptake inhibitors, tricyclic antidepressants, and monoamine oxidase inhibitors), mebicar, fabomotizole, selank, bromantane, emoxypine, azapirones, barbiturates, hydroxyzine, pregabalin, validol, and beta blockers.

Suitable antipsychotics include, but are not limited to, benperidol, bromoperidol, droperidol, haloperidol, moperone, pipamperone, timiperone, fluspirilene, penfluridol, pimozide, acepromazine, chlorpromazine, cyamemazine, dixyrazine, fluphenazine, levomepromazine, mesoridazine, perazine, pericyazine, perphenazine, pipotiazine, prochlorperazine, promazine, promethazine, prothipendyl, thioproperazine, thioridazine, trifluoperazine, triflupromazine, chlorprothixene, clopenthixol, flupentixol, thiothixene, zuclopenthixol, clotiapine, loxapine, prothipendyl, carpipramine, clocapramine, molindone, mosapramine, sulpiride, veralipride, amisulpride, amoxapine, aripiprazole, asenapine, clozapine, blonanserin, iloperidone, lurasidone, melperone, nemonapride, olanzapine, paliperidone, perospirone, quetiapine, remoxipride, risperidone, sertindole, trimipramine, ziprasidone, zotepine, alstonie, bifeprunox, bitopertin, brexpiprazole, cannabidiol, cariprazine, pimavanserin, pomaglumetad methionil, vabicaserin, xanomeline, and zicronapine.

Suitable analgesics include, but are not limited to, paracetamol/acetaminophen, nonsteroidal anti-inflammatoires (e.g., ibuprofen, naproxen, ketoprofen, and nimesulide), COX-2 inhibitors (e.g., rofecoxib, celecoxib, and etoricoxib), opioids (e.g., morphine, codeine, oxycodone, hydrocodone, dihydromorphine, pethidine, buprenorphine), tramadol, norepinephrine, flupirtine, nefopam, orphenadrine, pregabalin, gabapentin, cyclobenzaprine, scopolamine, methadone, ketobemidone, piritramide, and aspirin and related salicylates (e.g., choline salicylate, magnesium salicylate, and sodium salicylate).

Suitable antispasmodics include, but are not limited to, mebeverine, papaverine, cyclobenzaprine, carisoprodol, orphenadrine, tizanidine, metaxalone, methocarbamol, chlorzoxazone, baclofen, dantrolene, baclofen, tizanidine, and dantrolene. Suitable anti-inflammatories include, but are not limited to, prednisone, non-steroidal anti-inflammatoires (e.g., ibuprofen, naproxen, ketoprofen, and nimesulide), COX-2 inhibitors (e.g., rofecoxib, celecoxib, and etoricoxib), and immune selective anti-inflammatory derivatives (e.g., submandibular gland peptide-T and its derivatives)

Suitable anti-histamines include, but are not limited to, H1-receptor antagonists (e.g., acrivastine, azelastine, bilastine, brompheniramine, buclizine, bromodiphenhydramine, carbinoxamine, cetirizine, chlorpromazine, cyclizine, chlorpheniramine, clemastine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocetirizine, loratadine, meclizine, mirtazapine, olopatadine, orphenadrine, phenindamine, pheniramine, phenyltoloxamine, promethazine, pyrilamine, quetiapine, rupatadine, tripelennamine, and triprolidine), H2-receptor antagonists (e.g., cimetidine, famotidine, lafutidine, nizatidine, ranitidine, and roxatidine), tritoqualine, catechin, cromoglicate, nedocromil, and p2-adrenergic agonists.

Suitable anti-infectives include, but are not limited to, amebicides (e.g., nitazoxanide, paromomycin, metronidazole, tinidazole, chloroquine, miltefosine, amphotericin b, and iodoquinol), aminoglycosides (e.g., paromomycin, tobramycin, gentamicin, amikacin, kanamycin, and neomycin), anthelmintics (e.g., pyrantel, mebendazole, ivermectin, praziquantel, albendazole, thiabendazole, oxamniquine), antifungals (e.g., azole antifungals (e.g., itraconazole, fluconazole, parconazole, ketoconazole, clotrimazole, miconazole, and voriconazole), echinocandins (e.g., caspofungin, anidulafungin, and micafungin), griseofulvin, terbinafine, flucytosine, and polyenes (e.g., nystatin, and amphotericin b), antimalarial agents (e.g., pyrimethamine/sulfadoxine, artemether/lumefantrine, atovaquone/proguanil, quinine, hydroxychloroquine, mefloquine, chloroquine, doxycycline, pyrimethamine, and halofantrine), antituberculosis agents (e.g., aminosalicylates (e.g., aminosalicylic acid), isoniazid/rifampin, isoniazid/pyrazinamide/rifampin, bedaquiline, isoniazid, ethambutol, rifampin, rifabutin, rifapentine, capreomycin, and cycloserine), antivirals (e.g., amantadine, rimantadine, abacavir/lamivudine, emtricitabine/tenofovir, cobicistat/elvitegravir/emtricitabine/tenofovir, efavirenz/emtricitabine/tenofovir, abacavir/lamivudine/zidovudine, lamivudine/zidovudine, emtricitabine/tenofovir, emtricitabine/lopinavir/ritonavir/tenofovir, interferon alfa-2v/ribavirin, peginterferon alfa-2b, maraviroc, raltegravir, dolutegravir, enfuvirtide, foscarnet, fomivirsen, oseltamivir, zanamivir, nevirapine, efavirenz, etravirine, rilpivirine, delavirdine, nevirapine, entecavir, lamivudine, adefovir, sofosbuvir, didanosine, tenofovir, abacavir, zidovudine, stavudine, emtricitabine, zalcitabine, telbivudine, simeprevir, boceprevir, telaprevir, lopinavir/ritonavir, boceprevir, darunavir, ritonavir, tipranavir, atazanavir, nelfinavir, amprenavir, indinavir, saquinavir, ribavirin, valacyclovir, acyclovir, famciclovir, ganciclovir, and valganciclovir), carbapenems (e.g., doripenem, meropenem, ertapenem, and cilastatin/imipenem), cephalosporins (e.g., cefadroxil, cephradine, cefazolin, cephalexin, cefepime, cefazoline, loracarbef, cefotetan, cefuroxime, cefprozil, loracarbef, cefoxitin, cefaclor, ceftibuten, ceftriaxone, cefotaxime, cefpodoxime, cefdinir, cefixime, cefditoren, ceftizoxime, and ceftazidime), glycopeptide antibiotics (e.g., vancomycin, dalbavancin, oritavancin, and telavancin), glycylcyclines (e.g., tigecycline), leprostatics (e.g., clofazimine and thalidomide), lincomycin and derivatives thereof (e.g., clindamycin and lincomycin), macrolides and derivatives thereof (e.g., telithromycin, fidaxomicin, erythromycin, azithromycin, clarithromycin, dirithromycin, and troleandomycin), linezolid, sulfamethoxazole/trimethoprim, rifaximin, chloramphenicol, Fosfomycin, metronidazole, aztreonam, bacitracin, penicillin (amoxicillin, ampicillin, bacampicillin, carbenicillin, piperacillin, ticarcillin, amoxicillin/clavulanate, ampicillin/sulbactam, piperacillin/tazobactam, clavulanate/ticarcillin, penicillin, procaine penicillin, oxacillin, dicloxacillin, and nafcillin), quinolones (e.g., lomefloxacin, norfloxacin, ofloxacin, gatifloxacin, moxifloxacin, ciprofloxacin, levofloxacin, gemifloxacin, moxifloxacin, cinoxacin, nalidixic acid, enoxacin, grepafloxacin, gatifloxacin, trovafloxacin, and sparfloxacin), sulfonamides (e.g., sulfamethoxazole/trimethoprim, sulfasalazine, and sulfisoxazole), tetracyclines (e.g., doxycycline, demeclocycline, minocycline, doxycycline/salicylic acid, doxycycline/omega-3 polyunsaturated fatty acids, and tetracycline), and urinary anti-infectives (e.g., nitrofurantoin, methenamine, Fosfomycin, cinoxacin, nalidixic acid, trimethoprim, and methylene blue).

Suitable chemotherapeutics include, but are not limited to, paclitaxel, brentuximab vedotin, doxorubicin, 5-FU (fluorouracil), everolimus, pemetrexed, melphalan, pamidronate, anastrozole, exemestane, nelarabine, ofatumumab, bevacizumab, belinostat, tositumomab, carmustine, bleomycin, bosutinib, busulfan, alemtuzumab, irinotecan, vandetanib, bicalutamide, lomustine, daunorubicin, clofarabine, cabozantinib, dactinomycin, ramucirumab, cytarabine, Cytoxan, cyclophosphamide, decitabine, dexamethasone, docetaxel, hydroxyurea, dacarbazine, leuprolide, epirubicin, oxaliplatin, asparaginase, estramustine, cetuximab, vismodegib, asparaginase Erwinia chrysanthemi, amifostine, etoposide, flutamide, toremifene, fulvestrant, letrozole, degarelix, pralatrexate, methotrexate, floxuridine, obinutuzumab, gemcitabine, afatinib, imatinib mesylate, carmustine, eribulin, trastuzumab, altretamine, topotecan, ponatinib, idarubicin, ifosfamide, ibrutinib, axitinib, interferon alfa-2a, gefitinib, romidepsin, ixabepilone, ruxolitinib, cabazitaxel, ado-trastuzumab emtansine, carfilzomib, chlorambucil, sargramostim, cladribine, mitotane, vincristine, procarbazine, megestrol, trametinib, mesna, strontium-89 chloride, mechlorethamine, mitomycin, busulfan, gemtuzumab ozogamicin, vinorelbine, filgrastim, pegfilgrastim, sorafenib, nilutamide, pentostatin, tamoxifen, mitoxantrone, pegaspargase, denileukin diftitox, alitretinoin, carboplatin, pertuzumab, cisplatin, pomalidomide, prednisone, aldesleukin, mercaptopurine, zoledronic acid, lenalidomide, rituximab, octreotide, dasatinib, regorafenib, histrelin, sunitinib, siltuximab, omacetaxine, thioguanine (tioguanine), dabrafenib, erlotinib, bexarotene, temozolomide, thiotepa, thalidomide, Bacillus Calmette-Guerin (BCG), temsirolimus, bendamustine hydrochloride, triptorelin, arsenic trioxide, lapatinib, valrubicin, panitumumab, vinblastine, bortezomib, tretinoin, azacitidine, pazopanib, teniposide, leucovorin, crizotinib, capecitabine, enzalutamide, ipilimumab, goserelin, vorinostat, idelalisib, ceritinib, abiraterone, epothilone, tafluposide, azathioprine, doxifluridine, vindesine, and all-trans retinoic acid.

In embodiments where there is an auxiliary active agent contained in the pharmaceutical formulation in addition to the one or more of the polypeptides, polynucleotides, CRISPR-Cas complexes, vectors, cells, virus particles, nanoparticles, other delivery particles, and combinations thereof described herein, amount, such as an effective amount, of the auxiliary active agent will vary depending on the auxiliary active agent. In some embodiments, the amount of the auxiliary active agent ranges from 0.001 micrograms to about 1 milligram. In other embodiments, the amount of the auxiliary active agent ranges from about 0.01 IU to about 1000 IU. In further embodiments, the amount of the auxiliary active agent ranges from 0.001 mL to about 1 mL. In yet other embodiments, the amount of the auxiliary active agent ranges from about 1% w/w to about 50% w/w of the total pharmaceutical formulation. In additional embodiments, the amount of the auxiliary active agent ranges from about 1% v/v to about 50% v/v of the total pharmaceutical formulation. In still other embodiments, the amount of the auxiliary active agent ranges from about 1% w/v to about 50% w/v of the total pharmaceutical formulation.

### Dosage Forms

In some embodiments, the pharmaceutical formulations described herein may be in a dosage form. The dosage forms can be adapted for administration by any appropriate route. Appropriate routes include, but are not limited to, oral (including buccal or sublingual), rectal, epidural, intracranial, intraocular, inhaled, intranasal, topical (including buccal, sublingual, or transdermal), vaginal, intraurethral, parenteral, intracranial, subcutaneous, intramuscular, intravenous, intraperitoneal, intradermal, intraosseous, intracardiac, intraarticular, intracavernous, intrathecal, intravitreal, intracerebral, gingival, subgingival, intracerebroventricular, and intradermal. Such formulations may be prepared by any method known in the art.

Dosage forms adapted for oral administration can be discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as foam, spray, or liquid solution. In some embodiments, the oral dosage form can contain about 1 ng to 1000 g of a pharmaceutical formulation containing a therapeutically effective amount or an appropriate fraction thereof of the targeted effector fusion protein and/or complex thereof or composition containing the one or more of the polypeptides, polynucleotides, vectors, cells, and combinations thereof described herein. The oral dosage form can be administered to a subject in need thereof.

Where appropriate, the dosage forms described herein can be microencapsulated.

The dosage form can also be prepared to prolong or sustain the release of any ingredient. In some embodiments, the one or more of the polypeptides, polynucleotides, vectors, cells, and combinations thereof described herein can be the ingredient whose release is delayed. In other embodiments, the release of an optionally included auxiliary ingredient is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wilkins, Baltimore, MD, 2000, and “Pharmaceutical dosage forms and drug delivery systems”, 6th Edition, Ansel et al., (Media, PA: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, the one or more of the polypeptides, polynucleotides, vectors, cells, and combinations thereof described herein can be formulated with a paraffinic or water-miscible ointment base. In some embodiments, the active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, the one or more of the polypeptides, polynucleotides, vectors, cells, and combinations thereof described herein is contained in a dosage form adapted for inhalation is in a particle-size-reduced form that is obtained or obtainable by micronization. In some embodiments, the particle size of the size reduced (e.g., micronized) compound or salt or solvate thereof, is defined by a D50 value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active ingredient (e.g., the one or more of the polypeptides, polynucleotides, vectors, cells, and combinations thereof described herein and/or auxiliary active agent), which may be generated by various types of metered dose pressurized aerosols, nebulizers, or insufflators.

In some embodiments, the dosage forms can be aerosol formulations suitable for administration by inhalation. In some of these embodiments, the aerosol formulation can contain a solution or fine suspension of the one or more of the polypeptides, polynucleotides, vectors, cells, and combinations thereof described herein and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal, or an aerosol dispenser fitted with a metering valve (e.g., metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of one or more of the polypeptides, polynucleotides, vectors, cells, and combinations thereof described herein. In further embodiments, the aerosol formulation can also contain co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, or 3 doses are delivered each time.

For some dosage forms suitable and/or adapted for inhaled administration, the pharmaceutical formulation is a dry powder inhalable formulation. In addition to the one or more of the polypeptides, polynucleotides, vectors, cells, and combinations thereof described herein, an auxiliary active ingredient, and/or pharmaceutically acceptable salt thereof, such a dosage form can contain a powder base such as lactose, glucose, trehalose, mannitol, and/or starch. In some of these embodiments, the one or more of the polypeptides, polynucleotides, vectors, cells, and combinations thereof described herein is in a particle-size reduced form. In further embodiments, a performance modifier, such as L-leucine or another amino acid, cellobiose octaacetate, and/or metals salts of stearic acid, such as magnesium or calcium stearate.

In some embodiments, the aerosol dosage forms can be arranged so that each metered dose of aerosol contains a predetermined amount of an active ingredient, such as the one or more of the one or more of the polypeptides, polynucleotides, vectors, cells, and combinations thereof described herein.

Dosage forms adapted for parenteral administration and/or adapted for any type of injection (e.g., intravenous, intraperitoneal, subcutaneous, intramuscular, intradermal, intraosseous, epidural, intracardiac, intraarticular, intracavernous, gingival, subgingival, intrathecal, intravitreal, intracerebral, and intracerebroventricular) can include aqueous and/or non-aqueous sterile injection solutions, which can contain anti-oxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and resuspended in a sterile carrier to reconstitute the dose prior to administration. Extemporaneous injection solutions and suspensions can be prepared in some embodiments, from sterile powders, granules, and tablets.

Dosage forms adapted for ocular administration can include aqueous and/or nonaqueous sterile solutions that can optionally be adapted for injection, and which can optionally contain anti-oxidants, buffers, bacteriostats, solutes that render the composition isotonic with the eye or fluid contained therein or around the eye of the subject, and aqueous and nonaqueous sterile suspensions, which can include suspending agents and thickening agents.

For some embodiments, the dosage form contains a predetermined amount of the one or more of the polypeptides, polynucleotides, vectors, cells, and combinations thereof described herein per unit dose. In some embodiments, the predetermined amount of the such unit doses may therefore be administered once or more than once a day. Such pharmaceutical formulations may be prepared by any of the methods well known in the art.

### Kits

Also described herein are kits that contain one or more of the one or more of the compositions, polypeptides, polynucleotides, vectors, cells, or other components described herein and combinations thereof and pharmaceutical formulations described herein. In embodiments, one or more of the polypeptides, polynucleotides, vectors, cells, and combinations thereof described herein can be presented as a combination kit. As used herein, the terms “combination kit” or “kit of parts” refers to the compounds, or formulations and additional components that are used to package, screen, test, sell, market, deliver, and/or administer the combination of elements or a single element, such as the active ingredient, contained therein. Such additional components include but are not limited to, packaging, syringes, blister packages, bottles, and the like. The combination kit can contain one or more of the components (e.g., one or more of the one or more of the polypeptides, polynucleotides, vectors, cells, and combinations thereof) or formulation thereof can be provided in a single formulation (e.g., a liquid, lyophilized powder, etc.), or in separate formulations. The separate components or formulations can be contained in a single package or in separate packages within the kit. The kit can also include instructions in a tangible medium of expression that can contain information and/or directions regarding the content of the components and/or formulations contained therein, safety information regarding the content of the components(s) and/or formulation(s) contained therein, information regarding the amounts, dosages, indications for use, screening methods, component design recommendations and/or information, recommended treatment regimen(s) for the components(s) and/or formulations contained therein. As used herein, “tangible medium of expression” refers to a medium that is physically tangible or accessible and is not a mere abstract thought or an unrecorded spoken word. “Tangible medium of expression” includes, but is not limited to, words on a cellulosic or plastic material, or data stored in a suitable computer readable memory form. The data can be stored on a unit device, such as a flash memory drive or CD-ROM or on a server that can be accessed by a user via, e.g., a web interface.

In one embodiment, the invention provides a kit comprising one or more of the components described herein. In some embodiments, the kit comprises a vector system and instructions for using the kit. In some embodiments, the vector system includes a regulatory element operably linked to one or more engineered polynucleotides, such as those containing a selective n-mer, as described elsewhere herein and, optionally, a cargo molecule, which can optionally be operably linked to a regulatory element. The one or more engineered polynucleotides such as those containing a selective n-mer, as described elsewhere herein and, can be included on the same or different vectors as the cargo molecule in embodiments containing a cargo molecule within the kit.

In some embodiments, the kit comprises a vector system and instructions for using the kit. In some embodiments, the vector system comprises (a) a first regulatory element operably linked to a direct repeat sequence and one or more insertion sites for inserting one or more guide sequences up- or downstream (whichever applicable) of the direct repeat sequence, wherein when expressed, the guide sequence directs sequence-specific binding of a Cas9 CRISPR complex to a target sequence in a eukaryotic cell, wherein the Cas9 CRISPR complex comprises a Cas9 enzyme complexed with the guide sequence that is hybridized to the target sequence; and/or (b) a second regulatory element operably linked to an enzyme-coding sequence encoding said Cas9 enzyme comprising a nuclear localization sequence. Where applicable, a tracr sequence may also be provided. In some embodiments, the kit comprises components (a) and (b) located on the same or different vectors of the system. In some embodiments, component (a) further comprises two or more guide sequences operably linked to the first regulatory element, wherein when expressed, each of the two or more guide sequences direct sequence specific binding of a CRISPR complex to a different target sequence in a eukaryotic cell. In some embodiments, the Cas9 enzyme comprises one or more nuclear localization sequences of sufficient strength to drive accumulation of said CRISPR enzyme in a detectable amount in the nucleus of a eukaryotic cell. In some embodiments, the CRISPR enzyme is a type V or VI CRISPR system enzyme. In some embodiments, the CRISPR enzyme is a Cas9 enzyme. In some embodiments, the Cas9 enzyme is derived from Francisella tularensis 1, Francisella tularensis subsp. novicida, Prevotella albensis, Lachnospiraceae bacterium MC2017 1, Butyrivibrio proteoclasticus, Peregrinibacteria bacterium GW2011 GWA2 33_10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens, or Porphyromonas macacae Cas9 (e.g., modified to have or be associated with at least one DD), and may include further alteration or mutation of the Cas9, and can be a chimeric Cas9. In some embodiments, the DD-CRISPR enzyme is codon-optimized for expression in a eukaryotic cell. In some embodiments, the DD-CRISPR enzyme directs cleavage of one or two strands at the location of the target sequence. In some embodiments, the DD-CRISPR enzyme lacks or substantially DNA strand cleavage activity (e.g., no more than 5% nuclease activity as compared with a wild-type enzyme or enzyme not having the mutation or alteration that decreases nuclease activity). In some embodiments, the first regulatory element is a polymerase III promoter. In some embodiments, the second regulatory element is a polymerase II promoter. In some embodiments, the guide sequence is at least 16, 17, 18, 19, 20, 25 nucleotides, or between 16-30, or between 16-25, or between 16-20 nucleotides in length.

### Methods of Use for Delivery of Cargo to CNS

The compositions including one or more of the cell-selective targeting moieties, engineered AAV capsid system polynucleotides, polypeptides, vector(s), engineered cells, engineered AAV capsid particles can be used generally to package and/or deliver one or more cargos to neuron and glial cells of the CNS. In some embodiments, delivery is done in cell-selective manner based upon the selectivity of the targeting moiety. In some embodiments this is conferred by the tropism of the engineered AAV capsid, which can be influenced at least in part by the inclusion of one or n-mer motifs described elsewhere herein. In some embodiments, compositions including one or more of the CNS endothelial targeting moieties, engineered AAV capsid particles, can be administered to a subject or a cell, tissue, and/or organ and facilitate the transfer and/or integration of the cargo to the recipient cell. In other embodiments, engineered cells capable of producing compositions, such as polypeptides and other particles (e.g., engineered AAV capsids and viral particles), containing one or more of the targeting moieties can be generated from the polynucleotides, vectors, and vector systems etc., described herein. This includes without limitation, the engineered AAV capsid system molecules (e.g., polynucleotides, vectors, and vector systems, etc.). In some embodiments, the polynucleotides, vectors, and vector systems etc., described herein capable of generating the compositions, such as polypeptides and other particles (e.g., engineered AAV capsids and viral particles), containing one or more of the targeting moieties can be delivered to a cell or tissue, in vivo, ex vivo, or in vitro. In some embodiments, when delivered to a subject, the composition can transform a subject's cell in vivo or ex vivo to produce an engineered cell that can be capable of making a composition described herein that contains one or more of the cell-selective targeting moieties described herein, including but not limited to the engineered AAV capsid particles, which can be released from the engineered cell and deliver cargo molecule(s) to a recipient cell in vivo or produce personalized engineered compositions (e.g., AAV capsid particles) for reintroduction into the subject from which the recipient cell was obtained.

In some embodiments, an engineered cell can be delivered to a subject, where it can release produced compositions of the present invention (including but not limited to engineered AAV capsid particles) such that they can then deliver a cargo (e.g., a cargo polynucleotide(s)) to a recipient cell. These general processes can be used in a variety of ways to treat and/or prevent disease or a symptom thereof in a subject, generate model cells, generate modified organisms, provide cell selection and screening assays, in bioproduction, and in other various applications.

In some embodiments, the compositions, such as polypeptides and other particles (e.g., engineered AAV capsids and viral particles), containing one or more of the targeting moieties can be delivered to neural cells of the CNS. In another example embodiment, the compositions containing one or more targeting moieties can be delivered to glial cells of the CNS.

In some embodiments, the engineered AAV capsid polynucleotides, vectors, and systems thereof can be used to generate engineered AAV capsid variant libraries that can be mined for variants with a desired cell-selectivity. The description provided herein as supported by the various Examples can demonstrate that one having a desired cell-selectivity in mind could utilize the present invention as described herein to obtain a capsid with the desired cell-selectivity.

### Methods of Treatment

Provided herein are methods for treating a disease or disorder, the method comprising administering to a subject in need thereof, compositions as disclosed herein to cells of the CNS. In an aspect, the compositions used in methods disclosed herein are capable of increasing transduction of neurons and/or glial cells of the CNS, allowing for delivery of cargo and therapeutics directly to such cell types. In embodiments, a method is disclosed wherein the cargo is one or more polypeptides.

### Diseases or Disorders

Generally, the compositions described herein can be used in a therapy for treating or preventing a CNS disease, disorder, or a symptom thereof. It will be appreciated that a CNS disease or disorder refers to any disease or disorder whose pathology involves or affects one or more cell types of the central nervous system. In some embodiments, the CNS disease or disorder is one whose primary pathology involves one or more cell types of the CNS. In some embodiments, one or more other cell types outside of the CNS are involved in the pathology of the CNS disease, such as a muscle cell or a peripheral nervous system cell. In some embodiments, the CNS disease or disorder can be caused by one or more genetic abnormalities. In some embodiments, the CNS disease or disorder is not caused by a genetic abnormality. Non-genetic causes of diseases include infection, cancer, physical trauma and others that will be appreciated by those of skill in the art. It also will be apricated that gene modification approaches to treating disease can be applied to treat and/or prevent both genetic diseases and non-genetic diseases. For example, in the case of non-genetic diseases, a gene therapy approach can be used to modify the cause of the non-genetic disease (e.g., a cancer or infectious organism) such that the cause is no longer disease causing (e.g., by eliminating or rendering non-functional the cancer cells or infectious organism).

Exemplary CNS diseases and disorders include, without limitation, Friedreich's Ataxia, Dravet Syndrome, Spinocerebellar Ataxia Type 3, Niemann Pick Type C, Huntington's Disease, Pompe Disease, Myotonic Dystrophy Type 1, Glut1 Deficiency Syndrome (De Vivo Syndrome), Tay-Sachs, Spinal Muscular Atrophy, Alzheimer's disease, Amyotrophic lateral sclerosis (ALS), Danon disease, Rett Syndrome, Angleman Syndrome, infantile neuronal dystorpy, Gaucher's disease, Krabbe disease, metachromatic leukodystrophy, Salla disease, Farber disease or Spinal Musular Atrophy with progressive myoclonic Epilepsy (also reffered to as Jankovic-Rivera syndrome, Unverricht-Lundborg disease, AADC deficiency, Parkinson's disease, Batten disease, a neuronal ceroid lipofuscinosis disease, giant axonal neuropathy, a mucopolysaccharidosis disease (e.g., Hurler syndrome, MPS III A-D), neurofibromatosis, a spinocerebellar ataxia disease, Sandoff disease, GM2 gangliosidosis, Canavan disease, Cockayne syndrome, a pain disease or disorder, a neuropathy or nerve damage, or any combination thereof. Others are described elsewhere herein and/or will be appreciated by those of ordinary skill in the art in view of the description provided herein.

Other diseases can include, but are not limited to, any of the following: cancer (such as glioblastoma or other brain or CNS cancers), Acubetivacter infections, actinomycosis, African sleeping sickness, AIDS/HIV, ameobiasis, Anaplasmosis, Angiostrongyliasis, Anisakiasis, Anthrax, Acranobacterium haemolyticum infection, Argentine hemorrhagic fever, Ascariasis, Aspergillosis, Astrovirus infection, Babesiosis, Bacterial meningitis, Bacterial pneumonia, Bacterial vaginosis, Bacteroides infection, balantidiasis, Bartonellosis, Baylisascaris infection, BK virus infection, Black Piedra, Blastocytosis, Blastomycosis, Bolivian hemorrhagic fever, Botulism, Brazillian hemmorhagic fever, brucellosis, Bubonic plague, Burkholderia infection, buruli ulcer, calicivirus invention, campylobacteriosis, Candidasis, Capillariasis, Carrion's disease, Cat-scratch disease, cellulitis, Chagas Disease, Chancroid, Chickenpox, Chikungunya, Chlamydia, Chlamydia pneumoniae, Cholera, Chromoblastomycosis, Chytridiomycosis, Clonochiasis, Clostridium difficile colitis, Coccidioidomycosis, Colorado tick fever, rhinovirus/coronavirus invection (common cold), Cretzfeldt-Jakob disease, Crimean-congo hemorrhagic fever, Cryptococcosis, Cryptosporidosis, Cutaneous larva migrans (CLM), cyclosporiasis, cysticercosis, cytomegalovirus infection, Dengue fever, Desmodesmus infection, Dientamoebiasis, Diptheria, Diphylobothriasis, Dracunculiasis, Ebola, Echinococcosis, Ehrlichiosis, Enterobiasis, Enterococcus infection, Enterovirus infection, Epidemic typhus, Erthemia Infectisoum, Exanthem subitum, Fasciolasis, Fasciolopsiasis, fatal familial insomnia, filarisis, Clostridum perfingens infection, Fusobacterium infection, Gas gangrene (clostridial myonecrosis), Geotrichosis, Gerstmann-Straussler-Scheinker syndrome, Giardasis, Glanders, Gnathostomiasis, Gonorrhea, Granuloma inguinales, Group A streptococcal infection, Group B streptococcal infection, Haemophilus influenzae infection, Hand, foot, and mouth disease, hanta virus pulmonary syndrome, heartland virus disease, Helicobacter pylori infection, hemorrhagi fever with renal syndrome, Hendra virus infection, Hepatitis (all groups A, B, C, D, E), hepes simplex, histoplasmosis, hookworm infection, human bocavirus infection, human ewingii erlichosis, Human granulocytic anaplasmosis, human metapneymovirus infection, human monocytic ehrlichosis, human papaloma virus, Hymenolepiasis, Epstein-Barr infection, mononucleosis, influenza, isoporisis, Kawasaki disease, Kingell kingae infection, Kuru, Lasas fever, Leginollosis (Legionnaires's disease and Potomac Fever), Leishmaniasis, Leprosy, Leptospirosis, Listeriosis, Lyme disease, lymphatic filariasis, lymphocytic choriomeningitis, Malaria, Marburg hemorrhagic feaver, measals, Middle East respiratory syndrome, Meliodosis, menigitis, Menigococcal disease, Metagonimiasis, Microsporidosis, Molluscum contagiosum, Monkeypox, Mumps, Murine typhus, Mycoplasma pneumonia, Mycoplasma genitalium infection, Mycetoma, Myiasis, Conjunctivitis, Nipah virus infection, Norovirus, Variant Creutzfeldt-Jakob disease, Nocardosis, Onchocerciasis, Opisthorchiasis, Paracoccidioidomycosis, Paragonimiasis, Pasteurellosis, Pdiculosisi capitis, Pediculosis corpis, Pediculosis pubis, pelvic inflammatory disease, pertussis, plague, pneumococcal infection, pneumocystis pneumonia, pneumonia, poliomyelitis, prevotella infection, primary amoebic menigoencephalitis, progressive multifocal leukoencephalopathy, Psittacosis, Qfever, rabies, relapsing fever, respiratory syncytial virus infection, rhinovirus infection, rickettsial infection, Rickettsialpox, Rift Valley Fever, Rocky Mountain Spotted Fever, Rotavirus infection, Rubella, Salmonellosis, SARS, Scabies, Scarlet fever, Schistosomiais, Sepsis, Shigellosis, Shingles, Smallpox, Sporotrichosisi, Staphlococcol infection (including MRSA), strongyloidiasis, subacute sclerosing panecephalitis, Syphillis, Taeniasis, tetanus, Trichophyton species infection, Tocariasis, Toxoplasmosis, Trachoma, Trichinosis, Trichuriasis, Tuberculosis, Tularemia, Typhoid Fever, Typhus Fever, Ureaplasma urealyticum infection, Valley fever, Venezuelan equine encephalitis, Venezuelan hemorrhagic fever, Vibrio species infection, Viral pneumonia, West Nile Fever, White Piedra, Yersinia pseudotuberculosis, Yersiniosis, Yellow fever, Zeaspora, Zika fever, Zygomycosis and combinations thereof.

In example embodiments, a method is disclosed wherein the disease or disorder is a cancer, neurological disorder or infection.

In an embodiment, methods of treatment comprise administering a composition as detailed herein to a subject in need thereof. In an example embodiment, the cancer is a neuroepithelial cancer. In an embodiment, the cancer is a neuroepithelial tumor., for example, Astrocytic tumors, e.g., Diffuse Astrocytoma (fibrillary, protoplasmic, gemistocytic, mixed), Anaplastic (malignant) astrocytoma, Glioblastoma (giant cell, gliosarcoma variants), Pilocytic astrocytoma, Pleomorphic xanthoastrocytoma, or Subependymal giant cell astrocytoma; Oligodendroglial tumors, e.g., Oligodendroglioma, Anaplastic (malignant) Oligodendroglioma, Ependymal tumors, Ependymoma (cellular, papillary, clear cell, tanycytic), Anaplastic (malignant) ependymoma, ependymoma, Subependymoma; Mixed tumors, e.g., Oligoastrocytomaor Anaplastic (malignant) oligoastrocytoma; Choroid plexus tumors, e.g., Choroid Plexus papilloma or Choroid Plexus carcinoma; Neuronal and mixed neuronal-glial tumors, e.g., Gangliocytoma, Gangloglioma, Dysembryoplastic neuroepithelial tumor (DNET), Dysplastic gangliocytoma of the cerebellum (Lhermitte-Duclos), Desmoplastic infantile astrocytoma/ganglioglioma, Central neurocytoma, Anaplastic ganglioglioma, Cerebellar liponeurocytoma, Paraganglioma of the filum terminale; Pineal tumors, e.g., Pineocytoma, Pineoblastoma, Pineal parenchymal tumor of intermediate differentiation; Embryonal tumors, e.g., Medulloblastoma (desmoplastic, large cell, melanotic, medullomyoblastoma), Medulloepithelioma, Supratentorial primitive neuroectodermal tumors, PNETs such as Neuroblastoma, Ganglioneuroblastoma, Ependymoblastoma, or Atypical teratoid/rhabdoid tumor; Neuroblastic tumors, e.g., Olfactory (esthesioneuroblastoma), Olfactory neuroepithelioma, Neuroblastomas of the adrenal gland and sympathetic nervous system; Glial tumors of uncertain etiology, e.g., Astroblastoma, Gliomatosis cerebri, Chordoid glioma of the third ventricle.

In an embodiment, the cancer is a primary cancer metastasized to brain or other region of the central nervous system.

In an example embodiment, the neurological disorder is caused by a neurodegenerative or a neurodevelopmental disease. Example neurodegenerative diseases include, but are not limited to, Alzheimer's disease and other memory disorders, amyotrophic lateral sclerosis (ALS), ataxia, Huntington's disease, Parkinson's disease, motor neuron disease, multiple system atrophy, progressive supranuclear palsy. Examples of neurodevelopmental diseases include, but are not limited to, attention-deficit/hyperactivity disorder (ADHD), autism, learning disabilities, intellectual disability (also known as mental retardation), conduct disorders, cerebral palsy, speech and language disorders, Tourette syndrome, Schizophrenia, Fragile X syndrome, and impairments in vision and hearing.

Exemplary CNS, Brain, and/or Neuronal Disease-Associated Genes

Examples of CNS, brain, and/or neuronal disease-associated genes and polynucleotides that can be modified using the engineered delivery AAV delivery system molecules, vectors, capsids, engineered cells, and/or engineered delivery particles described herein are described below.

In some embodiments, a therapeutic or preventive, such as the engineered AAV capsids and systems thereof as described elsewhere herein, can be delivered to a subject in need thereof or a cell thereof to treat a brain, neuron, neurological, and/or central nervous system disease or disorder (CNS). In some embodiments the brain, neuron, neurological, and/or CNS disease or disorder can be caused, directly or indirectly, by one or mutations in one or more of the following genes as compared to normal or non-pathological variant of the same: in the case of Amyotrophic lateral sclerosis (ALS): SOD1, ALS2, STEX, FUS, TARDBP, VEGF (VEGF-a, VEGF-b, VEGF-c); in the case of Alzheimer's disease: E1, CHIP, UCH, UBB, Tau, LRP, PICALM, Clusterin, PS1, SORLI, CR1, Vldlr, Ubal, Uba3, CHIP28, Aqp1, Uchl1, Uch13, APP, AAA, CVAP, ADI, APOE, AD2, PSEN2, AD4, STM2, APBB2, FE65L1, NOS3, PLAU, URK, ACE, DCP1, ACEI, MPO, PACIPI, PAXIPIL, PTIP, A2M, BLMH, BMH, PSEN1, AD3); in the case of Autism: Mecp2, BZRAPI, MDGA2, Sema5A, Neurexin 1, GLO1, MECP2, RTT, PPMX, MRX16, MRX79, NLGN3, NLGN4, KIAA1260, AUTSX2; in the case of Fragile X Syndrome: FMR2, FXR1, FXR2, mGLUR5; in the case of Huntington's disease and disease like disorders: HD, IT15, PRNP, PRIP, JPH3, JP3, HDL2, TBP, SCA17); in the case of Parkinson's disease: NR4A2, NURRI, NOT, TINUR, SNCAIP, TBP, SCA17, SNCA, NACP, PARKI, PARK4, DJI, PARK7, LRRK2, PARK8, PINK1, PARK6, UCHLI, PARK5, SNCA, NACP, PARKI, PARK4, PRKN, PARK2, PDJ, DBH, NDUFV2, PINK 1, x-synuclein); in the case of Rett syndrome: MECP2, RTT, PPMX, MRX16, MRX79, CDKL5, STK9, MECP2, RTT, PPMX, MRX16, MRX79, x-Synuclein, DJ-1; in the case of Schizophrenia: Neuregulin1 (Nrg1), Erb4 (receptor for Neuregulin), Complexin1 (Cplx1), Tphl Tryptophan hydroxylase, Tph2, Tryptophan hydroxylase 2, Neurexin 1, GSK3, GSK3a, GSK3b, 5-HTT (Slc6a4), COMT, DRD (Drdla), SLC6A3, DAOA, DTNBPI, Dao (Daol)); in the case of Secretase Related Disorders (APH-1 (alpha and beta), Presenilin (Psen1), nicastrin, (Ncstn), PEN-2, Nos1, Parp1, Nat1, Nat2); in the case of Trinucleotide Repeat Disorders (HTT (Huntington's Dx), SBMA/SMAX1/AR (Kennedy's Dx), FXN/X25 (Friedrich's Ataxia), ATX3 (Machado-Joseph's Dx), ATXNI and ATXN2 (spinocerebellar ataxias), DMPK (myotonic dystrophy), Atrophin-1 and Atnl (DRPLA Dx), CBP (Creb-BP—global instability), VLDLR (Alzheimer's), Atxn7, Atxn10); in the case of diseases or disorders associated with or involving aberrant or abnormal axonal guidance signaling in the brain, neurons, and/or CNS: PRKCE; ITGAM; ROCK1; ITGA5; CXCR4; ADAM12; IGF1; RAC1; RAP1A; EIF4E; PRKCZ; NRP1; NTRK2; ARHGEF7; SMO; ROCK2; MAPK1; PGF; RAC2; PTPN11; GNAS; AKT2; PIK3CA; ERBB2; PRKCI; PTK2; CFL1; GNAQ; PIK3CB; CXCL12; PIK3C3; WNT11; PRKD1; GNB2L1; ABL1; MAPK3; ITGA1; KRAS; RHOA; PRKCD; PIK3C2A; ITGB7; GLI2; PXN; VASP; RAF1; FYN; ITGB1; MAP2K2; PAK4; ADAM17; AKT1; PIK3R1; GLII; WNT5A; ADAM10; MAP2K1; PAK3; ITGB3; CDC42; VEGFA; ITGA2; EPHA8; CRKL; RND1; GSK3B; AKT3; PRKCA; in the case of diseases or disorders associated with or involving aberrant or abnormal actin cytoskeleton signaling in the brain, neurons, and/or CNS: ACTN4; PRKCE; ITGAM; ROCK1; ITGA5; IRAKI; PRKAA2; EIF2AK2; RAC1; INS; ARHGEF7; GRK6; ROCK2; MAPK1; RAC2; PLK1; AKT2; PIK3CA; CDK8; PTK2; CFL1; PIK3CB; MYH9; DIAPH1; PIK3C3; MAPK8; F2R; MAPK3; SLC9A1; ITGAI; KRAS; RHOA; PRKCD; PRKAA1; MAPK9; CDK2; PIM1; PIK3C2A; ITGB7; PPPICC; PXN; VIL2; RAF1; GSN; DYRKIA; ITGB1; MAP2K2; PAK4; PIP5KIA; PIK3R1; MAP2K1; PAK3; ITGB3; CDC42; APC; ITGA2; TTK; CSNKIA1; CRKL; BRAF; VAV3; SGK; in the case of diseases or disorders associated with or involving Huntington's Disease signaling: PRKCE; IGF1; EP300; RCOR1; PRKCZ; HDAC4; TGM2; MAPK1; CAPNS1; AKT2; EGFR; NCOR2; SP1; CAPN2; PIK3CA; HDAC5; CREB1; PRKCI; HSPA5; REST; GNAQ; PIK3CB; PIK3C3; MAPK8; IGF1R; PRKD1; GNB2L1; BCL2L1; CAPN1; MAPK3; CASP8; HDAC2; HDAC7A; PRKCD; HDAC11; MAPK9; HDAC9; PIK3C2A; HDAC3; TP53; CASP9; CREBBP; AKT1; PIK3R1; PDPKI; CASP1; APAF1; FRAP1; CASP2; JUN; BAX; ATF4; AKT3; PRKCA; CLTC; SGK; HDAC6; CASP3; in the case of diseases or disorders associated with or involving aberrant, pathologic, and/or abnormal apoptosis regulation and/or signaling in the brain, neurons, and/or CNS and/or diseases or disorders thereof: PRKCE; ROCK1; BID; IRAK1; PRKAA2; EIF2AK2; BAK1; BIRC4; GRK6; MAPK1; CAPNS1; PLK1; AKT2; IKBKB; CAPN2; CDK8; FAS; NFKB2; BCL2; MAP3K14; MAPK8; BCL2L1; CAPN1; MAPK3; CASP8; KRAS; RELA; PRKCD; PRKAA1; MAPK9; CDK2; PIM1; TP53; TNF; RAF1; IKBKG; RELB; CASP9; DYRKIA; MAP2K2; CHUK; APAF1; MAP2K1; NFKB1; PAK3; LMNA; CASP2; BIRC2; TTK; CSNKIA1; BRAF; BAX; PRKCA; SGK; CASP3; BIRC3; PARP1; in the case of diseases or disorders associated with or involving aberrant, pathologic, and/or abnormal leukocyte extravasation signaling in the brain, neurons, and/or CNS and/or diseases or disorders thereof: ACTN4; CD44; PRKCE; ITGAM; ROCK1; CXCR4; CYBA; RAC1; RAP1A; PRKCZ; ROCK2; RAC2; PTPN11; MMP14; PIK3CA; PRKCI; PTK2; PIK3CB; CXCL12; PIK3C3; MAPK8; PRKD1; ABL1; MAPK10; CYBB; MAPK13; RHOA; PRKCD; MAPK9; SRC; PIK3C2A; BTK; MAPK14; NOX1; PXN; VIL2; VASP; ITGB1; MAP2K2; CTNND1; PIK3R1; CTNNB1; CLDNI; CDC42; F11R; ITK; CRKL; VAV3; CTTN; PRKCA; MMP1; MMP9; in the case of diseases or disorders associated with or involving aberrant, pathologic, and/or abnormal integrin signaling in the brain, neurons, and/or CNS and/or diseases or disorders thereof: ACTN4; ITGAM; ROCK1; ITGA5; RAC1; PTEN; RAP1A; TLN1; ARHGEF7; MAPK1; RAC2; CAPNS1; AKT2; CAPN2; PIK3CA; PTK2; PIK3CB; PIK3C3; MAPK8; CAV1; CAPN1; ABL1; MAPK3; ITGA1; KRAS; RHOA; SRC; PIK3C2A; ITGB7; PPPICC; ILK; PXN; VASP; RAF1; FYN; ITGB1; MAP2K2; PAK4; AKT1; PIK3R1; TNK2; MAP2K1; PAK3; ITGB3; CDC42; RND3; ITGA2; CRKL; BRAF; GSK3B; AKT3; in the case of diseases or disorders associated with or involving aberrant, pathologic, and/or abnormal acute phase response signaling in the brain, neurons, and/or CNS and/or diseases or disorders thereof: IRAK1; SOD2; MYD88; TRAF6; ELK1; MAPK1; PTPN11; AKT2; IKBKB; PIK3CA; FOS; NFKB2; MAP3K14; PIK3CB; MAPK8; RIPK1; MAPK3; IL6ST; KRAS; MAPK13; IL6R; RELA; SOCS1; MAPK9; FTL; NR3C1; TRAF2; SERPINE1; MAPK14; TNF; RAF1; PDK1; IKBKG; RELB; MAP3K7; MAP2K2; AKT1; JAK2; PIK3R1; CHUK; STAT3; MAP2K1; NFKB1; FRAP1; CEBPB; JUN; AKT3; ILIR1; IL6; in the case of diseases or disorders associated with or involving aberrant, pathologic, and/or abnormal PTEN signaling in the brain, neurons, and/or CNS and/or diseases or disorders thereof: ITGAM; ITGA5; RAC1; PTEN; PRKCZ; BCL2L11; MAPK1; RAC2; AKT2; EGFR; IKBKB; CBL; PIK3CA; CDKN1B; PTK2; NFKB2; BCL2; PIK3CB; BCL2L1; MAPK3; ITGA1; KRAS; ITGB7; ILK; PDGFRB; INSR; RAF1; IKBKG; CASP9; CDKN1A; ITGB1; MAP2K2; AKT1; PIK3R1; CHUK; PDGFRA; PDPK1; MAP2K1; NFKB1; ITGB3; CDC42; CCND1; GSK3A; ITGA2; GSK3B; AKT3; FOXO1; CASP3; RPS6KB1; in the case of diseases or disorders associated with or involving aberrant, pathologic, and/or abnormal p53 signaling in the brain, neurons, and/or CNS and/or diseases or disorders thereof: PTEN; EP300; BBC3; PCAF; FASN; BRCA1; GADD45A; BIRC5; AKT2; PIK3CA; CHEK1; TP53INP1; BCL2; PIK3CB; PIK3C3; MAPK8; THBS1; ATR; BCL2L1; E2F1; PMAIP1; CHEK2; TNFRSF10B; TP73; RB1; HDAC9; CDK2; PIK3C2A; MAPK14; TP53; LRDD; CDKN1A; HIPK2; AKT1; PIK3R1; RRM2B; APAF1; CTNNB1; SIRT1; CCND1; PRKDC; ATM; SFN; CDKN2A; JUN; SNAI2; GSK3B; BAX; AKT3; in the case of diseases or disorders associated with or involving aberrant, pathologic, and/or abnormal aryl hydrocarbon receptor signaling in the brain, neurons, and/or CNS and/or diseases or disorders thereof: HSPB1; EP300; FASN; TGM2; RXRA; MAPK1; NQO1; NCOR2; SP1; ARNT; CDKN1B; FOS; CHEK1; SMARCA4; NFKB2; MAPK8; ALDH1A1; ATR; E2F1; MAPK3; NRIP1; CHEK2; RELA; TP73; GSTP1; RB1; SRC; CDK2; AHR; NFE2L2; NCOA3; TP53; TNF; CDKN1A; NCOA2; APAF1; NFKB1; CCND1; ATM; ESRI; CDKN2A; MYC; JUN; ESR2; BAX; IL6; CYPIB1; HSP90AA1; in the case of diseases or disorders associated with or involving aberrant, pathologic, and/or abnormal xenobiotic metabolism signaling in the brain, neurons, and/or CNS and/or diseases or disorders thereof: PRKCE; EP300; PRKCZ; RXRA; MAPK1; NQO1; NCOR2; PIK3CA; ARNT; PRKCI; NFKB2; CAMK2A; PIK3CB; PPP2R1A; PIK3C3; MAPK8; PRKD1; ALDH1A1; MAPK3; NRIP1; KRAS; MAPK13; PRKCD; GSTP1; MAPK9; NOS2A; ABCB1; AHR; PPP2CA; FTL; NFE2L2; PIK3C2A; PPARGCIA; MAPK14; TNF; RAF1; CREBBP; MAP2K2; PIK3R1; PPP2R5C; MAP2K1; NFKB1; KEAP1; PRKCA; EIF2AK3; IL6; CYPIB1; HSP90AA1; in the case of diseases or disorders associated with or involving aberrant, pathologic, and/or abnormal SAPK/JNK signaling in the brain, neurons, and/or CNS and/or diseases or disorders thereof: PRKCE; IRAK1; PRKAA2; EIF2AK2; RAC1; ELK1; GRK6; MAPK1; GADD45A; RAC2; PLK1; AKT2; PIK3CA; FADD; CDK8; PIK3CB; PIK3C3; MAPK8; RIPK1; GNB2L1; IRS1; MAPK3; MAPK10; DAXX; KRAS; PRKCD; PRKAA1; MAPK9; CDK2; PIM1; PIK3C2A; TRAF2; TP53; LCK; MAP3K7; DYRKIA; MAP2K2; PIK3R1; MAP2K1; PAK3; CDC42; JUN; TTK; CSNKIA1; CRKL; BRAF; SGK; in the case of diseases or disorders associated with or involving aberrant, pathologic, and/or abnormal PPAr/RXR signaling in the brain, neurons, and/or CNS and/or diseases or disorders thereof: PRKAA2; EP300; INS; SMAD2; TRAF6; PPARA; FASN; RXRA; MAPK1; SMAD3; GNAS; IKBKB; NCOR2; ABCA1; GNAQ; NFKB2; MAP3K14; STAT5B; MAPK8; IRS1; MAPK3; KRAS; RELA; PRKAA1; PPARGCIA; NCOA3; MAPK14; INSR; RAF1; IKBKG; RELB; MAP3K7; CREBBP; MAP2K2; JAK2; CHUK; MAP2K1; NFKB1; TGFBR1; SMAD4; JUN; ILIR1; PRKCA; IL6; HSP90AA1; ADIPOQ; in the case of diseases or disorders associated with or involving aberrant, pathologic, and/or abnormal NF-kappaB signaling in the brain, neurons, and/or CNS and/or diseases or disorders thereof: IRAK1; EIF2AK2; EP300; INS; MYD88; PRKCZ; TRAF6; TBK1; AKT2; EGFR; IKBKB; PIK3CA; BTRC; NFKB2; MAP3K14; PIK3CB; PIK3C3; MAPK8; RIPK1; HDAC2; KRAS; RELA; PIK3C2A; TRAF2; TLR4; PDGFRB; TNF; INSR; LCK; IKBKG; RELB; MAP3K7; CREBBP; AKT1; PIK3R1; CHUK; PDGFRA; NFKB1; TLR2; BCL10; GSK3B; AKT3; TNFAIP3; ILIR1; in the case of diseases or disorders associated with or involving aberrant, pathologic, and/or abnormal neuregulin signaling in the brain, neurons, and/or CNS and/or diseases or disorders thereof: ERBB4; PRKCE; ITGAM; ITGA5; PTEN; PRKCZ; ELK1; MAPK1; PTPN11; AKT2; EGFR; ERBB2; PRKCI; CDKN1B; STAT5B; PRKD1; MAPK3; ITGAI; KRAS; PRKCD; STAT5A; SRC; ITGB7; RAF1; ITGB1; MAP2K2; ADAM17; AKT1; PIK3R1; PDPK1; MAP2K1; ITGB3; EREG; FRAP1; PSEN1; ITGA2; MYC; NRG1; CRKL; AKT3; PRKCA; HSP90AA1; RPS6KB1; in the case of diseases or disorders associated with or involving aberrant, pathologic, and/or abnormal wnt and beta catenin signaling in the brain, neurons, and/or CNS and/or diseases or disorders thereof: CD44; EP300; LRP6; DVL3; CSNK1E; GJA1; SMO; AKT2; PIN1; CDH1; BTRC; GNAQ; MARK2; PPP2R1A; WNT11; SRC; DKK1; PPP2CA; SOX6; SFRP2; ILK; LEF1; SOX9; TP53; MAP3K7; CREBBP; TCF7L2; AKT1; PPP2R5C; WNT5A; LRP5; CTNNB1; TGFBR1; CCND1; GSK3A; DVL1; APC; CDKN2A; MYC; CSNKIA1; GSK3B; AKT3; SOX2; in the case of diseases or disorders associated with or involving aberrant, pathologic, and/or abnormal insulin receptor signaling in the brain, neurons, and/or CNS and/or diseases or disorders thereof: PTEN; INS; EIF4E; PTPN1; PRKCZ; MAPK1; TSC1; PTPN11; AKT2; CBL; PIK3CA; PRKCI; PIK3CB; PIK3C3; MAPK8; IRS1; MAPK3; TSC2; KRAS; EIF4EBP1; SLC2A4; PIK3C2A; PPPICC; INSR; RAFI; FYN; MAP2K2; JAK1; AKT1; JAK2; PIK3R1; PDPK1; MAP2K1; GSK3A; FRAP1; CRKL; GSK3B; AKT3; FOXO1; SGK; RPS6KB1; in the case of diseases or disorders associated with or involving aberrant, pathologic, and/or abnormal IL-6 signaling in the brain, neurons, and/or CNS and/or diseases or disorders thereof: HSPB1; TRAF6; MAPKAPK2; ELK1; MAPK1; PTPN11; IKBKB; FOS; NFKB2; MAP3K14; MAPK8; MAPK3; MAPK10; IL6ST; KRAS; MAPK13; IL6R; RELA; SOCS1; MAPK9; ABCB1; TRAF2; MAPK14; TNF; RAF1; IKBKG; RELB; MAP3K7; MAP2K2; IL8; JAK2; CHUK; STAT3; MAP2K1; NFKB1; CEBPB; JUN; ILIR1; SRF; IL6; in the case of diseases or disorders associated with or involving aberrant, pathologic, and/or abnormal IGF-1 signaling in the brain, neurons, and/or CNS and/or diseases or disorders thereof: IGF1; PRKCZ; ELK1; MAPK1; PTPN11; NEDD4; AKT2; PIK3CA; PRKCI; PTK2; FOS; PIK3CB; PIK3C3; MAPK8; IGF1R; IRS1; MAPK3; IGFBP7; KRAS; PIK3C2A; YWHAZ; PXN; RAF1; CASP9; MAP2K2; AKT1; PIK3R1; PDPK1; MAP2K1; IGFBP2; SFN; JUN; CYR61; AKT3; FOXO1; SRF; CTGF; RPS6KB1; in the case of diseases or disorders associated with or involving aberrant, pathologic, and/or abnormal NRF2-mediated oxidative stress response pathway regulation or signaling in the brain, neurons, and/or CNS and/or diseases or disorders thereof: PRKCE; EP300; SOD2; PRKCZ; MAPK1; SQSTM1; NQO1; PIK3CA; PRKCI; FOS; PIK3CB; PIK3C3; MAPK8; PRKD1; MAPK3; KRAS; PRKCD; GSTP1; MAPK9; FTL; NFE2L2; PIK3C2A; MAPK14; RAF1; MAP3K7; CREBBP; MAP2K2; AKT1; PIK3R1; MAP2K1; PPIB; JUN; KEAP1; GSK3B; ATF4; PRKCA; EIF2AK3; HSP90AA1; PRDX1; in the case of diseases or disorders associated with or involving aberrant, pathologic, and/or abnormal PPAR (e.g. PPAR alpha, PPAR beta, PPAR delta, and/or PPAR gamma) regulation or signaling in the brain, neurons, and/or CNS and/or diseases or disorders thereof: EP300; INS; TRAF6; PPARA; RXRA; MAPK1; IKBKB; NCOR2; FOS; NFKB2; MAP3K14; STAT5B; MAPK3; NRIP1; KRAS; PPARG; RELA; STAT5A; TRAF2; PPARGCIA; PDGFRB; TNF; INSR; RAF1; IKBKG; RELB; MAP3K7; CREBBP; MAP2K2; CHUK; PDGFRA; MAP2K1; NFKB1; JUN; ILIR1; HSP90AA1; in the case of diseases or disorders associated with or involving aberrant, pathologic, and/or abnormal Fc Epsilon RI regulation or signaling in the brain, neurons, and/or CNS and/or diseases or disorders thereof: PRKCE; RAC1; PRKCZ; LYN; MAPK1; RAC2; PTPN11; AKT2; PIK3CA; SYK; PRKCI; PIK3CB; PIK3C3; MAPK8; PRKD1; MAPK3; MAPK10; KRAS; MAPK13; PRKCD; MAPK9; PIK3C2A; BTK; MAPK14; TNF; RAF1; FYN; MAP2K2; AKT1; PIK3R1; PDPK1; MAP2K1; AKT3; VAV3; PRKCA; in the case of diseases or disorders associated with or involving aberrant, pathologic, and/or abnormal G-protein coupled receptor regulation or signaling in the brain, neurons, and/or CNS and/or diseases or disorders thereof: PRKCE; RAP1A; RGS16; MAPK1; GNAS; AKT2; IKBKB; PIK3CA; CREB1; GNAQ; NFKB2; CAMK2A; PIK3CB; PIK3C3; MAPK3; KRAS; RELA; SRC; PIK3C2A; RAF1; IKBKG; RELB; FYN; MAP2K2; AKT1; PIK3R1; CHUK; PDPK1; STAT3; MAP2K1; NFKB1; BRAF; ATF4; AKT3; PRKCA; in the case of diseases or disorders associated with or involving aberrant, pathologic, and/or abnormal inositol phosphate metabolism regulation or signaling in the brain, neurons, and/or CNS and/or diseases or disorders thereof: PRKCE; IRAK1; PRKAA2; EIF2AK2; PTEN; GRK6; MAPK1; PLK1; AKT2; PIK3CA; CDK8; PIK3CB; PIK3C3; MAPK8; MAPK3; PRKCD; PRKAA1; MAPK9; CDK2; PIM1; PIK3C2A; DYRKIA; MAP2K2; PIP5K1A; PIK3R1; MAP2K1; PAK3; ATM; TTK; CSNKIA1; BRAF; SGK; in the case of diseases or disorders associated with or involving aberrant, pathologic, and/or abnormal PDGF regulation or signaling in the brain, neurons, and/or CNS and/or diseases or disorders thereof: EIF2AK2; ELK1; ABL2; MAPK1; PIK3CA; FOS; PIK3CB; PIK3C3; MAPK8; CAV1; ABL1; MAPK3; KRAS; SRC; PIK3C2A; PDGFRB; RAF1; MAP2K2; JAK1; JAK2; PIK3R1; PDGFRA; STAT3; SPHK1; MAP2K1; MYC; JUN; CRKL; PRKCA; SRF; STAT1; SPHK2; in the case of diseases or disorders associated with involving aberrant, pathologic, and/or abnormal VEGF regulation or signaling in the brain, neurons, and/or CNS and/or diseases or disorders thereof: ACTN4; ROCK1; KDR; FLT1; ROCK2; MAPK1; PGF; AKT2; PIK3CA; ARNT; PTK2; BCL2; PIK3CB; PIK3C3; BCL2L1; MAPK3; KRAS; HIF1A; NOS3; PIK3C2A; PXN; RAF1; MAP2K2; ELAVL1; AKT1; PIK3R1; MAP2K1; SFN; VEGFA; AKT3; FOXO1; PRKCA; in the case of diseases or disorders associated with or involving aberrant, pathologic, and/or abnormal natural killer cell regulation or signaling in the brain, neurons, and/or CNS and/or diseases or disorders thereof: PRKCE; RAC1; PRKCZ; MAPK1; RAC2; PTPN11; KIR2DL3; AKT2; PIK3CA; SYK; PRKCI; PIK3CB; PIK3C3; PRKD1; MAPK3; KRAS; PRKCD; PTPN6; PIK3C2A; LCK; RAFI; FYN; MAP2K2; PAK4; AKT1; PIK3R1; MAP2K1; PAK3; AKT3; VAV3; PRKCA; in the case of diseases or disorders associated with or involving aberrant, pathologic, and/or abnormal cell cycle G1/S checkpoint regulation or signaling in the brain, neurons, and/or CNS and/or diseases or disorders thereof: HDAC4; SMAD3; SUV39H1; HDAC5; CDKN1B; BTRC; ATR; ABL1; E2F1; HDAC2; HDAC7A; RB1; HDAC11; HDAC9; CDK2; E2F2; HDAC3; TP53; CDKN1A; CCND1; E2F4; ATM; RBL2; SMAD4; CDKN2A; MYC; NRG1; GSK3B; RBL1; HDAC6; in the case of diseases or disorders associated with or involving aberrant, pathologic, and/or abnormal T-cell receptor regulation or signaling in the brain, neurons, and/or CNS and/or diseases or disorders thereof: RAC1; ELK1; MAPK1; IKBKB; CBL; PIK3CA; FOS; NFKB2; PIK3CB; PIK3C3; MAPK8; MAPK3; KRAS; RELA; PIK3C2A; BTK; LCK; RAFI; IKBKG; RELB; FYN; MAP2K2; PIK3R1; CHUK; MAP2K1; NFKB1; ITK; BCL10; JUN; VAV3; in the case of diseases or disorders associated with or involving aberrant, pathologic, and/or abnormal death receptor regulation or signaling in the brain, neurons, and/or CNS and/or diseases or disorders thereof: CRADD; HSPB1; BID; BIRC4; TBK1; IKBKB; FADD; FAS; NFKB2; BCL2; MAP3K14; MAPK8; RIPK1; CASP8; DAXX; TNFRSF10B; RELA; TRAF2; TNF; IKBKG; RELB; CASP9; CHUK; APAF1; NFKB1; CASP2; BIRC2; CASP3; BIRC3; in the case of diseases or disorders associated with or involving aberrant, pathologic, and/or FGF regulation or signaling in the brain, neurons, and/or CNS and/or diseases or disorders thereof: RAC1; FGFR1; MET; MAPKAPK2; MAPK1; PTPN11; AKT2; PIK3CA; CREB1; PIK3CB; PIK3C3; MAPK8; MAPK3; MAPK13; PTPN6; PIK3C2A; MAPK14; RAF1; AKT1; PIK3R1; STAT3; MAP2K1; FGFR4; CRKL; ATF4; AKT3; PRKCA; HGF; in the case of diseases or disorders associated with or involving aberrant, pathologic, and/or GM-CSF regulation or signaling in the brain, neurons, and/or CNS and/or diseases or disorders thereof: LYN; ELK1; MAPK1; PTPN11; AKT2; PIK3CA; CAMK2A; STAT5B; PIK3CB; PIK3C3; GNB2L1; BCL2L1; MAPK3; ETS1; KRAS; RUNX1; PIM1; PIK3C2A; RAF1; MAP2K2; AKT1; JAK2; PIK3R1; STAT3; MAP2K1; CCND1; AKT3; STAT1; in the case of diseases or disorders associated with or involving aberrant, pathologic, and/or amyotrophic lateral sclerosis regulation or signaling in the brain, neurons, and/or CNS and/or diseases or disorders thereof: BID; IGF1; RAC1; BIRC4; PGF; CAPNS1; CAPN2; PIK3CA; BCL2; PIK3CB; PIK3C3; BCL2L1; CAPN1; PIK3C2A; TP53; CASP9; PIK3R1; RAB5A; CASP1; APAF1; VEGFA; BIRC2; BAX; AKT3; CASP3; BIRC3; in the case of diseases or disorders associated with or involving aberrant, pathologic, and/or JAK/Stat regulation or signaling in the brain, neurons, and/or CNS and/or diseases or disorders thereof: PTPN1; MAPK1; PTPN11; AKT2; PIK3CA; STAT5B; PIK3CB; PIK3C3; MAPK3; KRAS; SOCS1; STAT5A; PTPN6; PIK3C2A; RAFI; CDKN1A; MAP2K2; JAK1; AKT1; JAK2; PIK3R1; STAT3; MAP2K1; FRAP1; AKT3; STAT1; in the case of diseases or disorders associated with or involving aberrant, pathologic, and/or nicotinate and nicotinamide metabolism regulation or signaling in the brain, neurons, and/or CNS and/or diseases or disorders thereof: PRKCE; IRAK1; PRKAA2; EIF2AK2; GRK6; MAPK1; PLK1; AKT2; CDK8; MAPK8; MAPK3; PRKCD; PRKAA1; PBEF1; MAPK9; CDK2; PIM1; DYRKIA; MAP2K2; MAP2K1; PAK3; NT5E; TTK; CSNKIA1; BRAF; SGK; in the case of diseases or disorders associated with or involving aberrant, pathologic, and/or chemokine signaling regulation or signaling in the brain, neurons, and/or CNS and/or diseases or disorders thereof: CXCR4; ROCK2; MAPK1; PTK2; FOS; CFL1; GNAQ; CAMK2A; CXCL12; MAPK8; MAPK3; KRAS; MAPK13; RHOA; CCR3; SRC; PPPICC; MAPK14; NOX1; RAF1; MAP2K2; MAP2K1; JUN; CCL2; PRKCA; in the case of diseases or disorders associated with or involving aberrant, pathologic, and/or IL-2 signaling regulation or signaling in the brain, neurons, and/or CNS and/or diseases or disorders thereof: ELK1; MAPK1; PTPN11; AKT2; PIK3CA; SYK; FOS; STAT5B; PIK3CB; PIK3C3; MAPK8; MAPK3; KRAS; SOCS1; STAT5A; PIK3C2A; LCK; RAF1; MAP2K2; JAK1; AKT1; PIK3R1; MAP2K1; JUN; AKT3; in the case of diseases or disorders associated with or involving synaptic long term depression in the brain, neurons, and/or CNS and/or diseases or disorders thereof: PRKCE; IGF1; PRKCZ; PRDX6; LYN; MAPK1; GNAS; PRKCI; GNAQ; PPP2R1A; IGF1R; PRKD1; MAPK3; KRAS; GRN; PRKCD; NOS3; NOS2A; PPP2CA; YWHAZ; RAF1; MAP2K2; PPP2R5C; MAP2K1; PRKCA; in the case of diseases or disorders associated with or involving aberrant, pathologic, and/or estrogen receptor regulation or signaling in the brain, neurons, and/or CNS and/or diseases or disorders thereof: TAF4B; EP300; CARMI; PCAF; MAPK1; NCOR2; SMARCA4; MAPK3; NRIP1; KRAS; SRC; NR3C1; HDAC3; PPARGCIA; RBM9; NCOA3; RAF1; CREBBP; MAP2K2; NCOA2; MAP2K1; PRKDC; ESRI; ESR2; in the case of diseases or disorders associated with or involving aberrant, pathologic, and/or protein ubiquitination pathway activity, regulation, and/or signaling in the brain, neurons, and/or CNS and/or diseases or disorders thereof: TRAF6; SMURF1; BIRC4; BRCA1; UCHLI; NEDD4; CBL; UBE2I; BTRC; HSPA5; USP7; USP10; FBXW7; USP9X; STUB1; USP22; B2M; BIRC2; PARK2; USP8; USP1; VHL; HSP90AA1; BIRC3; in the case of diseases or disorders associated with or involving aberrant, pathologic, and/or IL-10 regulation or signaling in the brain, neurons, and/or CNS and/or diseases or disorders thereof: TRAF6; CCR1; ELK1; IKBKB; SP1; FOS; NFKB2; MAP3K14; MAPK8; MAPK13; RELA; MAPK14; TNF; IKBKG; RELB; MAP3K7; JAK1; CHUK; STAT3; NFKB1; JUN; ILIR1; IL6; in the case of diseases or disorders associated with or involving aberrant, pathologic, and/or Vitamin D receptor (VDR) and/or RXR regulation or signaling in the brain, neurons, and/or CNS and/or diseases or disorders thereof: PRKCE; EP300; PRKCZ; RXRA; GADD45A; HES1; NCOR2; SP1; PRKCI; CDKN1B; PRKD1; PRKCD; RUNX2; KLF4; YY1; NCOA3; CDKN1A; NCOA2; SPP1; LRP5; CEBPB; FOXO1; PRKCA; in the case of diseases or disorders associated with or involving aberrant, pathologic, and/or TGF-beta regulation or signaling in the brain, neurons, and/or CNS and/or diseases or disorders thereof: EP300; SMAD2; SMURF1; MAPK1; SMAD3; SMAD1; FOS; MAPK8; MAPK3; KRAS; MAPK9; RUNX2; SERPINE1; RAF1; MAP3K7; CREBBP; MAP2K2; MAP2K1; TGFBR1; SMAD4; JUN; SMAD5; in the case of diseases or disorders associated with or involving aberrant, pathologic, and/or Toll-like Receptor activity, regulation, and/or signaling in the brain, neurons, and/or CNS and/or diseases or disorders thereof: IRAK1; EIF2AK2; MYD88; TRAF6; PPARA; ELK1; IKBKB; FOS; NFKB2; MAP3K14; MAPK8; MAPK13; RELA; TLR4; MAPK14; IKBKG; RELB; MAP3K7; CHUK; NFKB1; TLR2; JUN; in the case of diseases or disorders associated with or involving aberrant, pathologic, and/or p38 MAPK activity, regulation, and/or signaling in the brain, neurons, and/or CNS and/or diseases or disorders thereof: HSPB1; IRAK1; TRAF6; MAPKAPK2; ELK1; FADD; FAS; CREB1; DDIT3; RPS6KA4; DAXX; MAPK13; TRAF2; MAPK14; TNF; MAP3K7; TGFBR1; MYC; ATF4; ILIR1; SRF; STAT1; in the case of diseases or disorders associated with or involving aberrant, pathologic, and/or neurotrophin/TRK activity, regulation, and/or signaling in the brain, neurons, and/or CNS and/or diseases or disorders thereof: NTRK2; MAPK1; PTPN11; PIK3CA; CREB1; FOS; PIK3CB; PIK3C3; MAPK8; MAPK3; KRAS; PIK3C2A; RAF1; MAP2K2; AKT1; PIK3R1; PDPK1; MAP2K1; CDC42; JUN; ATF4; in the case of diseases or disorders associated with or involving aberrant, pathologic, and/or FXR and/or RXR activity, regulation, and/or signaling in the brain, neurons, and/or CNS and/or diseases or disorders thereof: INS; PPARA; FASN; RXRA; AKT2; SDC1; MAPK8; APOB; MAPK10; PPARG; MTTP; MAPK9; PPARGCIA; TNF; CREBBP; AKT1; SREBF1; FGFR4; AKT3; FOXO1; in the case of diseases or disorders associated with or involving aberrant, pathologic, and/or synaptic long term potentiation in the brain, neurons, and/or CNS and/or diseases or disorders thereof: PRKCE; RAP1A; EP300; PRKCZ; MAPK1; CREB1; PRKCI; GNAQ; CAMK2A; PRKD1; MAPK3; KRAS; PRKCD; PPPICC; RAFI; CREBBP; MAP2K2; MAP2K1; ATF4; PRKCA; in the case of diseases or disorders associated with or involving aberrant, pathologic, and/or calcium regulation and/or signaling in the brain, neurons, and/or CNS and/or diseases or disorders thereof: RAP1A; EP300; HDAC4; MAPK1; HDAC5; CREB1; CAMK2A; MYH9; MAPK3; HDAC2; HDAC7A; HDAC11; HDAC9; HDAC3; CREBBP; CALR; CAMKK2; ATF4; HDAC6; in the case of diseases or disorders associated with or involving aberrant, pathologic, and/or EGF or EGFR regulation and/or signaling in the brain, neurons, and/or CNS and/or diseases or disorders thereof: ELK1; MAPK1; EGFR; PIK3CA; FOS; PIK3CB; PIK3C3; MAPK8; MAPK3; PIK3C2A; RAF1; JAK1; PIK3R1; STAT3; MAP2K1; JUN; PRKCA; SRF; STAT1; in the case of diseases or disorders associated with or involving aberrant, pathologic, and/or LPS/IL-1 mediated inhibition of RXR function, regulation and/or signaling in the brain, neurons, and/or CNS and/or diseases or disorders thereof: IRAK1; MYD88; TRAF6; PPARA; RXRA; ABCA1; MAPK8; ALDH1A1; GSTP1; MAPK9; ABCB1; TRAF2; TLR4; TNF; MAP3K7; NR1H2; SREBF1; JUN; ILIR1; in the case of diseases or disorders associated with or involving aberrant, pathologic, and/or LXR/RXR function, regulation and/or signaling in the brain, neurons, and/or CNS and/or diseases or disorders thereof: FASN; RXRA; NCOR2; ABCA1; NFKB2; IRF3; RELA; NOS2A; TLR4; TNF; RELB; LDLR; NR1H2; NFKB1; SREBF1; ILIR1; CCL2; IL6; MMP9; in the case of diseases or disorders associated with or involving aberrant, pathologic, and/or amyloid processing in the brain, neurons, and/or CNS and/or diseases or disorders thereof: PRKCE; CSNK1E; MAPK1; CAPNS1; AKT2; CAPN2; CAPN1; MAPK3; MAPK13; MAPT; MAPK14; AKT1; PSEN1; CSNK1A1; GSK3B; AKT3; APP; in the case of diseases or disorders associated with or involving aberrant, pathologic, and/or abnormal IL-4 activity, signaling, and/or regulation in the brain, neurons, and/or CNS and/or diseases or disorders thereof: AKT2; PIK3CA; PIK3CB; PIK3C3; IRS1; KRAS; SOCS1; PTPN6; NR3C1; PIK3C2A; JAK1; AKT1; JAK2; PIK3R1; FRAP1; AKT3; RPS6KB1; in the case of diseases or disorders associated with or involving aberrant, pathologic, and/or abnormal cell cycle: G2/M DNA damage checkpoint regulation activity, signaling, and/or regulation in the brain, neurons, and/or CNS and/or diseases or disorders thereof: EP300; PCAF; BRCA1; GADD45A; PLK1; BTRC; CHEK1; ATR; CHEK2; YWHAZ; TP53; CDKN1A; PRKDC; ATM; SFN; CDKN2A; in the case of diseases or disorders associated with or involving aberrant, pathologic, and/or abnormal purine metabolism signaling thereof, and/or regulation thereof in the brain, neurons, and/or CNS and/or diseases or disorders thereof: NME2; SMARCA4; MYH9; RRM2; ADAR; EIF2AK4; PKM2; ENTPD1; RAD51; RRM2B; TJP2; RAD51C; NT5E; POLD1; NME1; in the case of diseases or disorders associated with or involving aberrant, pathologic, and/or abnormal cAMP-mediated signaling and/or regulation in the brain, neurons, and/or CNS and/or diseases or disorders thereof: RAPIA; MAPK1; GNAS; CREB1; CAMK2A; MAPK3; SRC; RAFI; MAP2K2; STAT3; MAP2K1; BRAF; ATF4; in the case of diseases or disorders associated with or involving aberrant, pathologic, and/or abnormal mitochondrial function in the brain, neurons, and/or CNS and/or diseases or disorders thereof: SOD2; MAPK8; CASP8; MAPK10; MAPK9; CASP9; PARK7; PSEN1; PARK2; APP; CASP3; AIF; CytC; SMAC (Diablo); Aifm-1; Aifm-2; in the case of diseases or disorders associated with or involving aberrant, pathologic, and/or abnormal notch signaling and/or regulation in the brain, neurons, and/or CNS and/or diseases or disorders thereof: HES1; JAG1; NUMB; NOTCH4; ADAM17; NOTCH2; PSEN1; NOTCH3; NOTCH1; DLL4; in the case of diseases or disorders associated with or involving aberrant, pathologic, and/or abnormal endoplasmic reticulum stress pathway activity, signaling, and/or regulation in the brain, neurons, and/or CNS and/or diseases or disorders thereof: HSPA5; MAPK8; XBP1; TRAF2; ATF6; CASP9; ATF4; EIF2AK3; CASP3; in the case of diseases or disorders associated with or involving aberrant, pathologic, and/or abnormal pyrimidine metabolism, signaling thereof, and/or regulation thereof in the brain, neurons, and/or CNS and/or diseases or disorders thereof: NME2; AICDA; RRM2; EIF2AK4; ENTPD1; RRM2B; NT5E; POLD1; NME1; in the case of diseases or disorders associated with or involving aberrant, pathologic, and/or abnormal Parkinson's signaling in the brain, neurons, and/or CNS and/or diseases or disorders thereof: UCHL1; MAPK8; MAPK13; MAPK14; CASP9; PARK7; PARK2; CASP3; in the case of diseases or disorders associated with or involving aberrant, pathologic, and/or abnormal Glycolysis/Gluconeogenesis activity, signaling, and/or regulation in the brain, neurons, and/or CNS and/or diseases or disorders thereof: HK2; GCK; GPI; ALDH1A1; PKM2; LDHA; HK1; in the case of diseases or disorders associated with or involving aberrant, pathologic, and/or abnormal interferon activity, signaling, and/or regulation in the brain, neurons, and/or CNS and/or diseases or disorders thereof: IRF1; SOCS1; JAK1; JAK2; IFITM1; STAT1; IFIT3; in the case of diseases or disorders associated with or involving aberrant, pathologic, and/or abnormal sonic the hedgehog activity, signaling, and/or regulation in the brain, neurons, and/or CNS and/or diseases or disorders thereof: ARRB2; SMO; GLI2; DYRKIA; GLI1; GSK3B; DYRK1B; in the case of diseases or disorders associated with or involving aberrant, pathologic, and/or abnormal glycerophospholipid metabolism, signaling thereof, and/or regulation thereof in the brain, neurons, and/or CNS and/or diseases or disorders thereof: PLD1; GRN; GPAM; YWHAZ; SPHK1; SPHK2; in the case of diseases or disorders associated with or involving aberrant, pathologic, and/or abnormal phospholipid degradation, signaling thereof, and/or regulation thereof in the brain, neurons, and/or CNS and/or diseases or disorders thereof: PRDX6; PLD1; GRN; YWHAZ; SPHK1; SPHK2; in the case of diseases or disorders associated with or involving aberrant, pathologic, and/or abnormal tryptophan metabolism, signaling thereof, and/or regulation thereof in the brain, neurons, and/or CNS and/or diseases or disorders thereof: SIAH2; PRMT5; NEDD4; ALDH1A1; CYPIB1; SIAH1; in the case of diseases or disorders associated with or involving aberrant, pathologic, and/or abnormal lysine degradation, signaling thereof, and/or regulation thereof in the brain, neurons, and/or CNS and/or diseases or disorders thereof: SUV39H1; EHMT2; NSD1; SETD7; PPP2R5C; in the case of diseases or disorders associated with or involving aberrant, pathologic, and/or abnormal nucleotide excision repair pathway activity, signaling thereof, and/or regulation thereof in the brain, neurons, and/or CNS and/or diseases or disorders thereof: ERCC5; ERCC4; XPA; XPC; ERCC1; in the case of diseases or disorders associated with or involving aberrant, pathologic, and/or abnormal nucleotide starch and sucrose metabolism, signaling thereof, and/or regulation thereof in the brain, neurons, and/or CNS and/or diseases or disorders thereof: UCHL1; HK2; GCK; GPI; HK1; in the case of diseases or disorders associated with or involving aberrant, pathologic, and/or abnormal aminosugars metabolism, signaling thereof, and/or regulation thereof in the brain, neurons, and/or CNS and/or diseases or disorders thereof: NQO1; HK2; GCK; HK1; in the case of diseases or disorders associated with or involving aberrant, pathologic, and/or abnormal arachidonic acid metabolism, signaling thereof, and/or regulation thereof in the brain, neurons, and/or CNS and/or diseases or disorders thereof: PRDX6; GRN; YWHAZ; CYPIB1; in the case of diseases or disorders associated with or involving aberrant, pathologic, and/or abnormal circadian rhythm signaling and/or regulation in the brain, neurons, and/or CNS and/or diseases or disorders thereof: CSNK1E; CREB1; ATF4; NRID1; in the case of diseases or disorders associated with or involving aberrant, pathologic, and/or coagulation system activity signaling and/or regulation in the brain, neurons, and/or CNS and/or diseases or disorders thereof: BDKRB1; F2R; SERPINE1; F3; a PAR (e.g. PAR1, PAR2, etc.) PLC, aPC; in the case of diseases or disorders associated with or involving aberrant, pathologic, and/or abnormal dopamine receptor signaling and/or regulation in the brain, neurons, and/or CNS and/or diseases or disorders thereof: PPP2R1A; PPP2CA; PPPICC; PPP2R5C; in the case of diseases or disorders associated with or involving aberrant, pathologic, and/or abnormal Glutathione Metabolism signaling and/or regulation in the brain, neurons, and/or CNS and/or diseases or disorders thereof: IDH2; GSTP1; ANPEP; IDH1; in the case of diseases or disorders associated with or involving aberrant, pathologic, and/or abnormal Glycerolipid Metabolism signaling and/or regulation in the brain, neurons, and/or CNS and/or diseases or disorders thereof: ALDH1A1; GPAM; SPHK1; SPHK2; in the case of diseases or disorders associated with or involving aberrant, pathologic, and/or abnormal Linoleic Acid Metabolism signaling and/or regulation in the brain, neurons, and/or CNS and/or diseases or disorders thereof: PRDX6; GRN; YWHAZ; CYPIB1; in the case of diseases or disorders associated with or involving aberrant, pathologic, and/or abnormal Methionine Metabolism signaling and/or regulation in the brain, neurons, and/or CNS and/or diseases or disorders thereof: DNMT1; DNMT3B; AHCY; DNMT3A; in the case of diseases or disorders associated with or involving aberrant, pathologic, and/or abnormal Pyruvate Metabolism signaling and/or regulation in the brain, neurons, and/or CNS and/or diseases or disorders thereof: GLO1; ALDH1A1; PKM2; LDHA; in the case of diseases or disorders associated with or involving aberrant, pathologic, and/or abnormal Arginine and Proline Metabolism signaling and/or regulation in the brain, neurons, and/or CNS and/or diseases or disorders thereof: ALDH1A1; NOS3; NOS2A; in the case of diseases or disorders associated with or involving aberrant, pathologic, and/or abnormal Eicosanoid signaling and/or regulation in the brain, neurons, and/or CNS and/or diseases or disorders thereof: PRDX6; GRN; YWHAZ; in the case of diseases or disorders associated with or involving aberrant, pathologic, and/or abnormal fructose and mannose metabolism signaling and/or regulation in the brain, neurons, and/or CNS and/or diseases or disorders thereof: HK2; GCK; HK1; in the case of diseases or disorders associated with or involving aberrant, pathologic, and/or abnormal antigen presentation pathway activity, signaling and/or regulation in the brain, neurons, and/or CNS and/or diseases or disorders thereof: CALR; B2M; in the case of diseases or disorders associated with or involving aberrant, pathologic, and/or abnormal steroid biosynthesis in the brain, neurons, and/or CNS and/or diseases or disorders thereof: NQO1; DHCR7; in the case of diseases or disorders associated with or involving aberrant, pathologic, and/or abnormal butanoate metabolism in the brain, neurons, and/or CNS and/or diseases or disorders thereof: ALDH1A1; NLGN1; in the case of diseases or disorders associated with or involving an aberrant, pathologic, and/or abnormal citrate cycle in the brain, neurons, and/or CNS and/or diseases or disorders thereof: IDH2; IDH1; in the case of diseases or disorders associated with or involving aberrant, pathologic, and/or abnormal fatty acid metabolism in the brain, neurons, and/or CNS and/or diseases or disorders thereof: ALDH1A1; CYPIB1; in the case of diseases or disorders associated with or involving aberrant, pathologic, and/or abnormal Glycerophospholipid metabolism in the brain, neurons, and/or CNS and/or diseases or disorders thereof: PRDX6; CHKA; in the case of diseases or disorders associated with or involving aberrant, pathologic, and/or abnormal histidine metabolism in the brain, neurons, and/or CNS and/or diseases or disorders thereof: PRMT5; ALDH1A1; in the case of diseases or disorders associated with or involving aberrant, pathologic, and/or abnormal inositol metabolism in the brain, neurons, and/or CNS and/or diseases or disorders thereof: ERO1L; APEX1; in the case of diseases or disorders associated with or involving aberrant, pathologic, and/or abnormal Phenylalanine metabolism in the brain, neurons, and/or CNS and/or diseases or disorders thereof: PRDX6; PRDX1; in the case of diseases or disorders associated with or involving aberrant, pathologic, and/or abnormal Seleno amino acid metabolism in the brain, neurons, and/or CNS and/or diseases or disorders thereof: PRMT5; AHCY; in the case of diseases or disorders associated with or involving aberrant, pathologic, and/or abnormal Sphingolipid metabolism in the brain, neurons, and/or CNS and/or diseases or disorders thereof: SPHK1; SPHK2; in the case of diseases or disorders associated with or involving aberrant, pathologic, and/or abnormal Aminophosphonate metabolism in the brain, neurons, and/or CNS and/or diseases or disorders thereof: PRMT5; in the case of diseases or disorders associated with or involving aberrant, pathologic, and/or abnormal androgen and/or estrogen metabolism in the brain, neurons, and/or CNS and/or diseases or disorders thereof: PRMT5; in the case of diseases or disorders associated with or involving aberrant, pathologic, and/or abnormal Ascorbate and Aldarate metabolism in the brain, neurons, and/or CNS and/or diseases or disorders thereof: ALDH1A1; in the case of diseases or disorders associated with or involving aberrant, pathologic, and/or abnormal Cysteine Metabolism in the brain, neurons, and/or CNS and/or diseases or disorders thereof: LDHA; in the case of diseases or disorders associated with or involving aberrant, pathologic, and/or abnormal fatty acid biosynthesis in the brain, neurons, and/or CNS and/or diseases or disorders thereof: FASN; in the case of diseases or disorders associated with or involving aberrant, pathologic, and/or abnormal glutamate receptor signaling in the brain, neurons, and/or CNS and/or diseases or disorders thereof: GNB2L1; in the case of diseases or disorders associated with or involving aberrant, pathologic, and/or abnormal Pentose Phosphate pathway in the brain, neurons, and/or CNS and/or diseases or disorders thereof: GPI; in the case of diseases or disorders associated with or involving aberrant, pathologic, and/or abnormal retinol metabolism in the brain, neurons, and/or CNS and/or diseases or disorders thereof: ALDH1A1; in the case of diseases or disorders associated with or involving aberrant, pathologic, and/or abnormal Pentose and Glucuronate interconversions in the brain, neurons, and/or CNS and/or diseases or disorders thereof: UCHL1; in the case of diseases or disorders associated with or involving aberrant, pathologic, and/or abnormal Riboflavin Metabolism in the brain, neurons, and/or CNS and/or diseases or disorders thereof: TYR; in the case of diseases or disorders associated with or involving aberrant, pathologic, and/or abnormal Tyrosine Metabolism in the brain, neurons, and/or CNS and/or diseases or disorders thereof: PRMT5, TYR; in the case of diseases or disorders associated with or involving aberrant, pathologic, and/or abnormal Ubiquinone biosynthesis in the brain, neurons, and/or CNS and/or diseases or disorders thereof: PRMT5; in the case of diseases or disorders associated with or involving aberrant, pathologic, and/or abnormal Valine, leucine and isoleucine degradation in the brain, neurons, and/or CNS and/or diseases or disorders thereof: ALDH1A1; in the case of diseases or disorders associated with or involving aberrant, pathologic, and/or abnormal glycine, serine, and threonine metabolism in the brain, neurons, and/or CNS and/or diseases or disorders thereof: CHKA; in the case of diseases or disorders associated with or involving aberrant, pathologic, and/or abnormal lysine degradation in the brain, neurons, and/or CNS and/or diseases or disorders thereof: ALDH1A1; in the case of diseases or disorders associated with or involving aberrant, pathologic, and/or abnormal pain or pain signaling or pain signal generation in the brain, neurons, and/or CNS and/or diseases or disorders thereof: TRPM7; TRPC5; TRPC6; TRPC1; Cnr1; cnr2; Grk2; Trpal; Pomc; Cgrp; Crf; Pka; Era; Nr2b; TRPM5; Prkaca; Prkacb; Prkarla; Prkar2a; in the case of diseases or disorders associated with or involving aberrant, pathologic, and/or abnormal brain, neuron, and/or CNS development and/or diseases or disorders thereof: BMP-4; Chordin (Chrd); Noggin (Nog); WNT (Wnt2; Wnt2b; Wnt3a; Wnt4; Wnt5a; Wnt6; Wnt7b; Wnt8b; Wnt9a; Wnt9b; Wnt10a; Wnt10b; Wnt16); beta-catenin; Dkk-1; Frizzled related proteins; Otx-2; Gbx2; FGF-8; Reelin; Dab1; unc-86 (Pou4f1 or Brn3a); Numb; Re1n; in the case of diseases or disorders associated with or involving prion disorders of or in the brain, neuron, and/or CNS and/or diseases or disorders thereof: Prp; in the case of substance or activity additions involving activities of the brain, neuron, and/or CNS: Prkce (alcohol); Drd2; Drd4; ABAT (alcohol); GRIA2; Grm5; Grin1; Htr1b; Grin2a; Drd3; Pdyn; Gria1 (alcohol); in the case of diseases or disorders associated with or involving PI3K/AKT signaling and/or regulation thereof in the brain, neuron, and/or CNS and/or diseases or disorders thereof: PRKCE; ITGAM; ITGA5; IRAK1; PRKAA2; EIF2AK2; PTEN; EIF4E; PRKCZ; GRK6; MAPK1; TSC1; PLK1; AKT2; IKBKB; PIK3CA; CDK8; CDKN1B; NFKB2; BCL2; PIK3CB; PPP2R1A; MAPK8; BCL2L1; MAPK3; TSC2; ITGA1; KRAS; EIF4EBP1; RELA; PRKCD; NOS3; PRKAA1; MAPK9; CDK2; PPP2CA; PIM1; ITGB7; YWHAZ; ILK; TP53; RAFI; IKBKG; RELB; DYRKIA; CDKN1A; ITGB1; MAP2K2; JAK1; AKT1; JAK2; PIK3R1; CHUK; PDPK1; PPP2R5C; CTNNB1; MAP2K1; NFKB1; PAK3; ITGB3; CCND1; GSK3A; FRAP1; SFN; ITGA2; TTK; CSNKIA1; BRAF; GSK3B; AKT3; FOXO1; SGK; HSP90AA1; RPS6KB1; in the case of diseases or disorders associated with or involving ERK/MAPK signaling and/or regulation thereof in the brain, neuron, and/or CNS and/or diseases or disorders thereof: PRKCE; ITGAM; ITGA5; HSPB1; IRAK1; PRKAA2; EIF2AK2; RAC1; RAP1A; TLN1; EIF4E; ELK1; GRK6; MAPK1; RAC2; PLK1; AKT2; PIK3CA; CDK8; CREB1; PRKCI; PTK2; FOS; RPS6KA4; PIK3CB; PPP2R1A; PIK3C3; MAPK8; MAPK3; ITGAI; ETS1; KRAS; MYCN; EIF4EBP1; PPARG; PRKCD; PRKAA1; MAPK9; SRC; CDK2; PPP2CA; PIM1; PIK3C2A; ITGB7; YWHAZ; PPPICC; KSR1; PXN; RAF1; FYN; DYRKIA; ITGB1; MAP2K2; PAK4; PIK3R1; STAT3; PPP2R5C; MAP2K1; PAK3; ITGB3; ESR1; ITGA2; MYC; TTK; CSNKIA1; CRKL; BRAF; ATF4; PRKCA; SRF; STAT1; SGK; in the case of diseases or disorders associated with or involving glucocorticoid receptor signaling and/or regulation thereof in the brain, neuron, and/or CNS and/or diseases or disorders thereof: RAC1; TAF4B; EP300; SMAD2; TRAF6; PCAF; ELK1; MAPK1; SMAD3; AKT2; IKBKB; NCOR2; UBE2I; PIK3CA; CREB1; FOS; HSPA5; NFKB2; BCL2; MAP3K14; STAT5B; PIK3CB; PIK3C3; MAPK8; BCL2L1; MAPK3; TSC22D3; MAPK10; NRIP1; KRAS; MAPK13; RELA; STAT5A; MAPK9; NOS2A; PBX1; NR3C1; PIK3C2A; CDKN1C; TRAF2; SERPINE1; NCOA3; MAPK14; TNF; RAF1; IKBKG; MAP3K7; CREBBP; CDKN1A; MAP2K2; JAK1; IL8; NCOA2; AKT1; JAK2; PIK3R1; CHUK; STAT3; MAP2K1; NFKB1; TGFBR1; ESRI; SMAD4; CEBPB; JUN; AR; AKT3; CCL2; MMP1; STAT1; IL6; HSP90AA1; in the case of diseases or disorders associated with or involving ephrin receptor signaling and/or regulation thereof in the brain, neuron, and/or CNS and/or diseases or disorders thereof: PRKCE; ITGAM; ROCK1; ITGA5; CXCR4; IRAK1; PRKAA2; EIF2AK2; RAC1; RAP1A; GRK6; ROCK2; MAPK1; PGF; RAC2; PTPN11; GNAS; PLK1; AKT2; DOK1; CDK8; CREB1; PTK2; CFL1; GNAQ; MAP3K14; CXCL12; MAPK8; GNB2L1; ABL1; MAPK3; ITGAI; KRAS; RHOA; PRKCD; PRKAA1; MAPK9; SRC; CDK2; PIM1; ITGB7; PXN; RAF1; FYN; DYRKIA; ITGB1; MAP2K2; PAK4; AKT1; JAK2; STAT3; ADAM10; MAP2K1; PAK3; ITGB3; CDC42; VEGFA; ITGA2; EPHA8; TTK; CSNKIA1; CRKL; BRAF; PTPN13; ATF4; AKT3; SGK; in the case of diseases or disorders associated with or involving B cell receptor signaling and/or regulation thereof in the brain, neuron, and/or CNS and/or diseases or disorders thereof: RAC1; PTEN; LYN; ELK1; MAPK1; RAC2; PTPN11; AKT2; IKBKB; PIK3CA; CREB1; SYK; NFKB2; CAMK2A; MAP3K14; PIK3CB; PIK3C3; MAPK8; BCL2L1; ABL1; MAPK3; ETS1; KRAS; MAPK13; RELA; PTPN6; MAPK9; EGR1; PIK3C2A; BTK; MAPK14; RAF1; IKBKG; RELB; MAP3K7; MAP2K2; AKT1; PIK3R1; CHUK; MAP2K1; NFKB1; CDC42; GSK3A; FRAP1; BCL6; BCL10; JUN; GSK3B; ATF4; AKT3; VAV3; RPS6KB1; in the case of Infantile neuroaxonal dystroph: PLA2G6; in the case of Gaucher's disease: GBA; in the case of Krabbe disease: GALC; in the case of metachromatic leukodystrophy: ARSA and/or PRSP, isoform specific Saposin B replacement; in the case of Salla disease: SLC17A5; in the case of Farber disease or spinal muscular atrophy with progressive myoclonic epilepsy (also referred to as Jankovic-Rivera syndrome): ASAHI; in the case of Unverricht-Lundborg disease: CSTB; in the case of AADC deficiency: AADC; in the case of autosomal recessive forms of Parkinson's disease: PRKN, and others; in the case of Batten disease: CLN3; in the case of giant axonal neuropathy: GAN; in the case of mucopolysacchariodosis diseases (including MOS1H (Hurler syndrome), MPSII (Hunter syndrome), MPS III A-D: IDUA, IDS, SGSH, NAGLU, HGSNAT, GNS; in the case of Sandhoff disease (HEXB); in the case of GM2 gangliosidosis, AB variant: GM2A; in the case of Canavan disease: ASPA; in the case of cockayne syndrome: CSA or CSB; in the case of neurofibromatosis: NF1 or NF2; or any combination thereof.

Other diseases and disorders, as well as their associated genes, will be appreciated by those of skill in the art.

### EXAMPLES

### Example 1—AAV Capsids that Promote Transduction of CNS Tissue

Gene therapy has the potential to treat a wide range of central nervous system (CNS) diseases at their root cause. Adeno-associated viral (AAV) vectors can be used for gene replacement, gene editing, gene silencing, and to transactivate or repress gene transcription, and examples of each of these use cases have been validated in pre-clinical disease models. In addition, AAV vectors have been successfully used to delivery gene therapies to the CNS in humans. In a pioneering example of a systemically delivered gene therapy, AAV9 has been used to restore expression of the gene encoding the survival motor protein (SMN) protein to infants with a severe form of spinal muscular atrophy (SMA I). While this breakthrough gene therapy application has proven safe and effective in neonates, the wider implementation of systemic AAV9 for other CNS disorders is uncertain due to safety and efficacy concerns with high dose intravenous administration, especially in older individuals. Furthermore, even if high dose AAV-based therapy can be delivered safely, there is insufficient evidence that natural AAV serotypes such as AAV9 can achieve the wide-spread and efficient targeting of CNS cell types required for the treatment of many CNS diseases.

Applicants and others have shown that AAV capsids can be engineered to more efficiently cross the blood brain barrier (BBB) and transduce neurons and glia throughout the mouse CNS (Deverman, Chan, Kumar, Hanlon, Nonnenmacher). To date, none of these capsids developed through in vivo selections in mice has been reported to provide similar levels of enhanced transduction in non-human primates (NHPs).

Here, Applicants report the invention of novel 7-mer modified AAV capsids that more efficiently transduces cells throughout the CNS of the common marmoset, a species of new world primate. These capsids are engineered variants of AAV9-K449R (AA sequence: SEQ ID#1; NT sequence SEQ ID #2).

Through in vivo selection of AAV capsids that express the AAV cap mRNA in vivo, Applicants identified several capsid families that more efficiently transduce the CNS. First, A family of capsids defined by the 7-mer sequence motif (SEQ ID#3-16): 1 [ITQHKRVGEL] 2 [RNAKG] 3 [NIQMFLDTRVS] 4 [P] 5 [QNVST] 6 [E] 7 [FYH]. Second, Additional capsids with enhanced transduction of the marmoset CNS. Notably including AAV—BI—CNS-CJ13-17 (Tables 1, 2, and 3), which are enriched as compared with AAV9 in the marmoset and mouse CNS. These include 7-mer SEQ IDs 17-21.

A family of capsids, also known as the P×EF family, classified by a shared sequence motif within the engineered 7-mer insertion and exemplified by the AAV BI-103 capsid (also referenced as AAV-BI-CNS-CJ8, see Table 1), exhibits enhanced transduction of neurons and glia in all regions of the CNS relative to AAV9.

A core motif identified from an analysis of capsid 7-mers selected for transduction of the marmoset brain has the following features: a single positive charged amino acid (R or K) in position 1 OR 2, P at position 4, E at position 6, AND an amino acid with a single aromatic ring (F, Y, or H) at position 7. This is referred to as the [K/R] 1/2XPXE [FYH] motif. Additionally, certain amino acids are preferred at additional positions: 1 [ITQHKRVGEL], 2 [RNAKG], 3 [NIQMFLDTRVSP], 4 [P]. 5[QNVST]6 [E]. 7[FYH].

TABLE 1
[K/R]1/2-PXEF 7-mer insertion sequences
recovered from the brain and spinal
cord in round 3.

| | 7-mer | | Protein | Nucleotide |
|---|---|---|---|---|
| Capsid | SEQ ID# | 7-mer | SEQ ID# | SEQ ID#s |
| AAV-BI-CNS-CJ1 | SEQ ID #3 | ELNPREF | SEQ ID #22 | 41 to 50 |
| AAV-BI-CNS-CJ2 | SEQ ID #4 | GKLPREF | SEQ ID #23 | 51 to 60 |
| AAV-BI-CNS-CJ3 | SEQ ID #5 | HNFPNEF | SEQ ID #24 | 61 to 70 |
| AAV-BI-CNS-CJ4 | SEQ ID #6 | IRMPTEF | SEQ ID #25 | 71 to 80 |
| AAV-BI-CNS-CJ5 | SEQ ID #7 | IRQPQEF | SEQ ID #26 | 81 to 90 |
| AAV-BI-CNS-CJ6 | SEQ ID #8 | IRVPNEF | SEQ ID #27 | 91 to 100 |
| AAV-BI-CNS-CJ7 | SEQ ID #9 | KGPGYEH | SEQ ID #28 | 101 to 110 |
| AAV-BI-CNS-CJ8 | SEQ ID #10 | KNSPSEF | SEQ ID #29 | 111 to 120 |
| AAV-BI-CNS-CJ9 | SEQ ID #11 | LALPQEF | SEQ ID #30 | 121 to 130 |
| AAV-BI-CNS-CJ10 | SEQ ID #12 | QNIPSEY | SEQ ID #31 | 131 to 140 |
| AAV-BI-CNS-CJ11 | SEQ ID #13 | QRIPQEY | SEQ ID #32 | 141 to 150 |
| AAV-BI-CNS-CJ12 | SEQ ID #14 | RSQPQEH | SEQ ID #33 | 151 to 060 |
| AAV-BI-CNS-CJ13 | SEQ ID #15 | TARPNEY | SEQ ID #34 | 161 to 170 |
| AAV-BI-CNS-CJ14 | SEQ ID #16 | TRTPVEF | SEQ ID #35 | 171 to 180 |

TABLE 2
[K/R]1/2-PXEF 7-mer insertion sequences
enriched in the brain and spinal
cord in round 3.

| | 7-mer | | Protein | Nucleotide |
|---|---|---|---|---|
| Capsid | SEQ ID# | 7-mer | SEQ ID# | SEQ ID#s |
| AAV-BI-CNS-CJ3 | SEQ ID #5 | HNFPNEF | SEQ ID #24 | 61 to 70 |
| AAV-BI-CNS-CJ4 | SEQ ID #6 | IRMPTEF | SEQ ID #25 | 71 to 80 |
| AAV-BI-CNS-CJ5 | SEQ ID #7 | IRQPQEF | SEQ ID #26 | 81 to 90 |
| AAV-BI-CNS-CJ6 | SEQ ID #8 | IR VPNEF | SEQ ID #27 | 91 to 100 |
| AAV-BI-CNS-CJ7 | SEQ ID #9 | KGPGYEH | SEQ ID #28 | 101 to 110 |
| AAV-BI-CNS-CJ8 | SEQ ID #10 | KNSPSEF | SEQ ID #29 | 111 to 120 |
| AAV-BI-CNS-CJ11 | SEQ ID #13 | QRIPQEY | SEQ ID #32 | 141 to 150 |
| AAV-BI-CNS-CJ12 | SEQ ID #14 | RSQPQEH | SEQ ID #33 | 151 to 160 |
| AAV-BI-CNS-CJ14 | SEQ ID #16 | TRTPVEF | SEQ ID #35 | 171 to 180 |

TABLE 3
Additional 7-mer insertion sequences
enriched in the brain and spinal cord in
round 3.

| | 7-mer | | Protein | Nucleotide |
|---|---|---|---|---|
| Capsid | SEQ ID# | 7-mer | SEQ ID# | SEQ ID#s |
| AAV-BI-CNS-CJ15 | SEQ ID #17 | ENHTRDK | SEQ ID #36 | 181 to 190 |
| AAV-BI-CNS-CJ16 | SEQ ID #18 | GNDTRST | SEQ ID #37 | 191 to 200 |
| AAV-BI-CNS-CJ17 | SEQ ID #19 | MNSTISK | SEQ ID #38 | 201 to 210 |
| AAV-BI-CNS-CJ18 | SEQ ID #20 | QSRDLYR | SEQ ID #39 | 231 to 240 |
| AAV-BI-CNS-CJ19 | SEQ ID #21 | VNTVREF | SEQ ID #40 | 241 to 250 |

TABLE 4
SEQ ID Listing

| SEQ | | | |
|---|---|---|---|
| ID | | | |
| NO | Type | Organism | Sequence |
| 1 | PRT | Adeno- | MAADGYLPDWLEDNLSEGIREWWALKPGAPQPKANQQHQDNARGLVLPGY |
| | | Associated | KYLGPGNGLDKGEPVNAADAAALEHDKAYDQQLKAGDNPYLKYNHADAEF |
| | | Virus 9 | QERLKEDTSFGGNLGRAVFQAKKRLLEPLGLVEEAAKTAPGKKRPVEQSP |
| | | | QEPDSSAGIGKSGAQPAKKRLNFGQTGDTESVPDPQPIGEPPAAPSGVGS |
| | | | LTMASGGGAPVADNNEGADGVGSSSGNWHCDSQWLGDRVITTSTRTWALP |
| | | | TYNNHLYKQISNSTSGGSSNDNAYFGYSTPWGYFDFNRFHCHFSPRDWQR |
| | | | LINNNWGFRPKRLNFKLFNIQVKEVTDNNGVKTIANNLTSTVQVFTDSDY |
| | | | QLPYVLGSAHEGCLPPFPADVFMIPQYGYLTLNDGSQAVGRSSFYCLEYF |
| | | | PSQMLRTGNNFQFSYEFENVPFHSSYAHSQSLDRLMNPLIDQYLYYLSRT |
| | | | INGSGQNQQTLKFSVAGPSNMAVQGRNYIPGPSYRQQRVSTTVTQNNNSE |
| | | | FAWPGASSWALNGRNSLMNPGPAMASHKEGEDRFFPLSGSLIFGKQGTGR |
| | | | DNVDADKVMITNEEEIKTTNPVATESYGQVATNHQSAQAQAQTGWVQNQG |
| | | | ILPGMVWQDRDVYLQGPIWAKIPHTDGNFHPSPLMGGFGMKHPPPQILIK |
| | | | NTPVPADPPTAFNKDKLNSFITQYSTGQVSVEIEWELQKENSKRWNPEIQ |
| | | | YTSNYYKSNNVEFAVNTEGVYSEPRPIGTRYLTRNL |
| 2 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | | aaataccttggacccggcaacggactcgacaagggggagccggtcaacgc |
| | | | agcagacgcggcggccctcgagcacgacaaggcctacgaccagcagctca |
| | | | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCCCAAgcacaggcgcagaccggttgggttcaaaaccaagga |
| | | | atacttccgggtatggtttggcaggacagagatgtgtacctgcaaggacc |
| | | | catttgggccaaaattcctcacacggacggcaactttcacccttctccgc |
| | | | tgatgggagggtttggaatgaagcacccgcctcctcagatcctcatcaaa |
| | | | aacacacctgtacctgcggatcctccaacggccttcaacaaggacaagct |
| | | | gaactctttcatcacccagtattctactggccaagtcagcgtggagatcg |
| | | | agtgggagctgcagaaggaaaacagcaagcgctggaacccggagatccag |
| | | | tacacttccaactattacaagtctaataatgttgaatttgctgttaatac |
| | | | tgaaggtgtatatagtgaaccccgccccattggcaccagatacctgactc |
| | | | gtaatctg |
| 3 | PRT | Adeno- | ELNPREF |
| | | Associated | |
| | | Virus 9 | |
| 4 | PRT | Adeno- | GKLPREF |
| | | Associated | |
| | | Virus 9 | |
| 5 | PRT | Adeno- | HNFPNEF |
| | | Associated | |
| | | Virus 9 | |
| 6 | PRT | Adeno- | IRMPTEF |
| | | Associated | |
| | | Virus 9 | |
| 7 | PRT | Adeno- | IRQPQEF |
| | | Associated | |
| | | Virus 9 | |
| 8 | PRT | Adeno- | IRVPNEF |
| | | Associated | |
| | | Virus 9 | |
| 9 | PRT | Adeno- | KGPGYEH |
| | | Associated | |
| | | Virus 9 | |
| 10 | PRT | Adeno- | KNSPSEF |
| | | Associated | |
| | | Virus 9 | |
| 11 | PRT | Adeno- | LALPQEF |
| | | Associated | |
| | | Virus 9 | |
| 12 | PRT | Adeno- | QNIPSEY |
| | | Associated | |
| | | Virus 9 | |
| 13 | PRT | Adeno- | QRIPQEY |
| | | Associated | |
| | | Virus 9 | |
| 14 | PRT | Adeno- | RSQPQEH |
| | | Associated | |
| | | Virus 9 | |
| 15 | PRT | Adeno- | TARPNEY |
| | | Associated | |
| | | Virus 9 | |
| 16 | PRT | Adeno- | TRTPVEF |
| | | Associated | |
| | | Virus 9 | |
| 17 | PRT | Adeno- | ENHTRDK |
| | | Associated | |
| | | Virus 9 | |
| 18 | PRT | Adeno- | GNDTRST |
| | | Associated | |
| | | Virus 9 | |
| 19 | PRT | Adeno- | MNSTISK |
| | | Associated | |
| | | Virus 9 | |
| 20 | PRT | Adeno- | QSRDLYR |
| | | Associated | |
| | | Virus 9 | |
| 21 | PRT | Adeno- | VNTVREF |
| | | Associated | |
| | | Virus 9 | |
| 22 | PRT | Adeno- | MAADGYLPDWLEDNLSEGIREWWALKPGAPQPKANQQHQDNARGLVLPGY |
| | | Associated | KYLGPGNGLDKGEPVNAADAAALEHDKAYDQQLKAGDNPYLKYNHADAEF |
| | | Virus 9 | QERLKEDTSFGGNLGRAVFQAKKRLLEPLGLVEEAAKTAPGKKRPVEQSP |
| | | comprising | QEPDSSAGIGKSGAQPAKKRLNFGQTGDTESVPDPQPIGEPPAAPSGVGS |
| | | SEQ | LTMASGGGAPVADNNEGADGVGSSSGNWHCDSQWLGDRVITTSTRTWALP |
| | | ID NO: 3 | TYNNHLYKQISNSTSGGSSNDNAYFGYSTPWGYFDFNRFHCHFSPRDWQR |
| | | | LINNNWGFRPKRLNFKLFNIQVKEVTDNNGVKTIANNLTSTVQVFTDSDY |
| | | | QLPYVLGSAHEGCLPPFPADVFMIPQYGYLTLNDGSQAVGRSSFYCLEYF |
| | | | PSQMLRTGNNFQFSYEFENVPFHSSYAHSQSLDRLMNPLIDQYLYYLSRT |
| | | | INGSGQNQQTLKFSVAGPSNMAVQGRNYIPGPSYRQQRVSTTVTQNNNSE |
| | | | FAWPGASSWALNGRNSLMNPGPAMASHKEGEDRFFPLSGSLIFGKQGTGR |
| | | | DNVDADKVMITNEEEIKTTNPVATESYGQVATNHQSAQELNPREFAQAQT |
| | | | GWVQNQGILPGMVWQDRDVYLQGPIWAKIPHTDGNFHPSPLMGGFGMKHP |
| | | | PPQILIKNTPVPADPPTAFNKDKLNSFITQYSTGQVSVEIEWELQKENSK |
| | | | RWNPEIQYTSNYYKSNNVEFAVNTEGVYSEPRPIGTRYLTRNL |
| 23 | PRT | Adeno- | MAADGYLPDWLEDNLSEGIREWWALKPGAPQPKANQQHQDNARGLVLPGY |
| | | Associated | KYLGPGNGLDKGEPVNAADAAALEHDKAYDQQLKAGDNPYLKYNHADAEF |
| | | Virus 9 | QERLKEDTSFGGNLGRAVFQAKKRLLEPLGLVEEAAKTAPGKKRPVEQSP |
| | | comprising | QEPDSSAGIGKSGAQPAKKRLNFGQTGDTESVPDPQPIGEPPAAPSGVGS |
| | | SEQ | LTMASGGGAPVADNNEGADGVGSSSGNWHCDSQWLGDRVITTSTRTWALP |
| | | ID NO: 4 | TYNNHLYKQISNSTSGGSSNDNAYFGYSTPWGYFDFNRFHCHFSPRDWQR |
| | | | LINNNWGFRPKRLNFKLFNIQVKEVTDNNGVKTIANNLTSTVQVFTDSDY |
| | | | QLPYVLGSAHEGCLPPFPADVFMIPQYGYLTLNDGSQAVGRSSFYCLEYF |
| | | | PSQMLRTGNNFQFSYEFENVPFHSSYAHSQSLDRLMNPLIDQYLYYLSRT |
| | | | INGSGQNQQTLKFSVAGPSNMAVQGRNYIPGPSYRQQRVSTTVTQNNNSE |
| | | | FAWPGASSWALNGRNSLMNPGPAMASHKEGEDRFFPLSGSLIFGKQGTGR |
| | | | DNVDADKVMITNEEEIKTTNPVATESYGQVATNHQSAQGKLPREFAQAQT |
| | | | GWVQNQGILPGMVWQDRDVYLQGPIWAKIPHTDGNFHPSPLMGGFGMKHP |
| | | | PPQILIKNTPVPADPPTAFNKDKLNSFITQYSTGQVSVEIEWELQKENSK |
| | | | RWNPEIQYTSNYYKSNNVEFAVNTEGVYSEPRPIGTRYLTRNL |
| 24 | PRT | Adeno- | MAADGYLPDWLEDNLSEGIREWWALKPGAPQPKANQQHQDNARGLVLPGY |
| | | Associated | KYLGPGNGLDKGEPVNAADAAALEHDKAYDQQLKAGDNPYLKYNHADAEF |
| | | Virus 9 | QERLKEDTSFGGNLGRAVFQAKKRLLEPLGLVEEAAKTAPGKKRPVEQSP |
| | | comprising | QEPDSSAGIGKSGAQPAKKRLNFGQTGDTESVPDPQPIGEPPAAPSGVGS |
| | | SEQ | LTMASGGGAPVADNNEGADGVGSSSGNWHCDSQWLGDRVITTSTRTWALP |
| | | ID NO: 5 | TYNNHLYKQISNSTSGGSSNDNAYFGYSTPWGYFDFNRFHCHFSPRDWQR |
| | | | LINNNWGFRPKRLNFKLFNIQVKEVTDNNGVKTIANNLTSTVQVFTDSDY |
| | | | QLPYVLGSAHEGCLPPFPADVFMIPQYGYLTLNDGSQAVGRSSFYCLEYF |
| | | | PSQMLRTGNNFQFSYEFENVPFHSSYAHSQSLDRLMNPLIDQYLYYLSRT |
| | | | INGSGQNQQTLKFSVAGPSNMAVQGRNYIPGPSYRQQRVSTTVTQNNNSE |
| | | | FAWPGASSWALNGRNSLMNPGPAMASHKEGEDRFFPLSGSLIFGKQGTGR |
| | | | DNVDADKVMITNEEEIKTTNPVATESYGQVATNHQSAQHNFPNEFAQAQT |
| | | | GWVQNQGILPGMVWQDRDVYLQGPIWAKIPHTDGNFHPSPLMGGFGMKHP |
| | | | PPQILIKNTPVPADPPTAFNKDKLNSFITQYSTGQVSVEIEWELQKENSK |
| | | | RWNPEIQYTSNYYKSNNVEFAVNTEGVYSEPRPIGTRYLTRNL |
| 25 | PRT | Adeno- | MAADGYLPDWLEDNLSEGIREWWALKPGAPQPKANQQHQDNARGLVLPGY |
| | | Associated | KYLGPGNGLDKGEPVNAADAAALEHDKAYDQQLKAGDNPYLKYNHADAEF |
| | | Virus 9 | QERLKEDTSFGGNLGRAVFQAKKRLLEPLGLVEEAAKTAPGKKRPVEQSP |
| | | comprising | QEPDSSAGIGKSGAQPAKKRLNFGQTGDTESVPDPQPIGEPPAAPSGVGS |
| | | SEQ | LTMASGGGAPVADNNEGADGVGSSSGNWHCDSQWLGDRVITTSTRTWALP |
| | | ID NO: 6 | TYNNHLYKQISNSTSGGSSNDNAYFGYSTPWGYFDFNRFHCHFSPRDWQR |
| | | | LINNNWGFRPKRLNFKLFNIQVKEVTDNNGVKTIANNLTSTVQVFTDSDY |
| | | | QLPYVLGSAHEGCLPPFPADVFMIPQYGYLTLNDGSQAVGRSSFYCLEYF |
| | | | PSQMLRTGNNFQFSYEFENVPFHSSYAHSQSLDRLMNPLIDQYLYYLSRT |
| | | | INGSGQNQQTLKFSVAGPSNMAVQGRNYIPGPSYRQQRVSTTVTQNNNSE |
| | | | FAWPGASSWALNGRNSLMNPGPAMASHKEGEDRFFPLSGSLIFGKQGTGR |
| | | | DNVDADKVMITNEEEIKTTNPVATESYGQVATNHQSAQIRMPTEFAQAQT |
| | | | GWVQNQGILPGMVWQDRDVYLQGPIWAKIPHTDGNFHPSPLMGGFGMKHP |
| | | | PPQILIKNTPVPADPPTAFNKDKLNSFITQYSTGQVSVEIEWELQKENSK |
| | | | RWNPEIQYTSNYYKSNNVEFAVNTEGVYSEPRPIGTRYLTRNL |
| 26 | PRT | Adeno- | MAADGYLPDWLEDNLSEGIREWWALKPGAPQPKANQQHQDNARGLVLPGY |
| | | Associated | KYLGPGNGLDKGEPVNAADAAALEHDKAYDQQLKAGDNPYLKYNHADAEF |
| | | Virus 9 | QERLKEDTSFGGNLGRAVFQAKKRLLEPLGLVEEAAKTAPGKKRPVEQSP |
| | | comprising | QEPDSSAGIGKSGAQPAKKRLNFGQTGDTESVPDPQPIGEPPAAPSGVGS |
| | | SEQ | LTMASGGGAPVADNNEGADGVGSSSGNWHCDSQWLGDRVITTSTRTWALP |
| | | ID NO: 7 | TYNNHLYKQISNSTSGGSSNDNAYFGYSTPWGYFDFNRFHCHFSPRDWQR |
| | | | LINNNWGFRPKRLNFKLFNIQVKEVTDNNGVKTIANNLTSTVQVFTDSDY |
| | | | QLPYVLGSAHEGCLPPFPADVFMIPQYGYLTLNDGSQAVGRSSFYCLEYF |
| | | | PSQMLRTGNNFQFSYEFENVPFHSSYAHSQSLDRLMNPLIDQYLYYLSRT |
| | | | INGSGQNQQTLKFSVAGPSNMAVQGRNYIPGPSYRQQRVSTTVTQNNNSE |
| | | | FAWPGASSWALNGRNSLMNPGPAMASHKEGEDRFFPLSGSLIFGKQGTGR |
| | | | DNVDADKVMITNEEEIKTTNPVATESYGQVATNHQSAQIRQPQEFAQAQT |
| | | | GWVQNQGILPGMVWQDRDVYLQGPIWAKIPHTDGNFHPSPLMGGFGMKHP |
| | | | PPQILIKNTPVPADPPTAFNKDKLNSFITQYSTGQVSVEIEWELQKENSK |
| | | | RWNPEIQYTSNYYKSNNVEFAVNTEGVYSEPRPIGTRYLTRNL |
| 27 | PRT | Adeno- | MAADGYLPDWLEDNLSEGIREWWALKPGAPQPKANQQHQDNARGLVLPGY |
| | | Associated | KYLGPGNGLDKGEPVNAADAAALEHDKAYDQQLKAGDNPYLKYNHADAEF |
| | | Virus 9 | QERLKEDTSFGGNLGRAVFQAKKRLLEPLGLVEEAAKTAPGKKRPVEQSP |
| | | comprising | QEPDSSAGIGKSGAQPAKKRLNFGQTGDTESVPDPQPIGEPPAAPSGVGS |
| | | SEQ | LTMASGGGAPVADNNEGADGVGSSSGNWHCDSQWLGDRVITTSTRTWALP |
| | | ID NO: 8 | TYNNHLYKQISNSTSGGSSNDNAYFGYSTPWGYFDFNRFHCHFSPRDWQR |
| | | | LINNNWGFRPKRLNFKLFNIQVKEVTDNNGVKTIANNLTSTVQVFTDSDY |
| | | | QLPYVLGSAHEGCLPPFPADVFMIPQYGYLTLNDGSQAVGRSSFYCLEYF |
| | | | PSQMLRTGNNFQFSYEFENVPFHSSYAHSQSLDRLMNPLIDQYLYYLSRT |
| | | | INGSGQNQQTLKFSVAGPSNMAVQGRNYIPGPSYRQQRVSTTVTQNNNSE |
| | | | FAWPGASSWALNGRNSLMNPGPAMASHKEGEDRFFPLSGSLIFGKQGTGR |
| | | | DNVDADKVMITNEEEIKTTNPVATESYGQVATNHQSAQIRVPNEFAQAQT |
| | | | GWVQNQGILPGMVWQDRDVYLQGPIWAKIPHTDGNFHPSPLMGGFGMKHP |
| | | | PPQILIKNTPVPADPPTAFNKDKLNSFITQYSTGQVSVEIEWELQKENSK |
| | | | RWNPEIQYTSNYYKSNNVEFAVNTEGVYSEPRPIGTRYLTRNL |
| 28 | PRT | Adeno- | MAADGYLPDWLEDNLSEGIREWWALKPGAPQPKANQQHQDNARGLVLPGY |
| | | Associated | KYLGPGNGLDKGEPVNAADAAALEHDKAYDQQLKAGDNPYLKYNHADAEF |
| | | Virus 9 | QERLKEDTSFGGNLGRAVFQAKKRLLEPLGLVEEAAKTAPGKKRPVEQSP |
| | | comprising | QEPDSSAGIGKSGAQPAKKRLNFGQTGDTESVPDPQPIGEPPAAPSGVGS |
| | | SEQ | LTMASGGGAPVADNNEGADGVGSSSGNWHCDSQWLGDRVITTSTRTWALP |
| | | ID NO: 9 | TYNNHLYKQISNSTSGGSSNDNAYFGYSTPWGYFDFNRFHCHFSPRDWQR |
| | | | LINNNWGFRPKRLNFKLFNIQVKEVTDNNGVKTIANNLTSTVQVFTDSDY |
| | | | QLPYVLGSAHEGCLPPFPADVFMIPQYGYLTLNDGSQAVGRSSFYCLEYF |
| | | | PSQMLRTGNNFQFSYEFENVPFHSSYAHSQSLDRLMNPLIDQYLYYLSRT |
| | | | INGSGQNQQTLKFSVAGPSNMAVQGRNYIPGPSYRQQRVSTTVTQNNNSE |
| | | | FAWPGASSWALNGRNSLMNPGPAMASHKEGEDRFFPLSGSLIFGKQGTGR |
| | | | DNVDADKVMITNEEEIKTTNPVATESYGQVATNHQSAQKGPGYEHAQAQT |
| | | | GWVQNQGILPGMVWQDRDVYLQGPIWAKIPHTDGNFHPSPLMGGFGMKHP |
| | | | PPQILIKNTPVPADPPTAFNKDKLNSFITQYSTGQVSVEIEWELQKENSK |
| | | | RWNPEIQYTSNYYKSNNVEFAVNTEGVYSEPRPIGTRYLTRNL |
| 29 | PRT | Adeno- | MAADGYLPDWLEDNLSEGIREWWALKPGAPQPKANQQHQDNARGLVLPGY |
| | | Associated | KYLGPGNGLDKGEPVNAADAAALEHDKAYDQQLKAGDNPYLKYNHADAEF |
| | | Virus 9 | QERLKEDTSFGGNLGRAVFQAKKRLLEPLGLVEEAAKTAPGKKRPVEQSP |
| | | comprising | QEPDSSAGIGKSGAQPAKKRLNFGQTGDTESVPDPQPIGEPPAAPSGVGS |
| | | SEQ | LTMASGGGAPVADNNEGADGVGSSSGNWHCDSQWLGDRVITTSTRTWALP |
| | | ID NO: 10 | TYNNHLYKQISNSTSGGSSNDNAYFGYSTPWGYFDFNRFHCHFSPRDWQR |
| | | | LINNNWGFRPKRLNFKLFNIQVKEVTDNNGVKTIANNLTSTVQVFTDSDY |
| | | | QLPYVLGSAHEGCLPPFPADVFMIPQYGYLTLNDGSQAVGRSSFYCLEYF |
| | | | PSQMLRTGNNFQFSYEFENVPFHSSYAHSQSLDRLMNPLIDQYLYYLSRT |
| | | | INGSGQNQQTLKFSVAGPSNMAVQGRNYIPGPSYRQQRVSTTVTQNNNSE |
| | | | FAWPGASSWALNGRNSLMNPGPAMASHKEGEDRFFPLSGSLIFGKQGTGR |
| | | | DNVDADKVMITNEEEIKTTNPVATESYGQVATNHQSAQKNSPSEFAQAQT |
| | | | GWVQNQGILPGMVWQDRDVYLQGPIWAKIPHTDGNFHPSPLMGGFGMKHP |
| | | | PPQILIKNTPVPADPPTAFNKDKLNSFITQYSTGQVSVEIEWELQKENSK |
| | | | RWNPEIQYTSNYYKSNNVEFAVNTEGVYSEPRPIGTRYLTRNL |
| 30 | PRT | Adeno- | MAADGYLPDWLEDNLSEGIREWWALKPGAPQPKANQQHQDNARGLVLPGY |
| | | Associated | KYLGPGNGLDKGEPVNAADAAALEHDKAYDQQLKAGDNPYLKYNHADAEF |
| | | Virus 9 | QERLKEDTSFGGNLGRAVFQAKKRLLEPLGLVEEAAKTAPGKKRPVEQSP |
| | | comprising | QEPDSSAGIGKSGAQPAKKRLNFGQTGDTESVPDPQPIGEPPAAPSGVGS |
| | | SEQ | LTMASGGGAPVADNNEGADGVGSSSGNWHCDSQWLGDRVITTSTRTWALP |
| | | ID NO: 11 | TYNNHLYKQISNSTSGGSSNDNAYFGYSTPWGYFDFNRFHCHFSPRDWQR |
| | | | LINNNWGFRPKRLNFKLFNIQVKEVTDNNGVKTIANNLTSTVQVFTDSDY |
| | | | QLPYVLGSAHEGCLPPFPADVFMIPQYGYLTLNDGSQAVGRSSFYCLEYF |
| | | | PSQMLRTGNNFQFSYEFENVPFHSSYAHSQSLDRLMNPLIDQYLYYLSRT |
| | | | INGSGQNQQTLKFSVAGPSNMAVQGRNYIPGPSYRQQRVSTTVTQNNNSE |
| | | | FAWPGASSWALNGRNSLMNPGPAMASHKEGEDRFFPLSGSLIFGKQGTGR |
| | | | DNVDADKVMITNEEEIKTTNPVATESYGQVATNHQSAQLALPQEFAQAQT |
| | | | GWVQNQGILPGMVWQDRDVYLQGPIWAKIPHTDGNFHPSPLMGGFGMKHP |
| | | | PPQILIKNTPVPADPPTAFNKDKLNSFITQYSTGQVSVEIEWELQKENSK |
| | | | RWNPEIQYTSNYYKSNNVEFAVNTEGVYSEPRPIGTRYLTRNL |
| 31 | PRT | Adeno- | MAADGYLPDWLEDNLSEGIREWWALKPGAPQPKANQQHQDNARGLVLPGY |
| | | Associated | KYLGPGNGLDKGEPVNAADAAALEHDKAYDQQLKAGDNPYLKYNHADAEF |
| | | Virus 9 | QERLKEDTSFGGNLGRAVFQAKKRLLEPLGLVEEAAKTAPGKKRPVEQSP |
| | | comprising | QEPDSSAGIGKSGAQPAKKRLNFGQTGDTESVPDPQPIGEPPAAPSGVGS |
| | | SEQ | LTMASGGGAPVADNNEGADGVGSSSGNWHCDSQWLGDRVITTSTRTWALP |
| | | ID NO: 12 | TYNNHLYKQISNSTSGGSSNDNAYFGYSTPWGYFDFNRFHCHFSPRDWQR |
| | | | LINNNWGFRPKRLNFKLFNIQVKEVTDNNGVKTIANNLTSTVQVFTDSDY |
| | | | QLPYVLGSAHEGCLPPFPADVFMIPQYGYLTLNDGSQAVGRSSFYCLEYF |
| | | | PSQMLRTGNNFQFSYEFENVPFHSSYAHSQSLDRLMNPLIDQYLYYLSRT |
| | | | INGSGQNQQTLKFSVAGPSNMAVQGRNYIPGPSYRQQRVSTTVTQNNNSE |
| | | | FAWPGASSWALNGRNSLMNPGPAMASHKEGEDRFFPLSGSLIFGKQGTGR |
| | | | DNVDADKVMITNEEEIKTTNPVATESYGQVATNHQSAQQNIPSEYAQAQT |
| | | | GWVQNQGILPGMVWQDRDVYLQGPIWAKIPHTDGNFHPSPLMGGFGMKHP |
| | | | PPQILIKNTPVPADPPTAFNKDKLNSFITQYSTGQVSVEIEWELQKENSK |
| | | | RWNPEIQYTSNYYKSNNVEFAVNTEGVYSEPRPIGTRYLTRNL |
| 32 | PRT | Adeno- | MAADGYLPDWLEDNLSEGIREWWALKPGAPQPKANQQHQDNARGLVLPGY |
| | | Associated | KYLGPGNGLDKGEPVNAADAAALEHDKAYDQQLKAGDNPYLKYNHADAEF |
| | | Virus 9 | QERLKEDTSFGGNLGRAVFQAKKRLLEPLGLVEEAAKTAPGKKRPVEQSP |
| | | comprising | QEPDSSAGIGKSGAQPAKKRLNFGQTGDTESVPDPQPIGEPPAAPSGVGS |
| | | SEQ | LTMASGGGAPVADNNEGADGVGSSSGNWHCDSQWLGDRVITTSTRTWALP |
| | | ID NO: 13 | TYNNHLYKQISNSTSGGSSNDNAYFGYSTPWGYFDFNRFHCHFSPRDWQR |
| | | | LINNNWGFRPKRLNFKLFNIQVKEVTDNNGVKTIANNLTSTVQVFTDSDY |
| | | | QLPYVLGSAHEGCLPPFPADVFMIPQYGYLTLNDGSQAVGRSSFYCLEYF |
| | | | PSQMLRTGNNFQFSYEFENVPFHSSYAHSQSLDRLMNPLIDQYLYYLSRT |
| | | | INGSGQNQQTLKFSVAGPSNMAVQGRNYIPGPSYRQQRVSTTVTQNNNSE |
| | | | FAWPGASSWALNGRNSLMNPGPAMASHKEGEDRFFPLSGSLIFGKQGTGR |
| | | | DNVDADKVMITNEEEIKTTNPVATESYGQVATNHQSAQQRIPQEYAQAQT |
| | | | GWVQNQGILPGMVWQDRDVYLQGPIWAKIPHTDGNFHPSPLMGGFGMKHP |
| | | | PPQILIKNTPVPADPPTAFNKDKLNSFITQYSTGQVSVEIEWELQKENSK |
| | | | RWNPEIQYTSNYYKSNNVEFAVNTEGVYSEPRPIGTRYLTRNL |
| 33 | PRT | Adeno- | MAADGYLPDWLEDNLSEGIREWWALKPGAPQPKANQQHQDNARGLVLPGY |
| | | Associated | KYLGPGNGLDKGEPVNAADAAALEHDKAYDQQLKAGDNPYLKYNHADAEF |
| | | Virus 9 | QERLKEDTSFGGNLGRAVFQAKKRLLEPLGLVEEAAKTAPGKKRPVEQSP |
| | | comprising | QEPDSSAGIGKSGAQPAKKRLNFGQTGDTESVPDPQPIGEPPAAPSGVGS |
| | | SEQ | LTMASGGGAPVADNNEGADGVGSSSGNWHCDSQWLGDRVITTSTRTWALP |
| | | ID NO: 14 | TYNNHLYKQISNSTSGGSSNDNAYFGYSTPWGYFDFNRFHCHFSPRDWQR |
| | | | LINNNWGFRPKRLNFKLFNIQVKEVTDNNGVKTIANNLTSTVQVFTDSDY |
| | | | QLPYVLGSAHEGCLPPFPADVFMIPQYGYLTLNDGSQAVGRSSFYCLEYF |
| | | | PSQMLRTGNNFQFSYEFENVPFHSSYAHSQSLDRLMNPLIDQYLYYLSRT |
| | | | INGSGQNQQTLKFSVAGPSNMAVQGRNYIPGPSYRQQRVSTTVTQNNNSE |
| | | | FAWPGASSWALNGRNSLMNPGPAMASHKEGEDRFFPLSGSLIFGKQGTGR |
| | | | DNVDADKVMITNEEEIKTTNPVATESYGQVATNHQSAQRSQPQEHAQAQT |
| | | | GWVQNQGILPGMVWQDRDVYLQGPIWAKIPHTDGNFHPSPLMGGFGMKHP |
| | | | PPQILIKNTPVPADPPTAFNKDKLNSFITQYSTGQVSVEIEWELQKENSK |
| | | | RWNPEIQYTSNYYKSNNVEFAVNTEGVYSEPRPIGTRYLTRNL |
| 34 | PRT | Adeno- | MAADGYLPDWLEDNLSEGIREWWALKPGAPQPKANQQHQDNARGLVLPGY |
| | | Associated | KYLGPGNGLDKGEPVNAADAAALEHDKAYDQQLKAGDNPYLKYNHADAEF |
| | | Virus 9 | QERLKEDTSFGGNLGRAVFQAKKRLLEPLGLVEEAAKTAPGKKRPVEQSP |
| | | comprising | QEPDSSAGIGKSGAQPAKKRLNFGQTGDTESVPDPQPIGEPPAAPSGVGS |
| | | SEQ | LTMASGGGAPVADNNEGADGVGSSSGNWHCDSQWLGDRVITTSTRTWALP |
| | | ID NO: 15 | TYNNHLYKQISNSTSGGSSNDNAYFGYSTPWGYFDFNRFHCHFSPRDWQR |
| | | | LINNNWGFRPKRLNFKLFNIQVKEVTDNNGVKTIANNLTSTVQVFTDSDY |
| | | | QLPYVLGSAHEGCLPPFPADVFMIPQYGYLTLNDGSQAVGRSSFYCLEYF |
| | | | PSQMLRTGNNFQFSYEFENVPFHSSYAHSQSLDRLMNPLIDQYLYYLSRT |
| | | | INGSGQNQQTLKFSVAGPSNMAVQGRNYIPGPSYRQQRVSTTVTQNNNSE |
| | | | FAWPGASSWALNGRNSLMNPGPAMASHKEGEDRFFPLSGSLIFGKQGTGR |
| | | | DNVDADKVMITNEEEIKTTNPVATESYGQVATNHQSAQTARPNEYAQAQT |
| | | | GWVQNQGILPGMVWQDRDVYLQGPIWAKIPHTDGNFHPSPLMGGFGMKHP |
| | | | PPQILIKNTPVPADPPTAFNKDKLNSFITQYSTGQVSVEIEWELQKENSK |
| | | | RWNPEIQYTSNYYKSNNVEFAVNTEGVYSEPRPIGTRYLTRNL |
| 35 | PRT | Adeno- | MAADGYLPDWLEDNLSEGIREWWALKPGAPQPKANQQHQDNARGLVLPGY |
| | | Associated | KYLGPGNGLDKGEPVNAADAAALEHDKAYDQQLKAGDNPYLKYNHADAEF |
| | | Virus 9 | QERLKEDTSFGGNLGRAVFQAKKRLLEPLGLVEEAAKTAPGKKRPVEQSP |
| | | comprising | QEPDSSAGIGKSGAQPAKKRLNFGQTGDTESVPDPQPIGEPPAAPSGVGS |
| | | SEQ | LTMASGGGAPVADNNEGADGVGSSSGNWHCDSQWLGDRVITTSTRTWALP |
| | | ID NO: 16 | TYNNHLYKQISNSTSGGSSNDNAYFGYSTPWGYFDFNRFHCHFSPRDWQR |
| | | | LINNNWGFRPKRLNFKLFNIQVKEVTDNNGVKTIANNLTSTVQVFTDSDY |
| | | | QLPYVLGSAHEGCLPPFPADVFMIPQYGYLTLNDGSQAVGRSSFYCLEYF |
| | | | PSQMLRTGNNFQFSYEFENVPFHSSYAHSQSLDRLMNPLIDQYLYYLSRT |
| | | | INGSGQNQQTLKFSVAGPSNMAVQGRNYIPGPSYRQQRVSTTVTQNNNSE |
| | | | FAWPGASSWALNGRNSLMNPGPAMASHKEGEDRFFPLSGSLIFGKQGTGR |
| | | | DNVDADKVMITNEEEIKTTNPVATESYGQVATNHQSAQTRTPVEFAQAQT |
| | | | GWVQNQGILPGMVWQDRDVYLQGPIWAKIPHTDGNFHPSPLMGGFGMKHP |
| | | | PPQILIKNTPVPADPPTAFNKDKLNSFITQYSTGQVSVEIEWELQKENSK |
| | | | RWNPEIQYTSNYYKSNNVEFAVNTEGVYSEPRPIGTRYLTRNL |
| 36 | PRT | Adeno- | MAADGYLPDWLEDNLSEGIREWWALKPGAPQPKANQQHQDNARGLVLPGY |
| | | Associated | KYLGPGNGLDKGEPVNAADAAALEHDKAYDQQLKAGDNPYLKYNHADAEF |
| | | Virus 9 | QERLKEDTSFGGNLGRAVFQAKKRLLEPLGLVEEAAKTAPGKKRPVEQSP |
| | | comprising | QEPDSSAGIGKSGAQPAKKRLNFGQTGDTESVPDPQPIGEPPAAPSGVGS |
| | | SEQ | LTMASGGGAPVADNNEGADGVGSSSGNWHCDSQWLGDRVITTSTRTWALP |
| | | ID NO: 17 | TYNNHLYKQISNSTSGGSSNDNAYFGYSTPWGYFDFNRFHCHFSPRDWQR |
| | | | LINNNWGFRPKRLNFKLFNIQVKEVTDNNGVKTIANNLTSTVQVFTDSDY |
| | | | QLPYVLGSAHEGCLPPFPADVFMIPQYGYLTLNDGSQAVGRSSFYCLEYF |
| | | | PSQMLRTGNNFQFSYEFENVPFHSSYAHSQSLDRLMNPLIDQYLYYLSRT |
| | | | INGSGQNQQTLKFSVAGPSNMAVQGRNYIPGPSYRQQRVSTTVTQNNNSE |
| | | | FAWPGASSWALNGRNSLMNPGPAMASHKEGEDRFFPLSGSLIFGKQGTGR |
| | | | DNVDADKVMITNEEEIKTTNPVATESYGQVATNHQSAQENHTRDKAQAQT |
| | | | GWVQNQGILPGMVWQDRDVYLQGPIWAKIPHTDGNFHPSPLMGGFGMKHP |
| | | | PPQILIKNTPVPADPPTAFNKDKLNSFITQYSTGQVSVEIEWELQKENSK |
| | | | RWNPEIQYTSNYYKSNNVEFAVNTEGVYSEPRPIGTRYLTRNL |
| 37 | PRT | Adeno- | MAADGYLPDWLEDNLSEGIREWWALKPGAPQPKANQQHQDNARGLVLPGY |
| | | Associated | KYLGPGNGLDKGEPVNAADAAALEHDKAYDQQLKAGDNPYLKYNHADAEF |
| | | Virus 9 | QERLKEDTSFGGNLGRAVFQAKKRLLEPLGLVEEAAKTAPGKKRPVEQSP |
| | | comprising | QEPDSSAGIGKSGAQPAKKRLNFGQTGDTESVPDPQPIGEPPAAPSGVGS |
| | | SEQ | LTMASGGGAPVADNNEGADGVGSSSGNWHCDSQWLGDRVITTSTRTWALP |
| | | ID NO: 18 | TYNNHLYKQISNSTSGGSSNDNAYFGYSTPWGYFDFNRFHCHFSPRDWQR |
| | | | LINNNWGFRPKRLNFKLFNIQVKEVTDNNGVKTIANNLTSTVQVFTDSDY |
| | | | QLPYVLGSAHEGCLPPFPADVFMIPQYGYLTLNDGSQAVGRSSFYCLEYF |
| | | | PSQMLRTGNNFQFSYEFENVPFHSSYAHSQSLDRLMNPLIDQYLYYLSRT |
| | | | INGSGQNQQTLKFSVAGPSNMAVQGRNYIPGPSYRQQRVSTTVTQNNNSE |
| | | | FAWPGASSWALNGRNSLMNPGPAMASHKEGEDRFFPLSGSLIFGKQGTGR |
| | | | DNVDADKVMITNEEEIKTTNPVATESYGQVATNHQSAQGNDTRSTAQAQT |
| | | | GWVQNQGILPGMVWQDRDVYLQGPIWAKIPHTDGNFHPSPLMGGFGMKHP |
| | | | PPQILIKNTPVPADPPTAFNKDKLNSFITQYSTGQVSVEIEWELQKENSK |
| | | | RWNPEIQYTSNYYKSNNVEFAVNTEGVYSEPRPIGTRYLTRNL |
| 38 | PRT | Adeno- | MAADGYLPDWLEDNLSEGIREWWALKPGAPQPKANQQHQDNARGLVLPGY |
| | | Associated | KYLGPGNGLDKGEPVNAADAAALEHDKAYDQQLKAGDNPYLKYNHADAEF |
| | | Virus 9 | QERLKEDTSFGGNLGRAVFQAKKRLLEPLGLVEEAAKTAPGKKRPVEQSP |
| | | comprising | QEPDSSAGIGKSGAQPAKKRLNFGQTGDTESVPDPQPIGEPPAAPSGVGS |
| | | SEQ | LTMASGGGAPVADNNEGADGVGSSSGNWHCDSQWLGDRVITTSTRTWALP |
| | | ID NO: 19 | TYNNHLYKQISNSTSGGSSNDNAYFGYSTPWGYFDFNRFHCHFSPRDWQR |
| | | | LINNNWGFRPKRLNFKLFNIQVKEVTDNNGVKTIANNLTSTVQVFTDSDY |
| | | | QLPYVLGSAHEGCLPPFPADVFMIPQYGYLTLNDGSQAVGRSSFYCLEYF |
| | | | PSQMLRTGNNFQFSYEFENVPFHSSYAHSQSLDRLMNPLIDQYLYYLSRT |
| | | | INGSGQNQQTLKFSVAGPSNMAVQGRNYIPGPSYRQQRVSTTVTQNNNSE |
| | | | FAWPGASSWALNGRNSLMNPGPAMASHKEGEDRFFPLSGSLIFGKQGTGR |
| | | | DNVDADKVMITNEEEIKTTNPVATESYGQVATNHQSAQMNSTISKAQAQT |
| | | | GWVQNQGILPGMVWQDRDVYLQGPIWAKIPHTDGNFHPSPLMGGFGMKHP |
| | | | PPQILIKNTPVPADPPTAFNKDKLNSFITQYSTGQVSVEIEWELQKENSK |
| | | | RWNPEIQYTSNYYKSNNVEFAVNTEGVYSEPRPIGTRYLTRNL |
| 39 | PRT | Adeno- | MAADGYLPDWLEDNLSEGIREWWALKPGAPQPKANQQHQDNARGLVLPGY |
| | | Associated | KYLGPGNGLDKGEPVNAADAAALEHDKAYDQQLKAGDNPYLKYNHADAEF |
| | | Virus 9 | QERLKEDTSFGGNLGRAVFQAKKRLLEPLGLVEEAAKTAPGKKRPVEQSP |
| | | comprising | QEPDSSAGIGKSGAQPAKKRLNFGQTGDTESVPDPQPIGEPPAAPSGVGS |
| | | SEQ | LTMASGGGAPVADNNEGADGVGSSSGNWHCDSQWLGDRVITTSTRTWALP |
| | | ID NO: 20 | TYNNHLYKQISNSTSGGSSNDNAYFGYSTPWGYFDFNRFHCHFSPRDWQR |
| | | | LINNNWGFRPKRLNFKLFNIQVKEVTDNNGVKTIANNLTSTVQVFTDSDY |
| | | | QLPYVLGSAHEGCLPPFPADVFMIPQYGYLTLNDGSQAVGRSSFYCLEYF |
| | | | PSQMLRTGNNFQFSYEFENVPFHSSYAHSQSLDRLMNPLIDQYLYYLSRT |
| | | | INGSGQNQQTLKFSVAGPSNMAVQGRNYIPGPSYRQQRVSTTVTQNNNSE |
| | | | FAWPGASSWALNGRNSLMNPGPAMASHKEGEDRFFPLSGSLIFGKQGTGR |
| | | | DNVDADKVMITNEEEIKTTNPVATESYGQVATNHQSAQQSRDLYRAQAQT |
| | | | GWVQNQGILPGMVWQDRDVYLQGPIWAKIPHTDGNFHPSPLMGGFGMKHP |
| | | | PPQILIKNTPVPADPPTAFNKDKLNSFITQYSTGQVSVEIEWELQKENSK |
| | | | RWNPEIQYTSNYYKSNNVEFAVNTEGVYSEPRPIGTRYLTRNL |
| 40 | PRT | Adeno- | MAADGYLPDWLEDNLSEGIREWWALKPGAPQPKANQQHQDNARGLVLPGY |
| | | Associated | KYLGPGNGLDKGEPVNAADAAALEHDKAYDQQLKAGDNPYLKYNHADAEF |
| | | Virus 9 | QERLKEDTSFGGNLGRAVFQAKKRLLEPLGLVEEAAKTAPGKKRPVEQSP |
| | | comprising | QEPDSSAGIGKSGAQPAKKRLNFGQTGDTESVPDPQPIGEPPAAPSGVGS |
| | | SEQ | LTMASGGGAPVADNNEGADGVGSSSGNWHCDSQWLGDRVITTSTRTWALP |
| | | ID NO: 21 | TYNNHLYKQISNSTSGGSSNDNAYFGYSTPWGYFDFNRFHCHFSPRDWQR |
| | | | LINNNWGFRPKRLNFKLFNIQVKEVTDNNGVKTIANNLTSTVQVFTDSDY |
| | | | QLPYVLGSAHEGCLPPFPADVFMIPQYGYLTLNDGSQAVGRSSFYCLEYF |
| | | | PSQMLRTGNNFQFSYEFENVPFHSSYAHSQSLDRLMNPLIDQYLYYLSRT |
| | | | INGSGQNQQTLKFSVAGPSNMAVQGRNYIPGPSYRQQRVSTTVTQNNNSE |
| | | | FAWPGASSWALNGRNSLMNPGPAMASHKEGEDRFFPLSGSLIFGKQGTGR |
| | | | DNVDADKVMITNEEEIKTTNPVATESYGQVATNHQSAQVNTVREFAQAQT |
| | | | GWVQNQGILPGMVWQDRDVYLQGPIWAKIPHTDGNFHPSPLMGGFGMKHP |
| | | | PPQILIKNTPVPADPPTAFNKDKLNSFITQYSTGQVSVEIEWELQKENSK |
| | | | RWNPEIQYTSNYYKSNNVEFAVNTEGVYSEPRPIGTRYLTRNL |
| 41 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 3 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCCCAAGAATTGAACCCACGGGAGTTTGCACAAGCACAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 42 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 3 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctagctcacagccaaagcctggaccg |
| | | | actaatgaatccactcatcgaccaatacttgtactatctctcTaGAacta |
| | | | ttAACGGTTCTGGACAGAATCAAcaaacgctaaaattcagtgtggccgga |
| | | | cccagcaacatggctgtccagggaagaaactacatacctggacccagcta |
| | | | ccgacaacaacgtgtctcaaccactgtgactcaaaacaacaacagcgaat |
| | | | ttgcttggcctggagcttcttcttgggctctcaatggacgtaatagcttg |
| | | | atgaatcctggacctgctatggccagccacaaagaaggagaggaccgttt |
| | | | ctttcctttgtctggatctttaatttttggcaaacaaggTacCggCagag |
| | | | acaacgtggatgcggacaaagtcatgataaccaacgaagaagaaattaaa |
| | | | actactaacccggtagcaacggagtcctatggacaagtggccacaaacca |
| | | | ccagagtGCGCAGGAGCTTAATCCTAGAGAATTCGCTCAGGCGCAGaccg |
| | | | gttgggttcaaaaccaaggaatacttccgggtatggtttggcaggacaga |
| | | | gatgtgtacctgcaaggacccatttgggccaaaattcctcacacggacgg |
| | | | caactttcacccttctccgctgatgggagggtttggaatgaagcacccgc |
| | | | ctcctcagatcctcatcaaaaacacacctgtacctgcggatcctccaacg |
| | | | gccttcaacaaggacaagctgaactctttcatcacccagtattctactgg |
| | | | ccaagtcagcgtggagatcgagtgggagctgcagaaggaaaacagcaagc |
| | | | gctggaacccggagatccagtacacttccaactattacaagtctaataat |
| | | | gttgaatttgctgttaatactgaaggtgtatatagtgaaccccgccccat |
| | | | tggcaccagatacctgactcgtaatctg |
| 43 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 3 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCACAAGAGCTTAATCCTCGAGAATTTGCTCAGGCGCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 44 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 3 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCCCAGGAATTGAACCCAAGGGAGTTCGCTCAGGCTCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 45 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 3 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCCCAGGAGTTAAACCCCCGAGAGTTCGCACAAGCCCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 46 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 3 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctaggtgtgggatctc |
| | | | ttacaatggcttcaggtggtggcgcaccagtggcagacaataacgaaggt |
| | | | gccgatggagtgggtagttcctcgggaaattggcattgcgattcccaatg |
| | | | gctgggggacagagtcatcaccaccagcacccgaacctgggccctgccca |
| | | | cctacaacaatcacctctacaagcaaatctccaacagcacatctggagga |
| | | | tcttcaaatgacaacgcctacttcggctacagcaccccctgggggtattt |
| | | | tgacttcaacagattccactgccacttctcaccacgtgactggcagcgac |
| | | | tcatcaacaacaactggggattccggcctaagcgactcaacttcaagctc |
| | | | ttcaacattcaggtcaaagaggttacggacaacaatggagtcaagaccat |
| | | | cgccaataaccttaccagcacggtccaggtcttcacggactcagactatc |
| | | | agctcccgtacgtgctcgggtcggctcacgagggctgcctcccgccgttc |
| | | | ccagcggacgttttcatgattcctcagtacgggtatctgacgcttaatga |
| | | | tggaagccaggccgtgggtcgttcgtccttttactgcctggaatatttcc |
| | | | cgtcgcaaatgctaagaacgggtaacaacttccagttcagctacgagttt |
| | | | gagaacgtacctttccatagcagctacgctcacagccaaagcctggaccg |
| | | | actaatgaatccactcatcgaccaatacttgtactatctctcTaGAacta |
| | | | ttAACGGTTCTGGACAGAATCAAcaaacgctaaaattcagtgtggccgga |
| | | | cccagcaacatggctgtccagggaagaaactacatacctggacccagcta |
| | | | ccgacaacaacgtgtctcaaccactgtgactcaaaacaacaacagcgaat |
| | | | ttgcttggcctggagcttcttcttgggctctcaatggacgtaatagcttg |
| | | | atgaatcctggacctgctatggccagccacaaagaaggagaggaccgttt |
| | | | ctttcctttgtctggatctttaatttttggcaaacaaggTacCggCagag |
| | | | acaacgtggatgcggacaaagtcatgataaccaacgaagaagaaattaaa |
| | | | actactaacccggtagcaacggagtcctatggacaagtggccacaaacca |
| | | | ccagagtGCACAAGAGTTAAACCCTCGCGAATTCGCCCAAGCTCAGaccg |
| | | | gttgggttcaaaaccaaggaatacttccgggtatggtttggcaggacaga |
| | | | gatgtgtacctgcaaggacccatttgggccaaaattcctcacacggacgg |
| | | | caactttcacccttctccgctgatgggagggtttggaatgaagcacccgc |
| | | | ctcctcagatcctcatcaaaaacacacctgtacctgggatcctccaacgg |
| | | | ccttcaacaaggacaagctgaactctttcatcacccagtattctactggc |
| | | | caagtcagcgtggagatcgagtgggagctgcagaaggaaaacagcaagcg |
| | | | ctggaacccggagatccagtacacttccaactattacaagtctaataatg |
| | | | ttgaatttgctgttaatactgaaggtgtatatagtgaaccccgccccatt |
| | | | ggcaccagatacctgactcgtaatctg |
| 47 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 3 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCGCAGGAATTGAACCCAAGAGAGTTCGCCCAAGCACAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 48 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 3 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCACAAGAGCTAAATCCCCGCGAATTTGCACAAGCCCAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 49 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 3 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCGCAAGAACTGAATCCTAGGGAATTTGCGCAGGCACAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 50 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 3 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCTCAGGAACTCAATCCACGAGAGTTTGCGCAGGCGCAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 51 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 4 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCTCAAGGTAAGCTACCCAGGGAGTTCGCACAAGCGCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctgatggctgccgatggttatctt |
| | | | ccagattggctcgaggacaaccttagtgaaggaattcgcgagtggtgggc |
| | | | tttgaaacctg |
| 52 | DNA | Adeno- | gagcccctcaacccaaggcaaatcaacaacatcaagacaacgctcgaggt |
| | | Associated | cttgtgcttccgggttacaaataccttggacccggcaacggactcgacaa |
| | | Virus 9 | gggggagccggtcaacgcagcagacgcggcggccctcgagcacgacaagg |
| | | comprising | cctacgaccagcagctcaaggrcggagacaacccgtacctcaagtacaac |
| | | SEQ | cacgccgacgccgagttccaggagcggctcaaagaagatacgtcttttgg |
| | | ID NO: 4 | gggcaacctcgggcgagcagtcttccaggccaaaaagaggcttcttgaac |
| | | | ctcttggtctggttgaggaagcggctaagacggctcctggaaagaagagg |
| | | | cctgtagagcagtctcctcaggaaccggactcctccgcgggtattggcaa |
| | | | atcgggtgcacagcccgctaaaaagagactcaatttcggtcagactggcg |
| | | | acacagagtcagtcccagaccctcaaccaatcggagaacctcccgcagcc |
| | | | ccctcaggtgtgggatctcttacaatggcttcaggtggtggcgcaccagt |
| | | | ggcagacaataacgaaggtgccgatggagtgggtagttcctcgggaaatt |
| | | | ggcattgcgattcccaatggctgggggacagagtcatcaccaccagcacc |
| | | | cgaacctgggccctgcccacctacaacaatcacctctacaagcaaatctc |
| | | | caacagcacatctggaggatcttcaaatgacaacgcctacttcggctaca |
| | | | gcaccccctgggggtattttgacttcaacagattccactgccacttctca |
| | | | ccacgtgactggcagcgactcatcaacaacaactggggattccggcctaa |
| | | | gcgactcaacttcaagctcttcaacattcaggtcaaagaggttacggaca |
| | | | acaatggagtcaagaccatcgccaataaccttaccagcacggtccaggtc |
| | | | ttcacggactcagactatcagctcccgtacgtgctcgggtcggctcacga |
| | | | gggctgcctcccgccgttcccagcggacgttttcatgattcctcagtacg |
| | | | ggtatctgacgcttaatgatggaagccaggccgtgggtcgttcgtccttt |
| | | | tactgcctggaatatttcccgtcgcaaatgctaagaacgggtaacaactt |
| | | | ccagttcagctacgagtttgagaacgtacctttccatagcagctacgctc |
| | | | acagccaaagcctggaccgactaatgaatccactcatcgaccaatacttg |
| | | | tactatctctcTaGAactattAACGGTTCTGGACAGAATCAAcaaacgct |
| | | | aaaattcagtgtggccggacccagcaacatggctgtccagggaagaaact |
| | | | acatacctggacccagctaccgacaacaacgtgtctcaaccactgtgact |
| | | | caaaacaacaacagcgaatttgcttggcctggagcttcttcttgggctct |
| | | | caatggacgtaatagcttgatgaatcctggacctgctatggccagccaca |
| | | | aagaaggagaggaccgtttctttcctttgtctggatctttaatttttggc |
| | | | aaacaaggTacCggCagagacaacgtggatgcggacaaagtcatgataac |
| | | | caacgaagaagaaattaaaactactaacccggtagcaacggagtcctatg |
| | | | gacaagtggccacaaaccaccagagtGCACAGGGGAAACTTCCCAGGGAA |
| | | | TTCGCCCAGGCCCAAaccggttgggttcaaaaccaaggaatacttccggg |
| | | | tatggtttggcaggacagagatgtgtacctgcaaggacccatttgggcca |
| | | | aaattcctcacacggacggcaactttcacccttctccgctgatgggaggg |
| | | | tttggaatgaagcacccgcctcctcagatcctcatcaaaaacacacctgt |
| | | | acctgcggatcctccaacggccttcaacaaggacaagctgaactctttca |
| | | | tcacccagtattctactggccaagtcagcgtggagatcgagtgggagctg |
| | | | cagaaggaaaacagcaagcgctggaacccggagatccagtacacttccaa |
| | | | ctattacaagtctaataatgttgaatttgctgttaatactgaaggtgtat |
| | | | atagtgaaccccgccccattggcaccagatacctgactcgtaatctg |
| 53 | DNA | Adeno- | atggctgccgatggttatcttccagattggctcgaggacaaccttagtga |
| | | Associated | aggaattcgcgagtgggggctttgaaacctggagcccctcaacccaaggc |
| | | Virus 9 | aaatcaacaacatcaagacaacgctcgaggtcttgtgcttccgggttaca |
| | | comprising | aataccttggacccggcaacggactcgacaagggggagccggtcaacgca |
| | | SEQ | gcagacgcggcggccctcgagcacgacaaggcctacgaccagcagctcaa |
| | | ID NO: 4 | ggccggagacaacccgtacctcaagtacaaccacgccgacgccgagttcc |
| | | | aggagcggctcaaagaagatacgtcttttgggggcaacctcgggcgagca |
| | | | gtcttccaggccaaaaagaggcttcttgaacctcttggtctggttgagga |
| | | | agcggctaagacggctcctggaaagaagaggcctgtagagcagtctcctc |
| | | | aggaaccggactcctccgcgggtattggcaaatcgggtgcacagcccgct |
| | | | aaaaagagactcaatttcggtcagactggcgacacagagtcagtcccaga |
| | | | ccctcaaccaatcggagaacctcccgcagccccctcaggtgtgggatctc |
| | | | ttacaatggcttcaggtggtggcgcaccagtggcagacaataacgaaggt |
| | | | gccgatggagtgggtagttcctcgggaaattggcattgcgattcccaatg |
| | | | gctgggggacagagtcatcaccaccagcacccgaacctgggccctgccca |
| | | | cctacaacaatcacctctacaagcaaatctccaacagcacatctggagga |
| | | | tcttcaaatgacaacgcctacttcggctacagcaccccctgggggtattt |
| | | | tgacttcaacagattccactgccacttctcaccacgtgactggcagcgac |
| | | | tcatcaacaacaactggggattccggcctaagcgactcaacttcaagctc |
| | | | ttcaacattcaggtcaaagaggttacggacaacaatggagtcaagaccat |
| | | | cgccaataaccttaccagcacggtccaggtcttcacggactcagactatc |
| | | | agctcccgtacgtgctcgggtcggctcacgagggctgcctcccgccgttc |
| | | | ccagcggacgttttcatgattcctcagtacgggtatctgacgcttaatga |
| | | | tggaagccaggccgtgggtcgttcgtccttttactgcctggaatatttcc |
| | | | cgtcgcaaatgctaagaacgggtaacaacttccagttcagctacgagttt |
| | | | gagaacgtacctttccatagcagctacgctcacagccaaagcctggaccg |
| | | | actaatgaatccactcatcgaccaatacttgtactatctctcTaGAacta |
| | | | ttAACGGTTCTGGACAGAATCAAcaaacgctaaaattcagtgtggccgga |
| | | | cccagcaacatggctgtccagggaagaaactacatacctggacccagcta |
| | | | ccgacaacaacgtgtctcaaccactgtgactcaaaacaacaacagcgaat |
| | | | ttgcttggcctggagcttcttcttgggctctcaatggacgtaatagcttg |
| | | | atgaatcctggacctgctatggccagccacaaagaaggagaggaccgttt |
| | | | ctttcctttgtctggatctttaatttttggcaaacaaggTacCggCagag |
| | | | acaacgtggatgcggacaaagtcatgataaccaacgaagaagaaattaaa |
| | | | actactaacccggtagcaacggagtcctatggacaagtggccacaaacca |
| | | | ccagagtGCGCAAGGTAAGCTGCCTCGCGAGTTTGCACAAGCACAGaccg |
| | | | gttgggttcaaaaccaaggaatacttccgggtatggtttggcaggacaga |
| | | | gatgtgtacctgcaaggacccatttgggccaaaattcctcacacggacgg |
| | | | caactttcacccttctccgctgatgggagggtttggaatgaagcacccgc |
| | | | ctcctcagatcctcatcaaaaacacacctgtacctgcggatcctccaacg |
| | | | gccttcaacaaggacaagctgaactctttcatcacccagtattctactgg |
| | | | ccaagtcagcgtggagatcgagtgggagctgcagaaggaaaacagcaagc |
| | | | gctggaacccggagatccagtacacttccaactattacaagtctaataat |
| | | | gttgaatttgctgttaatactgaaggtgtatatagtgaaccccgccccat |
| | | | tggcaccagatacctgactcgtaatctg |
| 54 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 4 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCGCAAGGTAAACTCCCTAGAGAGTTCGCTCAAGCACAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 55 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 4 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCGCAGGGGAAACTTCCTCGGGAATTTGCGCAGGCTCAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 56 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 4 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtgcaaatgctaagaacgggtaacaacttccagttcagctacgagttt |
| | | | gagaacgtacctttccatagcagctacgctcacagccaaagcctggaccg |
| | | | actaatgaatccactcatcgaccaatacttgtactatctctcTaGAacta |
| | | | ttAACGGTTCTGGACAGAATCAAcaaacgctaaaattcagtgtggccgga |
| | | | cccagcaacatggctgtccagggaagaaactacatacctggacccagcta |
| | | | ccgacaacaacgtgtctcaaccactgtgactcaaaacaacaacagcgaat |
| | | | ttgcttggcctggagcttcttcttgggctctcaatggacgtaatagcttg |
| | | | atgaatcctggacctgctatggccagccacaaagaaggagaggaccgttt |
| | | | ctttcctttgtctggatctttaatttttggcaaacaaggTacCggCagag |
| | | | acaacgtggatgcggacaaagtcatgataaccaacgaagaagaaattaaa |
| | | | actactaacccggtagcaacggagtcctatggacaagtggccacaaacca |
| | | | ccagagtGCACAAGGCAAGTTGCCTAGAGAATTTGCGCAGGCACAGaccg |
| | | | gttgggttcaaaaccaaggaatacttccgggtatggtttggcaggacaga |
| | | | gatgtgtacctgcaaggacccatttgggccaaaattcctcacacggacgg |
| | | | caactttcacccttctccgctgatgggagggtttggaatgaagcacccgc |
| | | | ctcctcagatcctcatcaaaaacacacctgtacctgcggatcctccaacg |
| | | | gccttcaacaaggacaagctgaactctttcatcacccagtattctactgg |
| | | | ccaagtcagcgtggagatcgagtgggagctgcagaaggaaaacagcaagc |
| | | | gctggaacccggagatccagtacacttccaactattacaagtctaataat |
| | | | gttgaatttgctgttaatactgaaggtgtatatagtgaaccccgccccat |
| | | | tggcaccagatacctgactcgtaatctg |
| 57 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 4 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctaggtgtgggatctc |
| | | | ttacaatggcttcaggtggtggcgcaccagtggcagacaataacgaaggt |
| | | | gccgatggagtgggtagttcctcgggaaattggcattgcgattcccaatg |
| | | | gctgggggacagagtcatcaccaccagcacccgaacctgggccctgccca |
| | | | cctacaacaatcacctctacaagcaaatctccaacagcacatctggagga |
| | | | tcttcaaatgacaacgcctacttcggctacagcaccccctgggggtattt |
| | | | tgacttcaacagattccactgccacttctcaccacgtgactggcagcgac |
| | | | tcatcaacaacaactggggattccggcctaagcgactcaacttcaagctc |
| | | | ttcaacattcaggtcaaagaggttacggacaacaatggagtcaagaccat |
| | | | cgccaataaccttaccagcacggtccaggtcttcacggactcagactatc |
| | | | agctcccgtacgtgctcgggtcggctcacgagggctgcctcccgccgttc |
| | | | ccagcggacgttttcatgattcctcagtacgggtatctgacgcttaatga |
| | | | tggaagccaggccgtgggtcgttcgtccttttactgcctggaatatttcc |
| | | | cgtcgcaaatgctaagaacgggtaacaacttccagttcagctacgagttt |
| | | | gagaacgtacctttccatagcagctacgctcacagccaaagcctggaccg |
| | | | actaatgaatccactcatcgaccaatacttgtactatctctcTaGAacta |
| | | | ttAACGGTTCTGGACAGAATCAAcaaacgctaaaattcagtgtggccgga |
| | | | cccagcaacatggctgtccagggaagaaactacatacctggacccagcta |
| | | | ccgacaacaacgtgtctcaaccactgtgactcaaaacaacaacagcgaat |
| | | | ttgcttggcctggagcttcttcttgggctctcaatggacgtaatagcttg |
| | | | atgaatcctggacctgctatggccagccacaaagaaggagaggaccgttt |
| | | | ctttcctttgtctggatctttaatttttggcaaacaaggTacCggCagag |
| | | | acaacgtggatgcggacaaagtcatgataaccaacgaagaagaaattaaa |
| | | | actactaacccggtagcaacggagtcctatggacaagtggccacaaacca |
| | | | ccagagtGCTCAAGGGAAACTTCCTCGTGAGTTTGCCCAAGCTCAGaccg |
| | | | gttgggttcaaaaccaaggaatacttccgggtatggtttggcaggacaga |
| | | | gatgtgtacctgcaaggacccatttgggccaaaattcctcacacggacgg |
| | | | caactttcacccttctccgctgatgggagggtttggaatgaagcacccgc |
| | | | ctcctcagatcctcatcaaaaacacacctgtacctgcggatcctccaacg |
| | | | gccttcaacaaggacaagctgaactctttcatcacccagtattctactgg |
| | | | ccaagtcagcgtggagatcgagtgggagctgcagaaggaaaacagcaagc |
| | | | gctggaacccggagatccagtacacttccaactattacaagtctaataat |
| | | | gttgaatttgctgttaatactgaaggtgtatatagtgaaccccgccccat |
| | | | tggcaccagatacctgactcgtaatctg |
| 58 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 49 | aggrcggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCGCAGGGAAAGCTCCCCCGCGAATTCGCTCAGGCACAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 59 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 4 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCGCAGGGTAAACTGCCCCGAGAGTTCGCTCAAGCTCAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 60 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 4 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCCCAGGGAAAGCTGCCGCGCGAATTTGCCCAGGCGCAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 61 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 5 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCACAGCACAATTTCCCTAATGAATTTGCTCAAGCTCAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 62 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 5 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCCCAGCATAACTTTCCAAACGAATTCGCTCAGGCCCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 63 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggggccctcgagcacgacaaggcctacgaccagcagctcaa |
| | | ID NO: 5 | ggccggagacaacccgtacctcaagtacaaccacgccgacgccgagttcc |
| | | | aggagcggctcaaagaagatacgtcttttgggggcaacctcgggcgagca |
| | | | gtcttccaggccaaaaagaggcttcttgaacctcttggtctggttgagga |
| | | | agcggctaagacggctcctggaaagaagaggcctgtagagcagtctcctc |
| | | | aggaaccggactcctccgcgggtattggcaaatcgggtgcacagcccgct |
| | | | aaaaagagactcaatttcggtcagactggcgacacagagtcagtcccaga |
| | | | ccctcaaccaatcggagaacctcccgcagccccctcaggtgtgggatctc |
| | | | ttacaatggcttcaggtggtggcgcaccagtggcagacaataacgaaggt |
| | | | gccgatggagtgggtagttcctcgggaaattggcattgcgattcccaatg |
| | | | gctgggggacagagtcatcaccaccagcacccgaacctgggccctgccca |
| | | | cctacaacaatcacctctacaagcaaatctccaacagcacatctggagga |
| | | | tcttcaaatgacaacgcctacttcggctacagcaccccctgggggtattt |
| | | | tgacttcaacagattccactgccacttctcaccacgtgactggcagcgac |
| | | | tcatcaacaacaactggggattccggcctaagcgactcaacttcaagctc |
| | | | ttcaacattcaggtcaaagaggttacggacaacaatggagtcaagaccat |
| | | | cgccaataaccttaccagcacggtccaggtcttcacggactcagactatc |
| | | | agctcccgtacgtgctcgggtcggctcacgagggctgcctcccgccgttc |
| | | | ccagcggacgttttcatgattcctcagtacgggtatctgacgcttaatga |
| | | | tggaagccaggccgtgggtcgttcgtccttttactgcctggaatatttcc |
| | | | cgtcgcaaatgctaagaacgggtaacaacttccagttcagctacgagttt |
| | | | gagaacgtacctttccatagcagctacgctcacagccaaagcctggaccg |
| | | | actaatgaatccactcatcgaccaatacttgtactatctctcTaGAacta |
| | | | ttAACGGTTCTGGACAGAATCAAcaaacgctaaaattcagtgtggccgga |
| | | | cccagcaacatggctgtccagggaagaaactacatacctggacccagcta |
| | | | ccgacaacaacgtgtctcaaccactgtgactcaaaacaacaacagcgaat |
| | | | ttgcttggcctggagcttcttcttgggctctcaatggacgtaatagcttg |
| | | | atgaatcctggacctgctatggccagccacaaagaaggagaggaccgttt |
| | | | ctttcctttgtctggatctttaatttttggcaaacaaggTacCggCagag |
| | | | acaacgtggatgcggacaaagtcatgataaccaacgaagaagaaattaaa |
| | | | actactaacccggtagcaacggagtcctatggacaagtggccacaaacca |
| | | | ccagagtGCCCAGCATAATTTCCCGAACGAGTTTGCACAAGCTCAAaccg |
| | | | gttgggttcaaaaccaaggaatacttccgggtatggtttggcaggacaga |
| | | | gatgtgtacctgcaaggacccatttgggccaaaattcctcacacggacgg |
| | | | caactttcacccttctccgctgatgggagggtttggaatgaagcacccgc |
| | | | ctcctcagatcctcatcaaaaacacacctgtacctgcggatcctccaacg |
| | | | gccttcaacaaggacaagctgaactctttcatcacccagtattctactgg |
| | | | ccaagtcagcgtggagatcgagtgggagctgcagaaggaaaacagcaagc |
| | | | gctggaacccggagatccagtacacttccaactattacaagtctaataat |
| | | | gttgaatttgctgttaatactgaaggtgtatatagtgaaccccgccccat |
| | | | tggcaccagatacctgactcgtaatctg |
| 64 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 5 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCTCAACATAACTTTCCGAATGAATTCGCACAGGCTCAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 65 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 5 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCTCAACACAATTTCCCAAACGAATTCGCCCAAGCGCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 66 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 5 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCCCAGCACAATTTCCCAAACGAGTTTGCCCAAGCACAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctgatggctgccgatggttatctt |
| | | | ccagattggctcgaggacaaccttagtgaaggaattcgcgagtggtgggc |
| | | | tttgaaacctg |
| 67 | DNA | Adeno- | gagcccctcaacccaaggcaaatcaacaacatcaagacaacgctcgaggt |
| | | Associated | cttgtgcttccgggttacaaataccttggacccggcaacggactcgacaa |
| | | Virus 9 | gggggagccggtcaacgcagcagacgcggcggccctcgagcacgacaagg |
| | | comprising | cctacgaccagcagctcaaggccggagacaacccgtacctcaagtacaac |
| | | SEQ | cacgccgacgccgagttccaggagcggctcaaagaagatacgtcttttgg |
| | | ID NO: 5 | gggcaacctcgggcgagcagtcttccaggccaaaaagaggcttcttgaac |
| | | | ctcttggtctggttgaggaagcggctaagacggctcctggaaagaagagg |
| | | | cctgtagagcagtctcctcaggaaccggactcctccggggtattggcaaa |
| | | | tcgggtgcacagcccgctaaaaagagactcaatttcggtcagactggcga |
| | | | cacagagtcagtcccagaccctcaaccaatcggagaacctcccgcagccc |
| | | | cctcaggtgtgggatctcttacaatggcttcaggtggtggcgcaccagtg |
| | | | gcagacaataacgaaggtgccgatggagtgggtagttcctcgggaaattg |
| | | | gcattgcgattcccaatggctgggggacagagtcatcaccaccagcaccc |
| | | | gaacctgggccctgcccacctacaacaatcacctctacaagcaaatctcc |
| | | | aacagcacatctggaggatcttcaaatgacaacgcctacttcggctacag |
| | | | caccccctgggggtattttgacttcaacagattccactgccacttctcac |
| | | | cacgtgactggcagcgactcatcaacaacaactggggattccggcctaag |
| | | | cgactcaacttcaagctcttcaacattcaggtcaaagaggttacggacaa |
| | | | caatggagtcaagaccatcgccaataaccttaccagcacggtccaggtct |
| | | | tcacggactcagactatcagctcccgtacgtgctcgggtcggctcacgag |
| | | | ggctgcctcccgccgttcccagcggacgttttcatgattcctcagtacgg |
| | | | gtatctgacgcttaatgatggaagccaggccgtgggtcgttcgtcctttt |
| | | | actgcctggaatatttcccgtcgcaaatgctaagaacgggtaacaacttc |
| | | | cagttcagctacgagtttgagaacgtacctttccatagcagctacgctca |
| | | | cagccaaagcctggaccgactaatgaatccactcatcgaccaatacttgt |
| | | | actatctctcTaGAactattAACGGTTCTGGACAGAATCAAcaaacgcta |
| | | | aaattcagtgtggccggacccagcaacatggctgtccagggaagaaacta |
| | | | catacctggacccagctaccgacaacaacgtgtctcaaccactgtgactc |
| | | | aaaacaacaacagcgaatttgcttggcctggagcttcttcttgggctctc |
| | | | aatggacgtaatagcttgatgaatcctggacctgctatggccagccacaa |
| | | | agaaggagaggaccgtttctttcctttgtctggatctttaatttttggca |
| | | | aacaaggTacCggCagagacaacgtggatgcggacaaagtcatgataacc |
| | | | aacgaagaagaaattaaaactactaacccggtagcaacggagtcctatgg |
| | | | acaagtggccacaaaccaccagagtGCGCAACACAACTTTCCAAATGAAT |
| | | | TCGCTCAGGCACAGaccggttgggttcaaaaccaaggaatacttccgggt |
| | | | atggtttggcaggacagagatgtgtacctgcaaggacccatttgggccaa |
| | | | aattcctcacacggacggcaactttcacccttctccgctgatgggagggt |
| | | | ttggaatgaagcacccgcctcctcagatcctcatcaaaaacacacctgta |
| | | | cctgcggatcctccaacggccttcaacaaggacaagctgaactctttcat |
| | | | cacccagtattctactggccaagtcagcgtggagatcgagtgggagctgc |
| | | | agaaggaaaacagcaagcgctggaacccggagatccagtacacttccaac |
| | | | tattacaagtctaataatgttgaatttgctgttaatactgaaggtgtata |
| | | | tagtgaaccccgccccattggcaccagatacctgactcgtaatctg |
| 68 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 5 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCCCAGCATAACTTTCCGAATGAGTTTGCTCAGGCCCAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 69 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 5 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCCCAACATAACTTTCCGAACGAGTTCGCACAGGCCCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 70 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 5 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtgcaaatgctaagaacgggtaacaacttccagttcagctacgagttt |
| | | | gagaacgtacctttccatagcagctacgctcacagccaaagcctggaccg |
| | | | actaatgaatccactcatcgaccaatacttgtactatctctcTaGAacta |
| | | | ttAACGGTTCTGGACAGAATCAAcaaacgctaaaattcagtgtggccgga |
| | | | cccagcaacatggctgtccagggaagaaactacatacctggacccagcta |
| | | | ccgacaacaacgtgtctcaaccactgtgactcaaaacaacaacagcgaat |
| | | | ttgcttggcctggagcttcttcttgggctctcaatggacgtaatagcttg |
| | | | atgaatcctggacctgctatggccagccacaaagaaggagaggaccgttt |
| | | | ctttcctttgtctggatctttaatttttggcaaacaaggTacCggCagag |
| | | | acaacgtggatgcggacaaagtcatgataaccaacgaagaagaaattaaa |
| | | | actactaacccggtagcaacggagtcctatggacaagtggccacaaacca |
| | | | ccagagtGCGCAACACAATTTCCCGAATGAGTTTGCGCAAGCGCAAaccg |
| | | | gttgggttcaaaaccaaggaatacttccgggtatggtttggcaggacaga |
| | | | gatgtgtacctgcaaggacccatttgggccaaaattcctcacacggacgg |
| | | | caactttcacccttctccgctgatgggagggtttggaatgaagcacccgc |
| | | | ctcctcagatcctcatcaaaaacacacctgtacctgcggatcctccaacg |
| | | | gccttcaacaaggacaagctgaactctttcatcacccagtattctactgg |
| | | | ccaagtcagcgtggagatcgagtgggagctgcagaaggaaaacagcaagc |
| | | | gctggaacccggagatccagtacacttccaactattacaagtctaataat |
| | | | gttgaatttgctgttaatactgaaggtgtatatagtgaaccccgccccat |
| | | | tggcaccagatacctgactcgtaatctg |
| 71 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 6 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCACAGATACGTATGCCTACGGAGTTTGCGCAAGCTCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 72 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 6 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCTCAGATAAGGATGCCCACGGAGTTTGCACAGGCGCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 73 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 6 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCGCAAATTCGGATGCCTACGGAATTCGCGCAAGCTCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 74 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 6 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCCCAGATTAGAATGCCGACGGAGTTCGCACAAGCACAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 75 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 6 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCCCAAATCAGGATGCCAACCGAATTCGCCCAGGCACAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 76 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 6 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctaggtgtgggatctc |
| | | | ttacaatggcttcaggtggtggcgcaccagtggcagacaataacgaaggt |
| | | | gccgatggagtgggtagttcctcgggaaattggcattgcgattcccaatg |
| | | | gctgggggacagagtcatcaccaccagcacccgaacctgggccctgccca |
| | | | cctacaacaatcacctctacaagcaaatctccaacagcacatctggagga |
| | | | tcttcaaatgacaacgcctacttcggctacagcaccccctgggggtattt |
| | | | tgacttcaacagattccactgccacttctcaccacgtgactggcagcgac |
| | | | tcatcaacaacaactggggattccggcctaagcgactcaacttcaagctc |
| | | | ttcaacattcaggtcaaagaggttacggacaacaatggagtcaagaccat |
| | | | cgccaataaccttaccagcacggtccaggtcttcacggactcagactatc |
| | | | agctcccgtacgtgctcgggtcggctcacgagggctgcctcccgccgttc |
| | | | ccagcggacgttttcatgattcctcagtacgggtatctgacgcttaatga |
| | | | tggaagccaggccgtgggtcgttcgtccttttactgcctggaatatttcc |
| | | | cgtcgcaaatgctaagaacgggtaacaacttccagttcagctacgagttt |
| | | | gagaacgtacctttccatagcagctacgctcacagccaaagcctggaccg |
| | | | actaatgaatccactcatcgaccaatacttgtactatctctcTaGAacta |
| | | | ttAACGGTTCTGGACAGAATCAAcaaacgctaaaattcagtgtggccgga |
| | | | cccagcaacatggctgtccagggaagaaactacatacctggacccagcta |
| | | | ccgacaacaacgtgtctcaaccactgtgactcaaaacaacaacagcgaat |
| | | | ttgcttggcctggagcttcttcttgggctctcaatggacgtaatagcttg |
| | | | atgaatcctggacctgctatggccagccacaaagaaggagaggaccgttt |
| | | | ctttcctttgtctggatctttaatttttggcaaacaaggTacCggCagag |
| | | | acaacgtggatgcggacaaagtcatgataaccaacgaagaagaaattaaa |
| | | | actactaacccggtagcaacggagtcctatggacaagtggccacaaacca |
| | | | ccagagtGCACAAATCAGGATGCCGACGGAATTCGCGCAGGCTCAAaccg |
| | | | gttgggttcaaaaccaaggaatacttccgggtatggtttggcaggacaga |
| | | | gatgtgtacctgcaaggacccatttgggccaaaattcctcacacggacgg |
| | | | caactttcacccttctccgctgatgggagggtttggaatgaagcacccgc |
| | | | ctcctcagatcctcatcaaaaacacacctgtacctgcggatcctccaacg |
| | | | gccttcaacaaggacaagctgaactctttcatcacccagtattctactgg |
| | | | ccaagtcagcgtggagatcgagtgggagctgcagaaggaaaacagcaagc |
| | | | gctggaacccggagatccagtacacttccaactattacaagtctaataat |
| | | | gttgaatttgctgttaatactgaaggtgtatatagtgaaccccgccccat |
| | | | tggcaccagatacctgactcgtaatctg |
| 77 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 6 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCCCAAATCCGTATGCCGACCGAATTCGCGCAAGCCCAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 78 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 6 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCTCAGATCCGAATGCCCACAGAGTTTGCTCAGGCACAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 79 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 6 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccggggtattggcaaatcgggtgcacagcccgct |
| | | | aaaaagagactcaatttcggtcagactggcgacacagagtcagtcccaga |
| | | | ccctcaaccaatcggagaacctcccgcagccccctcaggtgtgggatctc |
| | | | ttacaatggcttcaggtggtggcgcaccagtggcagacaataacgaaggt |
| | | | gccgatggagtgggtagttcctcgggaaattggcattgcgattcccaatg |
| | | | gctgggggacagagtcatcaccaccagcacccgaacctgggccctgccca |
| | | | cctacaacaatcacctctacaagcaaatctccaacagcacatctggagga |
| | | | tcttcaaatgacaacgcctacttcggctacagcaccccctgggggtattt |
| | | | tgacttcaacagattccactgccacttctcaccacgtgactggcagcgac |
| | | | tcatcaacaacaactggggattccggcctaagcgactcaacttcaagctc |
| | | | ttcaacattcaggtcaaagaggttacggacaacaatggagtcaagaccat |
| | | | cgccaataaccttaccagcacggtccaggtcttcacggactcagactatc |
| | | | agctcccgtacgtgctcgggtcggctcacgagggctgcctcccgccgttc |
| | | | ccagcggacgttttcatgattcctcagtacgggtatctgacgcttaatga |
| | | | tggaagccaggccgtgggtcgttcgtccttttactgcctggaatatttcc |
| | | | cgtcgcaaatgctaagaacgggtaacaacttccagttcagctacgagttt |
| | | | gagaacgtacctttccatagcagctacgctcacagccaaagcctggaccg |
| | | | actaatgaatccactcatcgaccaatacttgtactatctctcTaGAacta |
| | | | ttAACGGTTCTGGACAGAATCAAcaaacgctaaaattcagtgtggccgga |
| | | | cccagcaacatggctgtccagggaagaaactacatacctggacccagcta |
| | | | ccgacaacaacgtgtctcaaccactgtgactcaaaacaacaacagcgaat |
| | | | ttgcttggcctggagcttcttcttgggctctcaatggacgtaatagcttg |
| | | | atgaatcctggacctgctatggccagccacaaagaaggagaggaccgttt |
| | | | ctttcctttgtctggatctttaatttttggcaaacaaggTacCggCagag |
| | | | acaacgtggatgcggacaaagtcatgataaccaacgaagaagaaattaaa |
| | | | actactaacccggtagcaacggagtcctatggacaagtggccacaaacca |
| | | | ccagagtGCTCAAATCCGAATGCCAACCGAATTTGCTCAGGCGCAAaccg |
| | | | gttgggttcaaaaccaaggaatacttccgggtatggtttggcaggacaga |
| | | | gatgtgtacctgcaaggacccatttgggccaaaattcctcacacggacgg |
| | | | caactttcacccttctccgctgatgggagggtttggaatgaagcacccgc |
| | | | ctcctcagatcctcatcaaaaacacacctgtacctgcggatcctccaacg |
| | | | gccttcaacaaggacaagctgaactctttcatcacccagtattctactgg |
| | | | ccaagtcagcgtggagatcgagtgggagctgcagaaggaaaacagcaagc |
| | | | gctggaacccggagatccagtacacttccaactattacaagtctaataat |
| | | | gttgaatttgctgttaatactgaaggtgtatatagtgaaccccgccccat |
| | | | tggcaccagatacctgactcgtaatctg |
| 80 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 6 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCCCAGATACGTATGCCAACCGAGTTTGCCCAAGCGCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 81 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 7 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCTCAAATACGTCAGCCACAGGAATTCGCTCAGGCCCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 82 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 7 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCACAAATTCGGCAACCACAAGAGTTCGCTCAAGCTCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 83 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 7 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCCCAGATCCGCCAGCCTCAGGAATTTGCACAGGCACAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 84 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 7 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCACAGATAAGACAACCTCAGGAATTTGCGCAGGCACAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 85 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 7 | aggrcggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccggggtattggcaaatcgggtgcacagcccgct |
| | | | aaaaagagactcaatttcggtcagactggcgacacagagtcagtcccaga |
| | | | ccctcaaccaatcggagaacctcccgcagccccctcaggtgtgggatctc |
| | | | ttacaatggcttcaggtggtggcgcaccagtggcagacaataacgaaggt |
| | | | gccgatggagtgggtagttcctcgggaaattggcattgcgattcccaatg |
| | | | gctgggggacagagtcatcaccaccagcacccgaacctgggccctgccca |
| | | | cctacaacaatcacctctacaagcaaatctccaacagcacatctggagga |
| | | | tcttcaaatgacaacgcctacttcggctacagcaccccctgggggtattt |
| | | | tgacttcaacagattccactgccacttctcaccacgtgactggcagcgac |
| | | | tcatcaacaacaactggggattccggcctaagcgactcaacttcaagctc |
| | | | ttcaacattcaggtcaaagaggttacggacaacaatggagtcaagaccat |
| | | | cgccaataaccttaccagcacggtccaggtcttcacggactcagactatc |
| | | | agctcccgtacgtgctcgggtcggctcacgagggctgcctcccgccgttc |
| | | | ccagcggacgttttcatgattcctcagtacgggtatctgacgcttaatga |
| | | | tggaagccaggccgtgggtcgttcgtccttttactgcctggaatatttcc |
| | | | cgtcgcaaatgctaagaacgggtaacaacttccagttcagctacgagttt |
| | | | gagaacgtacctttccatagcagctacgctcacagccaaagcctggaccg |
| | | | actaatgaatccactcatcgaccaatacttgtactatctctcTaGAacta |
| | | | ttAACGGTTCTGGACAGAATCAAcaaacgctaaaattcagtgtggccgga |
| | | | cccagcaacatggctgtccagggaagaaactacatacctggacccagcta |
| | | | ccgacaacaacgtgtctcaaccactgtgactcaaaacaacaacagcgaat |
| | | | ttgcttggcctggagcttcttcttgggctctcaatggacgtaatagcttg |
| | | | atgaatcctggacctgctatggccagccacaaagaaggagaggaccgttt |
| | | | ctttcctttgtctggatctttaatttttggcaaacaaggTacCggCagag |
| | | | acaacgtggatgcggacaaagtcatgataaccaacgaagaagaaattaaa |
| | | | actactaacccggtagcaacggagtcctatggacaagtggccacaaacca |
| | | | ccagagtGCCCAAATTCGTCAGCCCCAAGAGTTCGCCCAAGCTCAGaccg |
| | | | gttgggttcaaaaccaaggaatacttccgggtatggtttggcaggacaga |
| | | | gatgtgtacctgcaaggacccatttgggccaaaattcctcacacggacgg |
| | | | caactttcacccttctccgctgatgggagggtttggaatgaagcacccgc |
| | | | ctcctcagatcctcatcaaaaacacacctgtacctgcggatcctccaacg |
| | | | gccttcaacaaggacaagctgaactctttcatcacccagtattctactgg |
| | | | ccaagtcagcgtggagatcgagtgggagctgcagaaggaaaacagcaagc |
| | | | gctggaacccggagatccagtacacttccaactattacaagtctaataat |
| | | | gttgaatttgctgttaatactgaaggtgtatatagtgaaccccgccccat |
| | | | tggcaccagatacctgactcgtaatctg |
| 86 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 7 | aggrcggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCACAGATTCGACAACCCCAAGAGTTTGCCCAAGCACAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 87 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 7 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCTCAGATTAGACAACCTCAGGAGTTTGCGCAAGCGCAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 88 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 7 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCGCAAATCCGCCAGCCGCAAGAATTCGCACAGGCTCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 89 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 7 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCCCAGATTCGTCAGCCACAAGAGTTCGCACAAGCGCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 90 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 7 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCTCAAATACGCCAACCCCAGGAATTTGCCCAGGCCCAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 91 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 8 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCACAGATACGCGTACCGAACGAATTCGCGCAAGCTCAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 92 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 8 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCTCAAATAAGGGTCCCGAACGAGTTTGCGCAAGCTCAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 93 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 8 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCACAAATCAGGGTACCCAATGAATTTGCACAGGCTCAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 94 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 8 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCGCAGATCCGGGTGCCTAATGAATTCGCACAGGCACAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 95 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 8 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCGCAGATACGTGTGCCGAACGAGTTCGCCCAAGCACAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 96 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 8 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCGCAAATAAGAGTACCGAATGAGTTCGCCCAAGCCCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 97 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 8 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCCCAAATCCGAGTGCCAAACGAGTTTGCTCAGGCACAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 98 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 8 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCTCAAATCCGAGTCCCTAATGAGTTTGCCCAGGCGCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 99 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 8 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCACAGATAAGGGTACCCAATGAATTCGCGCAAGCGCAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 100 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 8 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCTCAGATTCGAGTGCCCAACGAATTTGCGCAGGCGCAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 101 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 9 | aggrcggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCCCAAAAAGGCCCGGGATACGAACACGCACAGGCACAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 102 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 9 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCCCAAAAAGGGCCAGGTTACGAACATGCTCAGGCACAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 103 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 9 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCTCAAAAGGGGCCAGGATACGAGCATGCGCAGGCCCAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 104 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 9 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCACAGAAGGGGCCTGGTTATGAGCATGCGCAAGCCCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 105 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 9 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCTCAAAAGGGACCTGGTTATGAACACGCCCAGGCCCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 106 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 9 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | agctcccgtacgtgctcgggtcggctcacgagggctgcctcccgccgttc |
| | | | ccagcggacgttttcatgattcctcagtacgggtatctgacgcttaatga |
| | | | tggaagccaggccgtgggtcgttcgtccttttactgcctggaatatttcc |
| | | | cgtcgcaaatgctaagaacgggtaacaacttccagttcagctacgagttt |
| | | | gagaacgtacctttccatagcagctacgctcacagccaaagcctggaccg |
| | | | actaatgaatccactcatcgaccaatacttgtactatctctcTaGAacta |
| | | | ttAACGGTTCTGGACAGAATCAAcaaacgctaaaattcagtgtggccgga |
| | | | cccagcaacatggctgtccagggaagaaactacatacctggacccagcta |
| | | | ccgacaacaacgtgtctcaaccactgtgactcaaaacaacaacagcgaat |
| | | | ttgcttggcctggagcttcttcttgggctctcaatggacgtaatagcttg |
| | | | atgaatcctggacctgctatggccagccacaaagaaggagaggaccgttt |
| | | | ctttcctttgtctggatctttaatttttggcaaacaaggTacCggCagag |
| | | | acaacgtggatgcggacaaagtcatgataaccaacgaagaagaaattaaa |
| | | | actactaacccggtagcaacggagtcctatggacaagtggccacaaacca |
| | | | ccagagtGCACAGAAAGGTCCGGGCTATGAACACGCACAAGCTCAGaccg |
| | | | gttgggttcaaaaccaaggaatacttccgggtatggtttggcaggacaga |
| | | | gatgtgtacctgcaaggacccatttgggccaaaattcctcacacggacgg |
| | | | caactttcacccttctccgctgatgggagggtttggaatgaagcacccgc |
| | | | ctcctcagatcctcatcaaaaacacacctgtacctgcggatcctccaacg |
| | | | gccttcaacaaggacaagctgaactctttcatcacccagtattctactgg |
| | | | ccaagtcagcgtggagatcgagtgggagctgcagaaggaaaacagcaagc |
| | | | gctggaacccggagatccagtacacttccaactattacaagtctaataat |
| | | | gttgaatttgctgttaatactgaaggtgtatatagtgaaccccgccccat |
| | | | tggcaccagatacctgactcgtaatctg |
| 107 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 9 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCTCAGAAGGGACCGGGGTATGAGCACGCACAAGCTCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 108 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 9 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccggggtattggcaaatcgggtgcacagcccgct |
| | | | aaaaagagactcaatttcggtcagactggcgacacagagtcagtcccaga |
| | | | ccctcaaccaatcggagaacctcccgcagccccctcaggtgtgggatctc |
| | | | ttacaatggcttcaggtggtggcgcaccagtggcagacaataacgaaggt |
| | | | gccgatggagtgggtagttcctcgggaaattggcattgcgattcccaatg |
| | | | gctgggggacagagtcatcaccaccagcacccgaacctgggccctgccca |
| | | | cctacaacaatcacctctacaagcaaatctccaacagcacatctggagga |
| | | | tcttcaaatgacaacgcctacttcggctacagcaccccctgggggtattt |
| | | | tgacttcaacagattccactgccacttctcaccacgtgactggcagcgac |
| | | | tcatcaacaacaactggggattccggcctaagcgactcaacttcaagctc |
| | | | ttcaacattcaggtcaaagaggttacggacaacaatggagtcaagaccat |
| | | | cgccaataaccttaccagcacggtccaggtcttcacggactcagactatc |
| | | | agctcccgtacgtgctcgggtcggctcacgagggctgcctcccgccgttc |
| | | | ccagcggacgttttcatgattcctcagtacgggtatctgacgcttaatga |
| | | | tggaagccaggccgtgggtcgttcgtccttttactgcctggaatatttcc |
| | | | cgtcgcaaatgctaagaacgggtaacaacttccagttcagctacgagttt |
| | | | gagaacgtacctttccatagcagctacgctcacagccaaagcctggaccg |
| | | | actaatgaatccactcatcgaccaatacttgtactatctctcTaGAacta |
| | | | ttAACGGTTCTGGACAGAATCAAcaaacgctaaaattcagtgtggccgga |
| | | | cccagcaacatggctgtccagggaagaaactacatacctggacccagcta |
| | | | ccgacaacaacgtgtctcaaccactgtgactcaaaacaacaacagcgaat |
| | | | ttgcttggcctggagcttcttcttgggctctcaatggacgtaatagcttg |
| | | | atgaatcctggacctgctatggccagccacaaagaaggagaggaccgttt |
| | | | ctttcctttgtctggatctttaatttttggcaaacaaggTacCggCagag |
| | | | acaacgtggatgcggacaaagtcatgataaccaacgaagaagaaattaaa |
| | | | actactaacccggtagcaacggagtcctatggacaagtggccacaaacca |
| | | | ccagagtGCCCAAAAAGGTCCGGGGTACGAACACGCCCAGGCTCAGaccg |
| | | | gttgggttcaaaaccaaggaatacttccgggtatggtttggcaggacaga |
| | | | gatgtgtacctgcaaggacccatttgggccaaaattcctcacacggacgg |
| | | | caactttcacccttctccgctgatgggagggtttggaatgaagcacccgc |
| | | | ctcctcagatcctcatcaaaaacacacctgtacctgcggatcctccaacg |
| | | | gccttcaacaaggacaagctgaactctttcatcacccagtattctactgg |
| | | | ccaagtcagcgtggagatcgagtgggagctgcagaaggaaaacagcaagc |
| | | | gctggaacccggagatccagtacacttccaactattacaagtctaataat |
| | | | gttgaatttgctgttaatactgaaggtgtatatagtgaaccccgccccat |
| | | | tggcaccagatacctgactcgtaatctg |
| 109 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 9 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCACAGAAAGGTCCAGGATACGAGCATGCACAAGCTCAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 110 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 9 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCGCAGAAGGGCCCAGGTTATGAGCATGCTCAAGCGCAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 111 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 10 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgcgttc |
| | | | ccagcggacgttttcatgattcctcagtacgggtatctgacgcttaatga |
| | | | tggaagccaggccgtgggtcgttcgtccttttactgcctggaatatttcc |
| | | | cgtcgcaaatgctaagaacgggtaacaacttccagttcagctacgagttt |
| | | | gagaacgtacctttccatagcagctacgctcacagccaaagcctggaccg |
| | | | actaatgaatccactcatcgaccaatacttgtactatctctcTaGAacta |
| | | | ttAACGGTTCTGGACAGAATCAAcaaacgctaaaattcagtgtggccgga |
| | | | cccagcaacatggctgtccagggaagaaactacatacctggacccagcta |
| | | | ccgacaacaacgtgtctcaaccactgtgactcaaaacaacaacagcgaat |
| | | | ttgcttggcctggagcttcttcttgggctctcaatggacgtaatagcttg |
| | | | atgaatcctggacctgctatggccagccacaaagaaggagaggaccgttt |
| | | | ctttcctttgtctggatctttaatttttggcaaacaaggTacCggCagag |
| | | | acaacgtggatgcggacaaagtcatgataaccaacgaagaagaaattaaa |
| | | | actactaacccggtagcaacggagtcctatggacaagtggccacaaacca |
| | | | ccagagtGCACAAAAGAATTCACCAAGTGAGTTTGCTCAAGCACAGaccg |
| | | | gttgggttcaaaaccaaggaatacttccgggtatggtttggcaggacaga |
| | | | gatgtgtacctgcaaggacccatttgggccaaaattcctcacacggacgg |
| | | | caactttcacccttctccgctgatgggagggtttggaatgaagcacccgc |
| | | | ctcctcagatcctcatcaaaaacacacctgtacctgcggatcctccaacg |
| | | | gccttcaacaaggacaagctgaactctttcatcacccagtattctactgg |
| | | | ccaagtcagcgtggagatcgagtgggagctgcagaaggaaaacagcaagc |
| | | | gctggaacccggagatccagtacacttccaactattacaagtctaataat |
| | | | gttgaatttgctgttaatactgaaggtgtatatagtgaaccccgccccat |
| | | | tggcaccagatacctgactcgtaatctg |
| 112 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 10 | aggrcggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgggacaaagtcatgataaccaacgaagaagaaattaaa |
| | | | actactaacccggtagcaacggagtcctatggacaagtggccacaaacca |
| | | | ccagagtGCCCAGAAAAATTCACCGTCTGAGTTCGCCCAGGCACAAaccg |
| | | | gttgggttcaaaaccaaggaatacttccgggtatggtttggcaggacaga |
| | | | gatgtgtacctgcaaggacccatttgggccaaaattcctcacacggacgg |
| | | | caactttcacccttctccgctgatgggagggtttggaatgaagcacccgc |
| | | | ctcctcagatcctcatcaaaaacacacctgtacctgcggatcctccaacg |
| | | | gccttcaacaaggacaagctgaactctttcatcacccagtattctactgg |
| | | | ccaagtcagcgtggagatcgagtgggagctgcagaaggaaaacagcaagc |
| | | | gctggaacccggagatccagtacacttccaactattacaagtctaataat |
| | | | gttgaatttgctgttaatactgaaggtgtatatagtgaaccccgccccat |
| | | | tggcaccagatacctgactcgtaatctg |
| 113 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 10 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCTCAAAAAAACAGTCCCTCCGAGTTTGCACAGGCTCAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 114 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 10 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCGCAGAAGAATTCACCGTCAGAATTCGCCCAAGCCCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 115 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 10 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | cgtcgcaaatgctaagaacgggtaacaacttccagttcagctacgagttt |
| | | | gagaacgtacctttccatagcagctacgctcacagccaaagcctggaccg |
| | | | actaatgaatccactcatcgaccaatacttgtactatctctcTaGAacta |
| | | | ttAACGGTTCTGGACAGAATCAAcaaacgctaaaattcagtgtggccgga |
| | | | cccagcaacatggctgtccagggaagaaactacatacctggacccagcta |
| | | | ccgacaacaacgtgtctcaaccactgtgactcaaaacaacaacagcgaat |
| | | | ttgcttggcctggagcttcttcttgggctctcaatggacgtaatagcttg |
| | | | atgaatcctggacctgctatggccagccacaaagaaggagaggaccgttt |
| | | | ctttcctttgtctggatctttaatttttggcaaacaaggTacCggCagag |
| | | | acaacgtggatgcggacaaagtcatgataaccaacgaagaagaaattaaa |
| | | | actactaacccggtagcaacggagtcctatggacaagtggccacaaacca |
| | | | ccagagtGCCCAGAAAAACAGTCCGTCGGAATTCGCCCAGGCCCAAaccg |
| | | | gttgggttcaaaaccaaggaatacttccgggtatggtttggcaggacaga |
| | | | gatgtgtacctgcaaggacccatttgggccaaaattcctcacacggacgg |
| | | | caactttcacccttctccgctgatgggagggtttggaatgaagcacccgc |
| | | | ctcctcagatcctcatcaaaaacacacctgtacctgcggatcctccaacg |
| | | | gccttcaacaaggacaagctgaactctttcatcacccagtattctactgg |
| | | | ccaagtcagcgtggagatcgagtgggagctgcagaaggaaaacagcaagc |
| | | | gctggaacccggagatccagtacacttccaactattacaagtctaataat |
| | | | gttgaatttgctgttaatactgaaggtgtatatagtgaaccccgccccat |
| | | | tggcaccagatacctgactcgtaatctg |
| 116 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 10 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctaggtgtgggatctc |
| | | | ttacaatggcttcaggtggtggcgcaccagtggcagacaataacgaaggt |
| | | | gccgatggagtgggtagttcctcgggaaattggcattgcgattcccaatg |
| | | | gctgggggacagagtcatcaccaccagcacccgaacctgggccctgccca |
| | | | cctacaacaatcacctctacaagcaaatctccaacagcacatctggagga |
| | | | tcttcaaatgacaacgcctacttcggctacagcaccccctgggggtattt |
| | | | tgacttcaacagattccactgccacttctcaccacgtgactggcagcgac |
| | | | tcatcaacaacaactggggattccggcctaagcgactcaacttcaagctc |
| | | | ttcaacattcaggtcaaagaggttacggacaacaatggagtcaagaccat |
| | | | cgccaataaccttaccagcacggtccaggtcttcacggactcagactatc |
| | | | agctcccgtacgtgctcgggtcggctcacgagggctgcctcccgccgttc |
| | | | ccagcggacgttttcatgattcctcagtacgggtatctgacgcttaatga |
| | | | tggaagccaggccgtgggtcgttcgtccttttactgcctggaatatttcc |
| | | | cgtcgcaaatgctaagaacgggtaacaacttccagttcagctacgagttt |
| | | | gagaacgtacctttccatagcagctacgctcacagccaaagcctggaccg |
| | | | actaatgaatccactcatcgaccaatacttgtactatctctcTaGAacta |
| | | | ttAACGGTTCTGGACAGAATCAAcaaacgctaaaattcagtgtggccgga |
| | | | cccagcaacatggctgtccagggaagaaactacatacctggacccagcta |
| | | | ccgacaacaacgtgtctcaaccactgtgactcaaaacaacaacagcgaat |
| | | | ttgcttggcctggagcttcttcttgggctctcaatggacgtaatagcttg |
| | | | atgaatcctggacctgctatggccagccacaaagaaggagaggaccgttt |
| | | | ctttcctttgtctggatctttaatttttggcaaacaaggTacCggCagag |
| | | | acaacgtggatgcggacaaagtcatgataaccaacgaagaagaaattaaa |
| | | | actactaacccggtagcaacggagtcctatggacaagtggccacaaacca |
| | | | ccagagtGCGCAAAAAAACTCGCCATCGGAATTTGCCCAGGCTCAGaccg |
| | | | gttgggttcaaaaccaaggaatacttccgggtatggtttggcaggacaga |
| | | | gatgtgtacctgcaaggacccatttgggccaaaattcctcacacggacgg |
| | | | caactttcacccttctccgctgatgggagggtttggaatgaagcacccgc |
| | | | ctcctcagatcctcatcaaaaacacacctgtacctgcggatcctccaacg |
| | | | gccttcaacaaggacaagctgaactctttcatcacccagtattctactgg |
| | | | ccaagtcagcgtggagatcgagtgggagctgcagaaggaaaacagcaagc |
| | | | gctggaacccggagatccagtacacttccaactattacaagtctaataat |
| | | | gttgaatttgctgttaatactgaaggtgtatatagtgaaccccgccccat |
| | | | tggcaccagatacctgactcgtaatctg |
| 117 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 10 | aggrcggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCACAGAAGAACTCCCCATCAGAGTTTGCCCAAGCCCAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 118 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 10 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCCCAAAAAAATAGTCCCAGCGAATTCGCGCAGGCGCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 119 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 10 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCACAGAAGAATAGCCCTAGCGAGTTCGCGCAAGCCCAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 120 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 10 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCGCAAAAGAACTCCCCATCAGAATTTGCGCAAGCCCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 121 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 11 | aggrcggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCACAGTTAGCCTTGCCTCAGGAGTTCGCCCAGGCACAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 122 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 11 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCCCAATTGGCCTTACCTCAGGAATTCGCGCAAGCGCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 123 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 11 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCTCAACTAGCGCTGCCACAAGAATTTGCACAAGCTCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 124 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 11 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCGCAGCTTGCCCTCCCGCAAGAGTTCGCCCAAGCACAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 125 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 11 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCTCAATTGGCACTACCGCAGGAGTTTGCACAGGCGCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 126 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 11 | aggrcggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCTCAACTAGCTTTACCACAGGAATTTGCTCAGGCGCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 127 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 11 | aggrcggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCTCAGCTAGCGCTACCCCAAGAGTTTGCTCAGGCCCAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 128 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 11 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCCCAACTTGCTCTTCCTCAGGAGTTTGCTCAAGCGCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 129 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 11 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCGCAGTTGGCGCTACCCCAAGAATTCGCCCAGGCTCAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 130 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 11 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCCCAGCTCGCCCTCCCACAAGAATTCGCTCAAGCCCAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 131 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 12 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCTCAACAGAATATCCCCTCTGAGTACGCACAAGCTCAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 132 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 12 | aggrcggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCACAGCAAAACATTCCGTCAGAATATGCCCAGGCACAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 133 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 12 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCCCAGCAAAACATACCCTCAGAATACGCACAGGCTCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 134 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 12 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCTCAACAGAATATACCGAGCGAGTACGCGCAAGCTCAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 135 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 12 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCACAGCAAAACATCCCCTCGGAATATGCACAGGCACAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 136 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgggggccctcgagcacgacaaggcctacgaccagcagctcaag |
| | | ID NO: 12 | gccggagacaacccgtacctcaagtacaaccacgccgacgccgagttcca |
| | | | ggagcggctcaaagaagatacgtcttttgggggcaacctcgggcgagcag |
| | | | tcttccaggccaaaaagaggcttcttgaacctcttggtctggttgaggaa |
| | | | gcggctaagacggctcctggaaagaagaggcctgtagagcagtctcctca |
| | | | ggaaccggactcctccgcgggtattggcaaatcgggtgcacagcccgcta |
| | | | aaaagagactcaatttcggtcagactggcgacacagagtcagtcccagac |
| | | | cctcaaccaatcggagaacctcccgcagccccctcaggtgtgggatctct |
| | | | tacaatggcttcaggtggtggcgcaccagtggcagacaataacgaaggtg |
| | | | ccgatggagtgggtagttcctcgggaaattggcattgcgattcccaatgg |
| | | | ctgggggacagagtcatcaccaccagcacccgaacctgggccctgcccac |
| | | | ctacaacaatcacctctacaagcaaatctccaacagcacatctggaggat |
| | | | cttcaaatgacaacgcctacttcggctacagcaccccctgggggtatttt |
| | | | gacttcaacagattccactgccacttctcaccacgtgactggcagcgact |
| | | | catcaacaacaactggggattccggcctaagcgactcaacttcaagctct |
| | | | tcaacattcaggtcaaagaggttacggacaacaatggagtcaagaccatc |
| | | | gccaataaccttaccagcacggtccaggtcttcacggactcagactatca |
| | | | gctcccgtacgtgctcgggtcggctcacgagggctgcctcccgccgttcc |
| | | | cagcggacgttttcatgattcctcagtacgggtatctgacgcttaatgat |
| | | | ggaagccaggccgtgggtcgttcgtccttttactgcctggaatatttccc |
| | | | gtcgcaaatgctaagaacgggtaacaacttccagttcagctacgagtttg |
| | | | agaacgtacctttccatagcagctacgctcacagccaaagcctggaccga |
| | | | ctaatgaatccactcatcgaccaatacttgtactatctctcTaGAactat |
| | | | tAACGGTTCTGGACAGAATCAAcaaacgctaaaattcagtgtggccggac |
| | | | ccagcaacatggctgtccagggaagaaactacatacctggacccagctac |
| | | | cgacaacaacgtgtctcaaccactgtgactcaaaacaacaacagcgaatt |
| | | | tgcttggcctggagcttcttcttgggctctcaatggacgtaatagcttga |
| | | | tgaatcctggacctgctatggccagccacaaagaaggagaggaccgtttc |
| | | | tttcctttgtctggatctttaatttttggcaaacaaggTacCggCagaga |
| | | | caacgtggatgcggacaaagtcatgataaccaacgaagaagaaattaaaa |
| | | | ctactaacccggtagcaacggagtcctatggacaagtggccacaaaccac |
| | | | cagagtGCCCAGCAAAACATTCCTTCAGAATATGCACAGGCGCAGaccgg |
| | | | ttgggttcaaaaccaaggaatacttccgggtatggtttggcaggacagag |
| | | | atgtgtacctgcaaggacccatttgggccaaaattcctcacacggacggc |
| | | | aactttcacccttctccgctgatgggagggtttggaatgaagcacccgcc |
| | | | tcctcagatcctcatcaaaaacacacctgtacctgcggatcctccaacgg |
| | | | ccttcaacaaggacaagctgaactctttcatcacccagtattctactggc |
| | | | caagtcagcgtggagatcgagtgggagctgcagaaggaaaacagcaagcg |
| | | | ctggaacccggagatccagtacacttccaactattacaagtctaataatg |
| | | | ttgaatttgctgttaatactgaaggtgtatatagtgaaccccgccccatt |
| | | | ggcaccagatacctgactcgtaatctg |
| 137 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 12 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCTCAACAGAATATTCCGTCGGAGTATGCGCAAGCCCAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 138 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 12 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCCCAGCAAAATATTCCTAGTGAATATGCACAAGCACAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 139 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 12 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctaggtgtgggatctc |
| | | | ttacaatggcttcaggtggtggcgcaccagtggcagacaataacgaaggt |
| | | | gccgatggagtgggtagttcctcgggaaattggcattgcgattcccaatg |
| | | | gctgggggacagagtcatcaccaccagcacccgaacctgggccctgccca |
| | | | cctacaacaatcacctctacaagcaaatctccaacagcacatctggagga |
| | | | tcttcaaatgacaacgcctacttcggctacagcaccccctgggggtattt |
| | | | tgacttcaacagattccactgccacttctcaccacgtgactggcagcgac |
| | | | tcatcaacaacaactggggattccggcctaagcgactcaacttcaagctc |
| | | | ttcaacattcaggtcaaagaggttacggacaacaatggagtcaagaccat |
| | | | cgccaataaccttaccagcacggtccaggtcttcacggactcagactatc |
| | | | agctcccgtacgtgctcgggtcggctcacgagggctgcctcccgccgttc |
| | | | ccagcggacgttttcatgattcctcagtacgggtatctgacgcttaatga |
| | | | tggaagccaggccgtgggtcgttcgtccttttactgcctggaatatttcc |
| | | | cgtcgcaaatgctaagaacgggtaacaacttccagttcagctacgagttt |
| | | | gagaacgtacctttccatagcagctacgctcacagccaaagcctggaccg |
| | | | actaatgaatccactcatcgaccaatacttgtactatctctcTaGAacta |
| | | | ttAACGGTTCTGGACAGAATCAAcaaacgctaaaattcagtgtggccgga |
| | | | cccagcaacatggctgtccagggaagaaactacatacctggacccagcta |
| | | | ccgacaacaacgtgtctcaaccactgtgactcaaaacaacaacagcgaat |
| | | | ttgcttggcctggagcttcttcttgggctctcaatggacgtaatagcttg |
| | | | atgaatcctggacctgctatggccagccacaaagaaggagaggaccgttt |
| | | | ctttcctttgtctggatctttaatttttggcaaacaaggTacCggCagag |
| | | | acaacgtggatgcggacaaagtcatgataaccaacgaagaagaaattaaa |
| | | | actactaacccggtagcaacggagtcctatggacaagtggccacaaacca |
| | | | ccagagtGCTCAACAGAACATACCGAGCGAGTACGCCCAGGCTCAAaccg |
| | | | gttgggttcaaaaccaaggaatacttccgggtatggtttggcaggacaga |
| | | | gatgtgtacctgcaaggacccatttgggccaaaattcctcacacggacgg |
| | | | caactttcacccttctccgctgatgggagggtttggaatgaagcacccgc |
| | | | ctcctcagatcctcatcaaaaacacacctgtacctgcggatcctccaacg |
| | | | gccttcaacaaggacaagctgaactctttcatcacccagtattctactgg |
| | | | ccaagtcagcgtggagatcgagtgggagctgcagaaggaaaacagcaagc |
| | | | gctggaacccggagatccagtacacttccaactattacaagtctaataat |
| | | | gttgaatttgctgttaatactgaaggtgtatatagtgaaccccgccccat |
| | | | tggcaccagatacctgactcgtaatctg |
| 140 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 12 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCCCAACAGAATATACCTAGCGAGTACGCTCAAGCTCAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 141 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 13 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCTCAACAACGTATACCTCAAGAGTATGCTCAAGCGCAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 142 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 13 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCACAGCAGCGAATTCCTCAGGAATACGCCCAGGCACAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 143 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 13 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCGCAACAGCGGATACCACAGGAGTACGCTCAGGCACAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 144 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 13 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCCCAACAAAGAATCCCCCAAGAGTATGCTCAAGCCCAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 145 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 13 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCCCAGCAGCGTATTCCGCAAGAGTATGCACAGGCCCAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 146 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 13 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCACAGCAGAGGATTCCCCAGGAATACGCCCAGGCACAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 147 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 13 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCGCAACAGAGAATCCCACAGGAGTATGCCCAAGCACAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 148 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 13 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCTCAACAACGAATACCACAGGAATACGCGCAAGCTCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 149 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 13 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCCCAGCAACGCATACCGCAAGAATACGCTCAGGCCCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 150 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 13 | aggrcggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCTCAGCAAAGAATCCCCCAAGAATATGCACAAGCGCAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 151 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 14 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCTCAGCGTTCACAACCGCAAGAACACGCTCAAGCGCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 152 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 14 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | cgtcgcaaatgctaagaacgggtaacaacttccagttcagctacgagttt |
| | | | gagaacgtacctttccatagcagctacgctcacagccaaagcctggaccg |
| | | | actaatgaatccactcatcgaccaatacttgtactatctctcTaGAacta |
| | | | ttAACGGTTCTGGACAGAATCAAcaaacgctaaaattcagtgtggccgga |
| | | | cccagcaacatggctgtccagggaagaaactacatacctggacccagcta |
| | | | ccgacaacaacgtgtctcaaccactgtgactcaaaacaacaacagcgaat |
| | | | ttgcttggcctggagcttcttcttgggctctcaatggacgtaatagcttg |
| | | | atgaatcctggacctgctatggccagccacaaagaaggagaggaccgttt |
| | | | ctttcctttgtctggatctttaatttttggcaaacaaggTacCggCagag |
| | | | acaacgtggatgcggacaaagtcatgataaccaacgaagaagaaattaaa |
| | | | actactaacccggtagcaacggagtcctatggacaagtggccacaaacca |
| | | | ccagagtGCACAGAGAAGTCAGCCACAGGAACATGCACAGGCCCAGaccg |
| | | | gttgggttcaaaaccaaggaatacttccgggtatggtttggcaggacaga |
| | | | gatgtgtacctgcaaggacccatttgggccaaaattcctcacacggacgg |
| | | | caactttcacccttctccgctgatgggagggtttggaatgaagcacccgc |
| | | | ctcctcagatcctcatcaaaaacacacctgtacctgcggatcctccaacg |
| | | | gccttcaacaaggacaagctgaactctttcatcacccagtattctactgg |
| | | | ccaagtcagcgtggagatcgagtgggagctgcagaaggaaaacagcaagc |
| | | | gctggaacccggagatccagtacacttccaactattacaagtctaataat |
| | | | gttgaatttgctgttaatactgaaggtgtatatagtgaaccccgccccat |
| | | | tggcaccagatacctgactcgtaatctg |
| 153 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 14 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCGCAACGGTCACAACCGCAAGAGCACGCCCAAGCACAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 154 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 14 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCTCAGCGTAGTCAACCCCAAGAGCATGCGCAAGCTCAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 155 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 14 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCCCAAAGAAGCCAGCCTCAGGAACACGCTCAGGCGCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 156 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 14 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCGCAACGATCTCAGCCTCAGGAGCATGCCCAGGCACAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 157 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 14 | aggrcggagacaacccgtacctcaagtacaaccacgccgacgccgagttc |
| | | | caggagggctcaaagaagatacgtcttttgggggcaacctcgggcgagca |
| | | | gtcttccaggccaaaaagaggcttcttgaacctcttggtctggttgagga |
| | | | agcggctaagacggctcctggaaagaagaggcctgtagagcagtctcctc |
| | | | aggaaccggactcctccgcgggtattggcaaatcgggtgcacagcccgct |
| | | | aaaaagagactcaatttcggtcagactggcgacacagagtcagtcccaga |
| | | | ccctcaaccaatcggagaacctcccgcagccccctcaggtgtgggatctc |
| | | | ttacaatggcttcaggtggtggcgcaccagtggcagacaataacgaaggt |
| | | | gccgatggagtgggtagttcctcgggaaattggcattgcgattcccaatg |
| | | | gctgggggacagagtcatcaccaccagcacccgaacctgggccctgccca |
| | | | cctacaacaatcacctctacaagcaaatctccaacagcacatctggagga |
| | | | tcttcaaatgacaacgcctacttcggctacagcaccccctgggggtattt |
| | | | tgacttcaacagattccactgccacttctcaccacgtgactggcagcgac |
| | | | tcatcaacaacaactggggattccggcctaagcgactcaacttcaagctc |
| | | | ttcaacattcaggtcaaagaggttacggacaacaatggagtcaagaccat |
| | | | cgccaataaccttaccagcacggtccaggtcttcacggactcagactatc |
| | | | agctcccgtacgtgctcgggtcggctcacgagggctgcctcccgccgttc |
| | | | ccagcggacgttttcatgattcctcagtacgggtatctgacgcttaatga |
| | | | tggaagccaggccgtgggtcgttcgtccttttactgcctggaatatttcc |
| | | | cgtcgcaaatgctaagaacgggtaacaacttccagttcagctacgagttt |
| | | | gagaacgtacctttccatagcagctacgctcacagccaaagcctggaccg |
| | | | actaatgaatccactcatcgaccaatacttgtactatctctcTaGAacta |
| | | | ttAACGGTTCTGGACAGAATCAAcaaacgctaaaattcagtgtggccgga |
| | | | cccagcaacatggctgtccagggaagaaactacatacctggacccagcta |
| | | | ccgacaacaacgtgtctcaaccactgtgactcaaaacaacaacagcgaat |
| | | | ttgcttggcctggagcttcttcttgggctctcaatggacgtaatagcttg |
| | | | atgaatcctggacctgctatggccagccacaaagaaggagaggaccgttt |
| | | | ctttcctttgtctggatctttaatttttggcaaacaaggTacCggCagag |
| | | | acaacgtggatgcggacaaagtcatgataaccaacgaagaagaaattaaa |
| | | | actactaacccggtagcaacggagtcctatggacaagtggccacaaacca |
| | | | ccagagtGCCCAACGCTCTCAGCCACAAGAGCATGCCCAAGCACAGaccg |
| | | | gttgggttcaaaaccaaggaatacttccgggtatggtttggcaggacaga |
| | | | gatgtgtacctgcaaggacccatttgggccaaaattcctcacacggacgg |
| | | | caactttcacccttctccgctgatgggagggtttggaatgaagcacccgc |
| | | | ctcctcagatcctcatcaaaaacacacctgtacctgcggatcctccaacg |
| | | | gccttcaacaaggacaagctgaactctttcatcacccagtattctactgg |
| | | | ccaagtcagcgtggagatcgagtgggagctgcagaaggaaaacagcaagc |
| | | | gctggaacccggagatccagtacacttccaactattacaagtctaataat |
| | | | gttgaatttgctgttaatactgaaggtgtatatagtgaaccccgccccat |
| | | | tggcaccagatacctgactcgtaatctg |
| 158 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 14 | aggccggagacaacccgtacctcaagtacaaccacgccgacgccgagttc |
| | | | caggagcggctcaaagaagatacgtcttttgggggcaacctgggcgagca |
| | | | gtcttccaggccaaaaagaggcttcttgaacctcttggtctggttgagga |
| | | | agcggctaagacggctcctggaaagaagaggcctgtagagcagtctcctc |
| | | | aggaaccggactcctccgcgggtattggcaaatcgggtgcacagcccgct |
| | | | aaaaagagactcaatttcggtcagactggcgacacagagtcagtcccaga |
| | | | ccctcaaccaatcggagaacctcccgcagccccctcaggtgtgggatctc |
| | | | ttacaatggcttcaggtggtggcgcaccagtggcagacaataacgaaggt |
| | | | gccgatggagtgggtagttcctcgggaaattggcattgcgattcccaatg |
| | | | gctgggggacagagtcatcaccaccagcacccgaacctgggccctgccca |
| | | | cctacaacaatcacctctacaagcaaatctccaacagcacatctggagga |
| | | | tcttcaaatgacaacgcctacttcggctacagcaccccctgggggtattt |
| | | | tgacttcaacagattccactgccacttctcaccacgtgactggcagcgac |
| | | | tcatcaacaacaactggggattccggcctaagcgactcaacttcaagctc |
| | | | ttcaacattcaggtcaaagaggttacggacaacaatggagtcaagaccat |
| | | | cgccaataaccttaccagcacggtccaggtcttcacggactcagactatc |
| | | | agctcccgtacgtgctcgggtcggctcacgagggctgcctcccgccgttc |
| | | | ccagcggacgttttcatgattcctcagtacgggtatctgacgcttaatga |
| | | | tggaagccaggccgtgggtcgttcgtccttttactgcctggaatatttcc |
| | | | cgtcgcaaatgctaagaacgggtaacaacttccagttcagctacgagttt |
| | | | gagaacgtacctttccatagcagctacgctcacagccaaagcctggaccg |
| | | | actaatgaatccactcatcgaccaatacttgtactatctctcTaGAacta |
| | | | ttAACGGTTCTGGACAGAATCAAcaaacgctaaaattcagtgtggccgga |
| | | | cccagcaacatggctgtccagggaagaaactacatacctggacccagcta |
| | | | ccgacaacaacgtgtctcaaccactgtgactcaaaacaacaacagcgaat |
| | | | ttgcttggcctggagcttcttcttgggctctcaatggacgtaatagcttg |
| | | | atgaatcctggacctgctatggccagccacaaagaaggagaggaccgttt |
| | | | ctttcctttgtctggatctttaatttttggcaaacaaggTacCggCagag |
| | | | acaacgtggatgcggacaaagtcatgataaccaacgaagaagaaattaaa |
| | | | actactaacccggtagcaacggagtcctatggacaagtggccacaaacca |
| | | | ccagagtGCGCAGCGGAGCCAACCTCAAGAGCACGCGCAAGCCCAGaccg |
| | | | gttgggttcaaaaccaaggaatacttccgggtatggtttggcaggacaga |
| | | | gatgtgtacctgcaaggacccatttgggccaaaattcctcacacggacgg |
| | | | caactttcacccttctccgctgatgggagggtttggaatgaagcacccgc |
| | | | ctcctcagatcctcatcaaaaacacacctgtacctgcggatcctccaacg |
| | | | gccttcaacaaggacaagctgaactctttcatcacccagtattctactgg |
| | | | ccaagtcagcgtggagatcgagtgggagctgcagaaggaaaacagcaagc |
| | | | gctggaacccggagatccagtacacttccaactattacaagtctaataat |
| | | | gttgaatttgctgttaatactgaaggtgtatatagtgaaccccgccccat |
| | | | tggcaccagatacctgactcgtaatctg |
| 159 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 14 | aggccggagacaacccgtacctcaagtacaaccacgccgacgccgagttc |
| | | | caggagcggctcaaagaagatacgtcttttgggggcaacctgggcgagca |
| | | | gtcttccaggccaaaaagaggcttcttgaacctcttggtctggttgagga |
| | | | agcggctaagacggctcctggaaagaagaggcctgtagagcagtctcctc |
| | | | aggaaccggactcctccgcgggtattggcaaatcgggtgcacagcccgct |
| | | | aaaaagagactcaatttcggtcagactggcgacacagagtcagtcccaga |
| | | | ccctcaaccaatcggagaacctcccgcagccccctcaggtgtgggatctc |
| | | | ttacaatggcttcaggtggtggcgcaccagtggcagacaataacgaaggt |
| | | | gccgatggagtgggtagttcctcgggaaattggcattgcgattcccaatg |
| | | | gctgggggacagagtcatcaccaccagcacccgaacctgggccctgccca |
| | | | cctacaacaatcacctctacaagcaaatctccaacagcacatctggagga |
| | | | tcttcaaatgacaacgcctacttcggctacagcaccccctgggggtattt |
| | | | tgacttcaacagattccactgccacttctcaccacgtgactggcagcgac |
| | | | tcatcaacaacaactggggattccggcctaagcgactcaacttcaagctc |
| | | | ttcaacattcaggtcaaagaggttacggacaacaatggagtcaagaccat |
| | | | cgccaataaccttaccagcacggtccaggtcttcacggactcagactatc |
| | | | agctcccgtacgtgctcgggtcggctcacgagggctgcctcccgccgttc |
| | | | ccagcggacgttttcatgattcctcagtacgggtatctgacgcttaatga |
| | | | tggaagccaggccgtgggtcgttcgtccttttactgcctggaatatttcc |
| | | | cgtcgcaaatgctaagaacgggtaacaacttccagttcagctacgagttt |
| | | | gagaacgtacctttccatagcagctacgctcacagccaaagcctggaccg |
| | | | actaatgaatccactcatcgaccaatacttgtactatctctcTaGAacta |
| | | | ttAACGGTTCTGGACAGAATCAAcaaacgctaaaattcagtgtggccgga |
| | | | cccagcaacatggctgtccagggaagaaactacatacctggacccagcta |
| | | | ccgacaacaacgtgtctcaaccactgtgactcaaaacaacaacagcgaat |
| | | | ttgcttggcctggagcttcttcttgggctctcaatggacgtaatagcttg |
| | | | atgaatcctggacctgctatggccagccacaaagaaggagaggaccgttt |
| | | | ctttcctttgtctggatctttaatttttggcaaacaaggTacCggCagag |
| | | | acaacgtggatgcggacaaagtcatgataaccaacgaagaagaaattaaa |
| | | | actactaacccggtagcaacggagtcctatggacaagtggccacaaacca |
| | | | ccagagtGCGCAGAGAAGCCAACCGCAGGAACACGCGCAGGCGCAAaccg |
| | | | gttgggttcaaaaccaaggaatacttccgggtatggtttggcaggacaga |
| | | | gatgtgtacctgcaaggacccatttgggccaaaattcctcacacggacgg |
| | | | caactttcacccttatccgctgatgggagggtttggaatgaagcacccgc |
| | | | ctcctcagatcctcatcaaaaacacacctgtacctgcggatcctccaacg |
| | | | gccttcaacaaggacaagctgaactctttcatcacccagtattctactgg |
| | | | ccaagtcagcgtggagatcgagtgggagctgcagaaggaaaacagcaagc |
| | | | gctggaacccggagatccagtacacttccaactattacaagtctaataat |
| | | | gttgaatttgctgttaatactgaaggtgtatatagtgaaccccgccccat |
| | | | tggcaccagatacctgactcgtaatctg |
| 160 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 14 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCCCAACGTAGTCAGCCACAGGAACATGCACAGGCACAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 161 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 15 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCCCAAACAGCTCGTCCAAACGAGTACGCACAAGCACAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 162 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 15 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagggacgttttcatgattcctcagtacgggtatctgacgcttaatga |
| | | | tggaagccaggccgtgggtcgttcgtccttttactgcctggaatatttcc |
| | | | cgtcgcaaatgctaagaacgggtaacaacttccagttcagctacgagttt |
| | | | gagaacgtacctttccatagcagctacgctcacagccaaagcctggaccg |
| | | | actaatgaatccactcatcgaccaatacttgtactatctctcTaGAacta |
| | | | ttAACGGTTCTGGACAGAATCAAcaaacgctaaaattcagtgtggccgga |
| | | | cccagcaacatggctgtccagggaagaaactacatacctggacccagcta |
| | | | ccgacaacaacgtgtctcaaccactgtgactcaaaacaacaacagcgaat |
| | | | ttgcttggcctggagcttcttcttgggctctcaatggacgtaatagcttg |
| | | | atgaatcctggacctgctatggccagccacaaagaaggagaggaccgttt |
| | | | ctttcctttgtctggatctttaatttttggcaaacaaggTacCggCagag |
| | | | acaacgtggatgcggacaaagtcatgataaccaacgaagaagaaattaaa |
| | | | actactaacccggtagcaacggagtcctatggacaagtggccacaaacca |
| | | | ccagagtGCACAAACAGCGCGTCCTAACGAGTACGCGCAAGCGCAGaccg |
| | | | gttgggttcaaaaccaaggaatacttccgggtatggtttggcaggacaga |
| | | | gatgtgtacctgcaaggacccatttgggccaaaattcctcacacggacgg |
| | | | caactttcacccttctccgctgatgggagggtttggaatgaagcacccgc |
| | | | ctcctcagatcctcatcaaaaacacacctgtacctgcggatcctccaacg |
| | | | gccttcaacaaggacaagctgaactctttcatcacccagtattctactgg |
| | | | ccaagtcagcgtggagatcgagtgggagctgcagaaggaaaacagcaagc |
| | | | gctggaacccggagatccagtacacttccaactattacaagtctaataat |
| | | | gttgaatttgctgttaatactgaaggtgtatatagtgaaccccgccccat |
| | | | tggcaccagatacctgactcgtaatctg |
| 163 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 15 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCTCAAACTGCCCGTCCTAATGAATACGCCCAAGCACAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 164 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 15 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattctcagtacgggtatctgacgcttaatga |
| | | | tggaagccaggccgtgggtcgttcgtccttttactgcctggaatatttcc |
| | | | cgtcgcaaatgctaagaacgggtaacaacttccagttcagctacgagttt |
| | | | gagaacgtacctttccatagcagctacgctcacagccaaagcctggaccg |
| | | | actaatgaatccactcatcgaccaatacttgtactatctctcTaGAacta |
| | | | ttAACGGTTCTGGACAGAATCAAcaaacgctaaaattcagtgtggccgga |
| | | | cccagcaacatggctgtccagggaagaaactacatacctggacccagcta |
| | | | ccgacaacaacgtgtctcaaccactgtgactcaaaacaacaacagcgaat |
| | | | ttgcttggcctggagcttcttcttgggctctcaatggacgtaatagcttg |
| | | | atgaatcctggacctgctatggccagccacaaagaaggagaggaccgttt |
| | | | ctttcctttgtctggatctttaatttttggcaaacaaggTacCggCagag |
| | | | acaacgtggatgcggacaaagtcatgataaccaacgaagaagaaattaaa |
| | | | actactaacccggtagcaacggagtcctatggacaagtggccacaaacca |
| | | | ccagagtGCACAGACAGCGAGACCAAACGAGTATGCGCAGGCTCAGaccg |
| | | | gttgggttcaaaaccaaggaatacttccgggtatggtttggcaggacaga |
| | | | gatgtgtacctgcaaggacccatttgggccaaaattcctcacacggacgg |
| | | | caactttcacccttctccgctgatgggagggtttggaatgaagcacccgc |
| | | | ctcctcagatcctcatcaaaaacacacctgtacctgcggatcctccaacg |
| | | | gccttcaacaaggacaagctgaactctttcatcacccagtattctactgg |
| | | | ccaagtcagcgtggagatcgagtgggagctgcagaaggaaaacagcaagc |
| | | | gctggaacccggagatccagtacacttccaactattacaagtctaataat |
| | | | gttgaatttgctgttaatactgaaggtgtatatagtgaaccccgccccat |
| | | | tggcaccagatacctgactcgtaatctg |
| 165 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 15 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCGCAGACGGCCCGACCTAACGAGTACGCCCAGGCACAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 166 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 15 | aggrcggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCGCAAACTGCTAGACCTAATGAGTACGCACAAGCGCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 167 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 15 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctaggtgtgggatctc |
| | | | ttacaatggcttcaggtggtggcgcaccagtggcagacaataacgaaggt |
| | | | gccgatggagtgggtagttcctcgggaaattggcattgcgattcccaatg |
| | | | gctgggggacagagtcatcaccaccagcacccgaacctgggccctgccca |
| | | | cctacaacaatcacctctacaagcaaatctccaacagcacatctggagga |
| | | | tcttcaaatgacaacgcctacttcggctacagcaccccctgggggtattt |
| | | | tgacttcaacagattccactgccacttctcaccacgtgactggcagcgac |
| | | | tcatcaacaacaactggggattccggcctaagcgactcaacttcaagctc |
| | | | ttcaacattcaggtcaaagaggttacggacaacaatggagtcaagaccat |
| | | | cgccaataaccttaccagcacggtccaggtcttcacggactcagactatc |
| | | | agctcccgtacgtgctcgggtcggctcacgagggctgcctcccgcgttcc |
| | | | cagcggacgttttcatgattcctcagtacgggtatctgacgcttaatgat |
| | | | ggaagccaggccgtgggtcgttcgtccttttactgcctggaatatttccc |
| | | | gtcgcaaatgctaagaacgggtaacaacttccagttcagctacgagtttg |
| | | | agaacgtacctttccatagcagctacgctcacagccaaagcctggaccga |
| | | | ctaatgaatccactcatcgaccaatacttgtactatctctcTaGAactat |
| | | | tAACGGTTCTGGACAGAATCAAcaaacgctaaaattcagtgtggccggac |
| | | | ccagcaacatggctgtccagggaagaaactacatacctggacccagctac |
| | | | cgacaacaacgtgtctcaaccactgtgactcaaaacaacaacagcgaatt |
| | | | tgcttggcctggagcttcttcttgggctctcaatggacgtaatagcttga |
| | | | tgaatcctggacctgctatggccagccacaaagaaggagaggaccgtttc |
| | | | tttcctttgtctggatctttaatttttggcaaacaaggTacCggCagaga |
| | | | caacgtggatgcggacaaagtcatgataaccaacgaagaagaaattaaaa |
| | | | ctactaacccggtagcaacggagtcctatggacaagtggccacaaaccac |
| | | | cagagtGCACAAACCGCACGTCCGAATGAATATGCTCAAGCGCAGaccgg |
| | | | ttgggttcaaaaccaaggaatacttccgggtatggtttggcaggacagag |
| | | | atgtgtacctgcaaggacccatttgggccaaaattcctcacacggacggc |
| | | | aactttcacccttctccgctgatgggagggtttggaatgaagcacccgcc |
| | | | tcctcagatcctcatcaaaaacacacctgtacctgcggatcctccaacgg |
| | | | ccttcaacaaggacaagctgaactctttcatcacccagtattctactggc |
| | | | caagtcagcgtggagatcgagtgggagctgcagaaggaaaacagcaagcg |
| | | | ctggaacccggagatccagtacacttccaactattacaagtctaataatg |
| | | | ttgaatttgctgttaatactgaaggtgtatatagtgaaccccgccccatt |
| | | | ggcaccagatacctgactcgtaatctg |
| 168 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 15 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCCCAGACCGCACGACCGAACGAATATGCGCAGGCACAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 169 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 15 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCGCAGACTGCAAGGCCCAATGAATATGCCCAGGCGCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 170 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 15 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCGCAGACCGCCCGGCCCAATGAATATGCCCAGGCACAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 171 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 16 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCTCAGACGAGGACACCAGTGGAATTCGCGCAAGCACAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 172 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 16 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCACAAACACGTACCCCTGTCGAATTTGCCCAAGCACAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 173 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 16 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCCCAGACGAGGACACCAGTAGAGTTCGCTCAGGCCCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 174 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 16 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCTCAAACGCGCACGCCGGTAGAGTTTGCACAAGCACAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 175 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 16 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctaggtgtgggatctc |
| | | | ttacaatggcttcaggtggtggcgcaccagtggcagacaataacgaaggt |
| | | | gccgatggagtgggtagttcctcgggaaattggcattgcgattcccaatg |
| | | | gctgggggacagagtcatcaccaccagcacccgaacctgggccctgccca |
| | | | cctacaacaatcacctctacaagcaaatctccaacagcacatctggagga |
| | | | tcttcaaatgacaacgcctacttcggctacagcaccccctgggggtattt |
| | | | tgacttcaacagattccactgccacttctcaccacgtgactggcagcgac |
| | | | tcatcaacaacaactggggattccggcctaagcgactcaacttcaagctc |
| | | | ttcaacattcaggtcaaagaggttacggacaacaatggagtcaagaccat |
| | | | cgccaataaccttaccagcacggtccaggtcttcacggactcagactatc |
| | | | agctcccgtacgtgctcgggtcggctcacgagggctgcctcccgccgttc |
| | | | ccagcggacgttttcatgattcctcagtacgggtatctgacgcttaatga |
| | | | tggaagccaggccgtgggtcgttcgtccttttactgcctggaatatttcc |
| | | | cgtcgcaaatgctaagaacgggtaacaacttccagttcagctacgagttt |
| | | | gagaacgtacctttccatagcagctacgctcacagccaaagcctggaccg |
| | | | actaatgaatccactcatcgaccaatacttgtactatctctcTaGAacta |
| | | | ttAACGGTTCTGGACAGAATCAAcaaacgctaaaattcagtgtggccgga |
| | | | cccagcaacatggctgtccagggaagaaactacatacctggacccagcta |
| | | | ccgacaacaacgtgtctcaaccactgtgactcaaaacaacaacagcgaat |
| | | | ttgcttggcctggagcttcttcttgggctctcaatggacgtaatagcttg |
| | | | atgaatcctggacctgctatggccagccacaaagaaggagaggaccgttt |
| | | | ctttcctttgtctggatctttaatttttggcaaacaaggTacCggCagag |
| | | | acaacgtggatgcggacaaagtcatgataaccaacgaagaagaaattaaa |
| | | | actactaacccggtagcaacggagtcctatggacaagtggccacaaacca |
| | | | ccagagtGCGCAAACTAGGACTCCTGTAGAATTTGCCCAAGCCCAAaccg |
| | | | gttgggttcaaaaccaaggaatacttccgggtatggtttggcaggacaga |
| | | | gatgtgtacctgcaaggacccatttgggccaaaattcctcacacggacgg |
| | | | caactttcacccttctccgctgatgggagggtttggaatgaagcacccgc |
| | | | ctcctcagatcctcatcaaaaacacacctgtacctgcggatcctccaacg |
| | | | gccttcaacaaggacaagctgaactctttcatcacccagtattctactgg |
| | | | ccaagtcagcgtggagatcgagtgggagctgcagaaggaaaacagcaagc |
| | | | gctggaacccggagatccagtacacttccaactattacaagtctaataat |
| | | | gttgaatttgctgttaatactgaaggtgtatatagtgaaccccgccccat |
| | | | tggcaccagatacctgactcgtaatctg |
| 176 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 16 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCCCAGACCCGGACTCCTGTTGAATTCGCCCAGGCCCAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 177 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 16 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctaggtgtgggatctc |
| | | | ttacaatggcttcaggtggtggcgcaccagtggcagacaataacgaaggt |
| | | | gccgatggagtgggtagttcctcgggaaattggcattgcgattcccaatg |
| | | | gctgggggacagagtcatcaccaccagcacccgaacctgggccctgccca |
| | | | cctacaacaatcacctctacaagcaaatctccaacagcacatctggagga |
| | | | tcttcaaatgacaacgcctacttcggctacagcaccccctgggggtattt |
| | | | tgacttcaacagattccactgccacttctcaccacgtgactggcagcgac |
| | | | tcatcaacaacaactggggattccggcctaagcgactcaacttcaagctc |
| | | | ttcaacattcaggtcaaagaggttacggacaacaatggagtcaagaccat |
| | | | cgccaataaccttaccagcacggtccaggtcttcacggactcagactatc |
| | | | agctcccgtacgtgctcgggtcggctcacgagggctgcctcccgccgttc |
| | | | ccagcggacgttttcatgattcctcagtacgggtatctgacgcttaatga |
| | | | tggaagccaggccgtgggtcgttcgtccttttactgcctggaatatttcc |
| | | | cgtcgcaaatgctaagaacgggtaacaacttccagttcagctacgagttt |
| | | | gagaacgtacctttccatagcagctacgctcacagccaaagcctggaccg |
| | | | actaatgaatccactcatcgaccaatacttgtactatctctcTaGAacta |
| | | | ttAACGGTTCTGGACAGAATCAAcaaacgctaaaattcagtgtggccgga |
| | | | cccagcaacatggctgtccagggaagaaactacatacctggacccagcta |
| | | | ccgacaacaacgtgtctcaaccactgtgactcaaaacaacaacagcgaat |
| | | | ttgcttggcctggagcttcttcttgggctctcaatggacgtaatagcttg |
| | | | atgaatcctggacctgctatggccagccacaaagaaggagaggaccgttt |
| | | | ctttcctttgtctggatctttaatttttggcaaacaaggTacCggCagag |
| | | | acaacgtggatgcggacaaagtcatgataaccaacgaagaagaaattaaa |
| | | | actactaacccggtagcaacggagtcctatggacaagtggccacaaacca |
| | | | ccagagtGCCCAAACAAGGACGCCGGTCGAATTTGCACAAGCTCAAaccg |
| | | | gttgggttcaaaaccaaggaatacttccgggtatggtttggcaggacaga |
| | | | gatgtgtacctgcaaggacccatttgggccaaaattcctcacacggacgg |
| | | | caactttcacccttctccgctgatgggagggtttggaatgaagcacccgc |
| | | | ctcctcagatcctcatcaaaaacacacctgtacctgcggatcctccaacg |
| | | | gccttcaacaaggacaagctgaactctttcatcacccagtattctactgg |
| | | | ccaagtcagcgtggagatcgagtgggagctgcagaaggaaaacagcaagc |
| | | | gctggaacccggagatccagtacacttccaactattacaagtctaataat |
| | | | gttgaatttgctgttaatactgaaggtgtatatagtgaaccccgccccat |
| | | | tggcaccagatacctgactcgtaatctg |
| 178 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 16 | aggrcggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCACAAACTCGTACGCCGGTTGAGTTTGCCCAGGCGCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 179 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 16 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCTCAGACGCGTACGCCGGTGGAGTTCGCTCAGGCGCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 180 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 16 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtgcaaatgctaagaacgggtaacaacttccagttcagctacgagttt |
| | | | gagaacgtacctttccatagcagctacgctcacagccaaagcctggaccg |
| | | | actaatgaatccactcatcgaccaatacttgtactatctctcTaGAacta |
| | | | ttAACGGTTCTGGACAGAATCAAcaaacgctaaaattcagtgtggccgga |
| | | | cccagcaacatggctgtccagggaagaaactacatacctggacccagcta |
| | | | ccgacaacaacgtgtctcaaccactgtgactcaaaacaacaacagcgaat |
| | | | ttgcttggcctggagcttcttcttgggctctcaatggacgtaatagcttg |
| | | | atgaatcctggacctgctatggccagccacaaagaaggagaggaccgttt |
| | | | ctttcctttgtctggatctttaatttttggcaaacaaggTacCggCagag |
| | | | acaacgtggatgcggacaaagtcatgataaccaacgaagaagaaattaaa |
| | | | actactaacccggtagcaacggagtcctatggacaagtggccacaaacca |
| | | | ccagagtGCGCAGACGCGCACCCCAGTTGAGTTCGCCCAGGCCCAGaccg |
| | | | gttgggttcaaaaccaaggaatacttccgggtatggtttggcaggacaga |
| | | | gatgtgtacctgcaaggacccatttgggccaaaattcctcacacggacgg |
| | | | caactttcacccttctccgctgatgggagggtttggaatgaagcacccgc |
| | | | ctcctcagatcctcatcaaaaacacacctgtacctgcggatcctccaacg |
| | | | gccttcaacaaggacaagctgaactctttcatcacccagtattctactgg |
| | | | ccaagtcagcgtggagatcgagtgggagctgcagaaggaaaacagcaagc |
| | | | gctggaacccggagatccagtacacttccaactattacaagtctaataat |
| | | | gttgaatttgctgttaatactgaaggtgtatatagtgaaccccgccccat |
| | | | tggcaccagatacctgactcgtaatctg |
| 181 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 17 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCGCAGGAGAACCATACTAGGGATAAAGCACAGGCCCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 182 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 17 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCACAGGAGAATCATACTCGTGATAAAGCTCAAGCCCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 183 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 17 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCACAAGAGAATCACACTAGAGATAAAGCACAAGCTCAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 184 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 17 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCGCAAGAAAACCACACTCGCGATAAAGCACAAGCACAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 185 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 17 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCTCAGGAAAATCATACTCGCGATAAGGCGCAAGCCCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 186 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 17 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagggctaagacggctcctggaaagaagaggcctgtagagcagtctcctc |
| | | | aggaaccggactcctccgcgggtattggcaaatcgggtgcacagcccgct |
| | | | aaaaagagactcaatttcggtcagactggcgacacagagtcagtcccaga |
| | | | ccctcaaccaatcggagaacctcccgcagccccctcaggtgtgggatctc |
| | | | ttacaatggcttcaggtggtggcgcaccagtggcagacaataacgaaggt |
| | | | gccgatggagtgggtagttcctcgggaaattggcattgcgattcccaatg |
| | | | gctgggggacagagtcatcaccaccagcacccgaacctgggccctgccca |
| | | | cctacaacaatcacctctacaagcaaatctccaacagcacatctggagga |
| | | | tcttcaaatgacaacgcctacttcggctacagcaccccctgggggtattt |
| | | | tgacttcaacagattccactgccacttctcaccacgtgactggcagcgac |
| | | | tcatcaacaacaactggggattccggcctaagcgactcaacttcaagctc |
| | | | ttcaacattcaggtcaaagaggttacggacaacaatggagtcaagaccat |
| | | | cgccaataaccttaccagcacggtccaggtcttcacggactcagactatc |
| | | | agctcccgtacgtgctcgggtcggctcacgagggctgcctcccgccgttc |
| | | | ccagcggacgttttcatgattctcagtacgggtatctgacgcttaatgat |
| | | | ggaagccaggccgtgggtcgttcgtccttttactgcctggaatatttccc |
| | | | gtcgcaaatgctaagaacgggtaacaacttccagttcagctacgagtttg |
| | | | agaacgtacctttccatagcagctacgctcacagccaaagcctggaccga |
| | | | ctaatgaatccactcatcgaccaatacttgtactatctctcTaGAactat |
| | | | tAACGGTTCTGGACAGAATCAAcaaacgctaaaattcagtgtggccggac |
| | | | ccagcaacatggctgtccagggaagaaactacatacctggacccagctac |
| | | | cgacaacaacgtgtctcaaccactgtgactcaaaacaacaacagcgaatt |
| | | | tgcttggcctggagcttcttcttgggctctcaatggacgtaatagcttga |
| | | | tgaatcctggacctgctatggccagccacaaagaaggagaggaccgtttc |
| | | | tttcctttgtctggatctttaatttttggcaaacaaggTacCggCagaga |
| | | | caacgtggatgcggacaaagtcatgataaccaacgaagaagaaattaaaa |
| | | | ctactaacccggtagcaacggagtcctatggacaagtggccacaaaccac |
| | | | cagagtGCTCAGGAAAACCATACAAGGGACAAGGCGCAGGCGCAGaccgg |
| | | | ttgggttcaaaaccaaggaatacttccgggtatggtttggcaggacagag |
| | | | atgtgtacctgcaaggacccatttgggccaaaattcctcacacggacggc |
| | | | aactttcacccttctccgctgatgggagggtttggaatgaagcacccgcc |
| | | | tcctcagatcctcatcaaaaacacacctgtacctgcggatcctccaacgg |
| | | | ccttcaacaaggacaagctgaactctttcatcacccagtattctactggc |
| | | | caagtcagcgtggagatcgagtgggagctgcagaaggaaaacagcaagcg |
| | | | ctggaacccggagatccagtacacttccaactattacaagtctaataatg |
| | | | ttgaatttgctgttaatactgaaggtgtatatagtgaaccccgccccatt |
| | | | ggcaccagatacctgactcgtaatctg |
| 187 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggggccctcgagcacgacaaggcctacgaccagcagctcaa |
| | | ID NO: 17 | ggccggagacaacccgtacctcaagtacaaccacgccgacgccgagttcc |
| | | | aggagcggctcaaagaagatacgtcttttgggggcaacctcgggcgagca |
| | | | gtcttccaggccaaaaagaggcttcttgaacctcttggtctggttgagga |
| | | | agcggctaagacggctcctggaaagaagaggcctgtagagcagtctcctc |
| | | | aggaaccggactcctccgcgggtattggcaaatcgggtgcacagcccgct |
| | | | aaaaagagactcaatttcggtcagactggcgacacagagtcagtcccaga |
| | | | ccctcaaccaatcggagaacctcccgcagccccctcaggtgtgggatctc |
| | | | ttacaatggcttcaggtggtggcgcaccagtggcagacaataacgaaggt |
| | | | gccgatggagtgggtagttcctcgggaaattggcattgcgattcccaatg |
| | | | gctgggggacagagtcatcaccaccagcacccgaacctgggccctgccca |
| | | | cctacaacaatcacctctacaagcaaatctccaacagcacatctggagga |
| | | | tcttcaaatgacaacgcctacttcggctacagcaccccctgggggtattt |
| | | | tgacttcaacagattccactgccacttctcaccacgtgactggcagcgac |
| | | | tcatcaacaacaactggggattccggcctaagcgactcaacttcaagctc |
| | | | ttcaacattcaggtcaaagaggttacggacaacaatggagtcaagaccat |
| | | | cgccaataaccttaccagcacggtccaggtcttcacggactcagactatc |
| | | | agctcccgtacgtgctcgggtcggctcacgagggctgcctcccgccgttc |
| | | | ccagcggacgttttcatgattcctcagtacgggtatctgacgcttaatga |
| | | | tggaagccaggccgtgggtcgttcgtccttttactgcctggaatatttcc |
| | | | cgtcgcaaatgctaagaacgggtaacaacttccagttcagctacgagttt |
| | | | gagaacgtacctttccatagcagctacgctcacagccaaagcctggaccg |
| | | | actaatgaatccactcatcgaccaatacttgtactatctctcTaGAacta |
| | | | ttAACGGTTCTGGACAGAATCAAcaaacgctaaaattcagtgtggccgga |
| | | | cccagcaacatggctgtccagggaagaaactacatacctggacccagcta |
| | | | ccgacaacaacgtgtctcaaccactgtgactcaaaacaacaacagcgaat |
| | | | ttgcttggcctggagcttcttcttgggctctcaatggacgtaatagcttg |
| | | | atgaatcctggacctgctatggccagccacaaagaaggagaggaccgttt |
| | | | ctttcctttgtctggatctttaatttttggcaaacaaggTacCggCagag |
| | | | acaacgtggatgcggacaaagtcatgataaccaacgaagaagaaattaaa |
| | | | actactaacccggtagcaacggagtcctatggacaagtggccacaaacca |
| | | | ccagagtGCCCAAGAGAACCACACGCGAGACAAAGCTCAGGCGCAAaccg |
| | | | gttgggttcaaaaccaaggaatacttccgggtatggtttggcaggacaga |
| | | | gatgtgtacctgcaaggacccatttgggccaaaattcctcacacggacgg |
| | | | caactttcacccttctccgctgatgggagggtttggaatgaagcacccgc |
| | | | ctcctcagatcctcatcaaaaacacacctgtacctgcggatcctccaacg |
| | | | gccttcaacaaggacaagctgaactctttcatcacccagtattctactgg |
| | | | ccaagtcagcgtggagatcgagtgggagctgcagaaggaaaacagcaagc |
| | | | gctggaacccggagatccagtacacttccaactattacaagtctaataat |
| | | | gttgaatttgctgttaatactgaaggtgtatatagtgaaccccgccccat |
| | | | tggcaccagatacctgactcgtaatctg |
| 188 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 17 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctaggtgtgggatctc |
| | | | ttacaatggcttcaggtggtggcgcaccagtggcagacaataacgaaggt |
| | | | gccgatggagtgggtagttcctcgggaaattggcattgcgattcccaatg |
| | | | gctgggggacagagtcatcaccaccagcacccgaacctgggccctgccca |
| | | | cctacaacaatcacctctacaagcaaatctccaacagcacatctggagga |
| | | | tcttcaaatgacaacgcctacttcggctacagcaccccctgggggtattt |
| | | | tgacttcaacagattccactgccacttctcaccacgtgactggcagcgac |
| | | | tcatcaacaacaactggggattccggcctaagcgactcaacttcaagctc |
| | | | ttcaacattcaggtcaaagaggttacggacaacaatggagtcaagaccat |
| | | | cgccaataaccttaccagcacggtccaggtcttcacggactcagactatc |
| | | | agctcccgtacgtgctcgggtcggctcacgagggctgcctcccgccgttc |
| | | | ccagcggacgttttcatgattcctcagtacgggtatctgacgcttaatga |
| | | | tggaagccaggccgtgggtcgttcgtccttttactgcctggaatatttcc |
| | | | cgtcgcaaatgctaagaacgggtaacaacttccagttcagctacgagttt |
| | | | gagaacgtacctttccatagcagctacgctcacagccaaagcctggaccg |
| | | | actaatgaatccactcatcgaccaatacttgtactatctctcTaGAacta |
| | | | ttAACGGTTCTGGACAGAATCAAcaaacgctaaaattcagtgtggccgga |
| | | | cccagcaacatggctgtccagggaagaaactacatacctggacccagcta |
| | | | ccgacaacaacgtgtctcaaccactgtgactcaaaacaacaacagcgaat |
| | | | ttgcttggcctggagcttcttcttgggctctcaatggacgtaatagcttg |
| | | | atgaatcctggacctgctatggccagccacaaagaaggagaggaccgttt |
| | | | ctttcctttgtctggatctttaatttttggcaaacaaggTacCggCagag |
| | | | acaacgtggatgcggacaaagtcatgataaccaacgaagaagaaattaaa |
| | | | actactaacccggtagcaacggagtcctatggacaagtggccacaaacca |
| | | | ccagagtGCTCAAGAAAATCACACGAGAGACAAGGCCCAAGCGCAAaccg |
| | | | gttgggttcaaaaccaaggaatacttccgggtatggtttggcaggacaga |
| | | | gatgtgtacctgcaaggacccatttgggccaaaattcctcacacggacgg |
| | | | caactttcacccttctccgctgatgggagggtttggaatgaagcacccgc |
| | | | ctcctcagatcctcatcaaaaacacacctgtacctgcggatcctccaacg |
| | | | gccttcaacaaggacaagctgaactctttcatcacccagtattctactgg |
| | | | ccaagtcagcgtggagatcg |
| 189 | DNA | Adeno- | agtgggagctgcagaaggaaaacagcaagcgctggaacccggagatccag |
| | | Associated | tacacttccaactattacaagtctaataatgttgaatttgctgttaatac |
| | | Virus 9 | tgaaggtgtatatagtgaaccccgccccattggcaccagatacctgactc |
| | | comprising | gtaatctgatggctgccgatggttatcttccagattggctcgaggacaac |
| | | SEQ | cttagtgaaggaattcgcgagtggtgggctttgaaacctggagcccctca |
| | | ID NO: 17 | acccaaggcaaatcaacaacatcaagacaacgctcgaggtcttgtgcttc |
| | | | cgggttacaaataccttggacccggcaacggactcgacaagggggagccg |
| | | | gtcaacgcagcagacgcggcggccctcgagcacgacaaggcctacgacca |
| | | | gcagctcaaggccggagacaacccgtacctcaagtacaaccacgccgacg |
| | | | ccgagttccaggagcggctcaaagaagatacgtcttttgggggcaacctc |
| | | | gggcgagcagtcttccaggccaaaaagaggcttcttgaacctcttggtct |
| | | | ggttgaggaagcggctaagacggctcctggaaagaagaggcctgtagagc |
| | | | agtctcctcaggaaccggactcctccgcgggtattggcaaatcgggtgca |
| | | | cagcccgctaaaaagagactcaatttcggtcagactggcgacacagagtc |
| | | | agtcccagaccctcaaccaatcggagaacctcccgcagccccctcaggtg |
| | | | tgggatctcttacaatggcttcaggtggtggcgcaccagtggcagacaat |
| | | | aacgaaggtgccgatggagtgggtagttcctcgggaaattggcattgcga |
| | | | ttcccaatggctgggggacagagtcatcaccaccagcacccgaacctggg |
| | | | ccctgcccacctacaacaatcacctctacaagcaaatctccaacagcaca |
| | | | tctggaggatcttcaaatgacaacgcctacttcggctacagcaccccctg |
| | | | ggggtattttgacttcaacagattccactgccacttctcaccacgtgact |
| | | | ggcagcgactcatcaacaacaactggggattccggcctaagcgactcaac |
| | | | ttcaagctcttcaacattcaggtcaaagaggttacggacaacaatggagt |
| | | | caagaccatcgccaataaccttaccagcacggtccaggtcttcacggact |
| | | | cagactatcagctcccgtacgtgctcgggtcggctcacgagggctgcctc |
| | | | ccgccgttcccagcggacgttttcatgattcctcagtacgggtatctgac |
| | | | gcttaatgatggaagccaggccgtgggtcgttcgtccttttactgcctgg |
| | | | aatatttcccgtcgcaaatgctaagaacgggtaacaacttccagttcagc |
| | | | tacgagtttgagaacgtacctttccatagcagctacgctcacagccaaag |
| | | | cctggaccgactaatgaatccactcatcgaccaatacttgtactatctct |
| | | | cTaGAactattAACGGTTCTGGACAGAATCAAcaaacgctaaaattcagt |
| | | | gtggccggacccagcaacatggctgtccagggaagaaactacatacctgg |
| | | | acccagctaccgacaacaacgtgtctcaaccactgtgactcaaaacaaca |
| | | | acagcgaatttgcttggcctggagcttcttcttgggctctcaatggacgt |
| | | | aatagcttgatgaatcctggacctgctatggccagccacaaagaaggaga |
| | | | ggaccgtttctttcctttgtctggatctttaatttttggcaaacaaggTa |
| | | | cCggCagagacaacgtggatgcggacaaagtcatgataaccaacgaagaa |
| | | | gaaattaaaactactaacccggtagcaacggagtcctatggacaagtggc |
| | | | cacaaaccaccagagtGCTCAAGAAAACCACACGCGAGACAAGGCCCAGG |
| | | | CGCAAaccggttgggttcaaaaccaaggaatacttccgggtatggtttgg |
| | | | caggacagagatgtgtacctgcaaggacccatttgggccaaaattcctca |
| | | | cacggacggcaactttcacccttctccgctgatgggagggtttggaatga |
| | | | agcacccgcctcctcagatcctcatcaaaaacacacctgtacctgcggat |
| | | | cctccaacggccttcaacaaggacaagctgaactctttcatcacccagta |
| | | | ttctactggccaagtcagcgtggagatcgagtgggagctgcagaaggaaa |
| | | | acagcaagcgctggaacccggagatccagtacacttccaactattacaag |
| | | | tctaataatgttgaatttgctgttaatactgaaggtgtatatagtgaacc |
| | | | ccgccccattggcaccagatacctgactcgtaatctg |
| 190 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 17 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCTCAGGAGAATCATACCCGGGACAAGGCGCAGGCCCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 191 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 18 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCGCAGGGAAACGATACGAGATCCACGGCGCAAGCACAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 192 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 18 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCCCAGGGAAACGACACACGTTCGACCGCACAGGCACAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 193 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 18 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCTCAAGGTAATGATACTCGTTCCACGGCACAAGCCCAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 194 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 18 | aggccggagacaacccgtacctcaagtacaaccacgccgacgccgagttc |
| | | | caggagcggctcaaagaagatacgtcttttgggggcaacctggggagcag |
| | | | tcttccaggccaaaaagaggcttcttgaacctcttggtctggttgaggaa |
| | | | gcggctaagacggctcctggaaagaagaggcctgtagagcagtctcctca |
| | | | ggaaccggactcctccgcgggtattggcaaatcgggtgcacagcccgcta |
| | | | aaaagagactcaatttcggtcagactggcgacacagagtcagtcccagac |
| | | | cctcaaccaatcggagaacctcccgcagccccctcaggtgtgggatctct |
| | | | tacaatggcttcaggtggtggcgcaccagtggcagacaataacgaaggtg |
| | | | ccgatggagtgggtagttcctcgggaaattggcattgcgattcccaatgg |
| | | | ctgggggacagagtcatcaccaccagcacccgaacctgggccctgcccac |
| | | | ctacaacaatcacctctacaagcaaatctccaacagcacatctggaggat |
| | | | cttcaaatgacaacgcctacttcggctacagcaccccctgggggtatttt |
| | | | gacttcaacagattccactgccacttctcaccacgtgactggcagcgact |
| | | | catcaacaacaactggggattccggcctaagcgactcaacttcaagctct |
| | | | tcaacattcaggtcaaagaggttacggacaacaatggagtcaagaccatc |
| | | | gccaataaccttaccagcacggtccaggtcttcacggactcagactatca |
| | | | gctcccgtacgtgctcgggtcggctcacgagggctgcctcccgccgttcc |
| | | | cagcggacgttttcatgattcctcagtacgggtatctgacgcttaatgat |
| | | | ggaagccaggccgtgggtcgttcgtccttttactgcctggaatatttccc |
| | | | gtcgcaaatgctaagaacgggtaacaacttccagttcagctacgagtttg |
| | | | agaacgtacctttccatagcagctacgctcacagccaaagcctggaccga |
| | | | ctaatgaatccactcatcgaccaatacttgtactatctctcTaGAactat |
| | | | tAACGGTTCTGGACAGAATCAAcaaacgctaaaattcagtgtggccggac |
| | | | ccagcaacatggctgtccagggaagaaactacatacctggacccagctac |
| | | | cgacaacaacgtgtctcaaccactgtgactcaaaacaacaacagcgaatt |
| | | | tgcttggcctggagcttcttcttgggctctcaatggacgtaatagcttga |
| | | | tgaatcctggacctgctatggccagccacaaagaaggagaggaccgtttc |
| | | | tttcctttgtctggatctttaatttttggcaaacaaggTacCggCagaga |
| | | | caacgtggatgcggacaaagtcatgataaccaacgaagaagaaattaaaa |
| | | | ctactaacccggtagcaacggagtcctatggacaagtggccacaaaccac |
| | | | cagagtGCACAGGGTAACGATACGCGAAGCACAGCTCAAGCACAAaccgg |
| | | | ttgggttcaaaaccaaggaatacttccgggtatggtttggcaggacagag |
| | | | atgtgtacctgcaaggacccatttgggccaaaattcctcacacggacggc |
| | | | aactttcacccttctccgctgatgggagggtttggaatgaagcacccgcc |
| | | | tcctcagatcctcatcaaaaacacacctgtacctgcggatcctccaacgg |
| | | | ccttcaacaaggacaagctgaactctttcatcacccagtattctactggc |
| | | | caagtcagcgtggagatcgagtgggagctgcagaaggaaaacagcaagcg |
| | | | ctggaacccggagatccagtacacttccaactattacaagtctaataatg |
| | | | ttgaatttgctgttaatactgaaggtgtatatagtgaaccccgccccatt |
| | | | ggcaccagatacctgactcgtaatctg |
| 195 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 18 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCACAGGGTAACGATACGCGGTCCACAGCACAGGCACAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 196 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 18 | aggrcggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCTCAAGGGAATGATACGAGAAGTACTGCTCAAGCGCAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 197 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 18 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCCCAGGGAAACGACACCAGGAGCACAGCTCAGGCACAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 198 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggggccctcgagcacgacaaggcctacgaccagcagctcaa |
| | | ID NO: 18 | ggccggagacaacccgtacctcaagtacaaccacgccgacgccgagttcc |
| | | | aggagcggctcaaagaagatacgtcttttgggggcaacctcgggcgagca |
| | | | gtcttccaggccaaaaagaggcttcttgaacctcttggtctggttgagga |
| | | | agcggctaagacggctcctggaaagaagaggcctgtagagcagtctcctc |
| | | | aggaaccggactcctccgcgggtattggcaaatcgggtgcacagcccgct |
| | | | aaaaagagactcaatttcggtcagactggcgacacagagtcagtcccaga |
| | | | ccctcaaccaatcggagaacctcccgcagccccctcaggtgtgggatctc |
| | | | ttacaatggcttcaggtggtggcgcaccagtggcagacaataacgaaggt |
| | | | gccgatggagtgggtagttcctcgggaaattggcattgcgattcccaatg |
| | | | gctgggggacagagtcatcaccaccagcacccgaacctgggccctgccca |
| | | | cctacaacaatcacctctacaagcaaatctccaacagcacatctggagga |
| | | | tcttcaaatgacaacgcctacttcggctacagcaccccctgggggtattt |
| | | | tgacttcaacagattccactgccacttctcaccacgtgactggcagcgac |
| | | | tcatcaacaacaactggggattccggcctaagcgactcaacttcaagctc |
| | | | ttcaacattcaggtcaaagaggttacggacaacaatggagtcaagaccat |
| | | | cgccaataaccttaccagcacggtccaggtcttcacggactcagactatc |
| | | | agctcccgtacgtgctcgggtcggctcacgagggctgcctcccgccgttc |
| | | | ccagcggacgttttcatgattcctcagtacgggtatctgacgcttaatga |
| | | | tggaagccaggccgtgggtcgttcgtccttttactgcctggaatatttcc |
| | | | cgtcgcaaatgctaagaacgggtaacaacttccagttcagctacgagttt |
| | | | gagaacgtacctttccatagcagctacgctcacagccaaagcctggaccg |
| | | | actaatgaatccactcatcgaccaatacttgtactatctctcTaGAacta |
| | | | ttAACGGTTCTGGACAGAATCAAcaaacgctaaaattcagtgtggccgga |
| | | | cccagcaacatggctgtccagggaagaaactacatacctggacccagcta |
| | | | ccgacaacaacgtgtctcaaccactgtgactcaaaacaacaacagcgaat |
| | | | ttgcttggcctggagcttcttcttgggctctcaatggacgtaatagcttg |
| | | | atgaatcctggacctgctatggccagccacaaagaaggagaggaccgttt |
| | | | ctttcctttgtctggatctttaatttttggcaaacaaggTacCggCagag |
| | | | acaacgtggatgcggacaaagtcatgataaccaacgaagaagaaattaaa |
| | | | actactaacccggtagcaacggagtcctatggacaagtggccacaaacca |
| | | | ccagagtGCGCAAGGAAATGACACTCGATCGACGGCGCAAGCTCAAaccg |
| | | | gttgggttcaaaaccaaggaatacttccgggtatggtttggcaggacaga |
| | | | gatgtgtacctgcaaggacccatttgggccaaaattcctcacacggacgg |
| | | | caactttcacccttctccgctgatgggagggtttggaatgaagcacccgc |
| | | | ctcctcagatcctcatcaaaaacacacctgtacctgcggatcctccaacg |
| | | | gccttcaacaaggacaagctgaactctttcatcacccagtattctactgg |
| | | | ccaagtcagcgtggagatcgagtgggagctgcagaaggaaaacagcaagc |
| | | | gctggaacccggagatccagtacacttccaactattacaagtctaataat |
| | | | gttgaatttgctgttaatactgaaggtgtatatagtgaaccccgccccat |
| | | | tggcaccagatacctgactcgtaatctg |
| 199 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 18 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCGCAAGGGAATGACACCAGATCGACTGCGCAGGCTCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 200 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 18 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | agctcccgtacgtgctcgggtcggctcacgagggctgcctcccgccgttc |
| | | | ccagcggacgttttcatgattcctcagtacgggtatctgacgcttaatga |
| | | | tggaagccaggccgtgggtcgttcgtccttttactgcctggaatatttcc |
| | | | cgtcgcaaatgctaagaacgggtaacaacttccagttcagctacgagttt |
| | | | gagaacgtacctttccatagcagctacgctcacagccaaagcctggaccg |
| | | | actaatgaatccactcatcgaccaatacttgtactatctctcTaGAacta |
| | | | ttAACGGTTCTGGACAGAATCAAcaaacgctaaaattcagtgtggccgga |
| | | | cccagcaacatggctgtccagggaagaaactacatacctggacccagcta |
| | | | ccgacaacaacgtgtctcaaccactgtgactcaaaacaacaacagcgaat |
| | | | ttgcttggcctggagcttcttcttgggctctcaatggacgtaatagcttg |
| | | | atgaatcctggacctgctatggccagccacaaagaaggagaggaccgttt |
| | | | ctttcctttgtctggatctttaatttttggcaaacaaggTacCggCagag |
| | | | acaacgtggatgcggacaaagtcatgataaccaacgaagaagaaattaaa |
| | | | actactaacccggtagcaacggagtcctatggacaagtggccacaaacca |
| | | | ccagagtGCTCAAGGTAATGACACACGCTCTACAGCCCAGGCTCAAaccg |
| | | | gttgggttcaaaaccaaggaatacttccgggtatggtttggcaggacaga |
| | | | gatgtgtacctgcaaggacccatttgggccaaaattcctcacacggacgg |
| | | | caactttcacccttctccgctgatgggagggtttggaatgaagcacccgc |
| | | | ctcctcagatcctcatcaaaaacacacctgtacctgcggatcctccaacg |
| | | | gccttcaacaaggacaagctgaactctttcatcacccagtattctactgg |
| | | | ccaagtcagcgtggagatcgagtgggagctgcagaaggaaaacagcaagc |
| | | | gctggaacccggagatccagtacacttccaactattacaagtctaataat |
| | | | gttgaatttgctgttaatactgaaggtgtatatagtgaaccccgccccat |
| | | | tggcaccagatacctgactcgtaatctg |
| 201 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 19 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCCCAGATGAACAGCACAATATCTAAAGCGCAGGCACAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 202 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 19 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCACAGATGAATAGCACCATATCGAAAGCACAAGCTCAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 203 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 19 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCTCAAATGAACTCAACAATTTCAAAGGCGCAAGCACAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 204 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 19 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCACAGATGAATAGTACCATATCGAAAGCCCAGGCTCAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 205 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 19 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCGCAAATGAATAGTACGATCAGTAAGGCTCAAGCGCAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 206 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 19 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCTCAGATGAATTCTACGATATCGAAGGCACAGGCACAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 207 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 19 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCTCAAATGAACTCGACGATCTCGAAGGCGCAAGCCCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 208 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 19 | aggrcggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCCCAAATGAACTCAACAATCTCCAAGGCTCAGGCGCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 209 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 19 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCCCAAATGAACAGCACAATTTCAAAAGCGCAAGCGCAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 210 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 19 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCCCAGATGAATTCAACTATTAGTAAAGCTCAGGCGCAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 211 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 20 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcaga\caataacgaag |
| | | | gtgccgatggagtgggtagttcctcgggaaattggcattgcgattcccaa |
| | | | tggctgggggacagagtcatcaccaccagcacccgaacctgggccctgcc |
| | | | cacctacaacaatcacctctacaagcaaatctccaacagcacatctggag |
| | | | gatcttcaaatgacaacgcctacttcggctacagcaccccctgggggtat |
| | | | tttgacttcaacagattccactgccacttctcaccacgtgactggcagcg |
| | | | actcatcaacaacaactggggattccggcctaagcgactcaacttcaagc |
| | | | tcttcaacattcaggtcaaagaggttacggacaacaatggagtcaagacc |
| | | | atcgccaataaccttaccagcacggtccaggtcttcacggactcagacta |
| | | | tcagctcccgtacgtgctcgggtcggctcacgagggctgcctcccgccgt |
| | | | tcccagcggacgttttcatgattcctcagtacgggtatctgacgcttaat |
| | | | gatggaagccaggccgtgggtcgttcgtccttttactgcctggaatattt |
| | | | cccgtcgcaaatgctaagaacgggtaacaacttccagttcagctacgagt |
| | | | ttgagaacgtacctttccatagcagctacgctcacagccaaagcctggac |
| | | | cgactaatgaatccactcatcgaccaatacttgtactatctctcTaGAac |
| | | | tattAACGGTTCTGGACAGAATCAAcaaacgctaaaattcagtgtggccg |
| | | | gacccagcaacatggctgtccagggaagaaactacatacctggacccagc |
| | | | taccgacaacaacgtgtctcaaccactgtgactcaaaacaacaacagcga |
| | | | atttgcttggcctggagcttcttcttgggctctcaatggacgtaatagct |
| | | | tgatgaatcctggacctgctatggccagccacaaagaaggagaggaccgt |
| | | | ttctttcctttgtctggatctttaatttttggcaaacaaggTacCggCag |
| | | | agacaacgtggatgcggacaaagtcatgataaccaacgaagaagaaatta |
| | | | aaactactaacccggtagcaacggagtcctatggacaagtggccacaaac |
| | | | caccagagtGCACAAAACGGGAAGGCAGACCCTCGGGCTCAAGCACAAac |
| | | | cggttgggttcaaaaccaaggaatacttccgggtatggtttggcaggaca |
| | | | gagatgtgtacctgcaaggacccatttgggccaaaattcctcacacggac |
| | | | ggcaactttcacccttctccgctgatgggagggtttggaatgaagcaccc |
| | | | gcctcctcagatcctcatcaaaaacacacctgtacctgcggatcctccaa |
| | | | cggccttcaacaaggacaagctgaactctttcatcacccagtattctact |
| | | | ggccaagtcagcgtggagatcgagtgggagctgcagaaggaaaacagcaa |
| | | | gcgctggaacccggagatccagtacacttccaactattacaagtctaata |
| | | | atgttgaatttgctgttaatactgaaggtgtatatagtgaaccccgcccc |
| | | | attggcaccagatacctgactcgtaatctg |
| 212 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 20 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCGCAGAACGGAAAGGCAGATCCACGAGCTCAGGCACAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 213 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 20 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccggggtattggcaaatcgggtgcacagcccgct |
| | | | aaaaagagactcaatttcggtcagactggcgacacagagtcagtcccaga |
| | | | ccctcaaccaatcggagaacctcccgcagccccctcaggtgtgggatctc |
| | | | ttacaatggcttcaggtggtggcgcaccagtggcagacaataacgaaggt |
| | | | gccgatggagtgggtagttcctcgggaaattggcattgcgattcccaatg |
| | | | gctgggggacagagtcatcaccaccagcacccgaacctgggccctgccca |
| | | | cctacaacaatcacctctacaagcaaatctccaacagcacatctggagga |
| | | | tcttcaaatgacaacgcctacttcggctacagcaccccctgggggtattt |
| | | | tgacttcaacagattccactgccacttctcaccacgtgactggcagcgac |
| | | | tcatcaacaacaactggggattccggcctaagcgactcaacttcaagctc |
| | | | ttcaacattcaggtcaaagaggttacggacaacaatggagtcaagaccat |
| | | | cgccaataaccttaccagcacggtccaggtcttcacggactcagactatc |
| | | | agctcccgtacgtgctcgggtcggctcacgagggctgcctcccgccgttc |
| | | | ccagcggacgttttcatgattcctcagtacgggtatctgacgcttaatga |
| | | | tggaagccaggccgtgggtcgttcgtccttttactgcctggaatatttcc |
| | | | cgtcgcaaatgctaagaacgggtaacaacttccagttcagctacgagttt |
| | | | gagaacgtacctttccatagcagctacgctcacagccaaagcctggaccg |
| | | | actaatgaatccactcatcgaccaatacttgtactatctctcTaGAacta |
| | | | ttAACGGTTCTGGACAGAATCAAcaaacgctaaaattcagtgtggccgga |
| | | | cccagcaacatggctgtccagggaagaaactacatacctggacccagcta |
| | | | ccgacaacaacgtgtctcaaccactgtgactcaaaacaacaacagcgaat |
| | | | ttgcttggcctggagcttcttcttgggctctcaatggacgtaatagcttg |
| | | | atgaatcctggacctgctatggccagccacaaagaaggagaggaccgttt |
| | | | ctttcctttgtctggatctttaatttttggcaaacaaggTacCggCagag |
| | | | acaacgtggatgcggacaaagtcatgataaccaacgaagaagaaattaaa |
| | | | actactaacccggtagcaacggagtcctatggacaagtggccacaaacca |
| | | | ccagagtGCGCAGAATGGCAAGGCAGACCCGCGAGCCCAGGCACAGaccg |
| | | | gttgggttcaaaaccaaggaatacttccgggtatggtttggcaggacaga |
| | | | gatgtgtacctgcaaggacccatttgggccaaaattcctcacacggacgg |
| | | | caactttcacccttctccgctgatgggagggtttggaatgaagcacccgc |
| | | | ctcctcagatcctcatcaaaaacacacctgtacctgcggatcctccaacg |
| | | | gccttcaacaaggacaagctgaactctttcatcacccagtattctactgg |
| | | | ccaagtcagcgtggagatcgagtgggagctgcagaaggaaaacagcaagc |
| | | | gctggaacccggagatccagtacacttccaactattacaagtctaataat |
| | | | gttgaatttgctgttaatactgaaggtgtatatagtgaaccccgccccat |
| | | | tggcaccagatacctgactcgtaatctg |
| 214 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 20 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCACAGAATGGAAAAGCAGACCCACGTGCCCAAGCACAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 215 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 20 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCCCAAAATGGTAAAGCCGATCCGCGTGCGCAAGCACAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 216 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 20 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCTCAGAATGGTAAAGCCGATCCACGAGCACAGGCTCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 217 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 20 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCGCAGAACGGGAAGGCAGACCCTCGAGCACAGGCCCAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 218 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 20 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCCCAAAACGGAAAAGCCGATCCACGGGCGCAAGCCCAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 219 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 20 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCACAAAATGGGAAAGCGGACCCGAGGGCCCAAGCCCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 220 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 20 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCTCAAAACGGGAAGGCCGATCCCCGCGCACAGGCCCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 221 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 21 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCACAGCCCAGTTCAGGCGATCCCCGAGCCCAGGCACAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 222 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 21 | aggrcggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctaggtgtgggatctc |
| | | | ttacaatggcttcaggtggtggcgcaccagtggcagacaataacgaaggt |
| | | | gccgatggagtgggtagttcctcgggaaattggcattgcgattcccaatg |
| | | | gctgggggacagagtcatcaccaccagcacccgaacctgggccctgccca |
| | | | cctacaacaatcacctctacaagcaaatctccaacagcacatctggagga |
| | | | tcttcaaatgacaacgcctacttcggctacagcaccccctgggggtattt |
| | | | tgacttcaacagattccactgccacttctcaccacgtgactggcagcgac |
| | | | tcatcaacaacaactggggattccggcctaagcgactcaacttcaagctc |
| | | | ttcaacattcaggtcaaagaggttacggacaacaatggagtcaagaccat |
| | | | cgccaataaccttaccagcacggtccaggtcttcacggactcagactatc |
| | | | agctcccgtacgtgctcgggtcggctcacgagggctgcctcccgccgttc |
| | | | ccagcggacgttttcatgattcctcagtacgggtatctgacgcttaatga |
| | | | tggaagccaggccgtgggtcgttcgtccttttactgcctggaatatttcc |
| | | | cgtcgcaaatgctaagaacgggtaacaacttccagttcagctacgagttt |
| | | | gagaacgtacctttccatagcagctacgctcacagccaaagcctggaccg |
| | | | actaatgaatccactcatcgaccaatacttgtactatctctcTaGAacta |
| | | | ttAACGGTTCTGGACAGAATCAAcaaacgctaaaattcagtgtggccgga |
| | | | cccagcaacatggctgtccagggaagaaactacatacctggacccagcta |
| | | | ccgacaacaacgtgtctcaaccactgtgactcaaaacaacaacagcgaat |
| | | | ttgcttggcctggagcttcttcttgggctctcaatggacgtaatagcttg |
| | | | atgaatcctggacctgctatggccagccacaaagaaggagaggaccgttt |
| | | | ctttcctttgtctggatctttaatttttggcaaacaaggTacCggCagag |
| | | | acaacgtggatgcggacaaagtcatgataaccaacgaagaagaaattaaa |
| | | | actactaacccggtagcaacggagtcctatggacaagtggccacaaacca |
| | | | ccagagtGCGCAACCTTCCAGCGGAGACCCTCGTGCTCAGGCGCAAaccg |
| | | | gttgggttcaaaaccaaggaatacttccgggtatggtttggcaggacaga |
| | | | gatgtgtacctgcaaggacccatttgggccaaaattcctcacacggacgg |
| | | | caactttcacccttctccgctgatgggagggtttggaatgaagcacccgc |
| | | | ctcctcagatcctcatcaaaaacacacctgtacctgcggatcctccaacg |
| | | | gccttcaacaaggacaagctgaactctttcatcacccagtattctactgg |
| | | | ccaagtcagcgtggagatcgagtgggagctgcagaaggaaaacagcaagc |
| | | | gctggaacccggagatccagtacacttccaactattacaagtctaataat |
| | | | gttgaatttgctgttaatactgaaggtgtatatagtgaaccccgccccat |
| | | | tggcaccagatacctgactcgtaatctg |
| 223 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 21 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCTCAACCGTCGAGCGGTGATCCCCGTGCTCAAGCGCAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 224 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 21 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCTCAGCCATCATCCGGCGATCCCCGGGCGCAAGCACAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 225 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 21 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCACAGCCCTCGTCGGGGGATCCTAGAGCCCAAGCGCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 226 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 21 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCCCAACCGAGCAGTGGAGACCCAAGGGCACAAGCGCAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 227 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 21 | aggrcggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCACAACCCAGCTCTGGGGATCCCAGGGCCCAGGCTCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 228 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 21 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCCCAGCCATCCAGTGGCGACCCACGCGCCCAGGCCCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 229 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 21 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCGCAGCCGTCAAGCGGCGACCCGCGGGCTCAAGCGCAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 230 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 21 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCGCAACCAAGTAGCGGTGACCCGCGCGCTCAGGCACAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 231 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 22 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCCCAGCAATCACGTGACTTGTATAGGGCTCAGGCCCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 232 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 22 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCGCAGCAGTCTAGGGACTTGTACCGTGCTCAAGCTCAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 233 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 22 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtgggcaccagtggcagacaataacgaaggt |
| | | | gccgatggagtgggtagttcctcgggaaattggcattgcgattcccaatg |
| | | | gctgggggacagagtcatcaccaccagcacccgaacctgggccctgccca |
| | | | cctacaacaatcacctctacaagcaaatctccaacagcacatctggagga |
| | | | tcttcaaatgacaacgcctacttcggctacagcaccccctgggggtattt |
| | | | tgacttcaacagattccactgccacttctcaccacgtgactggcagcgac |
| | | | tcatcaacaacaactggggattccggcctaagcgactcaacttcaagctc |
| | | | ttcaacattcaggtcaaagaggttacggacaacaatggagtcaagaccat |
| | | | cgccaataaccttaccagcacggtccaggtcttcacggactcagactatc |
| | | | agctcccgtacgtgctcgggtcggctcacgagggctgcctcccgccgttc |
| | | | ccagcggacgttttcatgattcctcagtacgggtatctgacgcttaatga |
| | | | tggaagccaggccgtgggtcgttcgtccttttactgcctggaatatttcc |
| | | | cgtcgcaaatgctaagaacgggtaacaacttccagttcagctacgagttt |
| | | | gagaacgtacctttccatagcagctacgctcacagccaaagcctggaccg |
| | | | actaatgaatccactcatcgaccaatacttgtactatctctcTaGAacta |
| | | | ttAACGGTTCTGGACAGAATCAAcaaacgctaaaattcagtgtggccgga |
| | | | cccagcaacatggctgtccagggaagaaactacatacctggacccagcta |
| | | | ccgacaacaacgtgtctcaaccactgtgactcaaaacaacaacagcgaat |
| | | | ttgcttggcctggagcttcttcttgggctctcaatggacgtaatagcttg |
| | | | atgaatcctggacctgctatggccagccacaaagaaggagaggaccgttt |
| | | | ctttcctttgtctggatctttaatttttggcaaacaaggTacCggCagag |
| | | | acaacgtggatgcggacaaagtcatgataaccaacgaagaagaaattaaa |
| | | | actactaacccggtagcaacggagtcctatggacaagtggccacaaacca |
| | | | ccagagtGCTCAGCAATCAAGGGACTTGTATAGGGCGCAGGCCCAAaccg |
| | | | gttgggttcaaaaccaaggaatacttccgggtatggtttggcaggacaga |
| | | | gatgtgtacctgcaaggacccatttgggccaaaattcctcacacggacgg |
| | | | caactttcacccttctccgctgatgggagggtttggaatgaagcacccgc |
| | | | ctcctcagatcctcatcaaaaacacacctgtacctgcggatcctccaacg |
| | | | gccttcaacaaggacaagctgaactctttcatcacccagtattctactgg |
| | | | ccaagtcagcgtggagatcgagtgggagctgcagaaggaaaacagcaagc |
| | | | gctggaacccggagatccagtacacttccaactattacaagtctaataat |
| | | | gttgaatttgctgttaatactgaaggtgtatatagtgaaccccgccccat |
| | | | tggcaccagatacctgactcgtaatctg |
| 234 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 22 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattctcagtacgggtatctgacgcttaatga |
| | | | tggaagccaggccgtgggtcgttcgtccttttactgcctggaatatttcc |
| | | | cgtcgcaaatgctaagaacgggtaacaacttccagttcagctacgagttt |
| | | | gagaacgtacctttccatagcagctacgctcacagccaaagcctggaccg |
| | | | actaatgaatccactcatcgaccaatacttgtactatctctcTaGAacta |
| | | | ttAACGGTTCTGGACAGAATCAAcaaacgctaaaattcagtgtggccgga |
| | | | cccagcaacatggctgtccagggaagaaactacatacctggacccagcta |
| | | | ccgacaacaacgtgtctcaaccactgtgactcaaaacaacaacagcgaat |
| | | | ttgcttggcctggagcttcttcttgggctctcaatggacgtaatagcttg |
| | | | atgaatcctggacctgctatggccagccacaaagaaggagaggaccgttt |
| | | | ctttcctttgtctggatctttaatttttggcaaacaaggTacCggCagag |
| | | | acaacgtggatgcggacaaagtcatgataaccaacgaagaagaaattaaa |
| | | | actactaacccggtagcaacggagtcctatggacaagtggccacaaacca |
| | | | ccagagtGCCCAGCAATCCCGTGATTTGTACAGAGCACAAGCACAAaccg |
| | | | gttgggttcaaaaccaaggaatacttccgggtatggtttggcaggacaga |
| | | | gatgtgtacctgcaaggacccatttgggccaaaattcctcacacggacgg |
| | | | caactttcacccttctccgctgatgggagggtttggaatgaagcacccgc |
| | | | ctcctcagatcctcatcaaaaacacacctgtacctgcggatcctccaacg |
| | | | gccttcaacaaggacaagctgaactctttcatcacccagtattctactgg |
| | | | ccaagtcagcgtggagatcgagtgggagctgcagaaggaaaacagcaagc |
| | | | gctggaacccggagatccagtacacttccaactattacaagtctaataat |
| | | | gttgaatttgctgttaatactgaaggtgtatatagtgaaccccgccccat |
| | | | tggcaccagatacctgactcgtaatctg |
| 235 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 22 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCCCAACAATCGCGGGATCTATATCGAGCCCAGGCGCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 236 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 22 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | agctcccgtacgtgctcgggtcggctcacgagggctgcctcccgccgttc |
| | | | ccagcggacgttttcatgattcctcagtacgggtatctgacgcttaatga |
| | | | tggaagccaggccgtgggtcgttcgtccttttactgcctggaatatttcc |
| | | | cgtcgcaaatgctaagaacgggtaacaacttccagttcagctacgagttt |
| | | | gagaacgtacctttccatagcagctacgctcacagccaaagcctggaccg |
| | | | actaatgaatccactcatcgaccaatacttgtactatctctcTaGAacta |
| | | | ttAACGGTTCTGGACAGAATCAAcaaacgctaaaattcagtgtggccgga |
| | | | cccagcaacatggctgtccagggaagaaactacatacctggacccagcta |
| | | | ccgacaacaacgtgtctcaaccactgtgactcaaaacaacaacagcgaat |
| | | | ttgcttggcctggagcttcttcttgggctctcaatggacgtaatagcttg |
| | | | atgaatcctggacctgctatggccagccacaaagaaggagaggaccgttt |
| | | | ctttcctttgtctggatctttaatttttggcaaacaaggTacCggCagag |
| | | | acaacgtggatgcggacaaagtcatgataaccaacgaagaagaaattaaa |
| | | | actactaacccggtagcaacggagtcctatggacaagtggccacaaacca |
| | | | ccagagtGCGCAACAGAGCAGAGACCTTTATCGTGCCCAGGCGCAGaccg |
| | | | gttgggttcaaaaccaaggaatacttccgggtatggtttggcaggacaga |
| | | | gatgtgtacctgcaaggacccatttgggccaaaattcctcacacggacgg |
| | | | caactttcacccttctccgctgatgggagggtttggaatgaagcacccgc |
| | | | ctcctcagatcctcatcaaaaacacacctgtacctgcggatcctccaacg |
| | | | gccttcaacaaggacaagctgaactctttcatcacccagtattctactgg |
| | | | ccaagtcagcgtggagatcgagtgggagctgcagaaggaaaacagcaagc |
| | | | gctggaacccggagatccagtacacttccaactattacaagtctaataat |
| | | | gttgaatttgctgttaatactgaaggtgtatatagtgaaccccgccccat |
| | | | tggcaccagatacctgactcgtaatctg |
| 237 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 22 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCGCAGCAGTCTCGAGACCTGTATCGTGCGCAAGCTCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactgtaatctg |
| 238 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 22 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCCCAACAATCACGCGATCTTTACCGCGCCCAGGCACAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 239 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 22 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCACAACAGTCGCGAGATCTGTACCGGGCGCAAGCGCAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 240 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 22 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCACAACAGAGCCGCGATTTATACCGGGCCCAAGCACAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 241 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 23 | aggrcggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCGCAGGTCAACACGGTGCGCGAGTTCGCTCAAGCCCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 242 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 23 | aggccggagacaacccgtacctcaagtacaaccacgccgacgccgagttc |
| | | | caggagggctcaaagaagatacgtcttttgggggcaacctcgggcgagca |
| | | | gtcttccaggccaaaaagaggcttcttgaacctcttggtctggttgagga |
| | | | agcggctaagacggctcctggaaagaagaggcctgtagagcagtctcctc |
| | | | aggaaccggactcctccggggtattggcaaatcgggtgcacagcccgcta |
| | | | aaaagagactcaatttcggtcagactggcgacacagagtcagtcccagac |
| | | | cctcaaccaatcggagaacctcccgcagccccctcaggtgtgggatctct |
| | | | tacaatggcttcaggtggtggcgcaccagtggcagacaataacgaaggtg |
| | | | ccgatggagtgggtagttcctcgggaaattggcattgcgattcccaatgg |
| | | | ctgggggacagagtcatcaccaccagcacccgaacctgggccctgcccac |
| | | | ctacaacaatcacctctacaagcaaatctccaacagcacatctggaggat |
| | | | cttcaaatgacaacgcctacttcggctacagcaccccctgggggtatttt |
| | | | gacttcaacagattccactgccacttctcaccacgtgactggcagcgact |
| | | | catcaacaacaactggggattccggcctaagcgactcaacttcaagctct |
| | | | tcaacattcaggtcaaagaggttacggacaacaatggagtcaagaccatc |
| | | | gccaataaccttaccagcacggtccaggtcttcacggactcagactatca |
| | | | gctcccgtacgtgctcgggtcggctcacgagggctgcctcccgccgttcc |
| | | | cagcggacgttttcatgattcctcagtacgggtatctgacgcttaatgat |
| | | | ggaagccaggccgtgggtcgttcgtccttttactgcctggaatatttccc |
| | | | gtcgcaaatgctaagaacgggtaacaacttccagttcagctacgagtttg |
| | | | agaacgtacctttccatagcagctacgctcacagccaaagcctggaccga |
| | | | ctaatgaatccactcatcgaccaatacttgtactatctctcTaGAactat |
| | | | tAACGGTTCTGGACAGAATCAAcaaacgctaaaattcagtgtggccggac |
| | | | ccagcaacatggctgtccagggaagaaactacatacctggacccagctac |
| | | | cgacaacaacgtgtctcaaccactgtgactcaaaacaacaacagcgaatt |
| | | | tgcttggcctggagcttcttcttgggctctcaatggacgtaatagcttga |
| | | | tgaatcctggacctgctatggccagccacaaagaaggagaggaccgtttc |
| | | | tttcctttgtctggatctttaatttttggcaaacaaggTacCggCagaga |
| | | | caacgtggatgcggacaaagtcatgataaccaacgaagaagaaattaaaa |
| | | | ctactaacccggtagcaacggagtcctatggacaagtggccacaaaccac |
| | | | cagagtGCACAAGTGAATACTGTCAGGGAATTTGCACAGGCACAAaccgg |
| | | | ttgggttcaaaaccaaggaatacttccgggtatggtttggcaggacagag |
| | | | atgtgtacctgcaaggacccatttgggccaaaattcctcacacggacggc |
| | | | aactttcacccttctccgctgatgggagggtttggaatgaagcacccgcc |
| | | | tcctcagatcctcatcaaaaacacacctgtacctgcggatcctccaacgg |
| | | | ccttcaacaaggacaagctgaactctttcatcacccagtattctactggc |
| | | | caagtcagcgtggagatcgagtgggagctgcagaaggaaaacagcaagcg |
| | | | ctggaacccggagatccagtacacttccaactattacaagtctaataatg |
| | | | ttgaatttgctgttaatactgaaggtgtatatagtgaaccccgccccatt |
| | | | ggcaccagatacctgactcgtaatctg |
| 243 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 23 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCGCAAGTTAACACTGTAAGAGAGTTTGCACAGGCTCAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcg |
| 244 | DNA | Adeno- | agtgggagctgcagaaggaaaacagcaagcgctggaacccggagatccag |
| | | Associated | tacacttccaactattacaagtctaataatgttgaatttgctgttaatac |
| | | Virus 9 | tgaaggtgtatatagtgaaccccgccccattggcaccagatacctgactc |
| | | comprising | gtaatctgatggctgccgatggttatcttccagattggctcgaggacaac |
| | | SEQ | cttagtgaaggaattcgcgagtggtgggctttgaaacctggagcccctca |
| | | ID NO: 23 | acccaaggcaaatcaacaacatcaagacaacgctcgaggtcttgtgcttc |
| | | | cgggttacaaataccttggacccggcaacggactcgacaagggggagccg |
| | | | gtcaacgcagcagacgcggcggccctcgagcacgacaaggcctacgacca |
| | | | gcagctcaaggrcggagacaacccgtacctcaagtacaaccacgccgacg |
| | | | ccgagttccaggagcggctcaaagaagatacgtcttttgggggcaacctc |
| | | | gggcgagcagtcttccaggccaaaaagaggcttcttgaacctcttggtct |
| | | | ggttgaggaagcggctaagacggctcctggaaagaagaggcctgtagagc |
| | | | agtctcctcaggaaccggactcctccgcgggtattggcaaatcgggtgca |
| | | | cagcccgctaaaaagagactcaatttcggtcagactggcgacacagagtc |
| | | | agtcccagaccctcaaccaatcggagaacctcccgcagccccctcaggtg |
| | | | tgggatctcttacaatggcttcaggtggtggcgcaccagtggcagacaat |
| | | | aacgaaggtgccgatggagtgggtagttcctcgggaaattggcattgcga |
| | | | ttcccaatggctgggggacagagtcatcaccaccagcacccgaacctggg |
| | | | ccctgcccacctacaacaatcacctctacaagcaaatctccaacagcaca |
| | | | tctggaggatcttcaaatgacaacgcctacttcggctacagcaccccctg |
| | | | ggggtattttgacttcaacagattccactgccacttctcaccacgtgact |
| | | | ggcagcgactcatcaacaacaactggggattccggcctaagcgactcaac |
| | | | ttcaagctcttcaacattcaggtcaaagaggttacggacaacaatggagt |
| | | | caagaccatcgccaataaccttaccagcacggtccaggtcttcacggact |
| | | | cagactatcagctcccgtacgtgctcgggtcggctcacgagggctgcctc |
| | | | ccgccgttcccagcggacgttttcatgattcctcagtacgggtatctgac |
| | | | gcttaatgatggaagccaggccgtgggtcgttcgtccttttactgcctgg |
| | | | aatatttcccgtcgcaaatgctaagaacgggtaacaacttccagttcagc |
| | | | tacgagtttgagaacgtacctttccatagcagctacgctcacagccaaag |
| | | | cctggaccgactaatgaatccactcatcgaccaatacttgtactatctct |
| | | | cTaGAactattAACGGTTCTGGACAGAATCAAcaaacgctaaaattcagt |
| | | | gtggccggacccagcaacatggctgtccagggaagaaactacatacctgg |
| | | | acccagctaccgacaacaacgtgtctcaaccactgtgactcaaaacaaca |
| | | | acagcgaatttgcttggcctggagcttcttcttgggctctcaatggacgt |
| | | | aatagcttgatgaatcctggacctgctatggccagccacaaagaaggaga |
| | | | ggaccgtttctttcctttgtctggatctttaatttttggcaaacaaggTa |
| | | | cCggCagagacaacgtggatgcggacaaagtcatgataaccaacgaagaa |
| | | | gaaattaaaactactaacccggtagcaacggagtcctatggacaagtggc |
| | | | cacaaaccaccagagtGCCCAGGTTAACACGGTACGGGAGTTTGCACAAG |
| | | | CCCAGaccggttgggttcaaaaccaaggaatacttccgggtatggtttgg |
| | | | caggacagagatgtgtacctgcaaggacccatttgggccaaaattcctca |
| | | | cacggacggcaactttcacccttctccgctgatgggagggtttggaatga |
| | | | agcacccgcctcctcagatcctcatcaaaaacacacctgtacctgcggat |
| | | | cctccaacggccttcaacaaggacaagctgaactctttcatcacccagta |
| | | | ttctactggccaagtcagcgtggagatcgagtgggagctgcagaaggaaa |
| | | | acagcaagcgctggaacccggagatccagtacacttccaactattacaag |
| | | | tctaataatgttgaatttgctgttaatactgaaggtgtatatagtgaacc |
| | | | ccgccccattggcaccagatacctgactcgtaatctg |
| 245 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 23 | aggrcggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCGCAGGTGAACACCGTAAGGGAGTTCGCCCAAGCCCAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactgtaatctg |
| 246 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 23 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCTCAGGTGAATACAGTCAGGGAATTCGCGCAAGCACAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 247 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 23 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCACAAGTAAATACAGTTCGCGAATTTGCTCAGGCACAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 248 | | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 23 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCACAGGTAAACACAGTGAGGGAATTTGCACAGGCTCAAacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 249 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 23 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactatctctcTaGAact |
| | | | attAACGGTTCTGGACAGAATCAAcaaacgctaaaattcagtgtggccgg |
| | | | acccagcaacatggctgtccagggaagaaactacatacctggacccagct |
| | | | accgacaacaacgtgtctcaaccactgtgactcaaaacaacaacagcgaa |
| | | | tttgcttggcctggagcttcttcttgggctctcaatggacgtaatagctt |
| | | | gatgaatcctggacctgctatggccagccacaaagaaggagaggaccgtt |
| | | | tctttcctttgtctggatctttaatttttggcaaacaaggTacCggCaga |
| | | | gacaacgtggatgcggacaaagtcatgataaccaacgaagaagaaattaa |
| | | | aactactaacccggtagcaacggagtcctatggacaagtggccacaaacc |
| | | | accagagtGCTCAAGTCAATACGGTACGTGAGTTCGCGCAAGCACAGacc |
| | | | ggttgggttcaaaaccaaggaatacttccgggtatggtttggcaggacag |
| | | | agatgtgtacctgcaaggacccatttgggccaaaattcctcacacggacg |
| | | | gcaactttcacccttctccgctgatgggagggtttggaatgaagcacccg |
| | | | cctcctcagatcctcatcaaaaacacacctgtacctgcggatcctccaac |
| | | | ggccttcaacaaggacaagctgaactctttcatcacccagtattctactg |
| | | | gccaagtcagcgtggagatcgagtgggagctgcagaaggaaaacagcaag |
| | | | cgctggaacccggagatccagtacacttccaactattacaagtctaataa |
| | | | tgttgaatttgctgttaatactgaaggtgtatatagtgaaccccgcccca |
| | | | ttggcaccagatacctgactcgtaatctg |
| 250 | DNA | Adeno- | atggctgccgatggttatcttccagattggctcgaggacaaccttagtga |
| | | Associated | aggaattcgcgagtggtgggctttgaaacctggagcccctcaacccaagg |
| | | Virus 9 | caaatcaacaacatcaagacaacgctcgaggtcttgtgcttccgggttac |
| | | comprising | aaataccttggacccggcaacggactcgacaagggggagccggtcaacgc |
| | | SEQ | agcagacgcggcggccctcgagcacgacaaggcctacgaccagcagctca |
| | | ID NO: 23 | aggccggagacaacccgtacctcaagtacaaccacgccgacgccgagttc |
| | | | caggagcggctcaaagaagatacgtcttttgggggcaacctcgggcgagc |
| | | | agtcttccaggccaaaaagaggcttcttgaacctcttggtctggttgagg |
| | | | aagcggctaagacggctcctggaaagaagaggcctgtagagcagtctcct |
| | | | caggaaccggactcctccgcgggtattggcaaatcgggtgcacagcccgc |
| | | | taaaaagagactcaatttcggtcagactggcgacacagagtcagtcccag |
| | | | accctcaaccaatcggagaacctcccgcagccccctcaggtgtgggatct |
| | | | cttacaatggcttcaggtggtggcgcaccagtggcagacaataacgaagg |
| | | | tgccgatggagtgggtagttcctcgggaaattggcattgcgattcccaat |
| | | | ggctgggggacagagtcatcaccaccagcacccgaacctgggccctgccc |
| | | | acctacaacaatcacctctacaagcaaatctccaacagcacatctggagg |
| | | | atcttcaaatgacaacgcctacttcggctacagcaccccctgggggtatt |
| | | | ttgacttcaacagattccactgccacttctcaccacgtgactggcagcga |
| | | | ctcatcaacaacaactggggattccggcctaagcgactcaacttcaagct |
| | | | cttcaacattcaggtcaaagaggttacggacaacaatggagtcaagacca |
| | | | tcgccaataaccttaccagcacggtccaggtcttcacggactcagactat |
| | | | cagctcccgtacgtgctcgggtcggctcacgagggctgcctcccgccgtt |
| | | | cccagcggacgttttcatgattcctcagtacgggtatctgacgcttaatg |
| | | | atggaagccaggccgtgggtcgttcgtccttttactgcctggaatatttc |
| | | | ccgtcgcaaatgctaagaacgggtaacaacttccagttcagctacgagtt |
| | | | tgagaacgtacctttccatagcagctacgctcacagccaaagcctggacc |
| | | | gactaatgaatccactcatcgaccaatacttgtactattctcTaGAacta |
| | | | ttAACGGTTCTGGACAGAATCAAcaaacgctaaaattcagtgtggccgga |
| | | | cccagcaacatggctgtccagggaagaaactacatacctggacccagcta |
| | | | ccgacaacaacgtgtctcaaccactgtgactcaaaacaacaacagcgaat |
| | | | ttgcttggcctggagcttcttcttgggctctcaatggacgtaatagcttg |
| | | | atgaatcctggacctgctatggccagccacaaagaaggagaggaccgttt |
| | | | ctttcctttgtctggatctttaatttttggcaaacaaggTacCggCagag |
| | | | acaacgtggatgcggacaaagtcatgataaccaacgaagaagaaattaaa |
| | | | actactaacccggtagcaacggagtcctatggacaagtggccacaaacca |
| | | | ccagagtGCTCAAGTAAATACCGTCAGAGAATTCGCTCAGGCGCAAaccg |
| | | | gttgggttcaaaaccaaggaatacttccgggtatggtttggcaggacaga |
| | | | gatgtgtacctgcaaggacccatttgggccaaaattcctcacacggacgg |
| | | | caactttcacccttctccgctgatgggagggtttggaatgaagcacccgc |
| | | | ctcctcagatcctcatcaaaaacacacctgtacctgcggatcctccaacg |
| | | | gccttcaacaaggacaagctgaactctttcatcacccagtattctactgg |
| | | | ccaagtcagcgtggagatcgagtgggagctgcagaaggaaaacagcaagc |
| | | | gctggaacccggagatccagtacacttccaactattacaagtctaataat |
| | | | gttgaatttgctgttaatactgaaggtgtatatagtgaaccccgccccat |
| | | | tggcaccagatacctgactcgtaatctg |

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. A composition comprising a targeting moiety effective to increase transduction of central nervous system (CNS) tissues, optionally further comprising a cargo coupled to or otherwise associated with the targeting moiety.

2. The composition of claim 1, wherein the targeting moiety comprises a n-mer motif, the n-mer motif comprising or consisting of is Y₁-Y₂-Y₃-P-Y₄-E-Y₅ wherein Y₁-Y₅ are independently selected amino acids;
optionally wherein Y₁ or Y₂ is independently selected from an amino acid with a single positive charge, and Y₅ is an amino acid with a single aromatic ring; and
optionally wherein Y₁ is selected from I, T, Q, H, K, R, V, G, E, or L; Y₂ is selected from R, N, A, K, or G; Y₃ is selected from N, I, Q, M, F, L, D, T, R, V, or S; Y₄ is selected from Q, N, V, S, or T; and Y₅ is selected from F, Y, or H.

3.-4. (canceled)

5. The composition of claim 2, wherein the n-mer motif is selected from the group consisting of QRIPQEY (SEQ ID NO: 13), KNSPSEF (SEQ ID NO: 10), TRTPVEF (SEQ ID NO: 16), IRVPNEF (SEQ ID NO: 8), IRMPTEF (SEQ ID NO: 6), IRQPQEF (SEQ ID NO: 7), GKLPREF (SEQ ID NO: 4), LALPQEF (SEQ ID NO: 11), ELNPREF (SEQ ID NO: 3), HNFPNEF (SEQ ID NO: 5), TARPNEY (SEQ ID NO: 15), QNIPSEY (SEQ ID NO: 12), RSQPQEH (SEQ ID NO: 14), KGPGYEH (SEQ ID NO: 9), QSRDLYR (SEQ ID NO: 20), MNSTISK (SEQ ID NO: 19), VNTVREF (SEQ ID NO: 21), ENHTRDK (SEQ ID NO: 17), and GNDTRST (SEQ ID NO: 18).

6.-7. (canceled)

8. The composition of claim 2, wherein the n-mer motif is selected from Table 1 to Table 3 or SEQ ID: 3-21.

9. (canceled)

10. The composition of claim 1, wherein the targeting moiety is part of a viral capsid protein;
optionally wherein the viral capsid protein is an AAV viral capsid protein;
optionally the AAV viral capsid is selected from SEQ ID: 1, 22-40; and
optionally the targeting moiety is inserted between two amino acids of one or more capsid proteins such that the targeting moiety is external to the AAV viral capsid,
optionally wherein the capsid protein is VP1, VP2, VP3, or a combination thereof, and
optionally wherein the targeting moiety is inserted between amino acids 588 and 589 of a capsid protein of AAV9, or in an analogous position of a capsid protein from AAV1, AAV2, AAV3, AAV4, AAV5, AAV6, AAV7, AAV8, AAVrh8, AAV rh.74, or AAV rh. 10.

11.-16. (canceled)

17. The composition of claim 1, wherein the cargo is a polynucleotide, one or more polypeptides, a ribonucleoprotein complex;
optionally wherein the cargo is encoded on a rAAV artificial genome operably linked to regulatory sequence and flanked by AAV ITR sequences; and
optionally wherein the polynucleotide encodes one or more polypeptides and/or a RNAi oligonucleotide,
optionally wherein the one or more polypeptides comprise enzymes or antibodies, and
optionally wherein the polynucleotide encodes a CRISPR-Cas system.

18.-21. (canceled)

22. A vector system comprising one or more vectors encoding a targeting moiety effective to increase transduction of central nervous system tissues (CNS), optionally further comprising a cargo couple to or otherwise associated with the targeting moiety.

23. The vector system of claim 22, wherein the targeting moiety comprises a n-mer motif, the n-mer motif comprising or consisting of is Y₁-Y₂-Y₃-P-Y₄-E-Y₅ wherein Y₁-Y₅ are independently selected amino acids;
optionally wherein Y₁ or Y₂ is independently selected from an amino acid with a single positive charge, and Y₅ is an amino acid with a single aromatic ring; and
optionally wherein Y₁ is selected from I, T, Q, H, K, R, V, G, E, or L; Y₂ is selected from R, N, A, K, or G; Y₃ is selected from N, I, Q, M, F, L, D, T, R, V, or S; Y₄ is selected from Q, N, V, S, or T; and Y₅ is selected from F, Y, or H.

24.-25. (canceled)

26. The vector system of claim 23, wherein the n-mer motif is selected from the group consisting of QRIPQEY (SEQ ID NO: 13), KNSPSEF (SEQ ID NO: 10), TRTPVEF (SEQ ID NO: 16), IRVPNEF (SEQ ID NO: 8), IRMPTEF (SEQ ID NO: 6), IRQPQEF (SEQ ID NO: 7), GKLPREF (SEQ ID NO: 4), LALPQEF (SEQ ID NO: 11), ELNPREF (SEQ ID NO: 3), HNFPNEF (SEQ ID NO: 5), TARPNEY (SEQ ID NO: 15), QNIPSEY (SEQ ID NO: 12), RSQPQEH (SEQ ID NO: 14), and KGPGYEH (SEQ ID NO: 9), QSRDLYR (SEQ ID NO: 20), MNSTISK (SEQ ID NO: 19), VNTVREF (SEQ ID NO: 21), ENHTRDK (SEQ ID NO: 17), and GNDTRST (SEQ ID NO: 18).

27.-28. (canceled)

29. The vector system of claim 23, wherein the n-mer motif is selected from Table 1 to Table 3 or SEQ ID: 3-21.

30. (canceled)

31. The vector system of claim 22, wherein the targeting moiety is part of a viral capsid protein;
optionally wherein the viral capsid protein is an AAV viral capsid protein;
optionally the AAV viral capsid is selected from SEQ ID: 1, 22-40; and
optionally the targeting moiety is inserted between two amino acids of one or more capsid proteins such that the targeting moiety is external to the AAV viral capsid,
optionally wherein the capsid protein is VP1, VP2, VP3, or a combination thereof, and
optionally wherein the targeting moiety is inserted between amino acids 588 and 589 of a capsid protein of AAV9, or in an analogous position of a capsid protein from AAV1, AAV2, AAV3, AAV4, AAV5, AAV6, AAV7, AAV8, AAVrh8. AAV rh.74, or AAV rh. 10.

32.-37. (canceled)

38. The vector system of claim 22, wherein the cargo is a polynucleotide, one or more polypeptides, a ribonucleoprotein complex;
optionally wherein the cargo is encoded on a rAAV artificial genome operably linked to regulatory sequence and flanked by AAV ITR sequences; and
optionally wherein the polynucleotide encodes one or more polypeptides and/or a RNAi oligonucleotide,
optionally wherein the one or more polypeptides comprise enzymes or antibodies, and
optionally wherein the polynucleotide encodes a CRISPR-Cas system.

39.-43. (canceled)

44. A particle produced or a polypeptide encoded or produced by the vector system of claim 22.

45. (canceled)

46. A cell comprising the composition of claim 1.

47. A method of delivering one or more cargos to the CNS, comprising:
administering, in vivo or in vitro, the engineered AAV capsid of claim 10.

48. The method of claim 47, wherein the cargo is an RNAi oligonucleotide, a polynucleotide encoding a polypeptide, or a polypeptide, optionally wherein the polypeptide includes an enzyme or antibody;
optionally wherein the cargo is a Cas polypeptide, a guide molecule, or both;
optionally wherein the cargo is a nuclease or a nucleic acid component of a RNA-guided nuclease; and
optionally wherein the cargo is one or more polynucleotides encoding the nuclease and nucleic acid component of a RNA-guided nuclease.

49.-52. (canceled)

53. A cell comprising the vector of claim 22.

54. A cell comprising the polypeptide, or particle of claim 44.

55. A method of delivering one or more cargos to the CNS, comprising:
administering, in vivo or in vitro, or the vector of claim 22.

56. The method of claim 55, wherein the cargo is an RNAi oligonucleotide, a polynucleotide encoding a polypeptide, or a polypeptide, optionally wherein the polypeptide includes an enzyme or antibody;
optionally wherein the cargo is a Cas polypeptide, a guide molecule, or both;
optionally wherein the cargo is a nuclease or a nucleic acid component of a RNA-guided nuclease; and
optionally wherein the cargo is one or more polynucleotides encoding the nuclease and nucleic acid component of a RNA-guided nuclease.
